(19) United States
(12) Patent Application Publication
(10) Pub. No.: US 2009/0311283 A1
(43) Pub. Date: Dec. 17, 2009

(54) Inducing cellular immune responses to hepatitis B virus using peptide and nucleic acid compositions

(75) Inventors: Alessandro Sette, La Jolla, CA (US); John Sidney, San Diego, CA (US); Scott Southwood, Santee, CA (US); Maria A. Vitiello, La Jolla, CA (US); Brian D. Livingston, San Diego, CA (US); Esteban Celis, Rochester, MN (US); Ralph T. Kubo, Carlsbad, CA (US); Howard M. Grey, La Jolla, CA (US); Robert W. Chesnut, Cardiff-by the Sea, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Pharmexa Inc., San Diego, CA (US)

(21) Appl. No.: 11/976,998

(22) Filed: Oct. 30, 2007

Related U.S. Application Data
(63) Continuation of application No. 09/350,401, filed on Jul. 8, 1999.
Application No. 09/350,401 is a continuation-in-part of application No. 09/239,043, filed on Jan. 27, 1999, now Pat. No. 6,689,363.
Application No. 09/239,043 is a continuation-in-part of application No. 08/347,610, filed on Dec. 1, 1994, now abandoned.
Application No. 08/347,610 is a continuation-in-part of application No. 08/344,824, filed on Nov. 23, 1994, now abandoned.
Application No. 08/344,824 is a continuation-in-part of application No. 08/205,713, filed on Mar. 4, 1994, now abandoned.
Application No. 08/205,713 is a continuation-in-part of application No. 09/189,702, filed on Nov. 10, 1998, now Pat. No. 7,252,829.
Application No. 09/189,702 is a continuation-in-part of application No. 08/820,360, filed on Mar. 12, 1997, now abandoned.
Application No. 08/820,360 is a continuation-in-part of application No. 08/197,484, filed on Feb. 16, 1994, now Pat. No. 6,419,931.
Application No. 08/197,484 is a continuation-in-part of application No. 07/935,811, filed on Aug. 26, 1992, now abandoned.
Application No. 07/935,811 is a continuation-in-part of application No. 07/874,491, filed on Apr. 27, 1992, now abandoned.
Application No. 07/874,491 is a continuation-in-part of application No. 07/827,682, filed on Jan. 29, 1992, now abandoned.
Application No. 08/347,610 is a continuation-in-part of application No. 08/159,339, filed on Nov. 29, 1993, now Pat. No. 6,037,135.
Application No. 08/159,339 is a continuation-in-part of application No. 08/103,396, filed on Aug. 6, 1993, now abandoned.
Application No. 08/103,396 is a continuation-in-part of application No. 08/027,746, filed on Mar. 5, 1993, now abandoned.
Application No. 08/027,746 is a continuation-in-part of application No. 07/926,666, filed on Aug. 7, 1992, now abandoned.
Application No. 08/344,824 is a continuation-in-part of application No. 08/278,634, filed on Jul. 21, 1994, now abandoned.
Application No. 08/205,713 is a continuation-in-part of application No. 08/159,184, filed on Nov. 29, 1993, now abandoned.
Application No. 08/159,184 is a continuation-in-part of application No. 08/073,205, filed on Jun. 4, 1993, now abandoned.
Application No. 08/073,205 is a continuation-in-part of application No. 08/027,146, filed on Mar. 5, 1993, now abandoned.
Application No. 09/189,702 is a continuation-in-part of application No. 08/205,713, filed on Mar. 4, 1994, now abandoned.
Application No. 08/205,713 is a continuation-in-part of application No. 08/978,291, filed on Nov. 25, 1997, now abandoned.
Application No. 08/978,291 is a continuation of application No. 08/461,603, filed on Jun. 5, 1995, now abandoned.
Application No. 08/461,603 is a continuation of application No. 07/935,811, filed on Aug. 26, 1992, now abandoned.
(60) Provisional application No. 60/013,363, filed on Mar. 13, 1996.

(51) Int. Cl. A61K 39/29; C07K 7/06; C07K 7/08; A61K 38/08; A61K 38/10; A61K 31/7088; C12Q 1/02

(52) U.S. Cl. 424/186.1; 530/328; 530/327; 514/16; 514/15; 514/14; 514/44 R; 435/29

(57) ABSTRACT

This invention uses our knowledge of the mechanisms by which antigen is recognized by T cells to develop epitope-based vaccines directed towards HBV. More specifically, this application communicates our discovery of pharmaceutical compositions and methods of use in the prevention and treatment of HBV infection.

### CROSS-REFERENCES TO RELATED APPLICATIONS

This application is a Continuation-In-Part (“CIP”) of U.S. Ser. No. 08/820,360 filed Mar. 12, 1997, which claims the benefit of U.S. Provisional Application No. 60/013,363 filed Mar. 13, 1996 and now abandoned. The present application is also a CIP of U.S. Ser. No. 09/189,702 filed Nov. 10, 1998, which is a CIP of U.S. Ser. No. 08/205,713 filed Mar. 4, 1994, which is a CIP of Ser. No. 08/159,184 filed Nov. 29, 1993 and now abandoned, which is a CIP of Ser. No. 08/073,205 filed Jun. 4, 1993 and now abandoned, which is a CIP of Ser. No. 08/027,146 filed Mar. 5, 1993 and now abandoned. The present application is also related to U.S. Ser. No. 08/197,484, U.S. Ser. No. 08/464,234, U.S. Ser. No. 08/464,496, U.S. Ser. No. 08/464,031, abandoned U.S. Ser. No. 08/464,433, and U.S. Ser. No. 08/461,603, which is a continuation of abandoned U.S. Ser. No. 07/935,811, which is a CIP of abandoned U.S. Ser. No. 07/874,491, which is a CIP of abandoned U.S. Ser. No. 07/827,682, which is a CIP of abandoned Ser. No. 07/749,568. The present application is also related to U.S. patent application entitled “Peptides and Methods for Creating Synthetic Peptides with Modulated Binding Affinity for HLA Molecules”, Attorney Docket No. 018623-009520, filed Jan. 6, 1999, which is a CIP of U.S. Ser. No. 08/815,396, which is a CIP of abandoned U.S. Ser. No. 60/013,113. Furthermore, the present application is related to U.S. Ser. No. 09/017,735, which is a CIP of abandoned U.S. Ser. No. 08/589,108; U.S. Ser. No. 08/753,622, U.S. Ser. No. 08/822,382, abandoned U.S. Ser. No. 60/013,980, U.S. Ser. No. 08/454,033, U.S. Ser. No. 09/116,424, U.S. Ser. No. 08/205,713, and U.S. Ser. No. 08/349,177, which is a CIP of abandoned U.S. Ser. No. 08/159,184, which is a CIP of abandoned U.S. Ser. No. 08/073,205, which is a CIP of abandoned U.S. Ser. No. 08/027,146. The present application is also related to U.S. Ser. No. 09/017,524, U.S. Ser. No. 08/821,739, abandoned U.S. Ser. No. 60/013,833, U.S. Ser. No. 08/758,409, U.S. Ser. No. 08/589,107, U.S. Ser. No. 08/451,913, U.S. Ser. No. 08/186,266, U.S. Ser. No. 09/116,061, and U.S. Ser. No. 08/347,610, which is a CIP of U.S. Ser. No. 08/159,339, which is a CIP of abandoned U.S. Ser. No. 08/103,396, which is a CIP of abandoned U.S. Ser. No. 08/027,746, which is a CIP of abandoned U.S. Ser. No. 07/926,666. The present application is also related to U.S. Ser. No. 09/017,743, U.S. Ser. No. 08/753,615; U.S. Ser. No. 08/590,298, U.S. Ser. No. 09/115,400, and U.S. Ser. No. 08/452,843, which is a CIP of U.S. Ser. No. 08/344,824, which is a CIP of abandoned U.S. Ser. No. 08/278,634. The present application is also related to provisional U.S. Ser. No. 60/087,192 and U.S. Ser. No. 09/009,953, which is a CIP of abandoned U.S. Ser. No. 60/036,713 and abandoned U.S. Ser. No. 60/037,432. In addition, the present application is related to U.S. Ser. No. 09/098,584 and to Provisional U.S. Ser. No. 60/117,486. All of the above applications are incorporated herein by reference.

### FEDERALLY SPONSORED RESEARCH AND DEVELOPMENT

This invention was funded, in part, by the United States government under grants with the National Institutes of Health. The U.S. government has certain rights in this invention.

### INDEX

### I. Background of the Invention

### II. Summary of the Invention

### III. Brief Description of the Figures

### V. Detailed Description of the Invention

A. Definitions
B. Stimulation of CTL and HTL responses against HBV
C. Binding Affinity of Peptide Epitopes for HLA Molecules
D. Peptide Epitope Binding Motifs and Supermotifs
   1. HLA-A1 supermotif
   2. HLA-A2 supermotif
   3. HLA-A3 supermotif
   4. HLA-A24 supermotif
   5. HLA-B7 supermotif
   6. HLA-B27 supermotif
   7. HLA-B44 supermotif
   8. HLA-B58 supermotif
   9. HLA-B62 supermotif
   10. HLA-A1 motif
   11. HLA-A2.1 motif
   12. HLA-A3 motif
   13. HLA-A11 motif
   14. HLA-A24 motif
   15. HLA-DR-1-4-7 supermotif
   16. HLA-DR3 motifs
E. Enhancing Population Coverage of the Vaccine
F. Immune Response Stimulating Peptide Analogs
G. Computer Screening of Protein Sequences from Disease-Related Antigens for Supermotif or Motif Containing Peptides
H. Preparation of Peptide Epitopes
I. Assays to Detect T-Cell Responses
J. Use of Peptide Epitopes for Evaluating Immune Responses
K. Vaccine Compositions
   1. Minigene Vaccines
   2. Combinations of CTL Peptides with Helper Peptides
L. Administration of Vaccines for Therapeutic or Prophylactic Purposes
M. Kits

### V. Examples

### VI. Claims

### VII. Abstract

### I. BACKGROUND OF THE INVENTION

Chronic infection by hepatitis B virus (HBV) affects at least 5% of the world's population and is a major cause of cirrhosis and hepatocellular carcinoma (Hooffnagle, J., N. Engl. J. Med. 323:337, 1990; Fields, B. and Knipe, D., In: Fields Virology 2:2137, 1990). The World Health Organization lists hepatitis B as a leading cause of death worldwide, close behind chronic pulmonary disease, and more prevalent than AIDS. Chronic HBV infection can range from an asymptomatic carrier state to continuous hepatocellular necrosis and inflammation, and can lead to hepatocellular carcinoma.

The immune response to HBV is believed to play an important role in controlling hepatitis B infection. A variety of humoral and cellular responses to different regions of HBV including the nucleocapsid core, polymerase, and surface antigens have been identified. T cell-mediated immunity, particularly involving class I human leukocyte antigen-restricted cytotoxic T lymphocytes (CTL), is believed to be crucial in combatting established HBV infection.

Class I human leukocyte antigen (HLA) molecules are expressed on the surface of almost all nucleated cells. CTL recognize peptide fragments, derived from intracellular processing of various antigens, in the form of a complex with class I HLA molecules. This recognition event then results in the destruction of the cell bearing the HLA-peptide complex directly or the activation of non-destructive mechanisms e.g., the production of interferon, that inhibit viral replication.

Several studies have emphasized the association between self-limiting acute hepatitis and multispecific CTL responses (Penna, A. et al., J. Exp. Med. 174:1565, 1991; Nayersina, R. et al., J. Immunol. 150:4659, 1993). Spontaneous and interferon-related clearance of chronic HBV infection is also associated with the resurgence of a vigorous CTL response (Guidotti, L. G. et al., Proc. Natl. Acad. Sci. USA 91:3764, 1994). In all such cases the CTL responses are polyclonal, and specific for multiple viral proteins including the HBV envelope, core and polymerase antigens. By contrast, in patients with chronic hepatitis, the CTL activity is usually absent or weak, and antigenically restricted.

The crucial role of CTL in resolution of HBV infection has been further underscored by studies using HBV transgenic mice. Adoptive transfer of HBV-specific CTL into mice transgenic for the HBV genome resulted in suppression of virus replication. This effect was primarily mediated by a non-lytic, lymphokine-based mechanism (Guidotti, L. G. et al., Proc. Natl. Acad. Sci. USA 91:3764, 1994; Guidotti, L. G., Guilhot, S., and Chisari, F. V. J. Virol. 68:1265, 1994; Guidotti, L. G. et al., J. Virol. 69:6158, 1995; Gilles, P. N., Fey, G., and Chisari, F. V., J. Virol. 66:3955, 1992).

As is the case for HLA class I restricted responses, HLA class II restricted T cell responses are usually detected in patients with acute hepatitis, and are absent or weak in patients with chronic infection (Chisari, F. V. and Ferrari, C., Annu. Rev. Immunol. 13:29, 1995). HLA Class II responses are tied to activation of helper T cells (HTLs) Helper T lymphocytes, which recognize Class II HLA molecules, may directly contribute to the clearance of HBV infection through the secretion of cytokines which suppress viral replication (Franco, A. et al., J. Immunol. 159:2001, 1997). However, their primary role in disease resolution is believed to be mediated by inducing activation and expansion of virus-specific CTL and B cells.

In view of the heterogeneous immune response observed with HBV infection, induction of a multi-specific cellular immune response directed simultaneously against multiple epitopes appears to be important for the development of an efficacious vaccine against HBV. There is a need to establish vaccine embodiments that elicit immune responses that correspond to responses seen in patients that clear HBV infection. Epitope-based vaccines appear useful.

Upon development of appropriate technology, the use of epitope-based vaccines has several advantages over current vaccines. The epitopes for inclusion in such a vaccine are to be selected from conserved regions of viral or tumor-associated antigens, in order to reduce the likelihood of escape mutants. The advantage of an epitope-based approach over the use of whole antigens is that there is evidence that the immune response to whole antigens is directed largely toward variable regions of the antigen, allowing for immune escape due to mutations. Furthermore, immunosuppressive epitopes that may be present in whole antigens can be avoided with the use of epitope-based vaccines.

Additionally, with an epitope-based vaccine approach, there is an ability to combine selected epitopes (CTL and HTL) and additionally to modify the composition of the epitopes, achieving, for example, enhanced immunogenicity. Accordingly, the immune response can be modulated, as appropriate, for the target disease. Similar engineering of the response is not possible with traditional approaches.

Another major benefit of epitope-based immune-stimulating vaccines is their safety. The possible pathological side effects caused by infectious agents or whole protein antigens, which might have their own intrinsic biological activity, is eliminated.

An epitope-based vaccine also provides the ability to direct and focus an immune response to multiple selected antigens from the same pathogen. Thus, patient-by-patient variability in the immune response to a particular pathogen may be alleviated by inclusion of epitopes from multiple antigens from that pathogen in a vaccine composition. A “pathogen” may be an infectious agent or a tumor associated molecule.

However, one of the most formidable obstacles to the development of broadly efficacious epitope-based immunotherapeutics has been the extreme polymorphism of HLA molecules. To date, effective non-genetically biased coverage of a population has been a task of considerable complexity; such coverage has required that epitopes be used specific for HLA molecules corresponding to each individual HLA allele, therefore, impractically large numbers of epitopes would have to be used in order to cover ethnically diverse populations. There has existed a need to develop peptide epitopes that are bound by multiple HLA antigen molecules for use in epitope-based vaccines. The greater the number of HLA antigen molecules bound, the greater the breadth of population coverage by the vaccine.

Furthermore, as described herein in greater detail, a need has existed to modulate peptide binding properties, for example so that peptides that are able to bind to multiple HLA antigens do so with an affinity that will stimulate an immune response. Identification of epitopes restricted by more than one HLA allele at an affinity that correlates with immunogenicity is important to provide thorough population coverage, and to allow the elicitation of responses of sufficient vigor whereby the natural immune responses noted in self-limiting acute hepatitis, or of spontaneous clearance of chronic HBV infection is induced in a diverse segment of the population. Such a response can also target a broad array of epitopes. The technology disclosed herein provides for such favored immune responses.

The information provided in this section is intended to disclose the presently understood state of the art as of the filing date of the present application. Information is included in this section which was generated subsequent to the priority date of this application. Accordingly, background in this section is not intended, in any way, to delineate the priority date for the invention.

### II. SUMMARY OF THE INVENTION

This invention applies our knowledge of the mechanisms by which antigen is recognized by T cells, for example, to develop epitope-based vaccines directed towards HBV. More specifically, this application communicates our discovery of specific epitope pharmaceutical compositions and methods of use in the prevention and treatment of HBV infection.

Upon development of appropriate technology, the use of epitope-based vaccines has several advantages over current vaccines, particularly when compared to the use of whole antigens in vaccine compositions. There is evidence that the immune response to whole antigens is directed largely toward variable regions of the antigen, allowing for immune escape due to mutations. The epitopes for inclusion in an epitope-based vaccine are selected from conserved regions of viral or tumor-associated antigens, which thereby reduces the likelihood of escape mutants. Furthermore, immunosuppressive epitopes that may be present in whole antigens can be avoided with the use of epitope-based vaccines.

An additional advantage of an epitope-based vaccine approach is the ability to combine selected epitopes (CTL and HTL), and further, to modify the composition of the epitopes, achieving, for example, enhanced immunogenicity. Accordingly, the immune response can be modulated, as appropriate, for the target disease. Similar engineering of the response is not possible with traditional approaches.

Another major benefit of epitope-based immune-stimulating vaccines is their safety. The possible pathological side effects caused by infectious agents or whole protein antigens, which might have their own intrinsic biological activity, is eliminated.

An epitope-based vaccine also provides the ability to direct and focus an immune response to multiple selected antigens from the same pathogen. Thus, patient-by-patient variability in the immune response to a particular pathogen may be alleviated by inclusion of epitopes from multiple antigens from that pathogen in a vaccine composition. A “pathogen” may be an infectious agent or a tumor associated molecule.

One of the most formidable obstacles to the development of broadly efficacious epitope-based immunotherapeutics, however, has been the extreme polymorphism of HLA molecules. To date, effective non-genetically biased coverage of a population has been a task of considerable complexity; such coverage has required that epitopes be used specific for HLA molecules corresponding to each individual HLA allele, therefore, impractically large numbers of epitopes would have to be used in order to cover ethnically diverse populations. Thus, there has existed a need to develop peptide epitopes that are bound by multiple HLA antigen molecules for use in epitope-based vaccines. The greater the number of HLA antigen molecules bound, the greater the breadth of population coverage by the vaccine.

Furthermore, as described herein in greater detail, a need has existed to modulate peptide binding properties, for example, so that peptides that are able to bind to multiple HLA antigens do so with an affinity that will stimulate an immune response. Identification of epitopes restricted by more than one HLA allele at an affinity that correlates with immunogenicity is important to provide thorough population coverage, and to allow the elicitation of responses of sufficient vigor to prevent or clear an infection in a diverse segment of the population. Such a response can also target a broad array of epitopes. The technology disclosed herein provides for such favored immune responses.

In a preferred embodiment, epitopes for inclusion in vaccine compositions of the invention are selected by a process whereby protein sequences of known antigens are evaluated for the presence of motif or supermotif-bearing epitopes. Peptides corresponding to a motif- or supermotif-bearing epitope are then synthesized and tested for the ability to bind to the HLA molecule that recognizes the selected motif. Those peptides that bind at an intermediate or high affinity i.e., an IC₅₀ (or a K_{D} value) of 500 nM or less for HLA class I molecules or 1000 nM or less for HLA class II molecules, are further evaluated for their ability to induce a CTL or HTL response. Immunogenic peptides are selected for inclusion in vaccine compositions.

Supermotif-bearing peptides may additionally be tested for the ability to bind to multiple alleles within the HLA supertype family. Moreover, peptide epitopes may be analogued to modify binding affinity and/or the ability to bind to multiple alleles within an HLA supertype.

The invention also includes an embodiment comprising a method for monitoring immunogenic activity of a vaccine for HBV in a patient having a known HLA-type, the method comprising incubating a T lymphocyte sample from the patient with a peptide composition comprising an HBV epitope consisting essentially of an amino acid sequence described in Tables VI to Table XX or Table XXII which binds the product of at least one HLA allele present in said patient, and detecting for the presence of a T lymphocyte that binds to the peptide. In a preferred embodiment, the peptide comprises a tetrameric complex.

An alternative modality for defining the peptides in accordance with the invention is to recite the physical properties, such as length; primary, potentially secondary and/or tertiary structure; or charge, which are correlated with binding to a particular allele-specific HLA molecule or group of allele-specific HLA molecules. A further modality for defining peptides is to recite the physical properties of an HLA binding pocket, or properties shared by several allele-specific HLA binding pockets (e.g. pocket configuration and charge distribution) and reciting that the peptide fits and binds to said pocket or pockets.

As will be apparent from the discussion below, other methods and embodiments are also contemplated. Further, novel synthetic peptides produced by any of the methods described herein are also part of the invention.

### III. BRIEF DESCRIPTION OF THE FIGURES

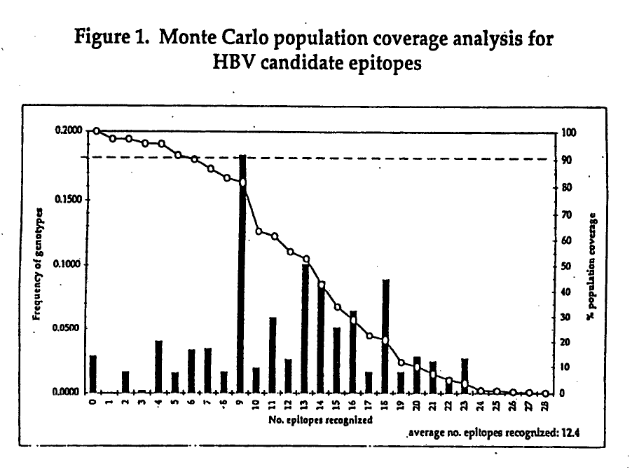

FIG. 1: FIG. 1 provides a graph of total frequency of genotypes as a function of the number of HBV candidate epitopes bound by HLA-A and B molecules, in an average population.

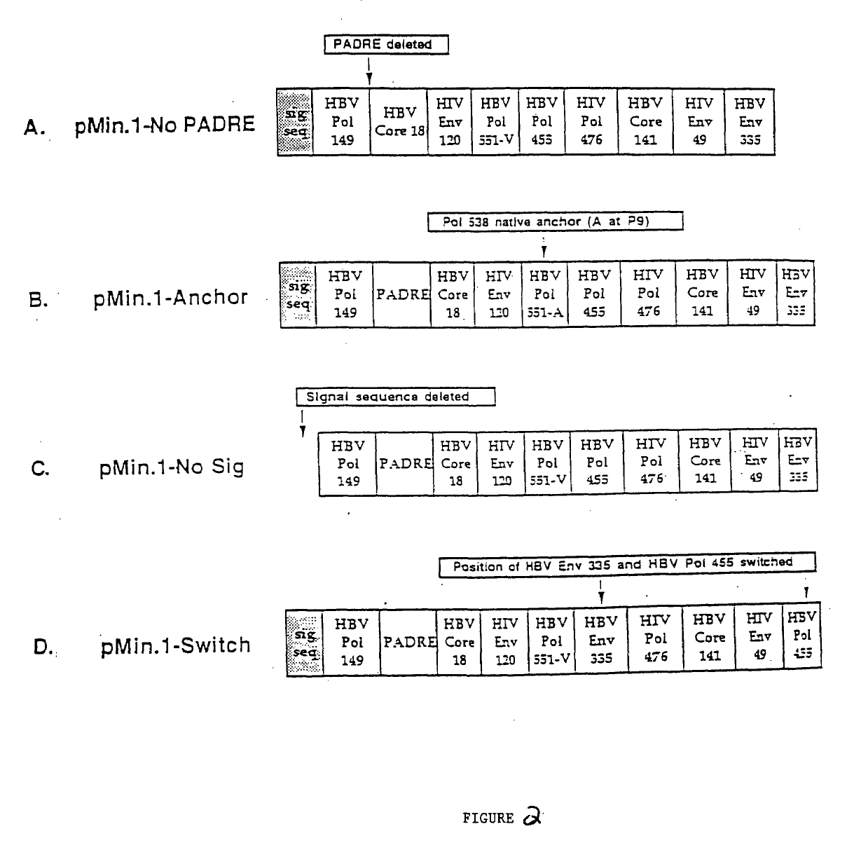

FIG. 2: FIG. 2 Illustrates the Position of Peptide Epitopes in Experimental Model Minigene Constructs

### IV. DETAILED DESCRIPTION OF THE INVENTION

The peptides and corresponding nucleic acid compositions of the present invention are useful for stimulating an immune response to HBV either by stimulating the production of CTL or HTL responses. The peptides, which are derived directly or indirectly from native HBV amino acid sequences, are able to bind to HLA molecules and stimulate an immune response to HBV. The complete polyprotein sequence from HBV and its variants can be obtained from Genbank. Peptides can also be readily determined from sequence information that may subsequently be discovered for heretofore unknown variants of HBV as will be clear from the disclosure provided below.

The peptides of the invention have been identified in a number of ways, as will be discussed below. Further, analog peptides have been derived and the binding activity for HLA molecules modulated by modifying specific amino acid residues to create peptide analogs exhibiting altered immunogenicity. Further, the present invention provides compositions and combinations of compositions that enable epitope-based vaccines that are capable of interacting with multiple HLA antigens to provide broader population coverage than prior vaccines.

### IV.A. DEFINITIONS

The invention can be better understood with reference to the following definitions, which are listed alphabetically.

“Cross-reactive binding” indicates that a peptide is bound by more than one HLA molecule; a synonym is degenerate binding.

A “cryptic epitope” elicits a response by immunization with an isolated peptide, but the response is not cross-reactive in vitro when intact whole protein which comprises the epitope is used as an antigen.

A “dominant epitope” is an epitope that induces an immune response upon immunization with a whole native antigen. (See, e.g., Sercarz, et al., Annu. Rev. Immunol. 11:729766 (1993)) Such a response is cross-reactive in vitro with an isolated peptide epitope.

With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) receptors. In an immune system setting, in vivo or in vitro, an epitope is the collective features of a molecule, such as primary, secondary and tertiary peptide structure, and charge, that together form a site recognized by an immunoglobulin, T cell receptor or HLA molecule.

“Human Leukocyte Antigen” or “HLA” is a human class I or class II Major Histocompatibility Complex (MHC) protein (see, Stites, et al., IMMUNOLOGY, 8^{TH} ED., Lange Publishing, Los Altos, Calif. (1994).

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, and HLA xx-like supertype molecules (where xx denotes a particular HLA type) are synonyms.

Throughout this disclosure, results are expressed in terms of “IC₅₀'s.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate K_{D} values. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand.

Assays for determining binding are described in detail in PCT publications WO 94/20127 and WO 94/03205. Alternatively, binding is expressed relative to a reference peptide. As a particular assay becomes more, or less, sensitive, the IC₅₀'s of the peptides tested may change somewhat. However, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good, intermediate, weak, or negative binder is generally based on its IC₅₀, relative to the IC₅₀ of a standard peptide.

Binding may also be determined using other assays including, for example, inhibition of antigen presentation (Sette et al., J. Immunol. 141:3893, 1991), in vitro assembly assays (Townsend et al., Cell 62:285, 1990), measures of dissociations rates (Parker et al., J. Immunol. 149:1896-1904, 1992), and FACS-based assays using mutated cells, such as RMA.S (Melief, et al., Eur. J. Immunol. 21:2963, 1991).

As used herein, high affinity with respect to HLA class I molecules is defined as binding with an IC₅₀ or K_{D} value of less than 50 nM; intermediate affinity is binding with an IC₅₀ (or K_{D}) of between about 50 and about 500 nM. High affinity with respect to binding to HLA class II molecules is defined as binding with an IC₅₀ or K_{D} value of less than 100 nM; intermediate affinity is binding with an IC₅₀ or K_{D} of between about 100 and about 1000 nM.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithms or by manual alignment and visual inspection.

An “immunogenic peptide” or “peptide epitope” is a peptide which comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major Histocompatibility Complex” or “MHC” is a cluster of genes that plays a role in control of the cellular interactions responsible for physiologic immune responses. In humans, the MHC complex is also known as the HLA complex. For a detailed description of the MHC and HLA complexes, see, Paul, FUNDAMENTAL IMMUNOLOGY, 3^{RD} ED., Raven Press, New York, 1993.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “negative binding residue” or “deleterious residue” is an amino acid which, if present at certain positions (typically not primary anchor positions) of a peptide epitope, results in decreased binding affinity of the peptide for the peptide's corresponding HLA molecule. Any residue that is not “deleterious” is a “non-deleterious” residue.

The term “peptide” is used interchangeably with “oligopeptide” in the present specification to designate a series of residues, typically L-amino acids, connected one to the other, typically by peptide bonds between the α-amino and carboxyl groups of adjacent amino acids. The preferred CTL-inducing oligopeptides of the invention are 13 residues or less in length and usually consist of between about 8 and about 11 residues, preferably 9 or 10 residues. The preferred HTL-inducing oligopeptides are less than about 50 residues in length and usually consist of between about 6 and about 30 residues, more usually between about 12 and 25, and often between about 15 and 20 residues.

“Pharmaceutically acceptable” refers to a non-toxic, inert, and physiologically compatible composition.

A “primary anchor residue” is an amino acid at a specific position along a peptide sequence which is understood to provide a contact point between the immunogenic peptide and the HLA molecule. One to three, usually two, primary anchor residues within a peptide of defined length generally defines a “motif” for an immunogenic peptide. These residues are understood to fit in close contact with peptide binding grooves of an HLA molecule, with their side chains buried in specific pockets of the binding grooves themselves. In one embodiment, the primary anchor residues are located at position 2 (from the amino terminal position) and at the carboxyl terminal position of a 9 residue peptide in accordance with the invention. The primary anchor positions for each motif and supermotif are set forth in Table I. For example, analog peptides can be created by altering the presence or absence of particular residues in these primary anchor positions. Such analogs are used to finely modulate the binding affinity of a peptide comprising a particular motif or supermotif.

“Promiscuous recognition” is where a distinct peptide is recognized by the same T cell clone in the context of multiple HLA molecules. Promiscuous binding is synonymous with cross-reactive binding.

A “protective immune response” or “therapeutic immune response” refers to a CTL and/or an HTL response to an antigen from an infectious agent or a tumor antigen from which an immunogenic peptide is derived, and thereby preventing or at least partially arresting disease symptoms or progression. The immune response may also include an antibody response which has been facilitated by the stimulation of helper T cells.

The term “residue” refers to an amino acid or amino acid mimetic incorporated into an oligopeptide by an amide bond or amide bond mimetic.

A “secondary anchor residue” is an amino acid at a position other than a primary anchor position in a peptide which may influence peptide binding. A secondary anchor residue occurs at a significantly higher frequency amongst bound peptides than would be expected by random distribution of amino acids at one position. The secondary anchor residues are said to occur at “secondary anchor positions.” A secondary anchor residue can be identified as a residue which is present at a higher frequency among high affinity binding peptides, or a residue otherwise associated with high affinity binding. For example, analog peptides can be created by altering the presence or absence of particular residues in these secondary anchor positions. Such analogs are used to finely modulate the binding affinity of a peptide comprising a particular motif or supermotif.

A “subdominant epitope” is an epitope which evokes little or no response upon immunization with whole antigens which comprise the epitope, but for which a response can be obtained by immunization with an isolated peptide, and this response (unlike the case of cryptic epitopes) is detected when whole protein is used to recall the response in vitro or in vivo.

A “supermotif” is a peptide binding specificity shared by HLA molecules encoded by two or more HLA alleles. A supermotif-bearing epitope is preferably is recognized with high or intermediate affinity (as defined herein) by two or more HLA antigens.

“Synthetic peptide” refers to a peptide that is not naturally occurring, but is man-made using such methods as chemical synthesis or recombinant DNA technology.

The nomenclature used to describe peptide compounds follows the conventional practice wherein the amino group is presented to the left (the N-terminus) and the carboxyl group to the right (the C-terminus) of each amino acid residue. When amino acid residue positions are referred to in a peptide epitope they are numbered in an amino to carboxyl direction with position one being the position closest to the amino terminal. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G. Symbols for the amino acids are shown below.

| Single Letter Symbol | Three Letter Symbol | Amino Acids |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

### IV.B. STIMULATION OF CTL AND HTL RESPONSES AGAINST HBV

The mechanism by which T cells recognize antigens has been delineated during the past ten years. Based on our new understanding of the immune system we have generated efficacious peptide epitope vaccine compositions that can induce a therapeutic or prophylactic immune response to HBV infection in a broad population. For an understanding of the value and efficacy of the claimed compositions, a brief review of the technology is provided.

A complex of an HLA molecule and a peptidic antigen acts as the ligand recognized by HLA-restricted T cells (Buus, S. et al., Cell 47:1071, 1986; Babbitt, B. P. et al., Nature 317:359, 1985; Townsend, A., and Bodmer, H., Annu. Rev. Immunol. 7:601, 1989; Germain, R. N., Annu. Rev. Immunol. 11:403, 1993). Through the study of single amino acid substituted antigen analogs and the sequencing of endogenously bound, naturally processed peptides, critical residues that correspond to motifs required for specific binding to HLA antigen molecules have been identified and are described here and set forth in Tables I, II, and III (see also, e.g., Sette, A. and Grey, H. M., Curr. Opin. Immunol. 4:79, 1992; Sinigaglia, F. and Hammer, J., Curr. Biol. 6:52, 1994; Engelhard, V. H., Curr. Opin. Immunol. 6:13, 1994). Furthermore, x-ray crystallographic analysis of HLA-peptide complexes has revealed pockets within the peptide binding cleft of HLA molecules which accommodate allele-specific residues borne by peptide ligands; these residues in turn determine the HLA binding capacity of the peptides in which they are present (Brown, J. H. et al., Nature 364:33, 1993; Guo, H. C. et al., Proc. Natl. Acad. Sci. USA 90:8053, 1993; Guo, H. C. et al., Nature 360:364, 1992; Silver, M. L. et al., Nature 360:367, 1992; Matsumura, M. et al., Science 257:927, 1992; Madden et al., Cell 70:1035, 1992; Fremont, D. H. et al., Science 257:919, 1992; Saper, M. A., Bjorkman, P. J. and Wiley, D. C., J. Mol. Biol. 219:277, 1991).

Accordingly, the definition of class I and class II allele-specific HLA binding motifs or class I supermotifs allows identification of regions within a protein that have the potential of binding particular HLA antigens (see also e.g., Sette, A. and Grey, H. M., Curr. Opin. Immunol. 4:79, 1992; Sinigaglia, F. and Hammer, J., Curr. Biol. 6:52, 1994; Engelhard, V. H., Curr. Opin. Immunol. 6:13, 1994; Kast, W. M. et al., J. Immunol., 152:3904, 1994).

Furthermore, a variety of assays to quantify the affinity of interaction between peptide and HLA have also been established. Such assays include, for example, measures of IC₅₀ values, inhibition of antigen presentation (Sette et al., J. Immunol. 141:3893, 1991), in vitro assembly assays (Townsend et al., Cell 62:285, 1990), measures of dissociations rates (Parker et al., J. Immunol. 149:1896-1904, 1992), and FACS-based assays using mutated cells, such as RMA.S (Melief, et al., Eur. J. Immunol. 21:2963, 1991).

The present inventors have found that the correlation of binding affinity with immunogenicity is an important factor to be considered when evaluating candidate peptides. Thus, by a combination of motif searches and HLA-peptide binding assays, candidates for epitope-based vaccines have been identified. After determining their binding affinity, additional confirmatory work can be performed to select, amongst these vaccine candidates, epitopes with preferred characteristics in terms of antigenicity and immunogenicity. Various strategies can be utilized to evaluate immunogenicity, including:

1) Evaluation of primary T cell cultures from normal individuals (Wentworth, P. A. et al., Mol. Immunol. 32:603, 1995; Celis, E. et al., Proc. Natl. Acad. Sci. USA 91:2105, 1994; Tsai, V. et al., J. Immunol. 158:1796, 1997; Kawashima, I. et al., Human Immunol. 59:1, 1998); This procedure involves the stimulation of PBL from normal subjects with a test peptide in the presence of antigen presenting cells in vitro over a period of several weeks. T cells specific for the peptide become activated during this time and are detected using a ⁵¹Cr-release assay involving peptide sensitized target cells.

2) Immunization of HLA transgenic mice (Wentworth, P. A. et al., J. Immunol. 26:97, 1996; Wentworth, P. A. et al., Int. Immunol. 8:651, 1996; Alexander, J. et al., J. Immunol. 159:4753, 1997); In this method, peptides in incomplete Freund's adjuvant are administered subcutaneously to HLA transgenic mice. Several weeks following immunization, splenocytes are removed and cultured in vitro in the presence of test peptide for approximately one week. Peptide-specific T cells are detected using a ⁵¹Cr-release assay involving peptide sensitized target cells and target cells expressing endogenously generated antigen.

3) Demonstration of recall T cell responses from immune individuals who have recovered from infection, and/or from chronically infected patients (Rehermann, B. et al., J. Exp. Med. 181:1047, 1995; Doolan, D. L. et al., Immunity 7:97, 1997; Bertoni, R. et al., J. Clin. Invest. 100:503, 1997; Threlkeld, S. C. et al., J. Immunol. 159:1648, 1997; Diepolder, H. M. et al., J. Virol. 71:6011, 1997). In applying this strategy, recall responses were detected by culturing PBL from subjects that had been naturally exposed to the antigen, for instance through infection, and thus had generated an immune response “naturally”. PBL from subjects were cultured in vitro for 1-2 weeks in the presence of test peptide plus antigen presenting cells (APC) to allow activation of “memory” T cells, as compared to “naive” T cells. At the end of the culture period, T cell activity is detected using assays for T cell activity including ⁵¹Cr release involving peptide-sensitized targets, T cell proliferation or lymphokine release.

The following describes the peptide epitopes and corresponding nucleic acids of the invention.

### IV.C. BINDING AFFINITY OF PEPTIDE EPITOPES FOR HLA MOLECULES

As indicated herein, the large degree of HLA polymorphism is an important factor to be taken into account with the epitope-based approach to vaccine development. To address this factor, epitope selection encompassing identification of peptides capable of binding at high or intermediate affinity to multiple HLA molecules is preferably utilized, most preferably these epitopes bind at high or intermediate affinity to two or more allele specific HLA molecules.

CTL-inducing peptides of interest for vaccine compositions preferably include those that have an IC₅₀ or binding affinity value for class I HLA molecules of 500 mM or less. HTL-inducing peptides preferably include those that have an IC₅₀ or binding affinity value for class II HLA molecules of 1000 nM or less. For example, peptide binding is assessed by testing the capacity of a candidate peptide to bind to a purified HLA molecule in vitro. Peptides exhibiting high or intermediate affinity are then considered for further analysis. Selected peptides are tested on other members of the supertype family. In preferred embodiments, peptides that exhibit cross-reactive binding are then used in vaccines or in cellular screening analyses.

As disclosed herein, high HLA binding affinity is correlated with greater immunogenicity. Greater immunogenicity can be manifested in several different ways. Immunogenicity corresponds to whether an immune response is elicited at all, and to the vigor of any particular response. For example, a peptide might elicit an immune response in a diverse array of the population, yet in no instance produce a vigorous response. In accordance with these principles, close to 90% of high binding peptides have been found to be immunogenic, as contrasted with about 50% of the peptides which bind with intermediate affinity. Moreover, higher binding affinity peptides leads to more vigorous immunogenic responses. As a result, less peptide is required to elicit a similar biological effect if a high affinity binding peptide is used. Thus, in preferred embodiments of the invention, high binding epitopes are particularly desired.

The relationship between binding affinity for HLA class I molecules and immunogenicity of discrete peptide epitopes on bound antigens has been determined for the first time in the art by the present inventors. The correlation between binding affinity and immunogenicity was analyzed in two different experimental approaches (Sette, et al., J. Immunol. 153:5586-5592, 1994). In the first approach, the immunogenicity of potential epitopes ranging in HLA binding affinity over a 10,000-fold range was analyzed in HLA-A*0201 transgenic mice. In the second approach, the antigenicity of approximately 100 different hepatitis B virus (HBV)-derived potential epitopes, all carrying A*0201 binding motifs, was assessed by using PBL (peripheral blood lymphocytes) of acute hepatitis patients. Pursuant to these approaches, it was determined that an affinity threshold of approximately 500 nM (preferably an IC₅₀ value of 500 nM or less) determines the capacity of a peptide epitope to elicit a CTL response. These data are true for class I binding affinity measurements for naturally processed peptides and for synthesized T cell epitopes. These data also indicate the important role of determinant selection in the shaping of T cell responses.

An affinity threshold associated with immunogenicity in the context of HLA class II DR molecules has also been delineated (Southwood et al. J. Immunology 160:3363-3373, 1998, and U.S. Ser. No. 60/087,192 filed May 29, 1998). In order to define a biologically significant threshold of DR binding affinity, a database of the binding affinities of 32 DR-restricted epitopes for their restricting element was compiled. In approximately half of the cases (15 of 32 epitopes), DR restriction was associated with high binding affinities, i.e., binding affinities of with an IC₅₀ value of 100 nM or less. In the other half of the cases (16 of 32), DR restriction was associated with intermediate affinity (binding affinities in the 100-1000 nM range). In only one of 32 cases was DR restriction associated with an IC₅₀ of 1000 nM or greater. Thus, 1000 nM can be defined as an affinity threshold associated with immunogenicity in the context of DR molecules.

The binding affinity of peptides for HLA molecules can be determined as described in Example 1, below.

### IV.D. PEPTIDE EPITOPE BINDING MOTIFS AND SUPERMOTIFS

In the past few years evidence has accumulated to demonstrate that a large fraction of HLA class I, and possibly class II molecules can be classified into a relatively few supertypes characterized by largely overlapping peptide binding repertoires, and consensus structures of the main peptide binding pockets.

For HLA molecule pocket analyses, the residues comprising the B and F pockets of HLA class I molecules as described in crystallographic studies (Guo, H. C. et al., Nature 360:364, 1992; Saper, M. A., Bjorkman, P. J. and Wiley, D. C., J. Mol. Biol. 219:277, 1991; Madden, D. R., Garboczi, D. N. and Wiley, D. C., Cell 75:693, 1993), have been compiled from the database of Parham, et al. (Parham, P., Adams, E. J., and Arnett, K. L., Immunol. Rev. 143:141, 1995). In these analyses, residues 9, 45, 63, 66, 67, 70, and 99 were considered to make up the B pocket, and to determine the specificity for the residue in the second position of peptide ligands. Similarly, residues 77, 80, 81, and 116 were considered to determine the specificity of the F pocket, and to determine the specificity for the C-terminal residue of a peptide ligand bound by the HLA molecule.

Through the study of single amino acid substituted antigen analogs and the sequencing of endogenously bound, naturally processed peptides, critical residues required for allele-specific binding to HLA molecules have been identified. The presence of these residues correlates with binding affinity for HLA molecules. The identification of motifs and/or supermotifs that correlate with high and intermediate affinity binding is an important issue with respect to the identification of immunogenic peptide epitopes for the inclusion in a vaccine. Kast et al. (J. Immunol. 152:3904-3912, 1994) have shown that motif-bearing peptides account for 90% of the epitopes that bind to allele-specific HLA class I molecules. In this study all possible peptides of 9 amino acids in length and overlapping by eight amino acids (240 peptides), which cover the entire sequence of the E6 and E7 proteins of human papillomavirus type 16, were evaluated for binding to five allele-specific HLA molecules that are expressed at high frequency among different ethnic groups. This unbiased set of peptides allowed an evaluation of the predictive value of HLA class I motifs. From the set of 240 peptides, 22 peptides were identified that bound to an allele-specific HLA molecules with high or intermediate affinity. Of these 22 peptides, 20, (i.e. 91%), were motif-bearing. Thus, this study demonstrates the value of motifs for the identification of peptide epitopes for inclusion in a vaccine: application of motif-based identification techniques eliminates screening of 90% of the potential epitopes.

Such peptide epitopes are identified in the Tables described below. The Tables for the HLA class I epitopes include over 90% of the peptides that will bind to an allele-specific HLA class I molecule with intermediate or high affinity.

Peptides of the present invention may also include epitopes that bind to MHC class II DR molecules. A significant difference between class I and class II HLA molecules is that, although a stringent size restriction exists for peptide binding to class I molecules, a greater degree of heterogeneity in both sizes and binding frame positions of the motif, relative to the N and C termini of the peptide, can be demonstrated for class II peptide ligands. This increased heterogeneity is due to the structure of the class II-binding groove which, unlike its class I counterpart, is open at both ends. Crystallographic analysis of DRB*0101-peptide complexes (see, e.g. Madden, D. R. Ann. Rev. Immunol. 13:587, 1995) showed that the residues occupying position 1 and position 6 of peptides complexed with DRB*0101 engage two complementary pockets on the DRBa*0101 molecules, with the P1 position corresponding to the most crucial anchor residue and the deepest hydrophobic pocket. Other studies have also pointed to the P6 position as a crucial anchor residue for binding to various other DR molecules.

Thus, peptides of the present invention are identified by any one of several HLA-specific amino acid motifs (see, e.g., Tables I-III). If the presence of the motif corresponds to the ability to bind several allele-specific HLA antigens it is referred to as a supermotif. The allele-specific HLA molecules that bind to peptides that possess a particular amino acid supermotif are collectively referred to as an HLA “supertype.”

The peptide motifs and supermotifs described below provide guidance for the identification and use of peptides in accordance with the invention.

Examples of peptide epitopes bearing the respective supermotif or motif are included in Tables as designated in the description of each motif or supermotif. The Tables include a binding affinity ratio listing for some of the peptide epitopes. The ratio may be converted to IC₅₀ by using the following formula: IC₅₀ of the standard peptide/ratio=IC₅₀ of the test peptide (i.e. the peptide epitope). The IC₅₀ values of standard peptides used to determine binding affinities for Class I peptides are shown in Table IV. The IC₅₀ values of standard peptides used to determine binding affinities for Class II peptides are shown in Table V. The peptides used as standards for the binding assay are examples of standards; alternative standard peptides can also be used when performing such an analysis.

To obtain the peptide epitope sequences listed in each Table, protein sequence data from twenty HBV strains (HPBADR, HPBADR1CG, HPBADRA, HPBADRC, HPBADRCG, HPBCGADR, HPBVADRM, HPBADW, HPBADW1, HPBADW2, HPBADW3, HPBADWZ, HPBHEPB, HPBVADW2, HPBAYR, HPBV, HPBVAYWC, HPBVAYWCI, NAD HPBVAYWE) were evaluated for the presence of the designated supermotif or motif. Peptide epitopes were also selected on the basis of their conservancy. A criterion for conservancy requires that the entire sequence of a peptide be totally conserved in 75% of the sequences available for a specific protein. The percent conservancy of the selected peptide epitopes is indicated on the Tables. The frequency, i.e. the number of strains of the 20 strains in which the peptide sequence was identified, is also shown. The “1^{st} position” column in the Tables designates the amino acid position of the HBV protein that corresponds to the first amino acid residue of the epitope. “Number of amino acids” indicates the number of residues in the epitope sequence.

### HLA Class I Motifs Indicative of CTL Inducing Peptide Epitopes:

The primary anchor residues of the HLA class I peptide epitope supermotifs and motifs delineated below are summarized in Table I. The HLA class I motifs set out in Table I(a) are those most particularly relevant to the invention claimed here. Primary and secondary anchor positions are summarized in Table II. Allele-specific HLA molecules that comprise HLA class I supertype families are listed in Table VI.

### IV.D1. HLA-A1 Supermotif

The HLA-A1 supermotif is characterized by the presence in peptide ligands of a small (T or S) or hydrophobic (L, I, V, M, or F) primary anchor residue in position 2, and an aromatic (Y, F, or W) primary anchor residue at the C-terminal position of the epitope. The corresponding family of HLA molecules that bind to the A1 supermotif (i.e., the HLA-A1 supertype) is comprised of at least A*0101, A*2601, A*2602, A*2501, and A*3201 (see, e.g., DiBrino, M. et al., J. Immunol. 151:5930, 1993; DiBrino, M. et al., J. Immunol. 152:620, 1994; Kondo, A. et al., Immunogenetics 45:249, 1997.). Other allele-specific HLA molecules predicted to be members of the A1 superfamily are shown in Table VI. Peptides binding to each of the individual HLA proteins can be modulated by substitutions at primary anchor positions, preferably choosing respective residues specified for the supermotif.

Representative peptide epitopes that comprise the A1 supermotif are set forth on the attached Table VII.

### IV.D.2. HLA-A2 Supermotif

Primary anchor specificities for allele-specific HLA A2.1 molecules (Falk et al., Nature 351:290-296, 1991; Hunt et al., Science 255:1261-1263, 1992) and cross-reactive binding within the HLA A2 family (Fruci et al., Human Immunol. 38:187-192, 1993; Tanigaki et al., Human Immunol. 39:155-162, 1994) have been described. The present inventors have defined additional primary anchor residues that determine cross-reactive binding to multiple allele-specific HLA A2 molecules (Del Guercio et al., J. Immunol. 154:685-693, 1995). The HLA-A2 supermotif comprises peptide ligands with L, I, V, M, A, T, or Q as primary anchor residues at position 2 and L, I, V, M, A, or T as a primary anchor residue at the C-terminal position of the epitope.

The corresponding family of HLA molecules (i.e., the HLA-A2 supertype that binds these peptides) is comprised of at least: A*0201, A*0202, A*0203, A*0204, A*0205, A*0206, A*0207, A*0209, a*0214, A*6802, and A*6901. Other allele-specific HLA molecules predicted to be members of the A2 superfamily are shown in Table VI. As explained in detail below, binding to each of the individual allele-specific HLA molecules can be modulated by substitutions at the primary anchor and/or secondary anchor positions, preferably choosing respective residues specified for the supermotif.

Representative peptide epitopes that comprise an A2 supermotif are set forth on the attached Table VIII. The motifs comprising the primary anchor residues V, A, T, or Q at position 2 and L, I, V, A, or T at the C-terminal position are those most particularly relevant to the invention claimed herein.

### IV.D.3. HLA-A3 Supermotif

The HLA-A3 supermotif is characterized by the presence in peptide ligands of A, L, I, V, M, S, or, T as a primary anchor at position 2, and a positively charged residue, R or K, at the C-terminal position of the epitope (e.g., in position 9 of 9-mers). Exemplary members of the corresponding family of HLA molecules (the HLA-A3 supertype) that bind the A3 supermotif include at least: A*0301, A*1101, A*3101, A*3301, and A*6801. Other allele-specific HLA molecules predicted to be members of the A3 superfamily are shown in Table VI. As explained in detail below, peptide binding to each of the individual allele-specific HLA proteins can be modulated by substitutions of amino acids at the primary and/or secondary anchor positions of the peptide, preferably choosing respective residues specified for the supermotif.

Representative peptide epitopes that comprise the A3 supermotif are set forth on the attached Table IX.

### IV.D.4. HLA-A24 Supermotif

The HLA-A24 supermotif is characterized by the presence in peptide ligands of an aromatic (F, W, or Y) residue as a primary anchor in position 2, and a hydrophobic (Y, F, L, I, V, or M) residue as primary anchor at the C-terminal position of the epitope. The corresponding family of HLA molecules that bind to the A24 supermotif (i.e., the A24 supertype) includes at least A*2402, A*3001, and A*2301. Other allele-specific HLA molecules predicted to be members of the A24 superfamily are shown in Table VI. Peptide binding to each of the allele-specific HLA molecules can be modulated by substitutions at primary anchor positions, preferably choosing respective residues specified for the supermotif.

Representative peptide epitopes that comprise the A24 supermotif are set forth on the attached Table X.

### IV.D.5. HLA-B7 Supermotif

The HLA-B7 supermotif is characterized by peptides bearing proline in position 2 as a primary anchor, and a hydrophobic or aliphatic amino acid (L, I, V, M, A, F, W, or Y) as the primary anchor at the C-terminal position of the epitope. The corresponding family of HLA molecules that bind the B7 supermotif (i.e., the HLA-B7 supertype) is comprised of at least twenty six HLA-B proteins including: B*0702, B*0703, B*0704, B*0705, B*1508, B*3501, B*3502, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, and B*7801 (see, e.g., Sidney, et al., J. Immunol. 154:247, 1995; Barber, et al., Curr. Biol. 5:179, 1995; Hill, et al., Nature 360:434, 1992; Rammensee, et al., Immunogenetics 41:178, 1995). Other allele-specific HLA molecules predicted to be members of the B7 superfamily are shown in Table VI. As explained in detail below, peptide binding to each of the individual allele-specific HLA proteins can be modulated by substitutions at the primary and/or secondary anchor positions of the peptide, preferably choosing respective residues specified for the supermotif.

Representative peptide epitopes that contain the B7 supermotif are set forth on the attached Table XI.

### IV.D.6. HLA-B27 Supermotif

The HLA-B27 supermotif is characterized by the presence in peptide ligands of a positively charged (R, H, or K) residue as a primary anchor at position 2, and a hydrophobic (F, Y, L, W, M, I, A, or V) residue as a primary anchor at the C-terminal position of the epitope. Exemplary members of the corresponding family of HLA molecules that bind to the B27 supermotif (i.e., the B27 supertype) include at least B*1401, B*1402, B*1509, B*2702, B*2703, B*2704, B*2705, B*2706, B*3801, B*3901, B*3902, and B*7301. Other allele-specific HLA molecules predicted to be members of the B27 superfamily are shown in Table VI. Peptide binding to each of the allele-specific HLA molecules can be modulated by substitutions at primary anchor positions, preferably choosing respective residues specified for the supermotif.

Representative peptide epitopes that comprise the B27 supermotif are set forth on the attached Table XII.

### IV.D.7. HLA-B44 Supermotif

The HLA-B44 supermotif is characterized by the presence in peptide ligands of negatively charged (D or E) residues as a primary anchor in position 2, and hydrophobic residues (F, W, Y, L, I, M, V, or A) as a primary anchor at the C-terminal position of the epitope. Exemplary members of the corresponding family of HLA molecules that bind to the B44 supermotif (i.e., the B44 supertype) include at least: B*1801, B*1802, B*3701, B*4001, B*4002, B*4006, B*4402, B*4403, and B*4006. Peptide binding to each of the allele-specific HLA molecules can be modulated by substitutions at primary anchor positions; preferably choosing respective residues specified for the supermotif.

### IV.D.8. HLA-B58 Supermotif

The HLA-B58 supermotif is characterized by the presence in peptide ligands of a small aliphatic residue (A, S, or T) as a primary anchor residue at position 2, and an aromatic or hydrophobic residue (F, W, Y, L, I, V, M, or A) as a primary anchor residue at the C-terminal position of the epitope. Exemplary members of the corresponding family of HLA molecules that bind to the B58 supermotif (i.e., the B58 supertype) include at least: B*1516, B*1517, B*5701, B*5702, and B*5801. Other allele-specific HLA molecules predicted to be members of the B58 superfamily are shown in Table VI. Peptide binding to each of the allele-specific HLA molecules can be modulated by substitutions at primary anchor positions, preferably choosing respective residues specified for the supermotif.

Representative peptide epitopes that comprise the B58 supermotif are set forth on the attached Table XIII.

### IV.D.9. HLA-B62 Supermotif

The HLA-B62 supermotif is characterized by the presence in peptide ligands of the polar aliphatic residue Q or a hydrophobic aliphatic residue (L, V, M, or I) as a primary anchor in position 2, and a hydrophobic residue (F, W, Y, M, I, V, L, or A) as a primary anchor at the C-terminal position of the epitope. Exemplary members of the corresponding family of HLA molecules that bind to the B62 supermotif (i.e., the B62 supertype) include at least: B*1501, B*1502, B*1513, and B5201. Other allele-specific HLA molecules predicted to be members of the B62 superfamily are shown in Table VI.

Peptide binding to each of the allele-specific HLA molecules can be modulated by substitutions at primary anchor positions, preferably choosing respective residues specified for the supermotif.

Representative peptide epitopes that comprise the B62 supermotif are set forth on the attached Table XIV.

### IV.D.10. HLA-A1 Motif

The allele-specific HLA-A1 motif is characterized by the presence in peptide ligands of T, S, or M as a primary anchor residue at position 2 and the presence of Y as a primary anchor residue at the C-terminal position of the epitope. An alternative allele-specific A1 motif (i.e., a “submotif”) is characterized by a primary anchor residue at position 3 rather than position 2. This submotif is characterized by the presence of D, E, A, or S as a primary anchor residue in position 3, and a Y as a primary anchor residue at the C-terminal position of the epitope. Peptide binding to HLA A1 can be modulated by substitutions at primary and/or secondary anchor positions, preferably choosing respective residues specified for the motif.

Representative peptide epitopes that comprise either A1 motif are set forth on the attached Table XV. Those epitopes comprising T, S, or M at position 2 and Y at the C-terminal position are also included in the listing of HLA-A1 supermotif-bearing peptide epitopes listed in Table VII.

### IV.D.11. HLA-A2.1 Motif

An allele-specific HLA-A2.1 motif was first determined to be characterized by the presence in peptide ligands of L or M as a primary anchor residue in position 2, and L or V as a primary anchor residue at the C-terminal position of a 9 amino acid epitope (Falk et al., Nature 351:290-296, 1991). Furthermore, the A2.1 motif was determined to further comprise an I at position 2 and I or A at the C-terminal position of a nine amino acid peptide (Hunt et al., Science 255:1261-1263, Mar. 6, 1992). Additionally, the A2.1 allele-specific motif has been found to comprise a T at the C-terminal position (Kast et al., J. Immunol. 152:3904-3912, 1994). Subsequently, the A2.1 allele-specific motif has been defined by the present inventors to additionally comprise V, A, T, or Q as a primary anchor residue at position 2, and M as a primary anchor residue at the C-terminal position of the epitope. Thus, the HLA-A2.1 motif comprises peptide ligands with L, I, V, M, A, T, or Q as primary anchor residues at position 2 and L, I, V, M, A, or T as a primary anchor residue at the C-terminal position of the epitope. The preferred and tolerated residues that characterize the primary anchor positions of the HLA-A2.1 motif are identical to the preferred residues of the A2 supermotif. (for reviews of relevant data, see, e.g., Del Guercio et al., J. Immunol. 154:685-693, 1995; Sidney et al., Immunol. Today 17:261-266, 1996; Sette and Sidney, Curr. Opin. in Immunol. 10:478-482, 1998). Secondary anchor residues that characterize the A2.1 motif have additionally been defined as disclosed herein. These are disclosed in Table II. Peptide binding to HLA-A2.1 molecules can be modulated by substitutions at primary and/or secondary anchor positions, preferably choosing respective residues specified for the motif.

Representative peptide epitopes that comprise an A2.1 motif are set forth on the attached Table VII. The A2.1 motifs comprising the primary anchor residues V, A, T, or Q at position 2 and L, I, V, A, or T at the C-terminal position are those most particularly relevant to the invention claimed herein.

### IV.D.12 HLA-A3 Motif

The allele-specific HLA-A3 motif is characterized by the presence in peptide ligands of L, M, V, I, S, A, T, F, C, G, or D as a primary anchor residue at position 2, and the presence of K, Y, R, H, F, or A as a primary anchor residue at the C-terminal position of the epitope. Peptide binding to HLA-A3 can be modulated by substitutions at primary and/or secondary anchor positions, preferably choosing respective residues specified for the motif.

Representative peptide epitopes that comprise the A3 motif are set forth on the attached Table XVI. Those peptide epitopes that also comprise the A3 supermotif are also listed in Table IX.

### IV.D.13. HLA-A11 Motif

The allele-specific HLA-A11 motif is characterized by the presence in peptide ligands of V, T, M, L, I, S, A, G, N, C, D, or F as a primary anchor residue in position 2, and K, R, Y, or H as a primary anchor residue at the C-terminal position of the epitope. Peptide binding to HLA-A 11 can be modulated by substitutions at primary and/or secondary anchor positions, preferably choosing respective residues specified for the motif.

Representative peptide epitopes that comprise the A11 motif are set forth on the attached Table XVII; peptide epitopes comprising the A3 allele-specific motif are also present in this Table because of the extensive overlap between the A3 and A11 motif primary anchor specificities. Further, those peptide epitopes that comprise the A3 supermotif are also listed in Table IX.

### IV.D.14. HLA-A24 Motif

The allele-specific HLA-A24 motif is characterized by the presence in peptide ligands of Y, F, W, or M as a primary anchor residue in position 2, and F, L, I, or W as a primary anchor residue at the C-terminal position of the epitope. Peptide binding to HLA-A24 molecules can be modulated by substitutions at primary and/or secondary anchor positions; preferably choosing respective residues specified for the motif.

Representative peptide epitopes that comprise the A24 motif are set forth on the attached Table XVIII. These epitopes are also listed in Table X, which sets forth HLA-A24-supermotif-bearing peptide epitopes.

### Motifs Indicative of Class II HTL Inducing Peptide Epitopes

The primary and secondary anchor residues of the HLA class II peptide epitope supermotifs and motifs delineated below are summarized in Table III.

### IV.D.15. HLA DR-14-7 Supermotif

Motifs have also been identified for peptides that bind to three common HLA class II allele-specific HLA molecules: HLA DRB1*0401, DRB1*0101, and DRB1*0701. Collectively, the common residues from these motifs delineate the HLA DR-1-4-7 supermotif. Peptides that bind to these DR molecules carry a supermotif characterized by a large aromatic or hydrophobic residue (Y, F, W, L, I, V, or M) as a primary anchor residue in position 1, and a small, non-charged residue (S, T, C, A, P, V, I, L, or M) as a primary anchor residue in position 6 of the epitope. Allele-specific secondary effects and secondary anchors for each of these HLA types have also been identified. These are set forth in Table III. Peptide binding to HLA-DR4, DR1, and/or DR7 can be modulated by substitutions at primary and/or secondary anchor positions, preferably choosing respective residues specified for the supermotif.

Conserved peptide epitopes (i.e. 75% conservancy in the 20 HBV strains used for the analysis), corresponding to a nine residue core comprising the DR-1-4-7 supermotif (wherein position 1 of the motif is at position I of the nine residue core) are set forth in Table XIXa (see, e.g., Madden, Annu. Rev. Immunol. 13:587-622, 1995). Respective exemplary peptide epitopes of 15 amino acid residues in length, each of which comprise a conserved nine residue core, are also shown in section “a” of the Table. Cross-reactive binding data for the exemplary 15-residue supermotif-bearing peptides denoted by a peptide number are shown in Table XIXb.

### IV.D.16. HLA DR3 Motifs

Two alternative motifs (i.e., submotifs) characterize peptide epitopes that bind to HLA-DR3 molecules. In the first motif (submotif DR3A) a large, hydrophobic residue (L, I, V, M, F, or Y) is present in anchor position 1, and D is present as an anchor at position 4, towards the carboxyl terminus of the epitope.

The alternative DR3 submotif provides for lack of the large, hydrophobic residue at anchor position 1, and/or lack of the negatively charged or amide-like anchor residue at position 4, by the presence of a positive charge at position 6 towards the carboxyl terminus of the epitope. Thus, for the alternative allele-specific DR3 motif (submotif DR3B): L, I, V, M, F, Y, A, or Y is present at anchor position 1; D, N, Q, E, S, or T is present at anchor position 4; and K, R, or H is present at anchor position 6. Peptide binding to HLA-DR3 can be modulated by substitutions at primary and/or secondary anchor positions, preferably choosing respective residues specified for the motif.

Conserved peptide epitopes (i.e., sequences that are 75% conservaned in the 20 HBV strains used for the analysis), corresponding to a nine residue core comprising the DR3A submotif (wherein position 1 of the motif is at position I of the nine residue core) set forth in Table XXa. Respective exemplary peptide epitopes of 15 amino acid residues in length, each of which comprise a conserved nine residue core, are also shown in section “a” of the Table. Table XXb shows binding data of the exemplary DR3 submotif A-bearing peptides denoted by a peptide number.

Conserved peptide epitopes (i.e., 75% conservancy in the 20 HBV strains used for the analysis), corresponding to a nine residue core comprising the DR3B submotif and respective exemplary 15-mer peptides comprising the DR3 submotif-B epitope are set forth in Table XXc. Table XXd shows binding data of the exemplary DR3 submotif B-bearing peptides denoted by a peptide number.

Each of the HLA class I or class II peptide epitopes set out in the Tables herein are deemed singly to be an inventive aspect of this application. Further, it is also an inventive aspect of this application that each peptide epitope may be used in combination with any other peptide epitope.

### IV.E. Enhancing Population Coverage of the Vaccine

Vaccines that have broad population coverage are preferred because they are more commercially viable and generally applicable to the most people. Broad population coverage can be obtained using the peptides of the invention (and nucleic acid compositions that encode such peptides) through selecting peptide epitopes that bind to HLA alleles which, when considered in total, are present in most of the population. Table XXI lists the overall frequencies of the HLA class I supertypes in various ethnicities (Table XXIa) and the combined population coverage achieved by the A2-, A3-, and B7-supertypes (Table XXIb). The A2-, A3-, and B7 supertypes are each present on the average of over 40% in each of these five major ethnic groups. Coverage in excess of 80% is achieved with a combination of these supermotifs. These results suggest that effective and non-ethnically biased population coverage is achieved upon use of a limited number of cross-reactive peptides. Although the population coverage reached with these three main peptide specificities is high, coverage can be expanded to reach 95% population coverage and above, and more easily achieve truly multispecific responses upon use of additional supermotif or allele-specific motif bearing peptides.

The B44-, A1-, and A24-supertypes are present, on average, in a range from 25% to 40% of these major ethnic populations (Table XXIa). While less prevalent overall, the B27-, B58-, and B62 supertypes are each present with a frequency>25% in at least one major ethnic group (Table XXIa). Table XXIB summarizes the estimated combined prevalence in five major ethnic groups of HLA supertypes that have been identified. The incremental coverage obtained by the inclusion of A1, -A24-, and B44-supertypes to the A2, A3, and B7 coverage, or all of the supertypes described herein is shown. By including epitopes from the six most frequent supertypes, an average population coverage of 99% is obtained for five major ethnic groups.

The data presented herein, together with the previous definition of the A2-, A3-, and B7-supertypes, indicates that all antigens, with the possible exception of A29, B8, and B46, can be classified into a total of nine HLA supertypes. Focusing on the six most common supertypes affords population coverage greater than 98% for all major ethnic populations.

### IV.F. IMMUNE RESPONSE STIMULATING PEPTIDE ANALOGS

Although peptides with suitable cross-reactivity among all alleles of a superfamily are identified by the screening procedures described above, cross-reactivity is not always complete and in such cases procedures to further increase cross-reactivity of peptides can be useful; such procedures can also be used to modify other properties of the peptides. Having established the general rules that govern cross-reactivity of peptides for HLA alleles within a given motif or supermotif, modification (i.e., analoging) of the structure of peptides of particular interest in order to achieve broader (or otherwise modified) HLA binding capacity can be performed. More specifically, peptides which exhibit the broadest cross-reactivity patterns, (both amongst the known T cell epitopes, as well as the more extended set of peptides that contain the appropriate supermotifs), can be produced in accordance with the teachings herein.

The strategy employed utilizes the motifs or supermotifs which correlate with binding to certain HLA molecules. The motifs or supermotifs are defined by having primary anchors, though secondary anchors can also be modified. Analog peptides can be created by substituting amino acids residues at primary anchor, secondary anchor, or at primary and secondary anchor positions. Generally, analogs are made for peptides that already bear a motif or supermotif. Preferred secondary anchor residues of supermotifs and motifs that have been defined for HLA class I and class II binding peptides are shown in Tables II and III, respectively.

For a number of the motifs or supermotifs in accordance with the invention, residues are defined which are deleterious to binding to allele-specific HLA molecules or members of HLA supertypes that bind to the respective motif or supermotif (Tables II and III). Accordingly, removal of residues that are detrimental to binding can be performed in accordance with the present invention. For example, in the case of the A3 supertype, when all peptides that have such deleterious residues are removed from the population of analyzed peptides, the incidence of cross-reactivity increases from 22% to 37% (see, e.g., Sidney, J. et al., Hu. Immunol. 45:79, 1996). Thus, one strategy to improve the cross-reactivity of peptides within a given supermotif is simply to delete one or more of the deleterious residues present within a peptide and substitute a small “neutral” residue such as Ala (that may not influence T cell recognition of the peptide). An enhanced likelihood of cross-reactivity is expected if, together with elimination of detrimental residues within a peptide, residues associated with high affinity binding to multiple alleles within a superfamily are inserted.

To ensure that an analog peptide, when used as a vaccine, actually elicits a CTL response to the native epitope in vivo (or, in the case of class II epitopes, elicits helper T cells that cross-react with the wild type peptides), the analog peptide may be used to immunize T cells in vitro from individuals of the appropriate HLA allele. Thereafter, the immunized cells' capacity to induce lysis of wild type peptide sensitized target cells is evaluated. It will be desirable to use as antigen presenting cells, cells that have been either infected, or transfected with the appropriate genes, or, in the cae of class II epitopes only, cells that have been pusled with whole protein antigens, to establish whether endogenously produced antigen is also recognized by the relevant T cells.

Another embodiment of the invention to ensure adequate numbers of cross-reactive cellular binders is to create analogs of weak binding peptides. Class I peptides exhibiting binding affinities of 500-50000 nM, and carrying an acceptable but suboptimal primary anchor residue at one or both positions can be “fixed” by substituting preferred anchor residues in accordance with the respective supertype. The analog peptides can then be tested for crossbinding activity.

Another embodiment for generating effective peptide analogs involves the substitution of residues that have an adverse impact on peptide stability or solubility in a liquid environment. This substitution may occur at any position of the peptide epitope. For example, a cysteine (C) can be substituted out in favor of α-amino butyric acid. Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances (Review: A. Sette et al., In: Persistent Viral Infections, Eds. R. Ahmed and I. Chen, John Wiley & Sons, England, 1999). Substitution of cysteine with α-amino butyric acid may occur at any residue of a peptide epitope, i.e. at either anchor or non-anchor positions.

In general, CTL and HTL responses are not directed against all possible epitopes. Rather, they are restricted to a few immunodominant determinants (Zinkemagel, et al., Adv. Immunol. 27:5159, 1979; Bennink, et al., J. Exp. Med. 168:19351939, 1988; Rawle, et al., J. Immunol. 146:3977-3984, 1991). It has been recognized that immunodominance (Benacerraf, et al., Science 175:273-279, 1972) could be explained by either the ability of a given epitope to selectively bind a particular HLA protein (determinant selection theory) (Vitiello, et al., J. Immunol. 131:1635, 1983); Rosenthal, et al., Nature 267:156-158, 1977), or being selectively recognized by the existing TCR (T cell receptor) specificities (repertoire theory) (Klein, J., IMMUNOLOGY, THE SCIENCE OF SELFNONSELF DISCRIMINATION, John Wiley & Sons, New York, pp. 270-310, 1982). It has been demonstrated that additional factors, mostly linked to processing events, can also play a key role in dictating, beyond strict immunogenicity, which of the many potential determinants will be presented as immunodominant (Sercarz, et al., Annu. Rev. Immunol. 11:729-766, 1993).

The concept of dominance and subdominance is relevant to immunotherapy of both infectious diseases and cancer. For example, in the course of chronic viral disease, recruitment of subdominant epitopes can be important for successful clearance of the infection, especially if dominant CTL or HTL specificities have been inactivated by functional tolerance, suppression, mutation of viruses and other mechanisms (Franco, et al., Curr. Opin. Immunol. 7:524-531, (1995)). In the case of cancer and tumor antigens, CTLs recognizing at least some of the highest binding affinity peptides might be functionally inactivated. Lower binding affinity peptides are preferentially recognized at these, times.

In particular, it has been noted that a significant number of epitopes derived from known non-viral tumor associated antigens (TAA) bind HLA class I with intermediate affinity (IC₅₀ in the 50-500 nM range). For example, it has been found that 8 of 15 known TAA peptides recognized by tumor infiltrating lymphocytes (TIL) or CTL bound in the 50-500 nM range. (These data are in contrast with estimates that 90% of known viral antigens that were recognized as peptides bound HLA with IC₅₀ of 50 nM or less, while only approximately 10% bound in the 50-500 nM range (Sette, et al., J. Immunol., 153:558-5592 (1994)). In the cancer setting this phenomenon is probably due to elimination, or functional inhibition of the CTL recognizing several of the highest binding peptides, presumably because of T cell tolerization events.

Without intending to be bound by theory, it is believed that because T cells to dominant epitopes may have been clonally deleted, selecting subdominant epitopes may allow extant T cells to be recruited, which will then lead to a therapeutic response. However, the binding of HLA molecules to subdominant epitopes is often less vigorous than to dominant ones. Accordingly, there is a need to be able to modulate the binding affinity of particular immunogenic epitopes for one or more HLA molecules, and thereby to modulate the immune response elicited by the peptide. Thus, a need exists to prepare analog peptides which elicit a more vigorous response. This ability would greatly enhance the usefulness of peptide-based vaccines and therapeutic agents.

Representative analog peptides are set forth in Table XXII. The Table indicates the length and sequence of the analog peptide as well as the motif or supermotif, if appropriate. The information in the “Fixed Nomenclature” column indicates the residues substituted at the indicated position numbers for the respective analog.

### IV.G. COMPUTER SCREENING OF PROTEIN SEQUENCES FROM DISEASE-RELATED ANTIGENS FOR SUPERMOTIF OR MOTIF CONTAINING PEPTIDES

Computer programs that allow the rapid screening of protein sequences for the occurrence of the subject supermotifs or motifs are encompassed by the present invention; as are programs that permit the generation of analog peptides. These programs are implemented to analyze any identified amino acid sequence or operate on an unknown sequence and simultaneously determine the sequence and identify motif-bearing epitopes thereof; analogs can be simultaneously determined as well. Generally, the identified sequences will be from a pathogenic organism or a tumor-associated peptide. For example, the target molecules considered herein include all of the HBV proteins (e.g. surface, core, polymerase, and X).

In cases where the sequence of multiple variants of the same target protein are available, peptides may also be selected on the basis of their conservancy. A presently preferred criterion for conservancy defines that the entire sequence of a peptide be totally conserved in 75% of the sequences evaluated for a specific protein; this definition of conservancy has been employed herein.

It is important that the selection criteria utilized for prediction of peptide binding are as accurate as possible, to correlate most efficiently with actual binding. Prediction of peptides that bind, for example, to HLA-A*0201, on the basis of the presence of the appropriate primary anchors, is positive at about a 30% rate (Ruppert, J. et al. Cell 74:929, 1993). However, by analyzing an extensive peptide-HLA binding database, the present inventors have developed a number of allele specific polynomial algorithms that dramatically increase the predictive value over identification on the basis of the presence of primary anchor residues alone. These algorithms take into account not only the presence or absence of the correct primary anchors, but also consider the positive or deleterious presence of secondary anchor residues (to account for the impact of different amino acids at different positions). The algorithms are essentially based on the premise that the overall affinity (or AG) of peptide-HLA interactions can be approximated as a linear polynomial function of the type:

ΔG=a₁ᵢ×a₂ᵢ×a₃ᵢ . . . ×aₙᵢ

where aᵢⱼ is a coefficient that represents the effect of the presence of a given amino acid (j) at a given position (i) along the sequence of a peptide of n amino acids. An important assumption of this method is that the effects at each position are essentially independent of each other. This assumption is justified by studies that demonstrated that peptides are bound to HLA molecules and recognized by T cells in essentially an extended conformation. Derivation of specific algorithm coefficients has been described in Gulukota et al. (Gulukota, K. et al., J. Mol. Biol. 267:1258, 1997).

Additional methods to identify preferred peptide sequences, which also make use of specific motifs, include the use of neural networks and molecular modeling programs (Gulukota, K. et al., J. Mol. Biol. 267:1258, 1997; Milik et al., Nature Biotechnology 16:753, 1998; Altuvia et al., Hum. Immunol. 58:1, 1997; Altuvia et al, J. Mol. Biol. 249:244, 1995; Buus, S. Curr. Opin. Immunol. 11:209-213, 1999; Brusic, V. et al., Bioinformatics 14:121-130, 1998).

For example, it has been shown that in sets of A*0201 motif peptides, 69% of the peptides containing at least one preferred secondary anchor residue while avoiding the presence of any deleterious secondary anchor residues, will bind A*0201 with an IC₅₀ less than 500 nM (Ruppert, J. et al. Cell 74:929, 1993). These algorithms are also flexible in that cut-off scores may be adjusted to select sets of peptides with greater or lower predicted binding properties, as desired.

In utilizing computer screening to identify peptide epitopes, all protein sequence or translated sequence may be analyzed using software developed to search for motifs, for example the “FINDPATTERNS’ program (Devereux, et al. Nucl. Acids Res. 12:387-395, 1984) or MotifSearch 1.4 software program (D. Brown, San Diego, Calif.) to identify potential peptide sequences containing appropriate HLA binding motifs. As appreciated by one of ordinary skill in the art a large array of software and hardware options are available which can be employed to implement the motifs of the invention relative to known or unknown peptide sequences. The identified peptides will then be scored using customized polynomial algorithms to predict their capacity to bind specific HLA class I or class II alleles.

In accordance with the procedures described above, HBV peptides and analogs thereof that are able to bind HLA supertype groups or allele-specific HLA molecules have been identified (Tables VII-XX; Table XXII).

### IV.H. PREPARATION OF PEPTIDE EPITOPES

Peptides in accordance with the invention can be prepared synthetically, by recombinant DNA technology, or from natural sources such as native tumors or pathogenic organisms. Peptide epitopes may be synthesized individually or as polyepitopic peptides. Although the peptide will preferably be substantially free of other naturally occurring host cell proteins and fragments thereof, in some embodiments the peptides may be synthetically conjugated to native fragments or particles.

The peptides in accordance with the invention can be a variety of lengths, and either in their neutral (uncharged) forms or in forms which are salts. The peptides in accordance with the invention are either free of modifications such as glycosylation, side chain oxidation, or phosphorylation; or they contain these modifications, subject to the condition that modifications do not destroy the biological activity of the peptides as described herein.

Desirably, the peptide will be as small as possible while still maintaining substantially all of the biological activity of the large peptide. When possible, it may be desirable to optimize HLA class I binding peptides of the invention to a length of about 8 to about 13 amino acid residues, preferably 9 to 10. HLA class II binding peptides may be optimized to a length of about 6 to about 25 amino acids in length, preferably to between about 13 and about 20 residues. Preferably, the peptides are commensurate in size with endogenously processed pathogen-derived peptides or tumor cell peptides that are bound to the relevant HLA molecules. Moreover, the identification and preparation of peptides of other lengths can be carried out using the techniques described herein (e.g., the disclosures regarding primary and secondary anchor positions). However, it is also preferred to identify a larger region of a native peptide that encompasses one and preferably two or more epitopes in accordance with the invention. This sequence is selected on the basis that it contains the greatest number of epitopes per amino acid length. It is to be appreciated that epitopes can be present in a frame-shifted manner, e.g. a 10 amino acid long peptide could contain two 9 amino acid long epitopes and one 10 amino acid long epitope; each epitope can be exposed and bound by an HLA molecule upon administration of a plurality of such peptides. This larger, preferably multi-epitopic, peptide can then be generated synthetically, recombinantly, or via cleavage from the native source.

The peptides of the invention can be prepared in a wide variety of ways. For the preferred relatively short size, the peptides can be synthesized in solution or on a solid support in accordance with conventional techniques. Various automatic synthesizers are commercially available and can be used in accordance with known protocols. See, for example, Stewart & Young, SOLID PHASE PEPTIDE SYNTHESIS, 2D. ED., Pierce Chemical Co. (1984). Further, individual peptides may be joined using chemical ligation to produce larger peptides.

Alternatively, recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Thus, recombinant polypeptides which comprise one or more peptide sequences of the invention can be used to present the appropriate T cell epitope.

As the nucleotide coding sequence for peptides of the preferred lengths contemplated herein can be synthesized by chemical techniques, for example, the phosphotriester method of Matteucci, et al., J. Am. Chem. Soc. 103:3185 (1981) modification can be made simply by substituting the appropriate and desired nucleic acid base(s) for those that encode the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast, insect or mammalian cell hosts may also be used, employing suitable vectors and control sequences.

### IV.I. ASSAYS TO DETECT T-CELL RESPONSES

Once HLA binding peptides are identified, they can be tested for the ability to elicit a T-cell response. The preparation and evaluation of motif-bearing peptides are described in PCT publications WO 94/20127 and WO 94/03205. Briefly, peptides comprising epitopes from a particular antigen are synthesized and tested for their ability to bind to the appropriate HLA proteins in assays using, for example, purified HLA class I molecules and radioiodonated peptides and/or cells expressing empty class I molecules (which lack peptide in their receptor) by, for instance, immunofluorescent staining and flow microfluorimetry, peptide-dependent class I assembly assays, and inhibition of CTL recognition by peptide competition. Those peptides that bind to the class I molecule are further evaluated for their ability to serve as targets for CTLs derived from infected or immunized individuals, as well as for their capacity to induce primary in vitro or in vivo CTL responses that can give rise to CTL populations capable of reacting with selected target cells associated with a disease. Corresponding assays are used for evaluation of HLA class II binding peptides.

Conventional assays utilized to detect CTL responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations. Antigen-presenting cells can be normal cells such as peripheral blood mononuclear cells or dendritic cells. Alternatively, mutant mammalian cell lines that are deficient in their ability to load class I molecules with internally processed peptides and that have been transfected with the appropriate human class I gene may be used to test for the capacity of the peptide to induce in vitro primary CTL responses.

Peripheral blood lymphocytes may be used as the responder cell source of CTL precursors. The appropriate antigen-presenting cells are incubated with peptide and the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the HBV antigen from which the peptide sequence was derived.

More recently, a method has also been devised which allows direct quantification of antigen-specific T cells by staining with Fluorescein-labelled HLA tetrameric complexes (Altman, J. D. et al., Proc. Natl. Acad. Sci. USA 90:10330, 1993; Altman, J. D. et al., Science 274:94, 1996). Other relatively recent technical developments include staining for intracellular lymphokines, and interferon release assays or ELISPOT assays. Tetramer staining, intracellular lymphokine staining and ELISPOT assays all appear to be at least 10-fold more sensitive than more conventional assays (Lalvani, A. et al., J. Exp. Med. 186:859, 1997; Dunbar, P. R. et al., Curr. Biol. 8:413, 1998; Murali-Krishna, K. et al., Immunity 8:177, 1998).

HTL activation may also be assessed using such techniques as T cell proliferation and secretion of lymphokines, e.g. IL-2.

Alternatively, immunization of HLA transgenic mice can be used to determine immunogenicity of peptide epitopes. Several transgenic mouse models including mice with human A2.1, A11, and B7 alleles have been characterized and others (e.g., transgenic mice for HLA-A1 and A24) are being developed. HLA-DR1 and HLA-DR3 mouse models have also been developed. Additional transgenic mouse models with other HLA alleles may be generated as necessary. Mice may be immunized with peptides emulsified in Incomplete Freund's Adjuvant and the resulting T cells tested for their capacity to recognize peptide-pulsed target cells and target cells transfected with appropriate genes. CTL responses may be analyzed using cytotoxicity assays described above. Similarly, HTL responses may be analyzed using such assays as T cell proliferation or secretion of lymphokines.

Immunogenic peptide epitopes are set out in Table XXIII.

### IV.J. USE OF PEPTIDE EPITOPES AS DIAGNOSTIC AGENTS AND FOR EVALUATING IMMUNE RESPONSES

HLA class I and class II binding peptides as described herein can be used, in one embodiment of the invention, as reagents to evaluate an immune response. The immune response to be evaluated may be induced by using as an immunogen any agent that would potentially result in the production of antigen-specific CTLs or HTLs to the peptide epitope(s) to be employed as the reagent. The peptide reagent is not used as the immunogen.

For example, a peptide of the invention may be used in a tetramer staining assay to assess peripheral blood mononuclear cells for the presence of antigen-specific CTLs following exposure to a pathogen or immunogen. The HLA-tetrameric complex is used to directly visualize antigen-specific CTLs (see, e.g., Ogg et al. Science 279:2103-2106, 1998; and Altman et al. Science 174:94-96, 1996) and determine the frequency of the antigen-specific CTL population in a sample of peripheral blood mononuclear cells. A tetramer reagent using a peptide of the invention may be generated as follows: A peptide that binds to an allele-specific HLA molecules, or supertype molecules, is refolded in the presence of the corresponding HLA heavy chain and β₂-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells may then be identified, for example, by flow cytometry. Such an analysis may be used for diagnostic or prognostic purposes.

Peptides of the invention may also be used as reagents to evaluate immune recall responses. (see, e.g., Bertoni et al. J. Clin. Invest. 100:503-513, 1997 and Penna et al. J. Exp. Med. 174:1565-1570, 1991.) For example, patient PBC samples from individuals with acute hepatitis B or who have recently recovered from acute hepatitis B may be analyzed for the presence of HBV antigen-specific CTLs using HBV-specific peptides. A blood sample containing mononuclear cells may be evaluated by cultivating the PBCs and stimulating the cells with a peptide of the invention. After an appropriate cultivation period, the expanded cell population may be analyzed for cytotoxic activity.

The peptides may also be used as reagents to evaluate the efficacy of a vaccine. PBMCs obtained from a patient vaccinated with an immunogen may be analyzed using, for example, either of the methods described above. A patient is HLA typed, and appropriate peptide reagents that recognize allele-specific molecules present in that patient may be selected for the analysis. The immunogenicity of the vaccine will be indicated by the presence of HBV epitope-specific CTLs in the PBMC sample.

The peptides of the invention may also be used to make antibodies using techniques well known in the art (see, e.g., CURRENT PROTOCOLS IN IMMUNOLOGY, Wiley/Greene, NY; and Antibodies A Laboratory Manual, Harlow and Lane, Cold Spring Harbor Laboratory Press, 1989). Such antibodies may be useful as reagents to diagnose HBV infection.

### IV.K. VACCINE COMPOSITIONS

Vaccines that contain an immunogenically effective amount of one or more peptides as described herein are a further embodiment of the invention. Once appropriately immunogenic epitopes have been defined, they can be sorted and delivered by various means, herein referred to as “vaccine” compositions. Such vaccine compositions can include, for example, lipopeptides (Vitiello, A. et al., J. Clin. Invest. 95:341, 1995), peptides compositions encapsulated in poly(DL-lactide-co-glycolide) (PLG) microspheres (see, e.g., Eldridge, et al. Molec. Immunol. 28:287-294, 1991: Alonso et al. Vaccine 12:299-306, 1994; Jones et al. Vaccine 13:675-681, 1995), peptide compositions encapsulated in immune stimulating complexes (ISCOMS) (see, e.g., Takahashi et al. Nature 344:873-875, 1990; Hu et al. Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (Reddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

Furthermore, vaccines in accordance with the invention encompass compositions of one or more of the claimed peptide(s) that can be introduced into a host, including humans, linked to its own carrier, or as a homopolymer or heteropolymer of active peptide units. Such a polymer has the advantage of increased immunological reaction and, where different peptides are used to make up the polymer, the additional ability to induce antibodies and/or CTLs that react with different antigenic determinants of the pathogenic organism or tumor-related peptide targetted for an immune response.

Furthermore, useful carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, or alum are examples of materials well known in the art. Additionally, as disclosed herein, CTL responses can be primed by conjugating peptides of the invention to lipids, such as tripalmitoyl-S-glycerylcysteinlyseryl-serine (P₃CSS).

As disclosed in greater detail herein, upon immunization with a peptide composition in accordance with the invention, via injection, aerosol, oral, transdermal, transmucosal, intrapleural, intrathecal, or other suitable routes, the immune system of the host responds to the vaccine by producing large amounts of CTLs specific for the desired antigen. Consequently, the host becomes at least partially immune to later infection, or at least partially resistant to developing an ongoing chronic infection, or derives at least some therapeutic benefit when the antigen was tumor-associated.

In some instances it may be desirable to combine the class I peptide vaccines of the invention with vaccines which induce or facilitate neutralizing antibody responses to the target antigen of interest, particularly to viral envelope antigens. A preferred embodiment of such a composition comprises class I and class II epitopes in accordance with the invention. An alternative embodiment of such a composition comprises a class I and/or class II epitope in accordance with the invention, along with a PADRE™ (Epimmune, San Diego, Calif.) molecule (described in the related U.S. Ser. No. 08/485,218, which is a CIP of U.S. Ser. No. 08/305,871, now U.S. Pat. No. 5,736,142, which is a CIP of abandoned application U.S. Ser. No. 08/121,101.) Furthermore, any of these embodiments can be administered as a nucleic acid mediated modality.

For therapeutic or immunization purposes, the peptides of the invention can also be expressed by viral or bacterial vectors. Examples of expression vectors include attenuated viral hosts, such as vaccinia or fowlpox. This approach involves the use of vaccinia virus as a vector to express nucleotide sequences that encode the peptides of the invention. Upon introduction into an acutely or chronically infected host or into a non-infected host, the recombinant vaccinia virus expresses the immunogenic peptide, and thereby elicits a host CTL and/or HTL response. Vaccinia vectors and methods useful in immunization protocols are described in, e.g., U.S. Pat. No. 4,722,848. Another vector is BCG (Bacille Calmette Guerin). BCG vectors are described in Stover, et al. Nature 351:456-460 (1991). A wide variety of other vectors useful for therapeutic administration or immunization of the peptides of the invention, e.g. adeno and adeno-associated virus vectors, retroviral vectors, Salmonella typhi vectors, detoxified anthrax toxin vectors, and the like, will be apparent to those skilled in the art from the description herein.

Antigenic peptides are used to elicit a CTL and/or HTL response ex vivo, as well. The resulting CTL or HTL cells, can be used to treat chronic infections, or tumors in patients that do not respond to other conventional forms of therapy, or will not respond to a therapeutic vaccine peptide or nucleic acid in accordance with the invention. Ex vivo CTL or HTL responses to a particular pathogen (infectious agent or tumor antigen) are induced by incubating in tissue culture the patient's CTL or HTL precursor cells together with a source of antigen-presenting cells (APC), such as dendritic cells, and the appropriate immunogenic peptide. After an appropriate incubation time (typically about 14 weeks), in which the precursor cells are activated, mature and expand into effector cells, the cells are infused back into the patient, where they will destroy (CTL) or facilitate destruction (HTL) of their specific target cell (an infected cell or a tumor cell). Transfected dendritic cells may also be used as antigen presenting cells. Alternatively, dendritic cells are transfected, e.g., with a minigene construct in accordance with the invention, in order to elicit immune responses. Minigenes will be discussed in greater detail in a following section.

DNA or RNA encoding one or more of the peptides of the invention can also be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) delivery.

Preferably, the following principles are utilized when selecting an array of epitopes for inclusion in a polyepitopic composition, or for selecting epitopes to be included in a vaccine composition and/or to be encoded by a minigene. It is preferred that each of the following principles are balanced in order to make the selection.

1.) Epitopes are selected which, upon administration, mimic immune responses that have been observed to be correlated with HBV clearance. For HLA Class I this includes 3-4 epitopes that come from at least one antigen of HBV. In other words, it has been observed that in patients who spontaneously clear HBV, that they had generated an immune response to at least 3 epitopes on at least one HBV antigen. For HLA Class II a similar rationale is employed; again 3-4 epitopes are selected from at least one HBV antigen (see e.g., Rosenberg et al. Science 278:1447-1450).

2.) Epitopes are selected that have the requisite binding affinity established to be correlated with immunogenicity: for HLA Class I an IC₅₀ of 500 nM or less, or for Class II an IC₅₀ of 1000 nM or less.

3.) Sufficient supermotif bearing peptides, or a sufficient array of allele-specific motif bearing peptides, are selected to give broad population coverage. For example, it is preferable to have at least 80% population coverage. A Monte Carlo analysis, a statistical evaluation known in the art, can be employed to assess population coverage.

4.) When selecting epitopes from cancer-related antigens it is often preferred to select analogs. When selecting epitopes for infectious disease-related antigens it is often preferable to select native epitopes. Therefore, of particular relevance for infectious disease vaccines (but for cancer-related vaccines as well), are epitopes referred to as “nested epitopes.” Nested epitopes occur where at least two epitopes overlap in a given peptide sequence. A peptide comprising “transcendent nested epitopes” is a peptide that has both HLA class I and HLA class II epitopes in it.

When providing nested epitopes, it is preferable to provide a sequence that has the greatest number of epitopes per provided sequence. A limitation on this principle is to avoid providing a peptide that is any longer than the amino terminus of the amino terminal epitope and the carboxyl terminus of the carboxyl terminal epitope in the peptide. When providing a longer peptide sequence, such as a sequence comprising nested epitopes, it is important to screen the sequence in order to insure that it does not have pathological or other deleterious biological properties.

5.) When creating a minigene, as disclosed in greater detail in the following section, an objective is to generate the smallest peptide possible that encompasses the epitopes of interest. The principles employed are similar, if not the same as those employed when selecting a peptide comprising nested epitopes. Thus, upon determination of the nucleic acid sequence to be provided as a minigene, the peptide encoded thereby is analyzed to determine whether any “junctional epitopes” have been created. A junctional epitope is an actual binding epitope, as predicted, e.g., by motif analysis. Junctional epitopes are to be avoided because the recipient may generate an immune response to that epitope. Of particular concern is a junctional epitope that is a “dominant epitope.” A dominant epitope may lead to such a zealous response that immune responses to other epitopes are diminished or suppressed.

### IV.K.1. Minigene Vaccines

A growing body of experimental evidence demonstrates that a number of different approaches are available which allow simultaneous delivery of multiple epitopes. Nucleic acids encoding the peptides of the invention are a particularly useful embodiment of the invention. Epitopes for inclusion in a minigene are preferably selected according to the guidelines set forth in the previous section. A preferred means of administering nucleic acids encoding the peptides of the invention uses minigene constructs encoding a peptide comprising one or multiple epitopes of the invention. The use of multi-epitope minigenes is described below and in, e.g. An, L. and Whitton, J. L., J. Virol. 71:2292, 1997; Thomson, S. A. et al., J. Immunol. 157:822, 1996; Whitton, J. L. et al., J. Virol. 67:348, 1993; Hanke, R. et al., Vaccine 16:426, 1998. For example, a multi-epitope DNA plasmid encoding nine dominant HLA-A*0201 and A11-restricted epitopes derived from the polymerase, envelope, and core proteins of HBV and HIV, the PADRE™ universal helper T cell (HTL) epitope, and an endoplasmic reticulum-translocating signal sequence was engineered. Immunization of HLA transgenic mice with this plasmid construct result in strong CTL induction responses against the nine epitopes tested, similar to those observed with a lipopeptide of known immunogenicity in humans, and significantly greater than immunization in oil-based adjuvants. Moreover, the immunogenicity of DNA-encoded epitopes in vivo correlated with the in vitro responses of specific CTL lines-against target cells transfected with the DNA plasmid. Thus, these data show that the minigene served to both: 1.) generate a CTL response and 2.) that the induced CTLs recognized cells expressing the encoded epitopes.

For example, to create a DNA sequence encoding the selected epitopes (minigene) for expression in human cells, the amino acid sequences of the epitopes may be reverse translated. A human codon usage table can be used to guide the codon choice for each amino acid. These epitope-encoding DNA sequences may be directly adjoined, so that when translated, a continuous polypeptide sequence is created. To optimize expression and/or immunogenicity, additional elements can be incorporated into the minigene design. Examples of amino acid sequences that could be reverse translated and included in the minigene sequence include: HLA class I epitopes, HLA class II epitopes, a ubiquitination signal sequence, and/or an endoplasmic reticulum targeting signal. In addition, HLA presentation of CTL and HTL epitopes may be improved by including synthetic (e.g. poly-alanine) or naturally-occurring flanking sequences adjacent to the CTL or HTL epitopes; these larger peptides comprising the epitope(s) are within the scope of the invention.

The minigene sequence may be converted to DNA by assembling oligonucleotides that encode the plus and minus strands of the minigene. Overlapping oligonucleotides (30-100 bases long) may be synthesized, phosphorylated, purified and annealed under appropriate conditions using well known techniques. The ends of the oligonucleotides can be joined, for example, using T4 DNA ligase. This synthetic minigene, encoding the epitope polypeptide, can then be cloned into a desired expression vector.

Standard regulatory sequences well known to those of skill in the art are preferably included in the vector to ensure expression in the target cells. Several vector elements are desirable: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences and sequences for replication in mammalian cells may also be considered for increasing minigene expression.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

In addition, immunostimulatory sequences (ISSs or CpGs) appear to play a role in the immunogenicity of DNA vaccines. These sequences may be included in the vector, outside the minigene coding sequence, if desired to enhance immunogenicity.

In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF) or costimulatory molecules. Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving CTL induction. In contrast to HTL or CTL induction, specifically decreasing the immune response by co-expression of immunosuppressive molecules (e.g. TGF-β) may be beneficial in certain diseases).

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins supplied by QIAGEN, Inc. (Valencia, Calif.). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. To maximize the immunotherapeutic effects of minigene DNA vaccines, an alternative method for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

Target cell sensitization can be used as a functional assay for expression and HLA class I presentation of minigene-encoded CTL epitopes. For example, the plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (⁵¹Cr) labeled and used as target cells for epitope-specific CTL lines; cytolysis, detected by ⁵¹Cr release, indicates both production of, and HLA presentation of, minigene-encoded CTL epitopes.

In vivo immunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (IP) for lipid-complexed DNA). Twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr labeled target cells using standard techniques. Lysis of target cells sensitized by HLA loading of peptides corresponding to minigene-encoded epitopes demonstrates DNA vaccine function for in vivo induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment, DNA can be adhered to particles, such as gold particles.

IV.K.2. Combinations of CTL Peptides with Helper Peptides

The peptides of the present invention, or analogs thereof, which have immunostimulatory activity may be modified to provide desired attributes, such as improved serum half life, or to enhance immunogenicity.

For instance, the ability of the peptides to induce CTL activity can be enhanced by linking the peptide to a sequence which contains at least one epitope that is capable of inducing a T helper cell response. The use of T helper epitopes in conjunction with CTL epitopes to enhance immunogenicity is illustrated, for example, in U.S. Ser. No. 08/820,360, U.S. Ser. No. 08/197,484, U.S. Ser. No. 08/464,234, U.S. Ser. No. 08/464,496, U.S. Ser. No. 08/464,031, abandoned U.S. Ser. No. 08/464,433, and U.S. Ser. No. 08/461,603.

Particularly preferred CTL epitope/HTL epitope conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the CTL peptide may be linked to the T helper peptide without a spacer.

The CTL peptide epitope may be linked to the HTL peptide epitope either directly or via a spacer either at the amino or carboxy terminus of the CTL peptide. The amino terminus of either the CTL epitope or the HTL peptide may be acylated. The HTL peptide epitopes used in the invention can be modified in the same manner as CTL peptides. For instance, they may be modified to include D-amino acids or be conjugated to other molecules such as lipids, proteins, sugars and the like. Exemplary T helper peptides include tetanus toxoid 830-843, influenza 307-319, and malarial circumsporozoite 382-398 and 378-389.

In certain embodiments, the T helper peptide is one that is recognized by T helper cells present in the majority of the population. This can be accomplished by selecting amino acid sequences that bind to many, most, or all of the HLA class II molecules. These are known as “loosely HLA-restricted” or “promiscuous” T helper sequences. Examples of amino acid sequences that are promiscuous include sequences from antigens such as tetanus toxoid at positions 830-843 (QYIKANSKFIGITE), Plasmodium falciparum CS protein at positions 378-398 (DIEKKIAKMEKASSVFNVVNS), and Streptococcus 18 kD protein at positions 116 (GAVDSILGGVATYGAA). Other examples include peptides bearing a DR 1-4-7 supermotif, or either of the DR3 motifs.

Alternatively, it is possible to prepare synthetic peptides capable of stimulating T helper lymphocytes, in a loosely HLA-restricted fashion, using amino acid sequences not found in nature (see, e.g., PCT publication WO 95/07707). These synthetic compounds called Pan-DR-binding epitopes (e.g., PADRE™, Epimmune, Inc., San Diego, Calif.) are designed on the basis of their binding activity to most HLA-DR (human HLA class II) molecules. For instance, a pan-DR-binding epitope peptide having the formula: aKXVWANTLKAAa, where “X” is either cyclohexylalanine, phenylalanine, or tyrosine, and a is either D-alanine or L-alanine, has been found to bind to most HLA-DR alleles, and to stimulate the response of T helper lymphocytes from most individuals, regardless of their HLA type.

HTL peptide epitopes can also be modified to alter their biological properties. For example, peptides comprising HTL epitopes can contain D-amino acids to increase their resistance to proteases and thus extend their serum half-life. Also, the epitope peptides of the invention can be conjugated to other molecules such as lipids, proteins or sugars, or any other synthetic compounds, to increase their biological activity. Specifically, the T helper peptide can be conjugated to one or more palmitic acid chains at either the amino or carboxyl termini.

In some embodiments it may be desirable to include in the pharmaceutical compositions of the invention at least one component which primes cytotoxic T lymphocytes. Lipids have been identified as agents capable of priming CTL in vivo against viral antigens. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. In a preferred embodiment, a particularly effective immunogenic comprises palmitic acid attached to ε- and α-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide. (See, Deres, et al., Nature 342:561, 1989). Peptides of the invention can be coupled to P₃CSS, for example, and the lipopeptide administered to an individual to specifically prime a CTL response to the target antigen. Moreover, because the induction of neutralizing antibodies can also be primed with P₃CSS-conjugated epitopes, two such compositions can be combined to more effectively elicit both humoral and cell-mediated responses to infection.

In addition, additional amino acids can be added to the termini of a peptide to provide for ease of linking peptides one to another, for coupling to a carrier support, or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide, particularly class I peptides. However, it is to be noted that modification at the carboxyl terminus of a CTL epitope may, in some cases, alter binding characteristics of the peptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxyl amidation, e.g., ammonia, methylamine, etc. In some instances these modifications may provide sites for linking to a support or other molecule.

### IV.L. ADMINISTRATION OF VACCINES FOR THERAPEUTIC OR PROPHYLACTIC PURPOSES

The peptides of the present invention and pharmaceutical and vaccine compositions of the invention are useful for administration to mammals, particularly humans, to treat and/or prevent HBV infection. Vaccine compositions containing the peptides of the invention are administered to a patient susceptible to or otherwise at risk for HBV infection to elicit an immune response against HBV antigens and thus enhance the patient's own immune response capabilities. In therapeutic applications, compositions are administered to a patient in an amount sufficient to elicit an effective CTL response to the virus antigen and to cure or at least partially arrest or slow symptoms and/or complications. An amount adequate to accomplish this is defined as “therapeutically effective dose.” Amounts effective for this use will depend on, e.g., the particular composition administered, the manner of administration, the stage and severity of the disease being treated, the weight and general state of health of the patient, and the judgment of the prescribing physician.

The vaccine compositions of the invention may also be used purely as prophylactic agents. Vaccine compositions containing the peptide epitopes of the invention are administered to a patient susceptible to, or otherwise at risk for, HBV infection to elicit an immune response against HBV antigens and thus enhance the patient's own immune response capabilities following exposure to HBV. Generally the dosage for an initial prophylactic immunization generally occurs in a unit dosage range where the lower value is about 1, 5, 50, 500, or 1000 μg and the higher value is about 10,000; 20,000; 30,000; or 50,000 μg. Dosage values for a human typically range from about 500 μg to about 50,000 μg per 70 kilogram patient. This is followed by boosting dosages of between about 1.0 μg to about 50,000 μg of peptide administered at defined intervals from about four weeks to six months after the initial administration of vaccine. The immunogenicity of the vaccine may be assessed by measuring the specific activity of CTL and/or HTL-obtained from a sample of the patient's blood.

As noted above, peptides comprising CTL or HTL epitopes of the invention induce immune responses when presented by HLA molecules and contacted with a CTL or HTL specific for an epitope comprised by the peptide. The manner in which the peptide is contacted with the CTL or HTL is not critical to the invention. For instance, the peptide can be contacted with the CTL or HTL either in vivo or in vitro. If the contacting occurs in vivo, the peptide itself can be administered to the patient, or other vehicles, e.g., DNA vectors encoding one or more peptides, viral vectors encoding the peptide(s), liposomes and the like, can be used, as described herein.

For pharmaceutical compositions, the immunogenic peptides of the invention, or DNA encoding them, are generally administered to an individual already infected with HBV. The peptides or DNA encoding them can be administered individually or as fusions of one or more peptide sequences. Those in the incubation phase or the acute phase of infection can be treated with the immunogenic peptides separately or in conjunction with other treatments, as appropriate.

For therapeutic use, administration should generally begin at the first diagnosis of HBV infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection, the compositions are particularly useful in methods for preventing the evolution from acute to chronic infection. Where susceptible individuals are identified prior to or during infection, the composition can be targeted to them, thus minimizing the need for administration to a larger population.

The peptide or other compositions used for the treatment or prophylaxis of HBV infection can be used, e.g., in persons who have not manifested symptoms of disease but who act as a disease vector. In this context, it is generally important to provide an amount of the peptide epitope delivered by a mode of administration sufficient to effectively stimulate a cytotoxic T cell response; compositions which stimulate helper T cell responses can also be given in accordance with this embodiment of the invention.

The dosage for an initial immunization (i.e., therapeutic or prophylactic administration) generally occurs in a unit dosage range where the lower value is about 1, 5, 50, 500, or 1000 μg and the higher value is about 10,000; 20,000; 30,000; or 50,000 μg. Dosage values for a human typically range from about 500 μg to about 50,000 μg per 70 kilogram patient. Boosting dosages of between about 1.0 μg to about 50000 μg of peptide pursuant to a boosting regimen over weeks to months may be administered depending upon the patient's response and condition as determined by measuring the specific activity of CTL and/or HTL obtained from the patient's blood. The peptides and compositions of the present invention may be employed in serious disease states, that is, life-threatening or potentially life threatening situations. In such cases, as a result of the minimal amounts of extraneous substances and the relative nontoxic nature of the peptides in preferred compositions of the invention, it is possible and may be felt desirable by the treating physician to administer substantial excesses of these peptide compositions relative to these stated dosage amounts.

Thus, for treatment of chronic infection, a representative dose is in the range disclosed above, namely where the lower value is about 1, 5, 50, 500, or 1000 μg and the higher value is about 10,000; 20,000; 30,000; or 50,000 μg, preferably from about 500 μg to about 50,000 μg per 70 kilogram patient. Initial doses followed by boosting doses at established intervals, e.g., from four weeks to six months, may be required, possibly for a prolonged period of time to effectively immunize an individual. In the case of chronic infection, administration should continue until at least clinical symptoms or laboratory tests indicate that the viral infection has been eliminated or substantially abated and for a period thereafter. The dosages, routes of administration, and dose schedules are adjusted in accordance with methodologies known in the art.

The pharmaceutical compositions for therapeutic treatment are intended for parenteral, topical, oral, intrathecal, or local administration. Preferably, the pharmaceutical compositions are administered parentally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the peptide composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, 17th Edition, A. Gennaro, Editor, Mack Publising Co., Easton, Pa., 1985)

The peptides of the invention may also be administered via liposomes, which serve to target the peptides to a particular tissue, such as lymphoid tissue, or to target selectively to infected cells, as well as to increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the peptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

### IV.M. KITS

The peptide and nucleic acid compositions of this invention can be provided in kit form together with instructions for vaccine administration. Typically the kit would include desired peptide compositions in a container, preferably in unit dosage form and instructions for administration. An alternative kit would include a minigene construct with desired nucleic acids of the invention in a container, preferably in unit dosage form together with instruction for administration. Lymphokines such as IL-2 or IL-12 may also be included in the kit. Other kit components that may also be desirable include, for example, a sterile syringe, booster dosages, and other desired excipients.

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments in accordance with the invention.

### V. EXAMPLES

### Example 1

### HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing.

Epstein-Barr virus (EBV)-transformed homozygous cell lines, fibroblasts, CIR, or 721.22 transfectants were used as sources of HLA class I molecules. These cells were maintained in vitro by culture in RPMI 1640 medium supplemented with 2 mM L-glutamine (GIBCO, Grand Island, N.Y.), 50 μM 2-ME, 100 μg/ml of streptomycin, 100 U/ml of penicillin (Irvine Scientific) and 10% heat-inactivated FCS (Irvine Scientific, Santa Ana, Calif.). Cells were grown in 225-cm² tissue culture flasks or, for large-scale cultures, in roller bottle apparatuses. The specific cell lines routinely used for purification of MHC class I and class II molecules are listed in Table XXIV.

Cell lysates were prepared and HLA molecules purified in accordance with disclosed protocols (Sidney et al., Current Protocols in Immunology 18.3.1 (1998); Sidney, et al., gJ. Immunol. 154:247 (1995); Sette, et al., Mol. Immunol. 31:813 (1994)). Briefly, cells were lysed at a concentration of 10⁸ cells/ml in 50 mM Tris-HCl, pH 8.5, containing 1% Nonidet P-40 (Fluka Biochemika, Buchs, Switzerland), 150 mM NaCl, 5 mM EDTA, and 2 mM PMSF. Lysates were cleared of debris and nuclei by centrifugation at 15,000×g for 30 min.

HLA molecules were purified from lysates by affinity chromatography. Lysates prepared as above were passed twice through two pre-columns of inactivated Sepharose CL4-B and protein A-Sepharose. Next, the lysate was passed over a column of Sepharose CL-4B beads coupled to an appropriate antibody. The antibodies used for the extraction of HLA from cell lysates are listed in Table XXV. The anti-HLA column was then washed with 10-column volumes of 10 mM Tris-HCL, pH 8.0, in 1% NP-40, PBS, 2-column volumes of PBS, and 2-column volumes of PBS containing 0.4% n-octylglucoside. Finally, MHC molecules were eluted with 50 mM diethylamine in 0.15M NaCl containing 0.4% n-octylglucoside, pH 11.5. A 1/25 volume of 2.0M Tris, pH 6.8, was added to the eluate to reduce the pH to ˜8.0. Eluates were then be concentrated by centrifugation in Centriprep 30 concentrators at 2000 rpm (Amicon, Beverly, Mass.). Protein content was evaluated by a BCA protein assay (Pierce Chemical Co., Rockford, Ill.) and confirmed by SDS-PAGE.

A detailed description of the protocol utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) were incubated with various unlabeled peptide inhibitors and 1-10 nM ¹²⁵I-radiolabeled probe peptides for 48 h in PBS containing 0.05% Nonidet P-40 (NP40) (or 20% w/v digitonin for H-2 IA assays) in the presence of a protease inhibitor cocktail. The final concentrations of protease inhibitors (each from CalBioChem, La Jolla, Calif.) were 1 mM PMSF, 1.3 nM 1.10 phenanthroline, 73 μM pepstatin A, 8 mM EDTA, 6 mM N-ethylmaleimide (for Class II assays), and 200 μM N alpha-p-tosyl-L-lysine chloromethyl ketone (TLCK). All assays were performed at pH 7.0 with the exception of DRB1*0301, which was performed at pH 4.5, and DRB1*1601 (DR2w21·1) and DRB4*0101 (DRw53), which were performed at pH 5.0. pH was adjusted as described elsewhere (see Sidney et al., in Current Protocols in Immunology, Margulies, Ed., John Wiley & Sons, New York, Section 18.3, 1998).

Following incubation, MHC-peptide complexes were separated from free peptide by gel filtration on 7.8 mm×15 cm TSK200 columns (TosoHaas 16215, Montgomeryville, Pa.), eluted at 1.2 mls/min with PBS pH 6.5 containing 0.5% NP40 and 0.1% NaN₃. Because the large size of the radiolabeled peptide used for the DRB1*1501 (DR2w2·1) assay makes separation of bound from unbound peaks more difficult under these conditions, all DRB1*1501 (DR2w2·1) assays were performed using a 7.8 mm×30 cm TSK2000 column eluted at 0.6 mls/min. The eluate from the TSK columns was passed through a Beckman 170 radioisotope detector, and radioactivity was plotted and integrated using a Hewlett-Packard 3396A integrator, and the fraction of peptide bound was determined.

Radiolabeled peptides were iodinated using the chloramine-T method. The specific radiolabeled probe peptide utilized in each assay, and its assay specific IC₅₀ nM, is summarized in Table XXIV. Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC50≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values are compiled. These values can subsequently be converted back into IC₅₀ nM values by dividing the IC₅₀ nM of the positive controls for inhibition by the relative binding of the peptide of interest. This method of data compilation has proven to be the most accurate and consistent for comparing peptides that have been tested on different days, or with different lots of purified MHC.

Because the antibody used for HLA-DR puriflcafion (LB3.1) is α-chain specific, β₁ molecules are not separated from β₃ (and/or β₄ and β₅) molecules. The β₁ specificity of the binding assay is obvious in the cases of DRB1*0101 (DR1), DRB1*0802 (DR8w2), and DRB1*0803 (DR8w3), where no P3 is expressed. It has also been demonstrated for DRB1*0801 (DR3) and DRB3*0101 (DR52a), DRB1*0401 (DR4w4), DRB1*0404 (DR4w14), DRB1*0405 (DR4w15), DRB1*1101 (DR5), DRB1*1201 (DR5w12), DRB1*1302 (DR6w19) and DRB1*0701 (DR7). The problem of β chain specificity for DRB1*1501 (DR2w2·1), DRB5*0101 (DR2w2·2), DRB1*1601 (DR2w21·1), DRB5*0201 (DR²w21·3), and DRB4*0101 (DRw53) assays is circumvented by the use of fibroblasts. Development and validation of assays with regard to DRβ molecule specificity have been described previously (see, e.g., Southwood et al., J. Immunol. 160:3363-3373, 1998).

Binding assays as outlined above may be used to analyze supermotif and/or motif-bearing epitopes as, for example, described in Example 2.

### Example 2

### Identification of Conserved HLA Supermotif CTL Candidate Epitopes

Vaccine compositions of the invention may include multiple epitopes that comprise multiple HLA supermotifs or motifs to achieve broad population coverage. This example illustrates the identification of supermotif-bearing epitopes for the inclusion in such a vaccine composition. Calculation of population coverage was performed using the strategy described below. Epitopes were then selected to bear an HLA-A2, -A3, or -B7 supermotif or an HLA-A1 or -A24 motif.

Computer Searches and Algorithms for Identification of Supermotif and/or Motif-Bearing Epitopes

Computer searches for epitopes bearing HLA Class I or Class II supermotifs or motifs were performed as follows. All translated HBV isolate sequences were analyzed using a text string search software program, e.g., MotifSearch 1.4 (D. Brown, San Diego) to identify potential peptide sequences containing appropriate HLA binding motifs; alternative programs are readily produced in accordance with information in the art in view of the motif/supermotif disclosure herein. Furthermore, such calculations can be made mentally. Identified A2-, A3-, and DR-supermotif sequences were scored using polynomial algorithms to predict their capacity to bind to specific HLA-Class I or Class II molecules. These polynomial algorithms take into account both extended and refined motifs (that is, to account for the impact of different amino acids at different positions), and are essentially based on the premise that the overall affinity (or AG) of peptide-HLA molecule interactions can be approximated as a linear polynomial function of the type:

“ΔG”=a₁ᵢ×a₂ᵢ×a₃ᵢ . . . aₙᵢ

where aⱼᵢ is a coefficient which represents the effect of the presence of a given amino acid (j) at a given position (i) along the sequence of a peptide of n amino acids. The crucial assumption of this method is that the effects at each position are essentially independent of each other (i.e., independent binding of individual side-chains). When residue j occurs at position i in the peptide, it is assumed to contribute a constant amount jᵢ to the free energy of binding of the peptide irrespective of the sequence of the rest of the peptide. This assumption is justified by studies from our laboratories that demonstrated that peptides are bound to MHC and recognized by T cells in essentially an extended conformation (data omitted herein).

The method of derivation of specific algorithm coefficients has been described in Gulukota et al., J; Mol. Biol. 267:1258-126, 1997; (see also Sidney et al., Human Immunol. 45:79-93, 1996; and Southwood et al., J. Immunol. 160:3363-3373, 1998). Briefly, for all i positions, anchor and non-anchor alike, the geometric mean of the average relative binding (ARB) of all peptides carrying j is calculated relative to the remainder of the group, and used as the estimate of jᵢ. For Class II peptides, if multiple alignments are possible, only the highest scoring alignment is utilized, following an iterative procedure. To calculate an algorithm score of a given peptide in a test set, the ARB values corresponding to the sequence of the peptide are multiplied. If this product exceeds a chosen threshold, the peptide is predicted to bind. Appropriate thresholds are chosen as a function of the degree of stringency of prediction desired.

### Selection of HLA-A2 Supertype Cross-Reactive Peptides

Complete sequences from 20 HBV isolates were aligned, then scanned, utilizing a customized computer program, to identify conserved 9- and 10-mer sequences containing the HLA-A2-supertype main anchor specificity.

A total of 150 conserved and motif-positive sequences were identified. These peptides were then evaluated for the presence of A*0201 preferred secondary anchor residues using an A*0201-specific polynomial algorithm. A total of 85 conserved, motif-positive sequences were selected and synthesized.

These 85 conserved, motif-containing 9- and 10-mer peptides were then tested for their capacity to bind purified HLA-A*0201 molecules in vitro. Thirty-four peptides were found to be good A*0201 binders (IC₅₀≦500 nM); 15 were high binders (IC₅₀≦50 nM) and 19 were intermediate binders (IC₅₀ of 50-500 nM) (Table XXVI).

In the course of independent analyses, 25 conserved, HBV-derived, 8 or 11-mer sequences with appropriate A2-supertype main anchors were also synthesized and tested for A*0201 binding. One peptide, HBV env 259 11-mer (peptide 1147.14), bound A*0201 with an IC₅₀ of 500 nM, or less, and has been included in Table XXVI. Also shown in Table XXVI is an analog peptide, representing a single substitution of the HBV pol 538 9-mer peptide, which binds A*0201 with an IC₅₀ of 5.1 nM (see below).

Thirty of the 36 A*0201 binders were subsequently tested for the capacity to bind to additional A2-supertype alleles (A*0202, A*0203, A*0206, and A*6802). As shown in Table XXVI, 15/30 (50%) peptides were found to be A2-supertype cross-reactive binders, binding at least 3 of the 5 A2-supertype alleles tested. These 15 peptides were selected for further analysis.

### Selection of HLA-A3 Supermotif-Bearing Epitopes

The sequences from the same 20 isolates were also examined for the presence of conserved peptides with the HLA-A3-supermotif primary anchors. A total of 80 conserved 9- or 10-mer motif-containing sequences were identified. Further analysis using the A03 and A11 algorithms identified 40 sequences which scored high in either or both algorithms. Thirty-six of the corresponding peptides were synthesized and tested for binding to HLA-A*03 and HLA-A*11, the two most prevalent A3-supertype alleles. Twenty-three peptides were identified which bound A3 and/or A11 with affinities or IC₅₀ values of ≦500 nM (Table XXVII).

In the course of an independent series of studies 30 HBV-derived 8-mer, and 24 11-mer sequences, conserved in 75% or more of the isolates, were selected and tested for A*03 and A*11 binding. Four 8-mers and 9 11-mers were found to bind either or both alleles (Table XXVII). Finally, four 9-mer, and one 10-mer, conserved HBV-derived peptides not selected using the search criteria outlined above, but which have been shown to bind A*03 and/or A*11, have been identified, and are included in Table XXVII. Two of these peptides contain non-canonical anchors (peptides 20.0131, and 20.0130), and the other 3 are algorithm negative (peptides 1142.05, 1099.03, and 1090.15).

Thirty-eight of the 41 pepfides binding A*03 and/or A*11 were subsequently tested for binding crossreactivity to the other common A3-supertype alleles (A*3101, A*3301, and A*6801). It was found that 17 of these peptides were A3-supertype cross-reactive, binding at least 3 of the 5 A3-supertype alleles tested (Table XXVII).

### Selection of HLA-B 7 Supermotif Bearing Epitopes

When the same 20 isolates were also analyzed for the presence of conserved 9- or 10-mer peptides with the HLA-B7-supermotif, 46 sequences were identified. Thirty-four of the corresponding peptides were synthesized and tested for binding to HLA-B*0702, the most common B7-supertype allele. Nine peptides bound B*0702 with an IC₅₀ value of ≦500 nM (Table XXVIII). These 9 peptides were then tested for binding to other common B7-supertype alleles (B*3501, B*51, B*5301, and B*5401). Five of the 9 B*0702 binders were capable of binding to 3 or more of the 5 B7-supertype alleles tested.

In separate studies investigating the secondary anchor requirements of B7-supertype alleles, all available peptides with the B7-supermotif were tested for binding to all B7 supertype alleles. As a result, all 34 peptides described above were also tested for binding to other B7-supertype alleles. These experiments identified an additional 10 peptides which bound at least one B7-supertype allele with an IC₅₀ value≦500 nM, including 2 peptides which bound 3 or more alleles. These 10 peptides are also shown in Table XXVIII.

Because of the low numbers of conserved B7-supertype degenerate HBV-derived 9- and 10-mer peptides, compared to the A2- and A3-supertypes, the 20 isolates were again examined to identify conserved, motif-containing 8- and 11-mers. This re-scan identified 51 peptides. Thirty-one of these were synthesized and tested for binding to each of the 5 most common B7-supertype alleles. Nineteen 8- and 11-mer peptides bound with high or intermediate affinity to at least one B7-supertype allele (IC₅₀≦500 nM) (Table XXVIII). Two peptides were degenerate binders, binding 3 of the 5 alleles tested.

In summary, a total of 9 HBV-derived peptides, conserved in 75% or more of the isolates analyzed, have been identified which are degenerate B7-supertype binders (Table XXV1H).

### Selection of A1 and A24 Motif-Bearing Epitopes

To further increase population coverage, HLA-A1 and -A24 epitopes have been incorporated into the present analysis. A1 is, on average, present in 12%, and A24 is present in approximately 29%, of the population across five different major ethnic groups (Caucasian, North American Black, Chinese, Japanese, and Hispanic). Combined, these alleles would be represented with an average frequency of 39% in these same populations. The total coverage across the major ethnicities when A1 and A24 are combined with the coverage of the A2-, A3- and B7-supertype alleles is >95.4%; by comparison, coverage by combing the A2-, A3-, and B7-supertypes is 86.2%.

Systematic analyses of HBV for A1 and A24 binders have yet to be completed. However, in the course of independent studies, 15 conserved HBV-derived peptides have been identified that bind A*0101 with IC50 less than 500 nM (Table XXIX); 7 of these bind with IC₅₀ less than 100 nM. In a similar context, 14 conserved A*2402 binding HBV-derived peptides have also been identified, 6 of which bind A*2402 with IC₅₀ less than 100 nM (Table XXIX).

### Example 3

### Confirmation of Immunogenicity

### Evaluation of A*0201 Immunogenicity

The immunogenicity analysis of the 15 HBV-derived HLA-A2 supertype cross-reactive peptides identified above is summarized in Table XXX. Peptides were screened for immunogenicity in at least one of three systems. Peptides were screened for the induction of primary antigen-specific CTL in vitro using human PBMC (Wentworth et al., Molec. Immunol. 32:603, 1995); this data is indicated as “primary CTL” in Table XXX.

The protocol for in vitro induction of primary antigen-specific CTL from human PBMC has been described by Wentworth et al (Wentworth et al., Molec. Immunol. 32:603, 1995). PBMC from normal donors which had been enriched for CD8+ T cells were incubated with peptide loaded antigen-presenting cells (SAC-I activated PBMCs) in the presence of IL-7. After seven days cultures were restimulated using irradiated autologous adherent cells pulsed with peptide and then tested for cytotoxic activity seven days later.

In addition, HLA transgenic mice were used to evaluate peptide immunogenicity; this data is indicated as “transgenic CTL” in Table XXX. Previous studies have shown that CTL induced in A*0201/Kb transgenic mice exhibit specificity similar to CTL induced in humans (Vitiello et al., J. Exp. Med. 173:1007, 1991; Wentworth et al., Eur. J. Immunol. 26:97, 1996).

CTL induction in transgenic mice following peptide immunization has been described by Vitiello et al. (Vitiello et al., J. Exp. Med. 173:1007, 1991) and Alexander et al. (Alexander et al., J. Immunol. 159:4753, 1997). Briefly, synthetic peptides (50 μg/mouse) and the helper epitope HBV core 128 (140 μg/mouse) were emulsified in incomplete Freund's adjuvant (IFA) and injected subcutaneously at the base of the tail. Eleven days after injection, splenocytes were incubated in the presence of peptide-loaded syngenic LPS blasts. After six days cultures were assayed for cytotoxic activity using peptide-pulsed targets.

Peptides were also tested for the ability to stimulate recall CTL responses in acutely infected HBV patients (Bertoni et al., J. Clin. Invest. 100:503, 1997; Rehermann et al., J. Clin. Invest. 97:1655-1665, 1996; Nayersina et al., J. Immunol. 150:4659, 1993); these data are indicated as “patient CTL” in Table XXX. Patient immunogenicity data is particularly informative as it indicates that a peptide is recognized during the course of a natural infection. These data demonstrate that a peptide is processed and presented in human cells that would represent the targets for CTL. Moreover, this data is especially relevant for vaccine design as the induction of CTL responses in patients has been correlated to the resolution of infection.

For the evaluation of recall CTL responses, screening was carried out as described by Bertoni et al. (Bertoni et al., J. Clin. Invest. 100:503, 1997). Briefly, PBMC from patients acutely infected with HBV were cultured in the presence of 10 μg/ml of synthetic peptide. After seven days, the cultures were restimulated with peptide. The cultures were assayed for cytotoxic activity on day 14 using target cells pulsed with peptide.

Of the 15 A2 supertype binding peptides, 11 were found to be immunogenic in at least one of the systems utilized. Five of the 11 peptides had previously been identified in the patients with acute HBV (Bertoni et al., J. Clin. Invest. 100:503, 1997). Five additional degenerate peptides (1069.06, 1090.77, 1147.14, 927.42 and 927.46) induced CTL responses in HLA-A*0201 transgenic mice. The 11 immunogenic supertype cross-reactive peptides are encoded by three HBV antigens; core, envelope and polymerase.

This set of 11 immunogenic A2-supermotif-bearing epitopes includes one analog peptide, 1090.77. The wild type peptide, 1090.14, from which this analog is derived is A2-supertype non-cross-reactive, but has been shown to be recognized in recall CTL responses from acute HBV patients, and to be immunogenic in HLA-A*0201 transgenic mice as well as primary human cultures (Table XXX). Further studies addressing the cross recognition of the wild type peptide 1090.14 and the 1090.77 analog are described in detail below.

In the course of independent analyses, 14 of the non-cross-reactive peptides shown in Table XXXb, including 1090.14, were found to be immunogenic in at least one system. Five peptides of these peptides were recognized in patients; 4 peptides induced CTL in transgenic mice.

In conclusion, 11 A2-supertype cross-reactive peptides have been identified that are capable of exhibiting immunogenicity in at least one of the three systems examined.

Evaluation of A*03/A11 immunogenicity

Seven of the 17 A3-supertype cross-reactive peptides have been evaluated for immunogenicity (Table XXXI). As described in the previous section, A3-supermotif-bearing peptides were screened using primary cultures, patient responses, or HLA-A11 transgenic mice (Alexander et al., J. Immunol. 159:4753, 1997). With the exception of peptide 1.0219, all of the conserved cross-reactive peptides listed in Table insert table XXXI were found to be immunogenic.

Additionally, a poorly conserved peptide (1150.51; 40% conserved) which exhibits cross-reactive supertype binding was found to be immunogenic in transgenic mice, and has been included in Table XXXI. Two other conserved, but non-cross-reactive, peptides have also been shown to be recognized in acutely infected HBV patients (Bertoni et al., J. Clin. Invest. 100:503, 1997). These epitiopes are shown in Table XXXI.

It is notable that for 7 of the 8 conserved immunogenic HBV-derived A3-supermotif-bearing epitopes, including all 6 of the cross-reactive peptides, positive data was obtained in patients. These epitopes are predominantly derived from the polymerase protein sequence, with only one epitope being derived from the core protein sequence. While a number of cross-reactive peptides have been identified in the X antigen (Table XXXI), to date these peptides have not been screened for immunogenicity.

In summary, 7 A3-supermotif-bearing, cross-reactive peptides have been identified that are recognized by CTL in acutely infected patients, or induce CTL in HLA-transgenic mice.

### Evaluation of B7 Immunogenicity

The immunogenicity studies involving the HBV-derived HLA-B7-supermotif-bearing, cross-reactive peptides is summarized in Table XXXII. HLA-B7 peptides were screened exclusively in human systems measuring responses in either primary cultures or acutely infected HBV patients. Of the 7 degenerate peptides screened, 4 were shown to be immunogenic. One non-crossreactive peptide (XRN<3), 1147.04, was also shown to be recognized in acutely infected HBV patients (Bertoni et al., J. Clin. Invest. 100:503, 1997; see Table XXXII).

In summary, 5 conserved HBV-derived B7-supermotif-bearing epitopes that are recognized in acutely infected HBV patients have been identified. These epitopes afford coverage of 4 different HBV antigens (core, envelope, polymerase and X).

### Example 4

### Implementation of the Extended Supermotif to Improve the Binding Capacity of Native Peptides by Creating Analogs

HLA motifs and supermotifs (comprising primary and/or secondary residues) are useful in preparing highly cross-reactive native peptides, as demonstrated herein. Moreover, the definition of HLA motifs and supermotifs also allows one to engineer highly cross-reactive epitopes by identifying residues within a native peptide sequence which can be analoged, or “fixed”, to confer upon a peptide certain characteristics, e.g., greater cross-reactivity within the group of HLA molecules that make-up the supertype, and/or greater binding affinity for some or all of those HLA molecules Examples of analog peptides that exhibit modulated binding affinity are provided.

### Analoging at Primary Anchor Residues

It has been shown that class I peptide ligands can be modified, or “fixed” to increase their binding affinity and/or degeneracy (Sidney et al., J. Immunol. 157:3480, 1996). These fixed peptides may also demonstrate increased immunogenicity and crossreactive recognition by T cells specific for the wild type epitope (Parkhurst et al., J. Immunol. 157:2539, 1996; Pogue et al., Proc. Natl. Acad. Sci. USA 92:8166, 1995). Specifically, the main anchors of A2 supertype peptides may be “fixed”, or analoged, to L or V (or M, if natural) at position 2, and V at the C-terminus. As indicated in Table XXVI, 9 of the 14 A2-supertype cross-reactive binding peptides are “fixable” by these criteria, as are 16 of the 21 non-cross-reactive binders. Ideal candidates for fixing would be peptides which bind at least 3 A2-supertype allele-specific molecules with IC₅₀≦5000 nM.

An example of the efficacy of this strategy to generate more broadly cross-reactive epitopes is provided by the case of peptide 1090.14 (Table XXVI). Previously, this peptide was shown to be highly immunogenic in each of the systems examined. However, it only exhibits binding to a single A2-supertype allele-specific molecule, A*0201. The non-crossreactive binding capacity of this epitope limits the population coverage and consequently the value of including this peptide in a candidate vaccine. In an effort to increase binding affinity and cross-reactivity the C-terminus of peptide 1090.14 was altered from ‘alanine’ to the A2-supermotif preferred residue ‘valine’. This change resulted in a dramatic (40-fold) increase in binding capacity for A*0201 (from 200 nM to 5.1 nM), but also produced a peptide capable of binding 3 other A2-supertype allele-specific molecules. (see peptide 1090.77, Table XXVI).

Studies with HLA-A*0201 transgenic mice have shown that the CTL response from mice immunized with the 1090.77 peptide recognize target cells loaded with either the naturally occurring peptide 1090.14 or the valine-substituted analog (i.e., 1090.77). In fact, the lysis effected by 1090.77 induced CTL was indistinguishable regardless whether the analog or the wild-type sequence was used to load the target cells (B. Livingston, unpublished data).

The relevance of these observations for the design of vaccine constructs is indicated by studies in which chronic HBV patients were treated with the potent viral replication inhibitor, lamivudine. Extended therapy with lamivudine resulted in the selection of drug-resistant strains of HBV that have a substitution of valine for methione at position 2 in the 1090.14 epitope, suggesting that epitope-based vaccines used in combination with lamivudine may need to have the ability to induce CTL responses that recognize both wild type and mutant sequences.

To demonstrate that cross-recognition is possible between the native peptide (1090.14), the analog peptide, and the lamivudine induced mutant M2 peptide, CTL were generated using the 1090.77 analog peptide. These CTL cultures were then stimulated with either the wild type peptide (1090.14), or the lamivudine induced mutant M2 peptide. The ability of these CTL to then lyse target cells loaded with either the wild type, or the lamivudine induced mutant peptide was then assayed. Target cells presenting either peptide were similarly lysed by either CTL culture (Table XXVI).

These studies demonstrate how analoging a peptide can result in dramatically increased HLA-A2 supertype degeneracy while still allowing cross-recognition between wildtype and mutant epitopes. More specifically, these results indicate that a vaccine utilizing the analog peptide 1090.77 should stimulate a response that will recognize both wild-type and lamivudine-resistant strains of HBV.

Similarly, analogs of HLA-A3 supermotif-bearing epitopes may also be generated. For example, peptides could be analogued to possess a preferred V at position 2, and R or K at the C-terminus. Twelve of the A3-supertype degenerate peptides identified in Table XXVII are candidates for main anchor fixing, as are 19 of the 24 non-cross-reactive binders.

Analog peptides are initially tested for binding to A*03 and A*11, and those that demonstrate equivalent, or improved, binding capacity relative to the parent peptide would then be tested for A3-supertype cross-reactivity. Analogs demonstrating improved cross-reactivity are then further evaluated for immunogenicity, as necessary.

Typically, it is more difficult to identify B7 supermotif-bearing epitopes. As in the cases of A2- and A3-supertype epitopes, a peptide analoguing strategy can be utilized to generate additional B7 supermotif-bearing epitopes with increased cross-reactive binding. In general, B7 supermotif-bearing peptides should be fixed to possess P in position 2, and I at their C-terminus.

Analogs representing primary anchor single amino acid residues substituted with I residues at the C-terminus of two different B7-like peptides (HBV env 313 and HBV pol 541) were synthesized and tested for their B7-supertype binding capacity. It was found that the I substitution had an overall positive effect on binding affinity and/or cross-reactivity in both cases. In the case of HBV env 313 the I9 (I at C-terminal position 9) replacement was effective in increasing cross-reactivity from 4 to 5 alleles bound by virtue of an almost 400-fold increase B*5401 binding affinity. In the case of HBV pol 541, increased cross-reactivity was similarly achieved by a substantial increase in B*5401 binding. Also, significant gains in binding affinity for B*0702, B51, and B*5301 were observed with the HBV pol 541 I9 analog.

### Analoging at Secondary Anchor Residues

Moreover, HLA superrnotifs are of value in engineering highly cross-reactive peptides by identifying particular residues at secondary anchor positions that are associated with such cross-reactive properties. Demonstrating this, the capacity of a second set of peptides representing discreet single amino acid substitutions at positions one and three of five different B7-supertype binding peptides were synthesized and tested for their B-7 supertype binding capacity. In 4/4 cases the effect of replacing the native residue at position 1 with the aromatic residue F (an “F1” substitution) resulted in an increase in cross-reactivity, compared to the parent peptide, and, in most instances, binding affinity was increased three-fold or better (Table XXVIII). More specifically, for HBV env 313, MAGE2 170, and HBV core 168 complete supertype cross-reactivity was achieved with the F1 substitution analogs. These gains were achieved by dramatically increasing B*5401 binding affinity. Also, gains in affinity were noted for other alleles in the cases of HBV core 168 (B*3501 and B*5301) and MAGE2 170 (B*3501, B51 and B*5301). Finally, in the case of MAGE3 196, the F1 replacement was effective in increasing cross-reactivity because of gains in B*0702 binding. An almost 70-fold increase in B51 binding capacity was also noted.

Two analogs were also made using the supermotif positive F substitution at position three (an “F3” substitution). In both instances increases in binding affinity and cross-reactivity were achieved. Specifically, in the case of HBV pol 541, the F3 substitution was effective in increasing cross-reactivity by virtue of its effect on B*5401 binding. In the case of MAGE3 196, complete supertype cross-reactivity was achieved by increasing B*0702 and B*3501 binding capacity. Also, in the case of MAGE3 196, it is notable that increases in binding capacity between 40- and 5000-fold were obtained for B*3501, B51, B*5301, and B*5401.

In conclusion, these data demonstrate that by the use of even single amino acid substitutions, it is possible to increase the binding affinity and/or cross-reactivity of peptide ligands for HLA supertype molecules.

### Example 5

### Identification of Conserved HBV-Derived Sequences with HLA-DR Binding Motifs

Peptide epitopes bearing an HLA class II supermotif or motif may also be identified as outlined below using methodology similar to that described in Examples 1-3.

### Selection of HLA-DR-Supermotif-Bearing Epitopes

HLA-Class II molecules bind peptides typically between 12 and 20 residues in length. However, similar to HLA-Class I, the specificity and energy of interaction is usually contained within a short core region of about 9 residues. Most DR molecules share an overlapping specificity within this 9-mer core in which a hydrophobic residue in position 1 (P1) is the main anchor (O'Sullivan et al., J. Immunol. 147:2663, 1991; Southwood et al., J. Immunol. 160:3363, 1998). The presence of small or hydrophobic residues in position 6 (P6) is also important for most DR-peptide interactions. This overlapping P1-P6 specificity, within a 9-mer core region, has been defined as the DR-supermotif. Unlike Class I molecules, DR molecules are open at both ends of the binding groove, and can therefore accommodate longer peptides of varying length. Indeed, while most of the energy of peptide-DR interactions appears to be contributed by the core region, flanking residues appear to be important for high affinity interactions. Also, although not strictly necessary for MHC binding, flanking residues are clearly necessary in most instances for T cell recognition.

To identify HBV-derived DR cross-reactive HTL epitopes, the same 20 HBV polyproteins that were scanned for the identification of HLA Class I motif sequences were scanned for the presence of sequences with motifs for binding HLA-DR. Specifically, 15-mer sequences comprised of a DR-supermotif containing 9-mer core, and three residue N- and C-terminal flanking regions, were selected. It was also required that 100% of the 15-mer sequence be conserved in at least 85% (17/20) of the HBV strains scanned. Using these criteria, 36 non-redundant sequences were identified. Thirty-five of these peptides were subsequently synthesized.

Algorithms for predicting peptide binding to DR molecules have also been developed (Southwood et al., J. Immunol. 160:3363, 1998). These algorithms, specific for individual DR molecules, allow the scoring and ranking of 9-mer core regions. Using selection tables, it has been found that these algorithms efficiently select peptide sequences with a high probability of binding the appropriate DR molecule. Additionally, it has been found that running algorithms, specifically those for DR1, DR4w4, and DR7, sequentially can efficiently select DR cross-reactive peptides.

To see if these algorithms would identify additional peptides, the same HBV polyproteins used above were re-scanned for the presence of 15-mer peptides where 100% of the 9-mer core region was ³85% (17/20 strains) conserved. Next, the 9-mer core region of each of these peptides was scored using the DR1, DR4w4, and DR7 algorithms. As a result, 8 additional sequences were identified and synthesized.

In summary, 44 15-mer peptides in which a 9-mer core region contained the DRsupermotif, or was selected using an algorithm predicting DR-binding sequences, were identified. Forty-three of these peptides were synthesized (Table XXXIII).

While performing the analyses of HBV-derived peptides described above, 9 peptides predicted on the basis of their DR1, DR4w4, and DR7 algorithm profiles to be DR-cross-reactive binding peptides, but which have 9-mer core regions that are only 80% conserved, were also identified. An additional peptide which contains a DR-supermotif core region that is 95% conserved, but is located only one residue removed from the N-terminus, was previously synthesized. These 10 peptides were also selected for further analysis, and are shown in Table XXXIII.

Finally, 2 peptides, CF-08 and 1186.25, which are redundant with a peptide selected above (27.0280), were considered for additional analysis. Peptide 1186.25 contains multiple DR-supermotif sequences. Peptide CF-08 is a 20-mer that nests both 27.0280 and 1186.25. These peptides are shown in Table XXXIII.

The 55 HBV-derived peptides identified above were tested for their capacity to bind common HLA-DR alleles. To maximize both population coverage, and the relationships between the binding repertoires of most DR alleles (see, e.g., Southwood et al., J. Immunol. 160:3363, 1998), peptides were screened for binding to sequential panels of DR assays. The composition of these screening panels, and the phenotypic frequency of associated antigens, are shown in Table XXXIV. All peptides were initially tested for binding to the alleles in the primary panel: DR1, DR4w4, and DR7. Only peptides binding at least 2 of these 3 alleles were then tested for binding in the secondary assays (DR2w2 β1, DR2w2 β2, DR6w19, and DR9). Finally, only peptides binding at least 2 of the 4 secondary panel alleles, and thus 4 of 7 alleles total, were screened for binding in the tertiary assays (DR4w15, DR5w11, and DR⁸w2).

Upon testing, it was found that 25 of the original 55 peptides (45%) bound two or more of the primary panel alleles. When these 25 peptides were subsequently tested in the secondary assays, 20 were found to bind at least 4 of the 7 DR alleles in the primary and secondary assay panels. Finally, 18 of the 20 peptides passing the secondary screening phase were tested for binding in the tertiary assays. As a result, 12 peptides were shown to bind at least 7 of 10 common HLA-DR alleles. The sequences of these 12 peptides, and their binding capacity for each assay in the primary through tertiary panels, are shown in Table XXXV. Also shown are peptides CF-08 and 857.02, which bound 5/5 and 5/6 of the alleles tested to date, respectively.

In summary, 14 peptides, derived from 12 independent regions of the HBV genome, have been identified that are capable of binding multiple HLA-DR alleles. This set of peptides includes at least 2 epitopes each from the Core (Nuc), Pol, and Env antigens.

### Selection of Conserved DR3 Motif Peptides

Because HLA-DR3 is an allele that is prevalent in Caucasian, Black, and Hispanic populations, DR3 binding capacity is an important criterion in the selection of HTL epitopes. However, data generated previously indicated that DR3 only rarely cross-reacts with other DR alleles (Sidney et al., J. Immunol. 149:2634-2640, 1992; Geluk et al., J. Immunol. 152:5742-5748, 1994; Southwood et al., J. Immunol. 160:3363-3373, 1998). This is not entirely surprising in that the DR3 peptide-binding motif appears to be distinct from the specificity of most other DR alleles.

To efficiently identify peptides that bind DR3, target proteins were analyzed for conserved sequences carrying one of the two DR3 specific binding motifs reported by Geluk et al. (J. Immunol. 152:5742-5748, 1994). Eighteen sequences were identified. Eight of these sequences were largely redundant with peptides shown in Table XXXVI, and 3 with peptides that had previously been synthesized for other studies. The 7 unique sequences were synthesized.

Seventeen of the eighteen peptides containing a DR3 motif have been tested for their DR3 binding capacity. Four peptides were found to bind DR3 with an affinity of 1000 nM or better (Table XXXVI).

### Example 6

### Calculation of Phenotypic Frequencies of HLA-Supertypes in Various Ethnic Backgrounds to Determine Breadth of Population Coverage

This example illustrates the assessment of the breadth of population coverage of a vaccine composition comprised of multiple epitopes comprising multiple supermotifs and/or motifs.

In order to analyze population coverage, gene frequencies of HLA alleles were determined. Gene frequencies for each HLA allele were calculated from antigen or allele frequencies utilizing the binomial distribution formulae gf=1−(SQRT(1−af)) (see, e.g., Sidney et al., Human Immunol. 45:79-93, 1996). To obtain overall phenotypic frequencies, cumulative gene frequencies were calculated, and the cumulative antigen frequencies derived by the use of the inverse formula [af−1−(1−Cgf)²].

Where frequency data was not available at the level of DNA typing, correspondence to the serologically defined antigen frequencies was assumed. To obtain total potential supertype population coverage no linkage disequilibrium was assumed, and only alleles confirmed to belong to each of the supertypes were included (minimal estimates). Estimates of total potential coverage achieved by inter-loci combinations were made by adding to the A coverage the proportion of the non-A covered population that could be expected to be covered by the B alleles considered (e.g., total=A+B*(1−A)). Confirmed members of the A3-like supertype are A3, A11, A31, A*3301, and A*6801. Although the A3-like supertype may potentially include A34, A66, and A*7401, these alleles were not included in overall frequency calculations. Likewise, confirmed members of the A2-like supertype family are A*0201, A*0202, A*0203, A*0204, A*0205, A*0206, A*0207, A*6802, and A*6901. Finally, the B7-like supertype-confirmed alleles are: B7, B*3501-03, B51, B*5301, B*5401, B*5501-2, B*5601, B*6701, and B*7801 (potentially also B*1401, B*3504-06, B*4201, and B*5602). Population coverage achieved by combining the A2-, A3- and B7-supertypes is approximately 86% in five major ethnic groups (see Table XXI). Coverage may be extended by including peptides bearing the A1 and A24 motifs. On average, A1 is present in 12% and A24 in 29% of the population across five different major ethnic groups (Caucasian, North American Black, Chinese, Japanese, and Hispanic). Together, these alleles are represented with an average frequency of 39% in these same ethnic populations. The total coverage across the major ethnicities when A1 and A24 are combined with the coverage of the A2-, A3- and B7-supertype alleles is >95%.

Population coverage for HLA class II molecules can be developed analagously based on the present disclosure.

Summary of Candidate HLA class I and class II Epitopes

In summary, on the basis of the data presented above, 34 conserved CTL epitopes were selected as vaccine candidates (Table XXXVII). Of these 34 epitopes, 7 are derived from core, 18 from polymerase, and 9 from envelope. No epitopes from the X antigen were included in the package as this protein is expressed in low amounts and is, therefore, of less immunological interest.

The population coverage afforded by this panel of CTL epitopes is estimated to exceed 95% in each of 5 major ethnic populations. Using a Monte Carlo analysis (FIG. 1), it is predicted that approximately 90% of the individuals in a population comprised of Caucasians, North American Blacks, Japanese, Chinese and Hispanics would recognize five or more of the vaccine candidate epitopes.

While preferred CTL epitopes includes 34 discrete peptides, two peptides are entirely nested within longer peptides, thus effectively reducing the numbers of peptides that would have to be included in a vaccine candidate. Specifically, the A2-restricted peptide 927.15 is nested within the B7-restricted peptide 26.0570 and the B7-restricted peptide 988.05 is nested within the A2-restricted peptide 924.07. Similarly, the A24-restricted peptide 20.0136 and the A2-restricted peptide 1013.01 contain the same core region, differing only at the first amino acid. On a related note, the A2-restricted peptide 1090.14 and the B7-restricted peptide 1147.05 overlap by two amino acids, raising the possibility of delivering these two epitopes as one contiguous peptide sequence.

The set of recommended vaccine candidates includes 9 A2-restricted CTL epitopes; four polymerase-derived epitopes, four envelope-derived epitopes and a core epitope. Seven of these 9 peptides are recognized in recall CTL assays from acute patients. Of the 7 peptides recognized in patients, 2 are non-crossreactive binding peptides. The inclusion of these peptides as potential vaccine candidates stems from the observation that HLA-A*0201 is the predominantly expressed A2-supertype allele in all ethnicities examined. As such, inclusion of non-crossreactive A*0201 binding peptides increases the redundancy of antigen coverage and population coverage. The only two A2-restricted peptides that lack patient immunogenicity data are peptides 1090.77 and 1069.06. The 1090.77 peptide is an analog of a highly immunogenic peptide recognized in acute HBV patients. Although recall responses in patients have not been tested for the ability to recognize the analog peptide, immunogenicity studies conducted in HLA transgenic mice have shown that CTL induced with 1090.77 are capable of recognizing target cells loaded with the naturally occurring sequence. This data indicates that CTL raised to the 1090.77 peptide are cross-reactive and should recognize HBV-infected cells. The 1069.06 peptide was included as a potential vaccine epitope because its high binding affinity for A*6802 results in a greater population coverage. While peptide 1069.06 has not been tested for recognition by acute HBV patients, the peptide is immunogenic in HLA-A2 transgenic mice and primary human cultures.

Preferred CTL epitopes include 7 A3-supertype-restricted peptides; 6 derived from the polymerase antigen, and one from the core region. All of the A3-supertype vaccine candidate peptides are immunogenic in patients. Although peptide 1142.05 is a non-crossreactive A3-restricted peptide, it has been included because it has been shown to be recognized in patients and is capable of binding HLA-A1.

Nine B7-restricted peptides are preferred CTL epitopes. Of this group, 3 epitopes have been shown to be recognized in patients. While one of these peptides, 1147.04, is a non-crossreactive binder, it binds 2 of the major B7 supertype alleles with an IC₅₀ or binding affinity value of less than 100 nM. Six B7-supertype epitopes were included as preferred epitopes based on supertype binding. Immunogenicity studies in humans (Bertoni et al., 1997; Doolan et al., 1997; Threlkeld et al., 1997) have demonstrated that highly cross-reactive peptides are almost always recognized as epitopes. Given these results, and in light of the limited immunogenicity data available, the use of B7-supertype binding affinity as a selection criterion was deemed appropriate.

Similarly, there is little immunogenicity data regarding A1- and A24-restricted peptides. One preferred CTL epitope, 1069.04, has been reported to be recognized in recall responses from acute HBV patients. As discussed in the preceding paragraph, a high percentage of the peptides with binding affinities<100 nM are found to be immunogenic. For this reason, all A1 and A24 peptides with binding affinities<100 nM were considered as preferred CTL epitopes. Using this selection criterion, 3 A1-restricted and 6 A24-restricted peptides are identified as candidate epitopes. Further analysis found that 3 core-derived peptides bound A24 with intermediate affinity. Since relatively few core epitopes were identified during the course of this study, the intermediate A24 binding core peptides were also included in the set of preferred epitopes to provide a greater degree of redundancy in antigen coverage.

The list of preferred HBV-derived HTL epitopes is summarized in Table XXXVII. The set of HTL epitopes includes 12 DR supermotif binding peptides and 4 DR3 binding peptides. The bulk of the HTL epitopes are derived from polymerase; 2 envelope and 2 core derived epitopes are also included in the set of preferred HTL epitopes. The total estimated population coverage represented by the panel of HTL epitopes is in excess of 91% in each of five major ethnic groups (Table XXXVIII)

### Example 7

### Recognition of Generation of Endogenous Processed Antigens after Priming

This example determines that CTL induced by native or analogued peptide epitopes identified and selected as described in Examples 1-5 recognize endogenously synthesized, i.e., native antigens.

Effector cells isolated from transgenic mice that are immunized with peptide epitopes as in Example 3 are re-stimulated in vitro using peptide-coated stimulator cells. Six days later, effector cells are assayed for cytotoxicity and the cell lines that contain peptide-specific cytotoxic activity are further re-stimulated. An additional six days later, these cell lines are tested for cytotoxic activity on ⁵¹Cr labeled 3A4-721.221-A11/K^{b} target cells, in the absence or presence of peptide, and also tested on ⁵¹Cr labeled target cells bearing the endogenously synthesized antigen, e.g., cells that are stably transfected with HBV expression vectors.

The result will demonstrate that CTL lines obtained from animals primed with peptide epitope recognize endogenously synthesized HBV antigen.

### Example 8

### Activity Of CTL-HTL Conjugated Epitopes in Transgenic Mice

This example illustrates the induction of CTLs in transgenic mice by use of an HBV CTL/HTL peptide conjugate. An analagous study may be found in Oseroffet al. Vaccine 16:823-833 (1998). The peptide composition can comprise multiple CTL and/or HTL epitopes. Such a peptide composition can comprise a lipidated HTL epitope conjugated to a preferred CTL epitope containing, for example, an A11 motif or an analog of that epitope.

Lipopeptides are prepared by coupling the appropriate fatty acid to the amino terminus of the resin bound peptide. A typical procedure is as follows: A dichloromethane solution of a four-fold excess of a pre-formed symmetrical anhydride of the appropriate fatty acid is added to the resin and the mixture is allowed to react for two hours. The resin is washed with dichloromethane and dried. The resin is then treated with trifluoroacetic acid in the presence of appropriate scavengers [e.g. 5% (v/v) water] for 60 minutes at 20° C. After evaporation of excess trifluoroacetic acid, the crude peptide is washed with diethyl ether, dissolved in methanol and precipitated by the addition of water. The peptide is collected by filtration and dried.

Preparation of Peptides for Immunization: Peptide Compositions are Typically resuspended in DMSO at a concentration of 20 mg/ml. Before use, peptides are prepared at the required concentration by dilution in saline or the appropriate medium.

Immunization procedures: A11/K^{b} mice, which are transgenic for the human HLA A11 allele, are primed subcutaneously (base of the tail) with 0.1 ml of peptide conjugate formulated in saline, or DMSO/saline. Seven days after priming, splenocytes obtained from these animals are restimulated with syngeneic irradiated LPS-activated lymphoblasts coated with peptide.

Media:

a. RPMI-1640 supplemented with 10% fetal calf serum (FCS) 2 mM Glutamine, 50 μg/ml Gentamicin and 5×10⁻⁵ M 2-mercaptoethanol serves as culture medium

b. RPMI-1640 containing 25 mM HEPES buffer and supplemented with 2% (FCS) is used as cell washing medium.

Cell lines: The 3A4-721.221-A11/K^{b} cell line is used as target cells. This cell line is an EBV transformed cell line that was mutagenized and selected to be Class I negative which was transfected with an HLA-A11/K^{b} gene.

LPS-activated lymphoblasts: Splenocytes obtained from transgenic mice are resuspended at a concentration of 1-1.5×10⁶/ml in culture medium supplemented with 25 μg/ml LPS and 7 μg/ml dextran sulfate in 75 cm² tissue culture flasks. After 72 hours at 37° C., the lymphoblasts are collected for use by centrifugation.

Peptide coating of lymphoblasts: Peptide coating of the LPS activated lymphoblasts is achieved by incubating 30×10⁶ irradiated (3000 rads) lymphoblasts with 100 μg of peptide in 1 ml of R10 medium for 1 hr at 37° C. Cells are then washed once and resuspended in culture medium at the desired concentration.

In vitro CTL activation: One week after priming, spleen cells (30×10⁶ cells/flask) are co-cultured at 37° C. with syngeneic, irradiated (3000 rads), peptide coated lymphoblasts (10×10⁶ cells/flask) in 10 ml of culture medium/T25 flask. After six days, the effector cells are harvested and assayed for cytotoxic activity.

Assay for cytotoxic activity: Target cells (1.0-1.5×10⁶) are incubated at 37° C. in the presence of 200 μl of sodium ⁵¹Cr chromate. After 60 minutes, cells are washed three times and resuspended in R10 medium. Peptide is added where required at a concentration of 1 μg/ml. For the assay, 104 ⁵¹Cr-labeled target cells are added to different concentrations of effector cells (final volume of 200 μl) in U-bottom 96-well plates. After a 6 hour incubation period at 37° C., a 0.1 ml aliquot of supernatant is removed from each well and radioactivity is determined in a Micromedic automatic gamma counter. The percent specific lysis is determined by the formula: percent specific release=100×(experimental release−spontaneous release)/(maximum release−spontaneous release). To facilitate comparison between separate CTL assays run under the same conditions, % ⁵¹Cr release data is expressed as lytic units/10⁶ cells. One lytic unit is arbitrarily defined as the number of effector cells required to achieve 30% lysis of 10,000 target cells in a 6 hour ⁵¹Cr release assay. To obtain specific lytic units/10⁶, the lytic units/10⁶ obtained in the absence of peptide is subtracted from the lytic units/10⁶ obtained in the presence of peptide. For example, if 30% ⁵¹Cr release is obtained at the E:T of 50:1 (i.e., 5×10⁵ effector cells for 10,000 targets) in the absence of peptide and 5:1 (i.e., 5×10⁴ effector cells for 10,000 targets) in the presence of peptide, the specific lytic units would be: (1×10⁶(5×10⁴)−(1×10⁶(5×10⁵)=18LU/10⁶.

The results are analyzed to assess the magnitude of the CTL responses of animals injected with the immunogenic CTL/HTL conjugate vaccine preparation and are compared to the magnitude of the CTL response achieved using the CTL epitope as outlined in Example 3. Analyses similar to this may be performed to evaluate the immunogenicity of peptide conjugates containing multiple CTL epitopes and/or multiple HTL epitopes. In accordance with these procedures it is found that a CTL response is induced, and concomitantly that an HTL response is induced upon administration of such compositions.

### Example 9

### Selection of CTL and HTL Epitopes for Inclusion in an HBV-Specific Vaccine

This example illustrates the procedure for the selection of peptide epitopes for vaccine compositions of the invention.

The following principles are utilized when selecting an array of epitopes for inclusion in a polyepitopic composition, or for selecting epitopes to be included in a vaccine composition and/or to be encoded by a minigene. Each of the following principles are balanced in order to make the selection.

1.) Epitopes are selected which, upon administration, mimic immune responses that have been observed to be correlated with HBV clearance. For HLA Class I this includes 3-4 epitopes that come from at least one antigen of HBV. In other words, it has been observed that in patients who spontaneously clear HBV, that they had generated an immune response to at least 3 epitopes on at least one HBV antigen. For HLA Class II a similar rationale is employed; again 3-4 epitopes are selected from at least one HBV antigen.

2.) Epitopes are selected that have the requisite binding affinity established to be correlated with immunogenicity: for HLA Class I an IC₅₀ of 500 nM or less, or for Class II an IC₅₀ of 1000 nM or less.

3.) Sufficient supermotif bearing peptides, or a sufficient array of allele-specific motif bearing peptides, are selected to give broad population coverage. For example, epitopes are selected to provide at least 80% population coverage. A Monte Carlo analysis, a statistical evaluation known in the art, is employed to assess population coverage.

4.) When selecting epitopes for HBV antigens it is often preferable to select native epitopes. Therefore, of particular relevance for infectious disease vaccines, are epitopes referred to as “nested epitopes.” Nested epitopes occur where at least two epitopes overlap in a given peptide sequence. A peptide comprising “transcendent nested epitopes” is a peptide that has both HLA class I and HLA class II epitopes in it.

When providing nested epitopes, a sequence that has the greatest number of epitopes per provided sequence is provided. A limitation on this principle is to avoid providing a peptide that is any longer than the amino terminus of the amino terminal epitope and the carboxyl terminus of the carboxyl terminal epitope in the peptide. When providing a longer peptide sequence, such as a sequence comprising nested epitopes, the sequence is screened in order to insure that it does not have pathological or other deleterious biological properties.

5.) When creating a minigene, as disclosed in greater detail in the Example 9, an objective is to generate the smallest peptide possible that encompasses the epitopes of interest. The principles employed are similar, if not the same as those employed when selecting a peptide comprising nested epitopes. Thus, upon determination of the nucleic acid sequence to be provided as a minigene, the peptide encoded thereby is analyzed to determine whether any “junctional epitopes” have been created. A junctional epitope is an actual binding epitope, as predicted, e.g., by motif analysis. Junctional epitopes are to be avoided because the recipient may generate an immune response to that epitope. Of particular concern is a junctional epitope that is a “dominant epitope.” A dominant epitope may lead to such a zealous response that immune responses to other epitopes are diminished or suppressed.

Peptide epitopes for inclusion in vaccine compositions are, for example, selected from those listed in Table XXXVIIa and b. A vaccine composition comprised of selected peptides, when administered, is safe, efficacious, and elicits an immune response similar in magnitude of an immune response that clears an acute HBV infection.

### Example 10

### Construction of Minigene Multi-Epitope DNA Plasmids

This example provides an illustration of the construction of a minigene expression plasmid. Minigene plasmids may, of course, contain various configurations of CTL and/or HTL epitopes or epitope analogs as described herein. Expression plasmids have been constructed and evaluated as described, for example, in U.S. Ser. No. 60/085,751 filed May 15, 1998 and U.S. Ser. No. 09/078,904 filed May 13, 1998. An example of such a plasmid is shown in FIG. 2, which illustrates the orientation of HBV epitopes in minigene constructs. Such a plasmid may, for example, also include multiple CTL and HTL peptide epitopes.

A minigene expression plasmid may include multiple CTL and HTL peptide epitopes. In the present example, HLA-A2, -A3, -B7 supermotif-bearing peptide epitopes and HLA-A1 and -A24 motif-bearing peptide epitopes are used in conjunction with DR supermotif-bearing epitopes and/or DR3 epitopes (FIG. 2). Preferred epitopes are identified, for example, in Tables XXVI-XXXIII, HLA class I supermotif or motif-bearing peptide epitopes derived from multiple HBV antigens, e.g., the core, polymerase, envelope and X proteins, are selected such that multiple supermotifs/motifs are represented to ensure broad population coverage. Similarly, HLA class II epitopes are selected from multiple HBV antigens to provide broad population coverage, i.e. both HLA DR-1-4-7 supermotif-bearing epitopes and HLA DR-3 motif-bearing epitopes are selected for inclusion in the minigene construct. The selected CTL and HTL epitopes are then incorporated into a minigene for expression in an expression vector.

This example illustrates the methods to be used for construction of such a minigene-bearing expression plasmid. Other expression vectors that may be used for minigene compositions are available and known to those of skill in the art.

The minigene DNA plasmid contains a consensus Kozak sequence and a consensus murine kappa Ig-light chain signal sequence followed by a string of CTL and/or HTL epitopes selected in accordance with principles disclosed herein.

Overlapping oligonucleotides, for example eight oligonucleotides, averaging approximately 70 nucleotides in length with 15 nucleotide overlaps, are synthesized and HPLC-purified. The oligonucleotides encode the selected peptide epitopes as well as appropriate linker nucleotides. The final multiepitope minigene is assembled by extending the overlapping oligonucleotides in three sets of reactions using PCR. A Perkin/Elmer 9600 PCR machine is used and a total of 30 cycles are performed using the following conditions: 95° C. for 15 sec, annealing temperature (50 below the lowest calculated Tm of each primer pair) for 30 sec, and 72° C. for 1 min.

For the first PCR reaction, 5 μg of each of two oligonucleotides are annealed and extended: Oligonucleotides 1+2, 3+4, 5+6, and 7+8 are combined in 100 μl reactions containing Pfu polymerase buffer (1×=10 mM KCL, 10 mM (NH₄)₂SO₄, 20 mM Tris-chloride, pH 8.75, 2 mM MgSO₄, 0.1% Triton X-100, 100 μg/ml BSA), 0.25 mM each dNTP, and 2.5 U of Pfu polymerase. The full-length dimer products are gel-purified, and two reactions containing the product of 1+2 and 3+4, and the product of 5+6 and 7+8 are mixed, annealed, and extended for 10 cycles. Half of the two reactions are then mixed, and 5 cycles of annealing and extension carried out before flanking primers are added to amplify the full length product for 25 additional cycles. The full-length product is gel-purified and cloned into pCR-blunt (Invitrogen) and individual clones are screened by sequencing.

### Example 11

### The Plasmid Construct and the Degree to which it Induces Immunogenicity

The degree to which the plasmid construct prepared using the methodology outlined in Example 9 is able to induce immunogenicity is evaluated through in vivo injections into transgenic mice and in vitro culture of CTL and HTL, which are subsequently analysed using cytotoxicity and proliferation assays, respectively, as detailed e.g., in U.S. Ser. No. 09/311,784 filed May 13, 1999 and Alexander et al., Immunity 1:751-761, 1994. To assess the capacity of the pMin minigene construct to induce CTLs in vivo, HLA-A11/K^{b} transgenic mice, for example, are immunized intramuscularly with 100 μg of plasmid cDNA. As a means of comparing the level of CTLs induced by DNA immunization, a control group of animals is also immunized with an actual peptide composition that comprises multiple epitopes synthesized as a single polypeptide as they would be encoded by the minigene.

Splenocytes from immunized animals are stimulated twice with each of the respective compositions (peptide epitopes encoded in the minigene or the polyepitopic peptide), then assayed for peptide-specific cytotoxic activity in a ⁵¹Cr release assay. The results indicate the magnitude of the CTL response directed against the A3-restricted epitope, thus indicating the in vivo immunogenicity of the minigene vaccine and polyepitopic vaccine. It is, therefore, found that the minigene elicits immune responses directed toward the HLA-A3 supermotif peptide epitopes as does the polyepitopic peptide vaccine. Such an analysis is also performed using other HLA-A2 and HLA-B7 transgenic mouse models to assess CTL induction by HLA-A2 and HLA-B7 motif or supermotif epitopes.

To assess the capacity of a class II epitope encoding minigene to induce HTLs in vivo, I-Ab restricted mice, for example, are immunized intramuscularly with 100 μg of plasmid DNA. As a means of comparing the level of HTLs induced by DNA immunization, a group of control animals is also immunized with an actual peptide composition emulsified in complete Freund's adjuvant.

CD4+ T cells, i.e. HTLs, are purified from splenocytes of immunized animals and stimulated with each of the respective compositions (peptides encoded in the minigene). The HTL response is measured using a ³H-thymidine incorporation proliferation assay, (see, e.g., Alexander et al. Immunity 1:751-761, 1994). the results indicate the magnitude of the HTL response, thus demonstrating the in vivo immunogenicity of the minigene.

### Example 12

### Peptide Composition for Prophylactic Uses

Vaccine compositions of the present invention are used to prevent HBV infection in persons who are at risk for such an infection. For example, a polyepitopic peptide epitope composition containing multiple CTL and HTL epitopes such as those selected in Examples 9 and/or 10, which are also selected to target greater than 80% of the population, is administered to individuals at risk for HBV infection. The composition is provided as a single lipidated polypeptide that encompasses multiple epitopes. The vaccine is administered in an aqueous carrier comprised of Freunds Incomplete Adjuvant. The dose of peptide for the initial immunization is from about 500 to about 50,000 μg for a 70 kg patient. The initial administration of vaccine is followed by booster dosages at 4 weeks followed by evaluation of the magnitude of the immune response in the patient by techriiques that determine the presence of epitope-specific CTL populations in a PBMC sample. Additional booster doses are administered as required. The composition is found to be both safe and efficacious as a prophylaxis against HBV infection.

Alternatively, the polyepitopic peptide composition can be administered as a nucleic acid in accordance with methodologies known in the art and disclosed herein.

### Example 13

### Polyepitopic Vaccine Compositions Derived from Native HBV Sequences

A native HBV polyprotein sequence is screened, preferably using computer algorithms defined for each class I and/or class II supermotif or motif, to identify “relatively short” regions of the polyprotein that comprise multiple epitopes. This relatively short sequence that contains multiple distinct, even overlapping, epitopes is selected and used to generate a minigene construct. The construct is engineered to express the peptide, which corresponds to the native protein sequence. The “relatively short” peptide is less than 250 amino acids in length, preferably less than 100 amino acids in length, and more preferably less than 75 or 50 amino acids in length. The protein sequence of the vaccine composition is selected because it has maximal number of epitopes contained within the sequence. As noted herein, epitope motifs may be overlapping (i.e., frame shifted relative to one another) with frame shifted overlapping epitopes, e.g. two 9-mer epitopes can be present in a 10 amino acid peptide. Such a vaccine composition is administered for therapeutic or prophylactic purposes.

The vaccine composition will preferably include, for example, three CTL epitopes and at least one HTL epitope from the source antigen. This polyepitopic native sequence is administered either as a peptide or as a nucleic acid sequence which encodes the peptide. Alternatively, an analog can be made of this native sequence, whereby one or more of the epitopes comprise substitutions that alter the cross-reactivity and/or binding affinity properties of the polyepitopic peptide.

The embodiment of this example provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (absent analogs) directs the immune response to peptide sequences that are present in native HBV antigens. Lastly, the embodiment provides an economy of scale when producing nucleic acid vaccine compositions.

Related to this embodiment, computer programs can be derived which identify, in a target sequence, the greatest number of epitopes per sequence length.

### Example 14

### Polyepitopic Vaccine Compositions Directed to Multiple Diseases

The HBV peptide epitopes of the present invention are used in conjunction with peptide epitopes from target antigens related to one or more other diseases, to create a vaccine composition that is useful for the prevention or treatment of HBV as well as another disease. Examples of other diseases include, but are not limited to, HIV, HCV, and HPV.

For example, a polyepitopic peptide composition comprising multiple CTL and HTL epitopes that target greater than 98% of the population may be created for administration to individuals at risk for both HBV and HIV infection. The composition can be provided as a single polypeptide that incorporates the multiple epitopes from the various disease-associated sources, or can be administered as a composition comprising one or more discrete epitopes.

### Example 15

### Use of Peptides to Evaluate an Immune Response

Peptides of the invention may be used to analyze an immune response for the presence of specific CTL populations corresponding to HBV. Such an analysis may be performed as described by Ogg et al., Science 279:2103-2106, 1998. In the following example, peptides in accordance with the invention are used as a reagent for diagnostic or prognostic purposes, not as an immunogen.

In this example highly sensitive human leukocyte antigen tetrameric complexes (“tetramers”) may be used for a cross-sectional analysis of, for example, HBV Env-specific CTL frequencies from untreated HLA A*0201-positive indiviuals at different stages of infection using an HBV Env peptide containing an A2.1 extended motif. Tetrameric complexes are synethesized as described (Musey et al., N. Engl. J. Med. 337:1267, 1997). Briefly, purified HLA heavy chain (A2.1 in this example) and β2-microglobulin are synthesized by means of a prokaryotic expression system. The heavy chain is modified by deletion of the transmembrane-cytosolic tail and COOH-terminal addition of a sequence containing a BirA enzymatic biotinylation site. The heavy chain, β2-microglobulin, and peptide are refolded by dilution. The 45-kD refolded product is isolated by fast protein liquid chromatography and then biotinylated by BirA in the presence of biotin (Sigma, St. Louis, Mo.), adenosine 5′triphosphate and magnesium. Streptavidin-phycoerythrin conjugate is added in a 1:4 molar ratio, and the tetrameric product is concentrated to 1 mg/ml. The resulting product is referred to as tetramer-phycoerythrin.

For the analysis of patient blood samples, approximately one million PBMCs are centrifuged at 300 g for 5 minutes and resuspended in 50 ul of cold phosphate-buffered saline. Tri-color analysis is performed with the tetramer-phycoerythrin, along with anti-CD8-Tricolor, and anti-CD38. The PBMCs are incubated with tetramer and antibodies on ice for 30 to 60 min and then washed twice before formaldehyde fixation. Gates are applied to contain>99.98% of control samples. Controls for the tetramers include both A*0201-negative individuals and A*0201-positive uninfected donors. The percentage of cells stained with the tetramer is then determined by flow cytometry. The results indicate the number of cells in the PBMC sample that contain epitope-restricted CTLs, thereby readily indicating the stage of infection with HBV or the status of exposure to HBV or to a vaccine that elicits a protective response.

### Example 16

### Use of Peptide Epitopes to Evaluate Recall Responses

The peptide epitopes of the invention are used as reagents to evaluate T cell responses such as acute or recall responses, in patients. Such an analysis may be performed on patients who have recovered from infection or who are chronically infected with HBV or who have been vaccinated with an HBV vaccine.

For example, the class I restricted CTL response of persons at risk for HBV infection who have been vaccinated may be analyzed. The vaccine may be any HBV vaccine. PBMC are collected from vaccinated individuals and HLA typed. Appropriate peptide reagents that, are highly conserved and, optimally, bear supermotifs to provide cross-reactivity with multiple HLA supertype family members are then used for analysis of samples derived from individuals who bear that HLA type.

PBMC from vaccinated individuals are separated on Ficoll-Histopaque density gradients (Sigma Chemical Co., St. Louis, Mo.), washed three times in HBSS (GIBCO Laboratories), resuspended in RPMI-1640 (GIBCO Laboratories) supplemented with L-glutamine (2 mM), penicillin (SOU/ml), streptomycin (50 μg/ml), and Hepes (10 mM) containing 10% heat-inactivated human AB serum (complete RPMI) and plated using microculture formats. Synthetic peptide is added at 10 μg/ml to each well and recombinant HBc Ag is added at 1 μg/ml to each well as a source of T cell help during the first week of stimulation.

In the microculture format, 4×10⁵ PBMC are stimulated with peptide in 8 replicate cultures in 96-well round bottom plate in 100 μl/well of complete RPMI. On days 3 and 10, 100 ml of complete RPMI and 20 U/ml final concentration of rIL-2 are added to each well. On day 7 the cultures are transferred into a 96-well flat-bottom plate and restimulated with peptide, rIL-2 and 105 irradiated (3,000 rad) autologous feeder cells. The cultures are tested for cytotoxic activity on day 14. A positive CTL response requires two or more of the eight replicate cultures to display greater than 10% specific ⁵¹Cr release, based on comparison with uninfected control subjects as previously described (Rehermann, et al., Nature Med. 2:1104, 1108, 1996; Rehermann et al., J. Clin. Invest. 97:1655-1665, 1996; and Rehermann et al. J. Clin. Invest. 98:1432-1440, 1996).

Target cell lines are autologous and allogeneic EBV-transformed B-LCL that are either purchased from the American Society for Histocompatibility and Immunogenetics (ASHI, Boston, Mass.) or established from the pool of patients as described (Guilhot, et al. J. Virol. 66:2670-2678, 1992).

Cytotoxicity assays are performed in the following manner. Target cells consist of either allogeneic HLA-matched or autologous EBV-transformed B lymphoblastoid cell line that are incubated overnight with synthetic peptide at 10 μM and labeled with 100 μCi of ⁵¹Cr (Amersham Corp., Arlington Heights, Ill.) for 1 hour after which they are washed four times with HBSS. Cytolytic activity is determined in a standard 4-h, split well ⁵¹Cr release assay using U-bottomed 96 well plates containing 3,000 targets/well. Stimulated PBMC are tested at E/T ratios of 20-50:1 on day 14. Percent cytotoxicity is determined from the formula: 100×[(experimental release−spontaneous release)/maximum release-spontaneous release)]. Maximum release is determined by lysis of targets by detergent (2% Triton X-100; Sigma Chemical Co., St. Louis, Mo.). Spontaneous release is <25% of maximum release for all experiments.

The results of such an analysis will indicate to what extent HLA-restricted CTL populations have been stimulated with the vaccine. Of course, this protocol can also be used to monitor prior HBV exposure.

The class II restricted HTL responses may also be analyzed. Purified PBMC are cultured in a 96-well flat bottom plate at a density of 1.5×15 cells/well and are stimulated with 10 μg/ml synthetic peptide, whole antigen, or PHA. Cells are routinely plated in replicates of 4-6 wells for each condition. After seven days of culture, the medium is removed and replaced with fresh medium containing 10 U/ml IL-2. Two days later, 1 μCi ³H-thymidine is added to each well and incubation is continued for an additional 18 hours. Cellular DNA is then harvested on glass fiber mats and analyzed for ³H-thymidine incorporation. Antigen-specific T cell proliferation is calculated as the ratio of ³H-thymidine incorporation in the presence of antigen divided by the ³H-thymidine incorporation in the absence of antigen.

The results of such an analysis will indicate to what extent HLA-restricted HTL populations have been stimulated with a vaccine or prior exposure to HBV.

### Example 17

### Induction of Specific CTL Response in Humans

A human clinical trial for an immunogenic composition comprising HBV CTL and HTL epitopes of the invention is set up as an IND Phase I, dose escalation study (5, 50 and 500 μg) and carried out as a randomized, double-blind, placebo-controlled trial. Such a trial is designed, for example, as follows:

A total of about 27 subjects are enrolled and divided into 3 groups:

Group I: 3 subjects are injected with placebo and 6 subjects are injected with 5 μg of peptide composition;

Group II: 3 subjects are injected with placebo and 6 subjects are injected with 50 μg peptide composition;

Group III: 3 subjects are injected with placebo and 6 subjects are injected with 500 μg of peptide composition.

After 4 weeks following the first injection, all subjects receive a booster inoculation at the same dosage.

The endpoints measured in this study relate to the safety and tolerability of the peptide composition as well as its immunogenicity. Cellular immune responses to the peptide composition are an index of the intrinsic activity of this the peptide composition, and can therefore be viewed as a measure of biological efficacy. The following summarize the clinical and laboratory data that relate to safety and efficacy endpoints.

Safety: The incidence of adverse events is monitored in the placebo and drug treatment group and assessed in terms of degree and reversibility.

Evaluation of Vaccine Efficacy: For evaluation of vaccine efficacy, subjects are bled before and after injection. Peripheral blood mononuclear cells are isolated from fresh heparinized blood by Ficoll-Hypaque density gradient centrifugation, aliquoted in freezing media and stored frozen. Samples are assayed for CTL and HTL activity.

Thus, the vaccine is found to be both safe and efficacious.

### Example 18

### Phase II Trials in Patients Infected with HBV

Phase II trials are performed to study the effect of administering the CTL-HTL peptide compositions to patients (male and female) having chronic HBV infection. A main objective of the trials is to determine an effective dose and regimen for inducing CTLs in chronically infected HBV patients, to establish the safety of inducing a CTL response in these patients, and to see to what extent activation of CTLs improves the clinical picture of chronically infected CTL patients, as manifested by a transient flare in alanine aminotransferase (ALT), normalization of ALT, and reduction in HBV DNA. Such a study is designed, for example, as follows:

The studies are performed in multiple centers in the U.S. and Canada. The trial design is an open-label, uncontrolled, dose escalation protocol wherein the peptide composition is administered as a single dose followed six weeks later by a single booster shot of the same dose. The dosages are 50, 500 and 5,000 micrograms per injection. Drug-associated adverse effects are recorded.

There are three patient groupings. The first group is injected with 50 micrograms of the peptide composition and the second and third groups with 500 and 5,000 micrograms of peptide composition, respectively. The patients within each group range in age from 21-65 and include both males and females. The patients represent diverse ethnic backgrounds. All of them are infected with HBV for over five years and are HIV, HCV and HDV negative, but have positive levels of HBe antigen and HBs antigen.

The magnitude and incidence of ALT flares and the levels of HBV DNA in the blood are monitored to assess the effects of administering the peptide compositions. The levels of HBV DNA in the blood are an indirect indication of the progress of treatment. The vaccine composition is found to be both safe and efficacious in the treatment of chronic HBV infection.

The examples herein are provided to illustrate the invention but not to limit its scope. For example, the human terminology for the Major Histocompatibility Complex, namely HLA, is used throughout this document. It is to be appreciated that these principles can be extended to other species as well. Moreover, peptide epitopes have been disclosed in the related application U.S. Ser. No. 08/820,360, which was previously incorporated by reference. Thus, other variants of the invention will be readily apparent to one of ordinary skill in the art and are encompassed by the appended claims. All publications, patents, and patent application cited herein are hereby incorporated by reference for all purposes.

TABLE I

| | | | POSITION |
|---|---|---|---|
| | POSITION | POSITION | C Terminus |
| | 2 | 3 (Primary | (Primary |
| | (Primary Anchor) | Anchor) | Anchor) |
| SUPERMOTIFS | | | |
| A1 | TILVMS | | FWY |
| A2 | LIVMATQ | | IVMATL |
| A3 | VSMATLI | | RK |
| A24 | YFWIVLMT | | FIYWLM |
| B7 | P | | VILFMWYA |
| B27 | RHK | | FYLWMIVA |
| B44 | ED | | FWYLIMVA |
| B58 | ATS | | FWYLIVMA |
| B62 | QLIVMP | | FWYMIVLA |
| MOTIFS | | | |
| A1 | TSM | | Y |
| A1 | | DEAS | Y |
| A2.1 | LMVQIAT | | VLIMAT |
| A3 | LMVISATFCGD | | KYRHFA |
| A11 | VTMLISAGNCDF | | KRYH |
| A24 | YFWM | | FLIW |
| A*3101 | MVTALIS | | RK |
| A*3301 | MVALFIST | | RK |
| A*6801 | AVTMSLI | | RK |
| B*0702 | P | | LMFWYAIV |
| B*3501 | P | | LMFWYIVA |
| B51 | P | | LIVFWYAM |
| B*5301 | P | | IMFWYALV |
| B*5401 | P | | ATIVLMFWY |

Bolded residues are preferred, italicized residues are less preferred: A peptide is considered motif-bearing if it has primary anchors at each primary anchor position for a motif or supermotif as specified in the above table.

TABLE Ia

| | | | POSITION |
|---|---|---|---|
| | POSITION | POSITION | C Terminus |
| | 2 | 3 (Primary | (Primary |
| | (Primary Anchor) | Anchor) | Anchor) |
| SUPERMOTIFS | | | |
| A1 | TILVMS | | FWY |
| A2 | VQAT | | VLIMAT |
| A3 | VSMATLI | | RK |
| A24 | YFWIVLMT | | FIYWLM |
| B7 | P | | VILFMWYA |
| B27 | RHK | | FYLWMIVA |
| B58 | ATS | | FWYLIVMA |
| B62 | QLIVMP | | FWYMIVLA |
| MOTIFS | | | |
| A1 | TSM | | Y |
| A1 | | DEAS | Y |
| A2.1 | VQAT* | | VLIMAT |
| A3.2 | LMVISATFCGD | | KYRHF |
| A11 | VTMLISAGNCDF | | KRH |
| A24 | YFW | | FLIW |

*If 2 is V, or Q, the C-term is not L
Bolded residues are preferred, italicized residues are less preferred: A peptide is considered motif-bearing if it has primary anchors at each primary anchor position for a motif or supermotif as specified in the above table.

TABLE II

| | | POSITION | | | | | | | | |
|---|---|---|---|---|---|---|---|---|---|---|
| | | 1 | 2 | 3 | 4 | 5 | 6 | 7 | 8 | C-terminus |
| SUPERMOTIFS | | | | | | | | | | |
| A1 | | | 1° Anchor | | | | | | | 1° Anchor |
| | | | TILVMS | | | | | | | FWY |
| A2 | | | 1° Anchor | | | | | | | 1° Anchor |
| | | | LIVMATQ | | | | | | | LIVMAT |
| A3 | preferred | | 1° Anchor | YFW (4/5) | | | YFW (3/5) | YFW (4/5) | P (4/5) | 1° Anchor |
| | | | VSMATLI | | | | | | | RK |
| | deleterious | DE (3/5); P (5/5) | | DE (4/5) | | | | | | |
| A24 | | | 1° Anchor | | | | | | | 1° Anchor |
| | | | YFWIVLMT | | | | | | | FIYWLM |
| B7 | preferred | FWY (5/5) | 1° Anchor | FWY (4/5) | | | | | FWY (3/5) | 1° Anchor |
| | | LIVM (3/5) | P | | | | | | | VILFMWYA |
| | deleterious | DE (3/5); P (5/5); | | | | DE (3/5) | G (4/5) | QN (4/5) | DE (4/5) | |
| | | G (4/5); A (3/5); | | | | | | | | |
| | | QN (3/5) | | | | | | | | |
| B27 | | | 1° Anchor | | | | | | | 1° Anchor |
| | | | RHK | | | | | | | FYLWMIVA |
| B44 | | | 1° Anchor | | | | | | | 1° Anchor |
| | | | ED | | | | | | | FWYLIMVA |
| B58 | | | 1° Anchor | | | | | | | 1° Anchor |
| | | | ATS | | | | | | | FWYLIVMA |
| B62 | | | 1° Anchor | | | | | | | 1° Anchor |
| | | | QLIVMP | | | | | | | FWYMIVLA |
| MOTIFS | | | | | | | | | | |
| A1 | preferred | GFYW | 1° Anchor | DEA | YFW | | P | DEQN | YFW | 1° Anchor |
| 9-mer | | | STM | | | | | | | Y |
| | deleterious | DE | | RHKLIVMP | A | G | A | | | |
| A1 | preferred | GRHK | ASTCLIVM | 1° Anchor | GSTC | | ASTC | LIVM | DE | 1° Anchor |
| 9-mer | | | | DEAS | | | | | | Y |
| | deleterious | A | RHKDEPY | | DE | PQN | RHK | PG | GP | |
| | | | FW | | | | | | | |

POSITION

| | | 1 | 2 | 3 | 4 | 5 |
|---|---|---|---|---|---|---|
| A1 | peferred | YFW | 1° Anchor | DEAQN | A | YFWQN |
| 10-mer | | | STM | | | |
| | deleterious | GP | | RHKGLIVM | DE | RHK |
| A1 | preferred | YFW | STCLIVM | 1° Anchor | A | YFW |
| 10-mer | | | | DEAS | | |
| | deleterious | RHK | RHKDEPY | | | P |
| | | | FW | | | |
| A2.1 | preferred | YFW | 1° Anchor | YFW | STC | YFW |
| 9-mer | | | LMIVQAT | | | |
| | deleterious | DEP | | DERKH | | |
| A2.1 | preferred | AYFW | 1° Anchor | LVIM | G | |
| 10-mer | | | LMIVQAT | | | |
| | deleterious | DEP | | DE | RKHA | P |
| A3 | preferred | RHK | 1° Anchor | YFW | PRHKYFW | A |
| | | | LMVISAT | | | |
| | | | FCGD | | | |
| | deleterious | DEP | | DE | | |
| A11 | preferred | A | 1° Anchor | YFW | YFW | A |
| | | | VTLMISA | | | |
| | | | GNCDF | | | |
| | deleterious | DEP | | | | |
| A24 | preferred | YFWRHK | 1° Anchor | | STC | |
| 9-mer | | | YFWM | | | |
| | deleterious | DEG | | DE | G | QNP |
| A24 | preferred | | 1° Anchor | | P | YFWP |
| 10-mer | | | YFWM | | | |
| | deleterious | | | GDE | QN | RHK |
| A3101 | preferred | RHK | 1° Anchor | YFW | P | |
| | | | MVTALIS | | | |
| | deleterious | DEP | | DE | | ADE |
| A3301 | preferred | | 1° Anchor | YFW | | |
| | | | MVALFIST | | | |
| | deleterious | GP | | DE | | |
| A6801 | preferred | YFWSTC | 1° Anchor | | | YFWLIVM |
| | | | AVTMSLI | | | |
| | deleterious | GP | | DEG | | RHK |
| B0702 | preferred | RHKFWY | 1° Anchor | RHK | | RHK |
| | | | P | | | |
| | deleterious | DEQNP | | DEP | DE | DE |
| B3501 | preferred | FWYLIVM | 1° Anchor | FWY | | |
| | | | P | | | |
| | deleterious | AGP | | | | G |
| B51 | preferred | LIVMFWY | 1° Anchor | FWY | STC | FWY |
| | | | P | | | |
| | deleterious | AGPDERHKSTC | | | | DE |
| B5301 | preferred | LIVMFWY | 1° Anchor | FWY | STC | FWY |
| | | | P | | | |
| | deleterious | AGPQN | | | | |
| B5401 | preferred | FWY | 1° Anchor | FWYLIVM | | LIVM |
| | | | P | | | |
| | deleterious | GPQNDE | | GDESTC | | RHKDE |

| | | POSITION | | | | |
|---|---|---|---|---|---|---|
| | | 6 | 7 | 8 | 9 or C-terminus | C-terminus |
| A1 | peferred | | PASTC | GDE | P | 1° Anchor |
| 10-mer | | | | | | Y |
| | deleterious | QNA | RHKYFW | RHK | A | |
| A1 | preferred | | PG | G | YFW | 1° Anchor |
| 10-mer | | | | | | Y |
| | deleterious | G | | PRHK | QN | |
| A2.1 | preferred | | A | P | 1° Anchor | |
| 9-mer | | | | | VLIMAT | |
| | deleterious | RKH | DERKH | | | |
| A2.1 | preferred | G | | FYWL | | 1° Anchor |
| 10-mer | | | | VIM | | VLIMAT |
| | deleterious | | RKH | DERKH | RKH | |
| A3 | preferred | YFW | | P | 1° Anchor | |
| | | | | | KYRHFA | |
| | deleterious | | | | | |
| A11 | preferred | YFW | YFW | P | 1° Anchor | |
| | | | | | KRYH | |
| | deleterious | | A | G | | |
| A24 | preferred | | YFW | YFW | 1° Anchor | |
| 9-mer | | | | | FLIW | |
| | deleterious | DERHK | G | AQN | | |
| A24 | preferred | | P | | | 1° Anchor |
| 10-mer | | | | | | FLIW |
| | deleterious | DE | A | QN | DEA | |
| A3101 | preferred | YFW | YFW | AP | 1° Anchor | |
| | | | | | RK | |
| | deleterious | DE | DE | DE | | |
| A3301 | preferred | | AYFW | | 1° Anchor | |
| | | | | | RK | |
| | deleterious | | | | | |
| A6801 | preferred | | YFW | P | 1° Anchor | |
| | | | | | RK | |
| | deleterious | | | A | | |
| B0702 | preferred | RHK | RHK | PA | 1° Anchor | |
| | | | | | LMFWYAIV | |
| | deleterious | GDE | QN | DE | | |
| B3501 | preferred | | FWY | | 1° Anchor | |
| | | | | | LMFWYIVA | |
| | deleterious | G | | | | |
| B51 | preferred | | G | FWY | 1° Anchor | |
| | | | | | LIVFWYAM | |
| | deleterious | G | DEQN | GDE | | |
| B5301 | preferred | | LIVMFWY | FWY | 1° Anchor | |
| | | | | | IMFWYALV | |
| | deleterious | G | RHKQN | DE | | |
| B5401 | preferred | | ALIVM | FWYAP | 1° Anchor | |
| | | | | | ATIVLMFWY | |
| | deleterious | DE | QNDGE | DE | | |

Italicized residues indicate less preferred or “tolerated” residues.
The information in Table II is specific for 9-mers unless otherwise specified.

TABLE III

| | | POSITION | | | | | | | | |
|---|---|---|---|---|---|---|---|---|---|---|
| MOTIFS | | 1° anchor 1 | 2 | 3 | 4 | 5 | 1° anchor 6 | 7 | 8 | 9 |
| DR4 | preferred | FMYLIVW | M | T | | I | VSTCPALIM | MH | | MH |
| | deleterious | | | | W | | | R | | WDE |
| DR1 | preferred | MFLIVWY | | | PAMQ | | VMATSPLIC | M | | AVM |
| | deleterious | | C | CH | FD | CWD | | GDE | D | |
| DR7 | preferred | MFLIVWY | M | W | A | | IVMSACTPL | M | | IV |
| | deleterious | | C | | G | | | GRD | N | G |

| | | |
|---|---|---|
| DR Supermotif | MFLIVWY | VMSTACPLI |

| DR3 MOTIFS | 1° anchor 1 | 2 | 3 | 1° anchor 4 | 5 | 1° anchor 6 |
|---|---|---|---|---|---|---|
| motif a | | | | | | |
| preferred | LIVMFY | | | D | | |
| motif b | | | | | | |
| preferred | LIVMFAY | | | DNQEST | | KRH |

Italicized residues indicate less preferred or “tolerated” residues.

TABLE IV
HLA Class I Standard Peptide Binding Affinity.

| | | | | BINDING |
|---|---|---|---|---|
| | STANDARD | | | AFFINITY |
| ALLELE | PEPTIDE | SEQUENCE | SEQ ID NO: | (nM) |
| A*0101 | 944.02 | YLEPAIAKY | 3475 | 25 |
| A*0201 | 941.01 | FLPSDYFPSV | 3476 | 5.0 |
| A*0202 | 941.01 | FLPSDYFPSV | 3476 | 4.3 |
| A*0203 | 941.01 | FLPSDYFPSV | 3476 | 10 |
| A*0206 | 941.01 | FLPSDYFPSV | 3476 | 3.7 |
| A*0207 | 941.01 | FLPSDYFPSV | 3476 | 23 |
| A*6802 | 1141.02 | FTQAGYPAL | 3477 | 40 |
| A*0301 | 941.12 | KVFPYALINK | 3478 | 11 |
| A*1101 | 940.06 | AVDLYHFLK | 3479 | 6.0 |
| A*3101 | 941.12 | KVFPYALINK | 3478 | 18 |
| A*3301 | 1083.02 | STLPETYVVRR | 3480 | 29 |
| A*6801 | 941.12 | KVFPYALINK | 3479 | 8.0 |
| A*2402 | 979.02 | AYIDNYNKF | 3481 | 12 |
| B*0702 | 1075.23 | APRTLVYLL | 3482 | 5.5 |
| B*3501 | 1021.05 | FPFKYAAAF | 3483 | 7.2 |
| B51 | 1021.05 | FPFKYAAAF | 3483 | 5.5 |
| B*5301 | 1021.05 | FPFKYAAAF | 3483 | 9.3 |
| B*5401 | 1021.05 | FPFKYAAAF | 3483 | 10 |

TABLE V
HLA Class II Standard Peptide Binding Affinity.

| | | | | | |
|---|---|---|---|---|---|
| | | | | | Binding |
| | | Standard | | SEQ ID | Affinity |
| Allele | Nomenclature | Peptide | Sequence | NO: | (nM) |

| | | | | | |
|---|---|---|---|---|---|
| DRB1*0101 | DR1 | 515.01 | PKYVKQNTLKLAT | 3484 | 5.0 |
| DRB1*0301 | DR3 | 829.02 | YKTIAFDEEARR | 3485 | 300 |
| DRB1*0401 | DR4w4 | 515.01 | PKYVKQNTLKLAT | 3484 | 45 |
| DRB1*0404 | DR4w14 | 717.01 | YARFQSQTTLKQKT | 3486 | 50 |
| DRB1*0405 | DR4w15 | 717.01 | YARFQSQTTLKQKT | 3486 | 38 |
| DRB1*0701 | DR7 | 553.01 | QYIKANSKFIGITE | 3487 | 25 |
| DRB1*0802 | DR8w2 | 553.01 | QYIKANSKFIGITE | 3487 | 49 |
| DRB1*0803 | DR8w3 | 553.01 | QYIKANSKFIGITE | 3487 | 1600 |
| DRB1*0901 | DR9 | 553.01 | QYIKANSKFIGITE | 3487 | 75 |
| DRB1*1101 | DR5w11 | 553.01 | QYIKANSKFIGITE | 3487 | 20 |
| DRB1*1201 | DR5w12 | 1200.05 | EALIHQLKINPYVLS | 3488 | 298 |
| DRB1*1302 | DR6w19 | 650.22 | QYIKANAKFIGITE | 3489 | 3.5 |
| DRB1*1501 | DR2w2β1 | 507.02 | GRTQDENPVVHFFKNI | 3490 | 9.1 |
| | | | VTPRTPPP | | |
| DRB3*0101 | DR52a | 511 | NGQIGNDPNRDIL | 3491 | 470 |
| DRB4*0101 | DRw53 | 717.01 | YARFQSQTTLKQKT | 3486 | 58 |
| DRB5*0101 | DR2w2β2 | 553.01 | QYIKANSKFIGITE | 3487 | 20 |

The “Nomenclature” column lists the allelic designations used in Tables XIX and XX.

TABLE VI

| HLA- | | |
|---|---|---|
| super- | Allele-specific HLA-supertype members | |
| type | Verified^{a} | Predicted^{b} |
| A1 | A*0101, A*2501, A*2601, A*2602, A*3201 | A*0102, A*2604, A*3601, A*4301, A*8001 |
| A2 | A*0201, A*0202, A*0203, A*0204, A*0205, A*0206, A*0207, | A*0208, A*0210, A*0211, A*0212, A*0213 |
| | A*0209, A*0214, A*6802, A*6901 | |
| A3 | A*0301, A*1101, A*3101, A*3301, A*6801 | A*0302, A*1102, A*2603, A*3302, A*3303, A*3401, A*3402, |
| | | A*6601, A*6602, A*7401 |
| A24 | A*2301, A*2402, A*3001 | A*2403, A*2404, A*3002, A*3003 |
| B7 | B*0702, B*0703, B*0704, B*0705, B*1508, B*3501, B*3502, | B*1511, B*4201, B*5901 |
| | B*3503, B*3504, B*3505, B*3506, B*3507, B*3508, B*5101, | |
| | B*5102, B*5103, B*5104, B*5105, B*5301, B*5401, B*5501, | |
| | B*5502, B*5601, B*5602, B*6701, B*7801 | |
| B27 | B*1401, B*1402, B*1509, B*2702, B*2703, B*2704, B*2705, | B*2701, B*2707, B*2708, B*3802, B*3903, B*3904, B*3905, |
| | B*2706, B*3801, B*3901, B*3902, B*7301 | B*4801, B*4802, B*1510, B*1518, B*1503 |
| B44 | B*1801, B*1802, B*3701, B*4402, B*4403, B*4404, B*4001, | B*4101, B*4501, B*4701, B*4901, B*5001 |
| | B*4002, B*4006 | |
| B58 | B*5701, B*5702, B*5801, B*5802, B*1516, B*1517 | |
| B62 | B*1501, B*1502, B*1513, B*5201 | B*1301, B*1302, B*1504, B*1505, B*1506, B*1507, B*1515, |
| | | B*1520, B*1521, B*1512, B*1514, B*1519 |

^{a}Verified alleles includes alleles whose specificity has been determined by pool sequencing analysis, peptide binding assays, or by analysis of the sequences of CTL epitopes.
^{b}Predicted alleles are alleles whose specificity is predicted on the basis of B and F pocket structure to overlap with the supertype specificity.

TABLE VII
HBV A01 SUPER MOTIF(With binding information)

| | | | | | | | |
|---|---|---|---|---|---|---|---|
| Conservancy | Freq. | Protein | Position | Sequence | SEQ ID NO: | String | A*0101 |

| | | | | | | | |
|---|---|---|---|---|---|---|---|
| 95 | 19 | POL | 521 | AICSVVRRAF | 1 | XIXXXXXXXF | |
| 95 | 19 | NUC | 54 | ALRQAILCW | 2 | XLXXXXXXW | |
| 80 | 16 | ENV | 108 | AMQWNSTTF | 3 | XMXXXXXXF | |
| 100 | 20 | POL | 166 | ASFCGSPY | 4 | XSXXXXXY | |
| 100 | 20 | POL | 166 | ASFCGSPYSW | 5 | XSXXXXXXXW | |
| 90 | 18 | NUC | 19 | ASKLCLGW | 6 | XSXXXXXW | |
| 85 | 17 | NUC | 19 | ASKLCLGWLW | 7 | XSXXXXXXXW | |
| 80 | 16 | POL | 822 | ASPLHVAW | 8 | XSXXXXXW | |
| 100 | 20 | ENV | 312 | CIPIPSSW | 9 | XIXXXXXW | |
| 100 | 20 | ENV | 312 | CIPIPSSWAF | 10 | XIXXXXXXXF | |
| 95 | 19 | ENV | 253 | CLIFLLVLLDY | 11 | XLXXXXXXXXY | |
| 95 | 19 | ENV | 239 | CLRRFIIF | 12 | XLXXXXXF | |
| 75 | 15 | ENV | 239 | CLRRFIIFLF | 13 | XLXXXXXXXF | |
| 95 | 19 | POL | 523 | CSVVRRAF | 14 | XSXXXXXF | |
| 100 | 20 | ENV | 310 | CTCIPIPSSW | 15 | XTXXXXXXXW | |
| 90 | 18 | NUC | 31 | DIDPYKEF | 16 | XIXXXXXF | |
| 85 | 17 | NUC | 29 | DLLDTASALY | 17 | XLXXXXXXXY | 11.1000 |
| 95 | 19 | ENV | 196 | DSWWTSLNF | 18 | XSXXXXXXF | |
| 95 | 19 | NUC | 43 | ELLSFLPSDF | 19 | XLXXXXXXXF | |
| 95 | 19 | NUC | 43 | ELLSFLPSDFF | 20 | XLXXXXXXXXF | |
| 95 | 19 | POL | 374 | ESRLVVDF | 21 | XSXXXXXF | |
| 95 | 19 | POL | 374 | ESRLVVDFSQF | 22 | XSXXXXXXXXF | |
| 80 | 16 | ENV | 248 | FILLLCLIF | 23 | XIXXXXXXF | |
| 80 | 16 | ENV | 246 | FLFILLLCLIF | 24 | XLXXXXXXXXF | |
| 95 | 19 | ENV | 256 | FLLVLLDY | 25 | XLXXXXXY | |
| 95 | 19 | POL | 658 | FSPTYKAF | 26 | XSXXXXXF | |
| 90 | 18 | X | 63 | FSSAGPCALRF | 27 | XSXXXXXXXXF | |
| 100 | 20 | ENV | 333 | FSWLSLLVPF | 28 | XSXXXXXXXF | |
| 95 | 19 | POL | 656 | FTFSPTYKAF | 29 | XTXXXXXXXF | |
| 95 | 19 | ENV | 346 | FVGLSPTVW | 30 | XVXXXXXXW | |
| 95 | 19 | POL | 627 | GLLGFAAPF | 31 | XLXXXXXXF | |
| 95 | 19 | POL | 509 | GLSPFLLAQF | 32 | XLXXXXXXXF | |
| 85 | 17 | NUC | 29 | GMDIDPYKEF | 33 | XMXXXXXXXF | |
| 95 | 19 | NUC | 123 | GVWIRTPPAY | 34 | XVXXXXXXXY | 0.0017 |
| 75 | 15 | POL | 569 | HLNPNKTKRW | 35 | XLXXXXXXXW | |
| 80 | 16 | POL | 491 | HLYSHPIILGF | 36 | XLXXXXXXXXF | |
| 85 | 17 | POL | 715 | HTAELLAACF | 37 | XTXXXXXXXF | |
| 95 | 19 | NUC | 52 | HTALRQAILCW | 38 | XTXXXXXXXXW | |
| 100 | 20 | POL | 149 | HTLWKAGILY | 39 | XTXXXXXXXY | 0.0300 |
| 100 | 20 | ENV | 249 | ILLLCLIF | 40 | XLXXXXXF | |
| 80 | 16 | POL | 760 | ILRGTSFVY | 41 | XLXXXXXXY | 0.0017 |
| 90 | 18 | ENV | 188 | ILTIPQSLDSW | 42 | XLXXXXXXXXW | |
| 90 | 18 | POL | 625 | IVGLLGFAAPF | 43 | XVXXXXXXXXF | |
| 80 | 16 | POL | 503 | KIPMGVGLSPF | 44 | XIXXXXXXXXF | |
| 85 | 17 | NUC | 21 | KLCLGWLW | 45 | XLXXXXXW | |
| 75 | 15 | POL | 108 | KLIMPARF | 46 | XLXXXXXF | |
| 75 | 15 | POL | 108 | KLIMPARFY | 47 | XLXXXXXXY | 0.0017 |
| 80 | 16 | POL | 610 | KLPVNRPIDW | 48 | XLXXXXXXXW | |
| 85 | 17 | POL | 574 | KTKRWGYSLNF | 49 | XTXXXXXXXXF | |
| 95 | 19 | POL | 55 | KVGNFTGLY | 50 | XVXXXXXXY | 0.0680 |
| 95 | 19 | ENV | 254 | LIFLLVLLDY | 51 | XIXXXXXXXY | 0.0084 |
| 100 | 20 | POL | 109 | LIMPARFY | 52 | XIXXXXXY | |
| 85 | 17 | NUC | 30 | LLDTASALY | 53 | XLXXXXXXY | 25.0000 |
| 80 | 16 | POL | 752 | LLGCAANW | 54 | XLXXXXXW | |
| 95 | 19 | POL | 628 | LLGFAAPF | 55 | XLXXXXXF | |
| 100 | 20 | ENV | 378 | LLPIFFCLW | 56 | XLXXXXXXW | |
| 100 | 20 | ENV | 378 | LLPIFFCLWVY | 57 | XLXXXXXXXXY | |
| 95 | 19 | NUC | 44 | LLSFLPSDF | 58 | XLXXXXXXF | |
| 95 | 19 | NUC | 44 | LLSFLPSDFF | 59 | XLXXXXXXXF | |
| 90 | 18 | POL | 407 | LLSSNLSW | 60 | XLXXXXXW | |
| 95 | 19 | ENV | 175 | LLVLQAGF | 61 | XLXXXXXF | |
| 95 | 19 | ENV | 175 | LLVLQAGFF | 62 | XLXXXXXXF | |
| 100 | 20 | ENV | 338 | LLVPFVQW | 63 | XLXXXXXW | |
| 100 | 20 | ENV | 338 | LLVPFVQWF | 64 | XLXXXXXXF | |
| 85 | 17 | NUC | 100 | LLWFHISCLTF | 65 | XLXXXXXXXXF | |
| 95 | 19 | NUC | 45 | LSFLPSDF | 66 | XSXXXXXF | |
| 95 | 19 | NUC | 45 | LSFLPSDFF | 67 | XSXXXXXXF | |
| 95 | 19 | POL | 415 | LSLDVSAAF | 68 | XSXXXXXXF | |
| 95 | 19 | POL | 415 | LSLDVSAAFY | 69 | XSXXXXXXXY | 4.2000 |
| 100 | 20 | ENV | 336 | LSLLVPFVQW | 70 | XSXXXXXXXW | |
| 100 | 20 | ENV | 336 | LSLLVPFVQWF | 71 | XSXXXXXXXXF | |
| 95 | 19 | X | 53 | LSLRGLPVCAF | 72 | XSXXXXXXXXF | |
| 95 | 19 | POL | 510 | LSPFLLAQF | 73 | XSXXXXXXF | |
| 75 | 15 | ENV | 349 | LSPTVWLSVIW | 74 | XSXXXXXXXXW | |
| 85 | 17 | POL | 742 | LSRKYTSF | 75 | XSXXXXXF | |
| 85 | 17 | POL | 742 | LSRKYTSFPW | 76 | XSXXXXXXXW | |
| 75 | 15 | ENV | 16 | LSVPNPLGF | 77 | XSXXXXXXF | |
| 75 | 15 | NUC | 137 | LTFGRETVLEY | 78 | XTXXXXXXXXY | |
| 90 | 18 | ENV | 189 | LTIPQSLDSW | 79 | XTXXXXXXXW | |
| 90 | 18 | ENV | 189 | LTIPQSLDSWW | 80 | XTXXXXXXXXW | |
| 90 | 18 | POL | 404 | LTNLLSSNLSW | 81 | XTXXXXXXXXW | |
| 95 | 19 | ENV | 176 | LVLQAGFF | 82 | XVXXXXXF | |
| 100 | 20 | ENV | 339 | LVPFVQWF | 83 | XVXXXXXF | |
| 100 | 20 | POL | 377 | LVVDFSQF | 84 | XVXXXXXF | |
| 85 | 17 | ENV | 360 | MMWYWGPSLY | 85 | XMXXXXXXXY | 0.0810 |
| 75 | 15 | X | 103 | MSTTDLEAY | 86 | XSXXXXXXY | 0.8500 |
| 75 | 15 | X | 103 | MSTTDLEAYF | 87 | XSXXXXXXXF | |
| 95 | 19 | POL | 42 | NLGNLNVSIPW | 88 | XLXXXXXXXXW | |
| 90 | 18 | POL | 406 | NLLSSNLSW | 89 | XLXXXXXXW | |
| 95 | 19 | POL | 45 | NLNVSIPW | 90 | XLXXXXXW | |
| 75 | 15 | ENV | 15 | NLSVPNPLGF | 91 | XLXXXXXXXF | |
| 90 | 18 | POL | 738 | NSVVLSRKY | 92 | XSXXXXXXY | 0.0005 |
| 100 | 20 | ENV | 380 | PIFFCLWVY | 93 | XIXXXXXXY | 0.0078 |
| 100 | 20 | ENV | 314 | PIPSSWAF | 94 | XIXXXXXF | |
| 100 | 20 | POL | 124 | PLDKGIKPY | 95 | XLXXXXXXY | 0.0190 |
| 100 | 20 | POL | 124 | PLDKGIKPYY | 96 | XLXXXXXXXY | 0.1600 |
| 100 | 20 | ENV | 377 | PLLPIFFCLW | 97 | XLXXXXXXXW | |
| 95 | 19 | ENV | 174 | PLLVLQAGF | 98 | XLXXXXXXF | |
| 95 | 19 | ENV | 174 | PLLVLQAGFF | 99 | XLXXXXXXXF | |
| 80 | 16 | POL | 505 | PMGVGLSPF | 100 | XMXXXXXXF | |
| 85 | 17 | POL | 797 | PTTGRTSLY | 101 | XTXXXXXXY | 0.7700 |
| 75 | 15 | ENV | 351 | PTVWLSVIW | 102 | XTXXXXXXW | |
| 85 | 17 | POL | 612 | PVNRPIDW | 103 | XVXXXXXW | |
| 95 | 19 | POL | 685 | QVFADATPTG | 104 | XVXXXXXXXXW | |
| 90 | 18 | POL | 624 | RIVGLLGF | 105 | XIXXXXXF | |
| 75 | 15 | POL | 106 | RLKLIMPARF | 106 | XLXXXXXXXF | |
| 75 | 15 | POL | 106 | RLKLIMPARFY | 107 | XLXXXXXXXXY | |
| 95 | 19 | POL | 376 | RLVVDFSQF | 108 | XLXXXXXXF | |
| 90 | 18 | POL | 353 | RTPARVTGGVF | 109 | XTXXXXXXXXF | |
| 100 | 20 | POL | 49 | SIPWTHKVGNF | 110 | XIXXXXXXXXF | |
| 95 | 19 | ENV | 194 | SLDSWWTSLNF | 111 | XLXXXXXXXXF | |
| 95 | 19 | POL | 416 | SLDVSAAF | 112 | XLXXXXXF | |
| 95 | 19 | POL | 416 | SLDVSAAFY | 113 | XLXXXXXXY | 17.2000 |
| 100 | 20 | ENV | 337 | SLLVPFVQW | 114 | XLXXXXXXW | |
| 100 | 20 | ENV | 337 | SLLVPFVQWF | 115 | XLXXXXXXXF | |
| 95 | 19 | X | 54 | SLRGLPVCAF | 116 | XLXXXXXXXF | |
| 90 | 18 | X | 64 | SSAGPCALRF | 117 | XSXXXXXXXF | |
| 75 | 15 | X | 104 | STTDLEAY | 118 | XTXXXXXY | |
| 75 | 15 | X | 104 | STTDLEAYF | 119 | XTXXXXXXF | |
| 75 | 15 | ENV | 17 | SVPNPLGF | 120 | XVXXXXXF | |
| 90 | 18 | POL | 739 | SVVLSRKY | 121 | XVXXXXXY | |
| 85 | 17 | POL | 739 | SVVLSRKYTSF | 122 | XVXXXXXXXXF | |
| 90 | 18 | ENV | 190 | TIPQSLDSW | 123 | XIXXXXXXW | |
| 90 | 18 | ENV | 190 | TIPQSLDSWW | 124 | XIXXXXXXXW | |
| 100 | 20 | POL | 150 | TLWKAGILY | 125 | XLXXXXXXY | 0.0017 |
| 75 | 15 | X | 105 | TTDLEAYF | 126 | XTXXXXXF | |
| 85 | 17 | POL | 798 | TTGRTSLY | 127 | XTXXXXXY | |
| 80 | 16 | NUC | 16 | TVQASKLCLGW | 128 | XVXXXXXXXXW | |
| 75 | 15 | ENV | 352 | TVWLSVIW | 129 | XVXXXXXW | |
| 85 | 17 | POL | 741 | VLSRKYTSF | 130 | XLXXXXXXF | |
| 85 | 17 | POL | 741 | VLSRKYTSFPW | 131 | XLXXXXXXXXW | |
| 85 | 17 | POL | 740 | VVLSRKYTSF | 132 | XVXXXXXXXF | |
| 80 | 16 | POL | 759 | WILRGTSF | 133 | XIXXXXXF | |
| 80 | 16 | POL | 759 | WILRGTSFVY | 134 | XIXXXXXXXY | 0.0023 |
| 95 | 19 | NUC | 125 | WIRTPPAY | 135 | XIXXXXXY | |
| 80 | 16 | POL | 751 | WLLGCAANW | 136 | XLXXXXXXW | |
| 95 | 19 | POL | 414 | WLSLDVSAAF | 137 | XLXXXXXXXF | |
| 95 | 19 | POL | 414 | WLSLDVSAAFY | 138 | XLXXXXXXXXY | |
| 100 | 20 | ENV | 335 | WLSLLVPF | 139 | XLXXXXXF | |
| 100 | 20 | ENV | 335 | WLSLLVPFVQW | 140 | XLXXXXXXXXW | |
| 85 | 17 | NUC | 26 | WLWGMDIDPY | 141 | XLXXXXXXXY | 0.0810 |
| 95 | 19 | ENV | 237 | WMCLRRFIIF | 142 | XMXXXXXXXF | |
| 85 | 17 | ENV | 359 | WMMWYWGPS | 143 | XMXXXXXXXXY | |
| 100 | 20 | POL | 52 | WTHKVGNF | 144 | XTXXXXXF | |
| 100 | 20 | POL | 122 | YLPLDKGIKPY | 145 | XLXXXXXXXXY | |
| 90 | 18 | NUC | 118 | YLVSFGVW | 146 | XLXXXXXW | |
| 80 | 16 | POL | 493 | YSHPIILGF | 147 | XSXXXXXXF | |
| 85 | 17 | POL | 580 | YSLNFMGY | 148 | XSXXXXXY | |

TABLE VIII
HBV A02 SUPER MOTIF (With binding information)

| | | | | | | | | | | | |
|---|---|---|---|---|---|---|---|---|---|---|---|
| | | | | | SEQ ID | | | | | | |
| Conservancy | Frequency | Protein | Position | Sequence | NO: | AA | A*0201 | A*0202 | A*0203 | A*0206 | A*6802 |

| | | | | | | | | | | | |
|---|---|---|---|---|---|---|---|---|---|---|---|
| 85 | 17 | POL | 721 | AACFARSRSGA | 149 | 11 | | | | | |
| 85 | 17 | POL | 431 | AAMPHLLV | 150 | 8 | | | | | |
| 80 | 16 | POL | 756 | AANWILRGT | 151 | 9 | | | | | |
| 95 | 19 | POL | 632 | AAPFTQCGYPA | 152 | 11 | | | | | |
| 95 | 19 | POL | 521 | AICSVVRRA | 153 | 9 | 0.0001 | | | | |
| 90 | 18 | NUC | 58 | AILCWGEL | 154 | 8 | | | | | |
| 90 | 18 | NUC | 58 | AILCWGELM | 155 | 9 | | | | | |
| 95 | 19 | POL | 642 | ALMPLYACI | 156 | 9 | 0.5000 | 0.0340 | 3.3000 | 0.2500 | 0.0470 |
| 80 | 16 | ENV | 108 | AMQWNSTT | 157 | 8 | | | | | |
| 75 | 15 | X | 102 | AMSTTDLEA | 158 | 9 | 0.0013 | | | | |
| 95 | 19 | POL | 516 | AQFTSAICSV | 159 | 10 | | | | | |
| 95 | 19 | POL | 516 | AQFTSAICSVV | 160 | 11 | | | | | |
| 95 | 19 | POL | 690 | ATPTGWGL | 161 | 8 | | | | | |
| 80 | 16 | POL | 690 | ATPTGWGLA | 162 | 9 | | | | | |
| 75 | 15 | POL | 690 | ATPTGWGLAI | 163 | 10 | | | | | |
| 95 | 19 | POL | 397 | AVPNLQSL | 164 | 8 | | | | | |
| 95 | 19 | POL | 397 | AVPNLQSLT | 165 | 9 | 0.0001 | | | | |
| 95 | 19 | POL | 397 | AVPNLQSLTNL | 166 | 11 | | | | | |
| 80 | 16 | POL | 755 | CAANWILRGT | 167 | 10 | | | | | |
| 95 | 19 | X | 61 | CAFSSAGPCA | 168 | 10 | 0.0001 | | | | |
| 95 | 19 | X | 61 | CAFSSAGPCAL | 169 | 11 | | | | | |
| 90 | 18 | X | 69 | CALRFTSA | 170 | 8 | | | | | |
| 100 | 20 | ENV | 312 | CIPIPSSWA | 171 | 9 | 0.0010 | | | | |
| 80 | 16 | ENV | 312 | CIPIPSSWAFA | 172 | 11 | | | | | |
| 90 | 18 | POL | 533 | CLAFSYMDDV | 173 | 10 | 0.0008 | | | | |
| 90 | 18 | POL | 533 | CLAFSYMDDVV | 174 | 11 | | | | | |
| 85 | 17 | NUC | 23 | CLGWLWGM | 175 | 8 | | | | | |
| 85 | 17 | NUC | 23 | CLGWLWGMDI | 176 | 10 | 0.0093 | | | | |
| 100 | 20 | ENV | 253 | CLIFLLVL | 177 | 8 | 0.0002 | | | | |
| 100 | 20 | ENV | 253 | CLIFLLVLL | 178 | 9 | 0.0006 | | | | |
| 95 | 19 | ENV | 239 | CLRRFIIFL | 179 | 9 | 0.0002 | | | | |
| 75 | 15 | ENV | 239 | CLRRFIIFLFI | 180 | 11 | 0.0004 | | | | |
| 90 | 18 | NUC | 107 | CLTFGRET | 181 | 8 | | | | | |
| 90 | 18 | NUC | 107 | CLTFGRETV | 182 | 9 | 0.0001 | | | | |
| 80 | 16 | X | 7 | CQLDPARDV | 183 | 9 | | | | | |
| 80 | 16 | X | 7 | CQLDPARDVL | 184 | 10 | | | | | |
| 85 | 17 | POL | 622 | CQRIVGLL | 185 | 8 | | | | | |
| 85 | 17 | POL | 622 | CQRIVGLLGFA | 186 | 11 | | | | | |
| 95 | 19 | POL | 684 | CQVFADAT | 187 | 8 | | | | | |
| 95 | 19 | POL | 684 | CQVFADATPT | 188 | 10 | | | | | |
| 100 | 20 | ENV | 310 | CTCIPIPSSWA | 189 | 11 | | | | | |
| 95 | 19 | POL | 689 | DATPTGWGL | 190 | 9 | 0.0001 | | | | |
| 80 | 16 | POL | 689 | DATPTGWGLA | 191 | 10 | | | | | |
| 75 | 15 | POL | 689 | DATPTGWGLAI | 192 | 11 | | | | | |
| 90 | 18 | NUC | 31 | DIDPYKEFGA | 193 | 10 | | | | | |
| 85 | 17 | NUC | 29 | DLLDTASA | 194 | 8 | | | | | |
| 85 | 17 | NUC | 29 | DLLDTASAL | 195 | 9 | 0.0001 | | | | |
| 95 | 19 | POL | 40 | DLNLGNLNV | 196 | 9 | 0.0004 | | | | |
| 95 | 19 | POL | 40 | DLNLGNLNVSI | 197 | 11 | | | | | |
| 80 | 16 | NUC | 32 | DTASALYREA | 198 | 10 | | | | | |
| 80 | 16 | NUC | 32 | DTASALYREAL | 199 | 11 | | | | | |
| 95 | 19 | X | 14 | DVLCLRPV | 200 | 8 | | | | | |
| 95 | 19 | X | 14 | DVLCLRPVGA | 201 | 10 | 0.0001 | | | | |
| 90 | 18 | POL | 541 | DVVLGAKSV | 202 | 9 | 0.0003 | | | | |
| 100 | 20 | POL | 17 | EAGPLEEEL | 203 | 9 | 0.0001 | | | | |
| 80 | 16 | X | 122 | ELGEEIRL | 204 | 8 | | | | | |
| 90 | 18 | POL | 718 | ELLAACFA | 205 | 8 | | | | | |
| 75 | 15 | NUC | 142 | ETVLEYLV | 206 | 8 | | | | | |
| 95 | 19 | POL | 687 | FADATPTGWGL | 207 | 11 | | | | | |
| 85 | 17 | POL | 724 | FARSRSGA | 208 | 8 | | | | | |
| 80 | 16 | POL | 821 | FASPLHVA | 209 | 8 | | | | | |
| 95 | 19 | POL | 396 | FAVPNLQSL | 210 | 9 | | | | | |
| 95 | 19 | POL | 396 | FAVPNLQSLT | 211 | 10 | 0.0003 | | | | |
| 80 | 16 | ENV | 243 | FIIFLFIL | 212 | 8 | 0.0006 | | | | |
| 80 | 16 | ENV | 243 | FIIFLFILL | 213 | 9 | 0.0002 | | | | |
| 80 | 16 | ENV | 243 | FIIFLFILLL | 214 | 10 | 0.0012 | | | | |
| 80 | 16 | ENV | 248 | FILLLCLI | 215 | 8 | 0.0003 | | | | |
| 80 | 16 | ENV | 248 | FILLLCLIFL | 216 | 10 | 0.0280 | | | | |
| 80 | 16 | ENV | 248 | FILLLCLIFLL | 217 | 11 | 0.0010 | | | | |
| 80 | 16 | ENV | 246 | FLFILLLCL | 218 | 9 | 0.0002 | | | | |
| 80 | 16 | ENV | 246 | FLFILLLCLI | 219 | 10 | 0.0013 | | | | |
| 75 | 15 | ENV | 171 | FLGPLLVL | 220 | 8 | | | | | |
| 75 | 15 | ENV | 171 | FLGPLLVLQA | 221 | 10 | 0.0190 | | | | |
| 95 | 19 | POL | 513 | FLLAQFTSA | 222 | 9 | 0.2400 | | | | |
| 95 | 19 | POL | 513 | FLLAQFTSAI | 223 | 10 | 0.2100 | 0.0320 | 7.0000 | 0.1100 | 0.0880 |
| 95 | 19 | POL | 562 | FLLSLGIHL | 224 | 9 | 0.6500 | 0.0010 | 0.0100 | 0.1100 | 0.0035 |
| 80 | 16 | ENV | 183 | FLLTRILT | 225 | 8 | | | | | |
| 80 | 16 | ENV | 183 | FLLTRILTI | 226 | 9 | 0.5100 | 0.0430 | 8.0000 | 0.2000 | 0.0010 |
| 95 | 19 | ENV | 256 | FLLVLLDYQGM | 227 | 11 | | | | | |
| 100 | 20 | POL | 363 | FLVDKNPHNT | 228 | 10 | 0.0012 | | | | |
| 95 | 19 | POL | 656 | FTFSPTYKA | 229 | 9 | 0.0056 | 0.0150 | 0.0031 | 0.8000 | 7.3000 |
| 95 | 19 | POL | 656 | FTFSPTYKAFL | 230 | 11 | | | | | |
| 95 | 19 | POL | 59 | FTGLYSST | 231 | 8 | | | | | |
| 90 | 18 | POL | 59 | FTGLYSSTV | 232 | 9 | 0.0005 | | | | |
| 95 | 19 | POL | 635 | FTQCGYPA | 233 | 8 | | | | | |
| 95 | 19 | POL | 635 | FTQCGYPAL | 234 | 9 | 0.0009 | | | | |
| 95 | 19 | POL | 635 | FTQCGYPALM | 235 | 10 | 0.0024 | | | | |
| 95 | 19 | POL | 518 | FTSAICSV | 236 | 8 | | | | | |
| 95 | 19 | POL | 518 | FTSAICSVV | 237 | 9 | 0.0090 | | | | |
| 95 | 19 | ENV | 346 | FVGLSPTV | 238 | 8 | | | | | |
| 95 | 19 | ENV | 346 | FVGLSPTVWL | 239 | 10 | 0.0008 | | | | |
| 90 | 18 | X | 132 | FVLGGCRHKL | 240 | 10 | 0.0030 | | | | |
| 90 | 18 | X | 132 | FVLGGCRHKLV | 241 | 11 | | | | | |
| 95 | 19 | ENV | 342 | FVQWFVGL | 242 | 8 | | | | | |
| 95 | 19 | ENV | 342 | FVQWFVGLSPT | 243 | 11 | | | | | |
| 90 | 18 | POL | 766 | FVYVPSAL | 244 | 8 | | | | | |
| 90 | 18 | POL | 766 | FVYVPSALNPA | 245 | 11 | | | | | |
| 95 | 19 | X | 50 | GAHLSLRGL | 246 | 9 | 0.0001 | | | | |
| 90 | 18 | X | 50 | GAHLSLRGLPV | 247 | 11 | | | | | |
| 85 | 17 | POL | 545 | GAKSVQHL | 248 | 8 | | | | | |
| 85 | 17 | POL | 545 | GAKSVQHLESL | 249 | 11 | | | | | |
| 75 | 15 | POL | 567 | GIHLNPNKT | 250 | 9 | | | | | |
| 90 | 18 | POL | 155 | GILYKRET | 251 | 8 | | | | | |
| 90 | 18 | POL | 155 | GILYKRETT | 252 | 9 | | | | | |
| 85 | 17 | POL | 682 | GLCQVFADA | 253 | 9 | 0.0024 | | | | |
| 85 | 17 | POL | 682 | GLCQVFADAT | 254 | 10 | | | | | |
| 95 | 19 | POL | 627 | GLLGFAAPFT | 255 | 10 | 0.0049 | | | | |
| 85 | 17 | ENV | 62 | GLLGWSPQA | 256 | 9 | 0.4000 | 0.0003 | 0.0350 | 0.2800 | 0.0005 |
| 95 | 19 | X | 57 | GLPVCAFSSA | 257 | 10 | 0.0008 | | | | |
| 95 | 19 | POL | 509 | GLSPFLLA | 258 | 8 | | | | | |
| 95 | 19 | POL | 509 | GLSPFLLAQFT | 259 | 11 | | | | | |
| 100 | 20 | ENV | 348 | GLSPTVWL | 260 | 8 | 0.0036 | | | | |
| 75 | 15 | ENV | 348 | GLSPTVWLSV | 261 | 10 | 0.2800 | | | | |
| 75 | 15 | ENV | 348 | GLSPTVWLSVI | 262 | 11 | 0.0036 | | | | |
| 90 | 18 | ENV | 265 | GMLPVCPL | 263 | 8 | | | | | |
| 90 | 18 | POL | 735 | GTDNSVVL | 264 | 8 | | | | | |
| 75 | 15 | ENV | 13 | GTNLSVPNPL | 265 | 10 | | | | | |
| 80 | 16 | POL | 763 | GTSFVYVPSA | 266 | 10 | | | | | |
| 80 | 16 | POL | 763 | GTSFVYVPSAL | 267 | 11 | | | | | |
| 80 | 16 | POL | 507 | GVGLSPFL | 268 | 8 | | | | | |
| 80 | 16 | POL | 507 | GVGLSPFLL | 269 | 9 | 0.0002 | | | | |
| 80 | 16 | POL | 507 | GVGLSPFLLA | 270 | 10 | | | | | |
| 95 | 19 | NUC | 123 | GVWIRTPPA | 271 | 9 | 0.0030 | | | | |
| 90 | 18 | NUC | 104 | HISCLTFGRET | 272 | 11 | | | | | |
| 80 | 16 | POL | 435 | HLLVGSSGL | 273 | 9 | 0.0031 | | | | |
| 90 | 18 | X | 52 | HLSLRGLPV | 274 | 9 | 0.0014 | | | | |
| 90 | 18 | X | 52 | HLSLRGLPVCA | 275 | 11 | | | | | |
| 80 | 16 | POL | 491 | HLYSHPII | 276 | 8 | | | | | |
| 80 | 16 | POL | 491 | HLYSHPIIL | 277 | 9 | 0.2200 | 0.0003 | 0.9300 | 0.1700 | 0.0530 |
| 85 | 17 | POL | 715 | HTAELLAA | 278 | 8 | | | | | |
| 85 | 17 | POL | 715 | HTAELLAACFA | 279 | 11 | | | | | |
| 100 | 20 | NUC | 52 | HTALRQAI | 280 | 8 | | | | | |
| 95 | 19 | NUC | 52 | HTALRQAIL | 281 | 9 | 0.0001 | | | | |
| 100 | 20 | POL | 149 | HTLWKAGI | 282 | 8 | | | | | |
| 100 | 20 | POL | 149 | HTLWKAGIL | 283 | 9 | 0.0001 | | | | |
| 80 | 16 | ENV | 244 | IIFLFILL | 284 | 8 | 0.0004 | | | | |
| 80 | 16 | ENV | 244 | IIFLFILLL | 285 | 9 | 0.0002 | | | | |
| 80 | 16 | ENV | 244 | IIFLFILLLCL | 286 | 11 | 0.0002 | | | | |
| 80 | 16 | POL | 497 | IILGFRKI | 287 | 8 | | | | | |
| 80 | 16 | POL | 497 | IILGFRKIPM | 288 | 10 | | | | | |
| 90 | 18 | NUC | 59 | ILCWGELM | 289 | 8 | | | | | |
| 80 | 16 | POL | 498 | ILGFRKIPM | 290 | 9 | 0.0002 | | | | |
| 100 | 20 | ENV | 249 | ILLLCLIFL | 291 | 9 | 0.0015 | | | | |
| 100 | 20 | ENV | 249 | ILLLCLIFLL | 292 | 10 | 0.0190 | 0.0001 | 0.0002 | 0.1300 | 0.0015 |
| 100 | 20 | ENV | 249 | ILLLCLIFLLV | 293 | 11 | 0.0056 | | | | |
| 80 | 16 | POL | 760 | ILRGTSFV | 294 | 8 | | | | | |
| 80 | 16 | POL | 760 | ILRGTSFVYV | 295 | 10 | 0.0160 | | | | |
| 100 | 20 | NUC | 139 | ILSTLPET | 296 | 8 | | | | | |
| 100 | 20 | NUC | 139 | ILSTLPETT | 297 | 9 | 0.0001 | | | | |
| 100 | 20 | NUC | 139 | ILSTLPETTV | 298 | 10 | 0.0210 | 0.0085 | 0.0770 | 0.3100 | 0.0067 |
| 100 | 20 | NUC | 139 | ILSTLPETTVV | 299 | 11 | | | | | |
| 95 | 19 | ENV | 188 | ILTIPQSL | 300 | 8 | | | | | |
| 90 | 18 | POL | 156 | ILYKRETT | 301 | 8 | | | | | |
| 90 | 18 | POL | 625 | IVGLLGFA | 302 | 8 | | | | | |
| 90 | 18 | POL | 625 | IVGLLGFAA | 303 | 9 | 0.0009 | | | | |
| 90 | 18 | POL | 153 | KAGILYKRET | 304 | 10 | | | | | |
| 90 | 18 | POL | 153 | KAGILYKRETT | 305 | 11 | | | | | |
| 80 | 16 | POL | 503 | KIPMGVGL | 306 | 8 | | | | | |
| 85 | 17 | NUC | 21 | KLCLGWLWGM | 307 | 10 | 0.0001 | | | | |
| 95 | 19 | POL | 489 | KLHLYSHPI | 308 | 9 | 0.0690 | 0.0340 | 2.7000 | 0.5900 | 0.0015 |
| 80 | 16 | POL | 489 | KLHLYSHPII | 309 | 10 | | | | | |
| 80 | 16 | POL | 489 | KLHLYSHPIIL | 310 | 11 | | | | | |
| 80 | 16 | POL | 610 | KLPVNRPI | 311 | 8 | | | | | |
| 95 | 19 | POL | 653 | KQAFTFSPT | 312 | 9 | | | | | |
| 95 | 19 | POL | 574 | KTKRWGYSL | 313 | 9 | 0.0001 | | | | |
| 85 | 17 | POL | 620 | KVCQRIVGL | 314 | 9 | 0.0003 | | | | |
| 85 | 17 | POL | 620 | KVCQRIVGLL | 315 | 10 | 0.0001 | | | | |
| 95 | 19 | POL | 55 | KVGNFTGL | 316 | 8 | | | | | |
| 85 | 17 | X | 91 | KVLHKRTL | 317 | 8 | | | | | |
| 85 | 17 | X | 91 | KVLHKRTLGL | 318 | 10 | 0.0004 | | | | |
| 90 | 18 | POL | 534 | LAFSYMDDV | 319 | 9 | 0.0002 | | | | |
| 90 | 18 | POL | 534 | LAFSYMDDVV | 320 | 10 | 0.0003 | | | | |
| 90 | 18 | POL | 534 | LAFSYMDDVVL | 321 | 11 | | | | | |
| 95 | 19 | POL | 515 | LAQFTSAI | 322 | 8 | | | | | |
| 95 | 19 | POL | 515 | LAQFTSAICSV | 323 | 11 | | | | | |
| 100 | 20 | ENV | 254 | LIFLLVLL | 324 | 8 | 0.0025 | | | | |
| 95 | 19 | POL | 514 | LLAQFTSA | 325 | 8 | | | | | |
| 95 | 19 | POL | 514 | LLAQFTSAI | 326 | 9 | 0.1000 | 0.2700 | 3.7000 | 0.2600 | 0.7900 |
| 100 | 20 | ENV | 251 | LLCLIFLL | 327 | 8 | 0.0004 | | | | |
| 100 | 20 | ENV | 251 | LLCLIFLLV | 328 | 9 | 0.0048 | | | | |
| 100 | 20 | ENV | 251 | LLCLIFLLVL | 329 | 10 | 0.0075 | | | | |
| 100 | 20 | ENV | 251 | LLCLIFLLVLL | 330 | 11 | 0.0013 | | | | |
| 85 | 17 | NUC | 30 | LLDTASAL | 331 | 8 | | | | | |
| 95 | 19 | ENV | 260 | LLDYQGML | 332 | 8 | 0.0004 | | | | |
| 90 | 18 | ENV | 260 | LLDYQGMLPV | 333 | 10 | 0.0980 | 0.0001 | 0.0200 | 0.6700 | 0.0009 |
| 80 | 16 | POL | 752 | LLGCAANWI | 334 | 9 | 0.0011 | | | | |
| 80 | 16 | POL | 752 | LLGCAANWIL | 335 | 10 | 0.0140 | | | | |
| 95 | 19 | POL | 628 | LLGFAAPFT | 336 | 9 | 0.0008 | | | | |
| 85 | 17 | ENV | 63 | LLGWSPQA | 337 | 8 | | | | | |
| 75 | 15 | ENV | 63 | LLGWSPQAQGI | 338 | 11 | | | | | |
| 100 | 20 | ENV | 250 | LLLCLIFL | 339 | 8 | 0.0006 | | | | |
| 100 | 20 | ENV | 250 | LLLCLIFLL | 340 | 9 | 0.0065 | | | | |
| 100 | 20 | ENV | 250 | LLLCLIFLLV | 341 | 10 | 0.0036 | | | | |
| 100 | 20 | ENV | 250 | LLLCLIFLLVL | 342 | 11 | 0.0005 | | | | |
| 100 | 20 | ENV | 378 | LLPIFFCL | 343 | 8 | 0.0055 | | | | |
| 100 | 20 | ENV | 378 | LLPIFFCLWV | 344 | 10 | 0.0320 | 0.0008 | 0.0150 | 0.8000 | 0.0005 |
| 95 | 19 | POL | 563 | LLSLGIHL | 345 | 8 | | | | | |
| 90 | 18 | POL | 407 | LLSSNLSWL | 346 | 9 | 0.0110 | 0.0780 | 3.9000 | 0.2700 | 0.0100 |
| 90 | 18 | POL | 407 | LLSSNLSWLSL | 347 | 11 | | | | | |
| 80 | 16 | ENV | 184 | LLTRILTI | 348 | 8 | 0.0026 | | | | |
| 80 | 16 | POL | 436 | LLVGSSGL | 349 | 8 | | | | | |
| 95 | 19 | ENV | 257 | LLVLLDYQGM | 350 | 10 | 0.0050 | | | | |
| 95 | 19 | ENV | 257 | LLVLLDYQGML | 351 | 11 | | | | | |
| 90 | 18 | ENV | 175 | LLVLQAGFFL | 352 | 10 | 0.0310 | 0.0037 | 0.0045 | 0.1500 | 0.0110 |
| 90 | 18 | ENV | 175 | LLVLQAGFFLL | 353 | 11 | 0.0074 | | | | |
| 95 | 19 | ENV | 338 | LLVPFVQWFV | 354 | 10 | 0.6700 | 0.3800 | 1.7000 | 0.2900 | 0.1400 |
| 90 | 18 | NUC | 100 | LLWFHISCL | 355 | 9 | 0.0130 | 0.0002 | 0.0420 | 0.3100 | 0.0098 |
| 85 | 17 | NUC | 100 | LLWFHISCLT | 356 | 10 | | | | | |
| 95 | 19 | POL | 643 | LMPLYACI | 357 | 8 | | | | | |
| 95 | 19 | ENV | 178 | LQAGFFLL | 358 | 8 | | | | | |
| 95 | 19 | ENV | 178 | LQAGFFLLT | 359 | 9 | | | | | |
| 80 | 16 | ENV | 178 | LQAGFFLLTRI | 360 | 11 | | | | | |
| 100 | 20 | POL | 401 | LQSLTNLL | 361 | 8 | | | | | |
| 95 | 19 | NUC | 108 | LTFGRETV | 362 | 8 | | | | | |
| 75 | 15 | NUC | 137 | LTFGRETVL | 363 | 9 | | | | | |
| 90 | 18 | POL | 404 | LTNLLSSNL | 364 | 9 | | | | | |
| 80 | 16 | ENV | 185 | LTRILTIPQSL | 365 | 11 | | | | | |
| 85 | 17 | POL | 99 | LTVNEKRRL | 366 | 9 | | | | | |
| 100 | 20 | POL | 364 | LVDKNPHNT | 367 | 9 | 0.0001 | | | | |
| 95 | 19 | ENV | 258 | LVLLDVQGM | 368 | 9 | 0.0001 | | | | |
| 95 | 19 | ENV | 258 | LVLLDYQGML | 369 | 10 | 0.0001 | | | | |
| 90 | 18 | ENV | 176 | LVLQAGFFL | 370 | 9 | 0.0096 | | | | |
| 90 | 18 | ENV | 176 | LVLQAGFFLL | 371 | 10 | 0.0022 | | | | |
| 90 | 18 | ENV | 176 | LVLQAGFFLLT | 372 | 11 | | | | | |
| 95 | 19 | ENV | 339 | LVPFVQWFV | 373 | 9 | 0.0420 | 0.0150 | 0.0048 | 0.7900 | 2.8000 |
| 95 | 19 | ENV | 339 | LVPFVQWFVGL | 374 | 11 | | | | | |
| 90 | 18 | NUC | 119 | LVSFGVWI | 375 | 8 | 0.0004 | | | | |
| 90 | 18 | NUC | 119 | LVSFGVWIRT | 376 | 10 | | | | | |
| 85 | 17 | ENV | 360 | MMWYWGPSL | 377 | 9 | 0.6400 | | | | |
| 75 | 15 | NUC | 1 | MQLFHLCL | 378 | 8 | | | | | |
| 100 | 20 | NUC | 136 | NAPILSTL | 379 | 8 | | | | | |
| 100 | 20 | NUC | 136 | NAPILSTLPET | 380 | 11 | | | | | |
| 95 | 19 | POL | 42 | NLGNLNVSI | 381 | 9 | 0.0047 | | | | |
| 90 | 18 | POL | 406 | NLLSSNLSWL | 382 | 10 | 0.0016 | | | | |
| 95 | 19 | POL | 45 | NLNVSIPWT | 383 | 9 | 0.0005 | | | | |
| 100 | 20 | POL | 400 | NLQSLTNL | 384 | 8 | | | | | |
| 100 | 20 | POL | 400 | NLQSLTNLL | 385 | 9 | 0.0047 | | | | |
| 75 | 15 | ENV | 15 | NLSVPNPL | 386 | 8 | | | | | |
| 90 | 18 | POL | 411 | NLSWLSLDV | 387 | 9 | 0.0650 | 0.0051 | 0.6400 | 0.1600 | 0.0990 |
| 90 | 18 | POL | 411 | NLSWLSLDVSA | 388 | 11 | | | | | |
| 100 | 20 | POL | 47 | NVSIPWTHKV | 389 | 10 | 0.0001 | | | | |
| 100 | 20 | POL | 430 | PAAMPHLL | 390 | 8 | | | | | |
| 85 | 17 | POL | 430 | PAAMPHLLV | 391 | 9 | | | | | |
| 90 | 18 | POL | 775 | PADDPSRGRL | 392 | 10 | | | | | |
| 90 | 18 | ENV | 131 | PAGGSSSGT | 393 | 9 | | | | | |
| 90 | 18 | ENV | 131 | PAGGSSSGTV | 394 | 10 | | | | | |
| 95 | 19 | POL | 641 | PALMPLYA | 395 | 8 | | | | | |
| 95 | 19 | POL | 641 | PALMPLYACI | 396 | 10 | 0.0001 | | | | |
| 75 | 15 | X | 145 | PAPCNFFT | 397 | 8 | | | | | |
| 75 | 15 | X | 145 | PAPCNFFTSA | 398 | 10 | | | | | |
| 80 | 16 | X | 11 | PARDVLCL | 399 | 8 | | | | | |
| 75 | 15 | X | 11 | PARDVLCLRPV | 400 | 11 | | | | | |
| 90 | 18 | POL | 355 | PARVTGGV | 401 | 8 | | | | | |
| 90 | 18 | POL | 355 | PARVTGGVFL | 402 | 10 | | | | | |
| 90 | 18 | POL | 355 | PARVTGGVFLV | 403 | 11 | | | | | |
| 95 | 19 | NUC | 130 | PAYRPPNA | 404 | 8 | | | | | |
| 95 | 19 | NUC | 130 | PAYRPPNAPI | 405 | 10 | 0.0001 | | | | |
| 95 | 19 | NUC | 130 | PAYRPPNAPIL | 406 | 11 | | | | | |
| 85 | 17 | POL | 616 | PIDWKVCQRI | 407 | 10 | 0.0001 | | | | |
| 85 | 17 | POL | 616 | PIDWKVCQRIV | 408 | 11 | | | | | |
| 100 | 20 | ENV | 380 | PIFFCLWV | 409 | 8 | | | | | |
| 100 | 20 | ENV | 380 | PIFFCLWVYI | 410 | 10 | 0.0004 | | | | |
| 85 | 17 | POL | 713 | PIHTAELL | 411 | 8 | | | | | |
| 85 | 17 | POL | 713 | PIHTAELLA | 412 | 9 | | | | | |
| 85 | 17 | POL | 713 | PIHTAELLAA | 413 | 10 | | | | | |
| 80 | 16 | POL | 496 | PIILGFRKI | 414 | 9 | 0.0001 | | | | |
| 80 | 16 | POL | 496 | PIILGFRKIPM | 415 | 11 | | | | | |
| 100 | 20 | NUC | 138 | PILSTLPET | 416 | 9 | 0.0001 | | | | |
| 100 | 20 | NUC | 138 | PILSTLPETT | 417 | 10 | 0.0001 | | | | |
| 100 | 20 | NUC | 138 | PILSTLPETTV | 418 | 11 | 0.0001 | | | | |
| 80 | 16 | ENV | 314 | PIPSSWAFA | 419 | 9 | | | | | |
| 95 | 19 | POL | 20 | PLEEELPRL | 420 | 9 | 0.0003 | | | | |
| 90 | 18 | POL | 20 | PLEEELPRLA | 421 | 10 | 0.0001 | | | | |
| 95 | 19 | ENV | 10 | PLGFFPDHQL | 422 | 10 | 0.0002 | | | | |
| 100 | 20 | POL | 427 | PLHPAAMPHL | 423 | 10 | 0.0001 | | | | |
| 100 | 20 | POL | 427 | PLHPAAMPHLL | 424 | 11 | | | | | |
| 100 | 20 | ENV | 377 | PLLPIFFCL | 425 | 9 | 0.0650 | 0.0001 | 0.0018 | 0.1100 | 0.0047 |
| 100 | 20 | ENV | 377 | PLLPIFFCLWV | 426 | 11 | | | | | |
| 90 | 18 | ENV | 174 | PLLVLQAGFFL | 427 | 11 | 0.0008 | | | | |
| 80 | 16 | POL | 711 | PLPIHTAEL | 428 | 9 | 0.0004 | | | | |
| 80 | 16 | POL | 711 | PLPIHTAELL | 429 | 10 | 0.0001 | | | | |
| 80 | 16 | POL | 711 | PLPIHTAELLA | 430 | 11 | | | | | |
| 75 | 15 | POL | 2 | PLSYQHFRKL | 431 | 10 | 0.0001 | | | | |
| 75 | 15 | POL | 2 | PLSYQHFRKLL | 432 | 11 | | | | | |
| 85 | 17 | POL | 98 | PLTVNEKRRL | 433 | 10 | 0.0001 | | | | |
| 80 | 16 | POL | 505 | PMGVGLSPFL | 434 | 10 | 0.0001 | | | | |
| 80 | 16 | POL | 505 | PMGVGLSPFLL | 435 | 11 | | | | | |
| 95 | 19 | ENV | 106 | PQAMQWNST | 436 | 9 | | | | | |
| 80 | 16 | ENV | 106 | PQAMQWNSTT | 437 | 10 | | | | | |
| 90 | 18 | ENV | 192 | PQSLDSWWT | 438 | g | | | | | |
| 90 | 18 | ENV | 192 | PQSLDSWWTSL | 439 | 11 | | | | | |
| 75 | 15 | POL | 692 | PTGWGLAI | 440 | 8 | | | | | |
| 80 | 16 | ENV | 219 | PTSNHSPT | 441 | 8 | | | | | |
| 85 | 17 | POL | 797 | PTTGRTSL | 442 | 8 | | | | | |
| 85 | 17 | POL | 797 | PTTGRTSLYA | 443 | 10 | | | | | |
| 80 | 16 | NUC | 15 | PTVQASKL | 444 | 8 | | | | | |
| 80 | 16 | NUC | 15 | PTVQASKLCL | 445 | 10 | | | | | |
| 75 | 15 | ENV | 351 | PTVWLSVI | 446 | 8 | | | | | |
| 75 | 15 | ENV | 351 | PTVWLSVIWM | 447 | 10 | | | | | |
| 95 | 19 | X | 59 | PVCAFSSA | 448 | 8 | | | | | |
| 85 | 17 | POL | 612 | PVNRPIDWKV | 449 | 10 | 0.0002 | | | | |
| 95 | 19 | POL | 654 | QAFTFSPT | 450 | 8 | | | | | |
| 95 | 19 | POL | 654 | QAFTFSPTYKA | 451 | 11 | | | | | |
| 95 | 19 | ENV | 179 | QAGFFLLT | 452 | 8 | | | | | |
| 80 | 16 | ENV | 179 | QAGFFLLTRI | 453 | 10 | | | | | |
| 80 | 16 | ENV | 179 | QAGFFLLTRIL | 454 | 11 | | | | | |
| 90 | 18 | NUC | 57 | QAILCWGEL | 455 | 9 | | | | | |
| 90 | 18 | NUC | 57 | QAILCWGELM | 456 | 10 | | | | | |
| 95 | 19 | ENV | 107 | QAMQWNST | 457 | 8 | | | | | |
| 80 | 16 | ENV | 107 | QAMQWNSTT | 458 | 9 | | | | | |
| 80 | 16 | NUC | 18 | QASKLCLGWL | 459 | 10 | | | | | |
| 80 | 16 | X | 8 | QLDPARDV | 460 | 8 | 0.0001 | | | | |
| 80 | 16 | X | 8 | QLDPARDVL | 461 | 9 | 0.0001 | | | | |
| 80 | 16 | X | 8 | QLDPARDVLCL | 462 | 11 | 0.0001 | | | | |
| 90 | 18 | NUC | 99 | QLLWFHISCL | 463 | 10 | 0.0060 | | | | |
| 85 | 17 | NUC | 99 | QLLWFHISCLT | 464 | 11 | | | | | |
| 95 | 19 | POL | 685 | QVFADATPT | 465 | 9 | 0.0001 | | | | |
| 95 | 19 | POL | 528 | RAFPHCLA | 466 | 8 | | | | | |
| 80 | 16 | ENV | 187 | RILTIPQSL | 467 | 9 | 0.0010 | | | | |
| 90 | 18 | POL | 624 | RIVGLLGFA | 468 | 9 | | | | | |
| 90 | 18 | POL | 624 | RIVGLLGFAA | 469 | 10 | | | | | |
| 75 | 15 | POL | 106 | RLKLIMPA | 470 | 8 | | | | | |
| 90 | 18 | NUC | 56 | RQAILCWGEL | 471 | 10 | | | | | |
| 90 | 18 | NUC | 56 | RQAILCWGELM | 472 | 11 | | | | | |
| 90 | 18 | NUC | 98 | RQLLWFHI | 473 | 8 | | | | | |
| 90 | 18 | NUC | 98 | RQLLWFHISCL | 474 | 11 | | | | | |
| 85 | 17 | ENV | 88 | RQSGRQPT | 475 | 8 | | | | | |
| 90 | 18 | POL | 353 | RTPARVTGGV | 476 | 10 | | | | | |
| 95 | 19 | NUC | 127 | RTPPAYRPPNA | 477 | 11 | | | | | |
| 95 | 19 | POL | 36 | RVAEDLNL | 478 | 8 | | | | | |
| 90 | 18 | POL | 36 | RVAEDLNLGNL | 479 | 11 | | | | | |
| 80 | 16 | POL | 818 | RVHFASPL | 480 | 8 | | | | | |
| 75 | 15 | POL | 818 | RVHFASPLHV | 481 | 10 | 0.0001 | | | | |
| 75 | 15 | POL | 818 | RVHFASPLHVA | 482 | 11 | | | | | |
| 100 | 20 | POL | 357 | RVTGGVFL | 483 | 8 | | | | | |
| 100 | 20 | POL | 357 | RVTGGVFLV | 484 | 9 | 0.0041 | | | | |
| 90 | 18 | X | 65 | SAGPCALRFT | 485 | 10 | | | | | |
| 95 | 19 | POL | 520 | SAICSVVRRA | 486 | 10 | 0.0001 | | | | |
| 90 | 18 | NUC | 35 | SALYREAL | 487 | 8 | | | | | |
| 100 | 20 | POL | 49 | SIPWTHKV | 488 | 8 | | | | | |
| 95 | 19 | ENV | 194 | SLDSWWTSL | 489 | 9 | | | | | |
| 75 | 15 | POL | 565 | SLGIHLNPNKT | 490 | 11 | | | | | |
| 95 | 19 | ENV | 337 | SLLVPFVQWFV | 491 | 11 | | | | | |
| 75 | 15 | POL | 581 | SLNFMGYV | 492 | 8 | | | | | |
| 75 | 15 | POL | 581 | SLNFMGYVI | 493 | 9 | 0.0038 | | | | |
| 95 | 19 | X | 54 | SLRGLPVCA | 494 | 9 | 0.0007 | | | | |
| 90 | 18 | POL | 403 | SLTNLLSSNL | 495 | 10 | 0.0014 | | | | |
| 75 | 15 | ENV | 216 | SQSPTSNHSPT | 496 | 11 | | | | | |
| 75 | 15 | ENV | 280 | STGPCKTCT | 497 | 9 | | | | | |
| 100 | 20 | NUC | 141 | STLPETTV | 498 | 8 | | | | | |
| 100 | 20 | NUC | 141 | STLPETTVV | 499 | 9 | 0.0019 | | | | |
| 80 | 16 | ENV | 85 | STNRQSGRQPT | 500 | 11 | | | | | |
| 85 | 17 | POL | 548 | SVQHLESL | 501 | 8 | | | | | |
| 80 | 16 | ENV | 330 | SVRFSWLSL | 502 | 9 | 0.0001 | | | | |
| 80 | 16 | ENV | 330 | SVRFSWLSLL | 503 | 10 | 0.0004 | | | | |
| 80 | 16 | ENV | 330 | SVRFSWLSLLV | 504 | 11 | | | | | |
| 90 | 18 | POL | 739 | SVVLSRKYT | 505 | 9 | | | | | |
| 95 | 19 | POL | 524 | SVVRRAFPHCL | 506 | 11 | | | | | |
| 85 | 17 | POL | 716 | TAELLAACFA | 507 | 10 | | | | | |
| 95 | 19 | NUC | 53 | TALRQAIL | 508 | 8 | | | | | |
| 80 | 16 | NUC | 33 | TASALYREA | 509 | 9 | | | | | |
| 80 | 16 | NUC | 33 | TASALYREAL | 510 | 10 | | | | | |
| 90 | 18 | ENV | 190 | TIPQSLDSWWT | 511 | 11 | | | | | |
| 100 | 20 | NUC | 142 | TLPETTVV | 512 | 8 | | | | | |
| 100 | 20 | POL | 150 | TLWKAGIL | 513 | 8 | | | | | |
| 95 | 19 | POL | 636 | TQCGYPAL | 514 | 8 | | | | | |
| 95 | 19 | POL | 636 | TQCGYPALM | 515 | 9 | | | | | |
| 95 | 19 | POL | 636 | TQCGYPALMPL | 516 | 11 | | | | | |
| 85 | 17 | POL | 798 | TTGRTSLYA | 517 | 9 | | | | | |
| 75 | 15 | ENV | 278 | TTSTGPCKT | 518 | 9 | | | | | |
| 75 | 15 | ENV | 278 | TTSTGPCKTCT | 519 | 11 | | | | | |
| 85 | 17 | POL | 100 | TVNEKRRL | 520 | 8 | | | | | |
| 80 | 16 | NUC | 16 | TVQASKLCL | 521 | 9 | 0.0002 | | | | |
| 75 | 15 | ENV | 352 | TVWLSVIWM | 522 | 9 | 0.0002 | | | | |
| 95 | 19 | POL | 37 | VAEDLNLGNL | 523 | 10 | 0.0001 | | | | |
| 95 | 19 | X | 15 | VLCLRPVGA | 524 | 9 | 0.0014 | | | | |
| 85 | 17 | POL | 543 | VLGAKSVQHL | 525 | 10 | 0.0001 | | | | |
| 90 | 18 | X | 133 | VLGGCRHKL | 526 | 9 | 0.0009 | | | | |
| 90 | 18 | X | 133 | VLGGCRHKLV | 527 | 10 | 0.0001 | | | | |
| 85 | 17 | X | 92 | VLHKRTLGL | 528 | 9 | 0.0012 | | | | |
| 95 | 19 | ENV | 259 | VLLDYQGM | 529 | 8 | | | | | |
| 95 | 19 | ENV | 259 | VLLDYQGML | 530 | 9 | 0.0440 | 0.0001 | 0.0210 | 0.9000 | 0.0002 |
| 90 | 18 | ENV | 259 | VLLDYQGMLPV | 531 | 11 | 0.5800 | 0.2200 | 4.9000 | 0.3400 | 0.0170 |
| 95 | 19 | ENV | 177 | VLQAGFFL | 532 | 8 | 0.0019 | | | | |
| 95 | 19 | ENV | 177 | VLQAGFFLL | 533 | 9 | 0.0660 | | | | |
| 95 | 19 | ENV | 177 | VLQAGFFLLT | 534 | 10 | 0.0011 | | | | |
| 80 | 16 | NUC | 17 | VQASKLCL | 535 | 8 | | | | | |
| 80 | 16 | NUC | 17 | VQASKLCLGWL | 536 | 11 | | | | | |
| 95 | 19 | ENV | 343 | VQWFVGLSPT | 537 | 10 | | | | | |
| 95 | 19 | ENV | 343 | VQWFVGLSPTV | 538 | 11 | | | | | |
| 100 | 20 | POL | 358 | VTGGVFLV | 539 | 8 | | | | | |
| 90 | 18 | POL | 542 | VVLGAKSV | 540 | 8 | | | | | |
| 80 | 16 | POL | 542 | VVLGAKSVQHL | 541 | 11 | | | | | |
| 90 | 18 | POL | 740 | VVLSRKYT | 542 | 8 | | | | | |
| 95 | 19 | POL | 525 | VVRRAFPHCL | 543 | 10 | 0.0003 | | | | |
| 95 | 19 | POL | 525 | VVRRAFPHCLA | 544 | 11 | | | | | |
| 80 | 16 | POL | 759 | WILRGTSFV | 545 | 9 | 0.0270 | | | | |
| 80 | 16 | POL | 759 | WILRGTSFVYV | 546 | 11 | | | | | |
| 80 | 16 | POL | 751 | WLLGCAANWI | 547 | 10 | 0.0053 | | | | |
| 80 | 16 | POL | 751 | WLLGCAANWIL | 548 | 11 | | | | | |
| 100 | 20 | POL | 414 | WLSLDVSA | 549 | 8 | | | | | |
| 95 | 19 | POL | 414 | WLSLDVSAA | 550 | 9 | 0.0059 | | | | |
| 100 | 20 | ENV | 335 | WLSLLVPFV | 551 | 9 | 1.1000 | 0.0380 | 7.2000 | 0.3600 | 0.0310 |
| 95 | 19 | ENV | 237 | WMCLRRFI | 552 | 8 | | | | | |
| 95 | 19 | ENV | 237 | WMCLRRFII | 553 | 9 | 0.0005 | | | | |
| 95 | 19 | ENV | 237 | WMCLRRFIIFL | 554 | 11 | 0.0019 | | | | |
| 85 | 17 | ENV | 359 | WMMWYWGPSL | 555 | 10 | 0.0009 | | | | |
| 100 | 20 | POL | 52 | WTHKVGNFT | 556 | 9 | 0.0001 | | | | |
| 95 | 19 | POL | 52 | WTHKVGNFTGL | 557 | 11 | | | | | |
| 100 | 20 | POL | 147 | YLHTLWKA | 558 | 8 | | | | | |
| 100 | 20 | POL | 147 | YLHTLWKAGI | 559 | 10 | 0.0160 | 0.0005 | 0.5600 | 0.1000 | 0.0320 |
| 100 | 20 | POL | 147 | YLHTLWKAGIL | 560 | 11 | | | | | |
| 100 | 20 | POL | 122 | YLPLDKGI | 561 | 8 | | | | | |
| 90 | 18 | NUC | 118 | YLVSFGVWI | 562 | 9 | 0.3800 | | | | |
| 90 | 18 | NUC | 118 | YLVSFGVWIRT | 563 | 11 | | | | | |
| 90 | 18 | POL | 538 | YMDDVVLGA | 564 | 9 | 0.0250 | 0.0001 | 0.0024 | 0.1000 | 0.0002 |
| 90 | 18 | ENV | 263 | YQGMLPVCPL | 565 | 10 | | | | | |
| 75 | 15 | POL | 5 | YQHFRKLL | 566 | 8 | | | | | |
| 75 | 15 | POL | 5 | YQHFRKLLL | 567 | 9 | | | | | |
| 75 | 15 | POL | 5 | YQHFRKLLLL | 568 | 10 | | | | | |
| 85 | 17 | POL | 746 | YTSFPWLL | 569 | 8 | | | | | |
| 75 | 15 | POL | 746 | YTSFPWLLGCA | 570 | 11 | | | | | |
| 90 | 18 | POL | 768 | YVPSALNPA | 571 | 9 | 0.0039 | | | | |

TABLE IX
HBV A03 SUPER MOTIF (With binding information)

| | | | | | | | | | | | | | |
|---|---|---|---|---|---|---|---|---|---|---|---|---|---|
| Conserv- | | | | | | C- | | | | | | | SEQ ID |
| ancy | Frequency | Protein | Position | Sequence | P2 | term | AA | A*0301 | A*1101 | A*3101 | A*3301 | A*6801 | NO: |

| | | | | | | | | | | | | | |
|---|---|---|---|---|---|---|---|---|---|---|---|---|---|
| 85 | 17 | POL | 721 | AACFARSR | A | R | 8 | 0.0004 | 0.0003 | 0.0056 | 0.0035 | 0.0014 | 572 |
| 95 | 19 | POL | 521 | AICSVVRR | I | R | 8 | −0.0002 | 0.0003 | 0.0014 | −0.0009 | 0.0006 | 573 |
| 90 | 18 | POL | 772 | ALNPADDPSR | L | R | 10 | 0.0003 | 0.0001 | | | | 574 |
| 85 | 17 | X | 70 | ALRFTSAR | L | R | 8 | 0.0047 | 0.0009 | 0.0450 | 0.0230 | 0.0004 | 575 |
| 80 | 16 | POL | 822 | ASPLHVAWR | S | R | 9 | | | | | | 576 |
| 75 | 15 | ENV | 84 | ASTNRQSGR | S | R | 9 | 0.0009 | 0.0002 | 0.0088 | 0.0008 | 0.0001 | 577 |
| 80 | 16 | POL | 755 | CAANWILR | A | R | 8 | | | | | | 578 |
| 85 | 17 | X | 69 | CALRFTSAR | A | R | 9 | 0.0034 | 0.0230 | 1.5000 | 8.0000 | 0.7300 | 579 |
| 90 | 18 | X | 17 | CLRPVGAESR | L | R | 10 | 0.0011 | 0.0001 | | | | 580 |
| 100 | 20 | NUC | 48 | CSPHHTALR | S | R | 9 | 0.0029 | 0.0001 | 0.0520 | 0.0250 | 0.0440 | 581 |
| 85 | 17 | NUC | 29 | DLLDTASALYR | L | R | 11 | 0.0042 | −0.0003 | −0.0012 | 3.7000 | 0.0410 | 582 |
| 85 | 17 | NUC | 32 | DTASALYR | T | R | 8 | 0.0004 | −0.0002 | −0.0009 | 0.0018 | 0.0009 | 583 |
| 95 | 19 | POL | 17 | EAGPLEEELPR | A | R | 11 | −0.0009 | −0.0003 | −0.0012 | 0.0015 | 0.0110 | 584 |
| 90 | 18 | POL | 718 | ELLAACFAR | L | R | 9 | 0.0002 | 0.0004 | | | | 585 |
| 85 | 17 | POL | 718 | ELLAACFARSR | L | R | 11 | 0.0062 | 0.0016 | 0.0200 | 0.2000 | 0.1600 | 586 |
| 95 | 19 | NUC | 174 | ETTVVRRR | T | R | 8 | 0.0003 | −0.0002 | −0.0009 | 0.1400 | 0.0027 | 587 |
| 80 | 16 | NUC | 174 | ETTVVRRRGR | T | R | 10 | 0.0003 | 0.0001 | | | | 588 |
| 80 | 16 | POL | 821 | FASPLHVAWR | A | R | 10 | | | | | | 589 |
| 90 | 18 | X | 63 | FSSAGPCALR | S | R | 10 | | | | | | 590 |
| 95 | 19 | POL | 656 | FTFSPTYK | T | K | 8 | 0.0100 | 0.0100 | 0.0023 | 0.2100 | 0.0590 | 591 |
| 95 | 19 | POL | 518 | FTSAICSVVR | T | R | 10 | 0.0003 | 0.0003 | | | | 592 |
| 95 | 19 | POL | 518 | FTSAICSVVRR | T | R | 11 | 0.0065 | 0.0092 | 0.0170 | 0.0350 | 1.5000 | 593 |
| 90 | 18 | X | 132 | FVLGGCRHK | V | K | 9 | 0.0430 | 0.0090 | | | | 594 |
| 75 | 15 | POL | 567 | GIHLNPNK | I | K | 8 | | | | | | 595 |
| 75 | 15 | POL | 567 | GIHLNPNKTK | I | K | 10 | 0.0025 | 0.0011 | 0.0009 | 0.0009 | 0.0003 | 596 |
| 75 | 15 | POL | 567 | GIHLNPNKTKR | I | R | 11 | | | | | | 597 |
| 85 | 17 | NUC | 29 | GMDIDPYK | M | K | 8 | 0.0006 | 0.0004 | −0.0009 | −0.0009 | 0.0001 | 598 |
| 90 | 18 | POL | 735 | GTDNSVVLSR | T | R | 10 | 0.0010 | 0.0420 | 0.0030 | 0.0019 | 0.0008 | 599 |
| 90 | 18 | POL | 735 | GTDNSVVLSRK | T | K | 11 | 0.0140 | 0.5600 | −0.0002 | −0.0006 | 0.0001 | 600 |
| 95 | 19 | NUC | 123 | GVWIRTPPAYR | V | R | 11 | 0.1900 | 0.1700 | 6.8000 | 0.7300 | 0.6600 | 601 |
| 90 | 18 | NUC | 104 | HISCLTFGR | I | R | 9 | 0.0160 | 0.0065 | | | | 602 |
| 75 | 15 | POL | 569 | HLNPNKTK | L | K | 8 | | | | | | 603 |
| 75 | 15 | POL | 569 | HLNPNKTKR | L | R | 9 | 0.0025 | 0.0001 | | | | 604 |
| 100 | 20 | POL | 149 | HTLWKAGILYK | T | K | 11 | 0.5400 | 0.4400 | 0.0370 | 0.0720 | 0.1900 | 605 |
| 90 | 18 | NUC | 105 | ISCLTFGR | S | R | 8 | 0.0004 | 0.0002 | 0.0017 | −0.0009 | 0.0017 | 606 |
| 100 | 20 | POL | 153 | KAGILYKR | A | R | 8 | 0.0002 | −0.0002 | 0.0015 | −0.0009 | 0.0001 | 607 |
| 80 | 16 | POL | 610 | KLPVNRPIDWK | L | K | 11 | | | | | | 608 |
| 75 | 15 | X | 130 | KVFVLGGCR | V | R | 9 | 0.0420 | 0.0820 | 0.6000 | 0.0710 | 0.0030 | 609 |
| 85 | 17 | POL | 720 | LAACFARSR | A | R | 9 | 0.0058 | 0.0065 | | | | 610 |
| 90 | 18 | POL | 719 | LLAACFAR | L | R | 8 | 0.0024 | 0.0003 | 0.0015 | 0.0029 | 0.0064 | 611 |
| 85 | 17 | POL | 719 | LLAACFARSR | L | R | 10 | | | | | | 612 |
| 85 | 17 | NUC | 30 | LLDTASALYR | L | R | 10 | 0.0050 | 0.0002 | | | | 613 |
| 80 | 16 | POL | 752 | LLGCAANWILR | L | R | 11 | | | | | | 614 |
| 75 | 15 | POL | 564 | LSLGIHLNPNK | S | K | 11 | | | | | | 615 |
| 95 | 19 | NUC | 169 | LSTLPETTVVR | S | R | 11 | −0.0009 | 0.0008 | −0.0012 | −0.0023 | 0.0078 | 616 |
| 75 | 15 | POL | 3 | LSYQHFRK | S | K | 8 | | | | | | 617 |
| 85 | 17 | POL | 99 | LTVNEKRR | T | R | 8 | −0.0002 | −0.0002 | −0.0009 | −0.0009 | 0.0001 | 618 |
| 90 | 18 | NUC | 119 | LVSFGVWIR | V | R | 9 | 0.0028 | 0.0120 | | | | 619 |
| 100 | 20 | POL | 377 | LVVDFSQFSR | V | R | 10 | 0.0016 | 0.3600 | 0.0260 | 0.2300 | 0.4900 | 620 |
| 75 | 15 | X | 103 | MSTTDLEAYFK | S | K | 11 | | | | | | 621 |
| 90 | 18 | NUC | 75 | NLEDPASR | L | R | 8 | −0.0002 | −0.0002 | −0.0009 | −0.0009 | 0.0001 | 622 |
| 95 | 19 | POL | 45 | NLNVSIPWTHK | L | K | 11 | −0.0009 | 0.0005 | −0.0012 | −0.0023 | 0.0019 | 623 |
| 90 | 18 | POL | 738 | NSVVLSRK | S | K | 8 | 0.0006 | 0.0010 | −0.0009 | −0.0009 | 0.0007 | 624 |
| 100 | 20 | POL | 47 | NVSIPWTHK | V | K | 9 | 0.0820 | 0.0570 | 0.0002 | 0.0100 | 0.0320 | 625 |
| 90 | 18 | POL | 775 | PADDPSRGR | A | R | 9 | 0.0008 | 0.0002 | 0.0004 | 0.0015 | 0.0002 | 626 |
| 80 | 16 | X | 11 | PARDVLCLR | A | R | 9 | 0.0002 | 0.0002 | 0.0100 | 0.0180 | 0.0002 | 627 |
| 75 | 15 | ENV | 83 | PASTNRQSGR | A | R | 10 | | | | | | 628 |
| 90 | 18 | POL | 616 | PIDWKVCQR | I | R | 9 | 0.0002 | 0.0005 | | | | 629 |
| 80 | 16 | POL | 496 | PIILGFRK | I | K | 8 | | | | | | 630 |
| 95 | 19 | POL | 20 | PLEEELPR | L | R | 8 | 0.0002 | −0.0002 | −0.0009 | −0.0009 | 0.0001 | 631 |
| 100 | 20 | POL | 2 | PLSYQHFR | L | R | 8 | −0.0002 | −0.0002 | −0.0009 | −0.0009 | 0.0001 | 632 |
| 75 | 15 | POL | 2 | PLSYQHFRK | L | K | 9 | 0.0011 | 0.0031 | 0.0006 | 0.0008 | 0.0002 | 633 |
| 85 | 17 | POL | 98 | PLTVNEKR | L | R | 8 | 0.0002 | −0.0002 | −0.0009 | −0.0009 | 0.0001 | 634 |
| 85 | 17 | POL | 98 | PLTVNEKRR | L | R | 9 | 0.0008 | 0.0005 | 0.0004 | 0.0027 | 0.0002 | 635 |
| 90 | 18 | X | 20 | PVGAESRGR | V | R | 9 | 0.0002 | 0.0005 | 0.0004 | 0.0043 | 0.0002 | 636 |
| 85 | 17 | POL | 612 | PVNRPIDWK | V | K | 9 | 0.0310 | 0.1400 | 0.0002 | 0.0006 | 0.0009 | 637 |
| 95 | 19 | POL | 654 | QAFTFSPTYK | A | K | 10 | 0.0450 | 0.5400 | 0.0010 | 0.0057 | 1.2000 | 638 |
| 80 | 16 | ENV | 179 | QAGFFLLTR | A | R | 9 | | | | | | 639 |
| 75 | 15 | NUC | 169 | QSPRRRRSQSR | S | R | 11 | | | | | | 640 |
| 80 | 16 | POL | 189 | QSSGILSR | S | R | 8 | | | | | | 641 |
| 75 | 15 | POL | 106 | RLKLIMPAR | L | R | 9 | 0.0950 | 0.0002 | 3.1000 | 0.0490 | 0.0002 | 642 |
| 75 | 15 | X | 128 | RLKVFVLGGCR | L | R | 11 | | | | | | 643 |
| 95 | 19 | POL | 376 | RLVVDFSQFSR | L | R | 11 | 0.2800 | 3.8000 | 2.6000 | 1.2000 | 6.1000 | 644 |
| 95 | 19 | NUC | 183 | RSPRRRTPSPR | S | R | 11 | −0.0007 | −0.0003 | 0.0190 | −0.0023 | 0.0003 | 645 |
| 75 | 15 | NUC | 167 | RSQSPRRR | S | R | 8 | | | | | | 646 |
| 75 | 15 | NUC | 167 | RSQSPRRRR | S | R | 9 | | | | | | 647 |
| 95 | 19 | NUC | 188 | RTPSPRRR | T | R | 8 | −0.0002 | −0.0002 | 0.0033 | 0.0014 | 0.0002 | 648 |
| 95 | 19 | NUC | 188 | RTPSPRRRR | T | R | 9 | 0.0054 | 0.0005 | 0.2000 | 0.0016 | 0.0003 | 649 |
| 100 | 20 | POL | 357 | RVTGGVFLVDK | V | K | 11 | 0.0190 | 0.0290 | −0.0002 | −0.0003 | 0.0001 | 650 |
| 90 | 18 | X | 65 | SAGPCALR | A | R | 8 | −0.0002 | 0.0020 | 0.0029 | 0.0024 | 0.0360 | 651 |
| 95 | 19 | POL | 520 | SAICSVVR | A | R | 8 | −0.0002 | 0.0071 | 0.0280 | 0.0081 | 0.0690 | 652 |
| 95 | 19 | POL | 520 | SAICSVVRR | A | R | 9 | 0.0058 | 0.2100 | 0.1500 | 0.0650 | 0.3800 | 653 |
| 90 | 18 | POL | 771 | SALNPADDPSR | A | R | 11 | −0.0004 | −0.0003 | −0.0012 | −0.0023 | 0.0003 | 654 |
| 75 | 15 | POL | 565 | SLGIHLNPNK | L | K | 10 | | | | | | 655 |
| 90 | 18 | X | 64 | SSAGPCALR | S | R | 9 | 0.0080 | 0.1400 | 0.3300 | 0.1600 | 0.7500 | 656 |
| 95 | 19 | NUC | 170 | STLPETTVVR | T | R | 10 | 0.0007 | 0.0600 | 0.0080 | 0.0240 | 0.0250 | 657 |
| 95 | 19 | NUC | 170 | STLPETTVVRR | T | R | 11 | 0.0150 | 1.4000 | 0.1000 | 0.1600 | 0.3100 | 658 |
| 80 | 16 | ENV | 85 | STNRQSGR | T | R | 8 | | | | | | 659 |
| 75 | 15 | X | 104 | STTDLEAYFK | T | K | 10 | 0.0066 | 2.7000 | | | | 660 |
| 85 | 17 | POL | 716 | TAELLAACFAR | A | R | 11 | 0.0006 | 0.0023 | 0.0066 | 0.1600 | 0.0590 | 661 |
| 95 | 19 | NUC | 171 | TLPETTVVR | L | R | 9 | 0.0008 | 0.0002 | 0.0009 | 0.0024 | 0.0180 | 662 |
| 95 | 19 | NUC | 171 | TLPETTVVRR | L | R | 10 | 0.0007 | 0.0230 | 0.0006 | 0.0120 | 0.0440 | 663 |
| 95 | 19 | NUC | 171 | TLPETTVVRRR | L | R | 11 | 0.0005 | 0.0160 | 0.0061 | 0.0710 | 0.6400 | 664 |
| 100 | 20 | POL | 150 | TLWKAGILYK | L | K | 10 | 5.3000 | 0.3600 | 0.0051 | 0.0010 | 0.0130 | 665 |
| 100 | 20 | POL | 150 | TLWKAGILYKR | L | R | 11 | 0.0082 | 0.0095 | 0.1000 | 0.1100 | 0.0640 | 666 |
| 95 | 19 | POL | 519 | TSAICSVVR | S | R | 9 | 0.0005 | 0.0008 | 0.0600 | 0.0200 | 0.0820 | 667 |
| 95 | 19 | POL | 519 | TSAICSVVRR | S | R | 10 | 0.0018 | 0.0006 | 0.0030 | 0.0066 | 0.0048 | 668 |
| 75 | 15 | X | 105 | TTDLEAYFK | T | K | 9 | 0.0006 | 0.9200 | 0.0006 | 0.0012 | 0.0170 | 669 |
| 75 | 15 | ENV | 278 | TTSTGPCK | T | K | 8 | | | | | | 670 |
| 80 | 16 | NUC | 175 | TTVVRRRGR | T | R | 9 | 0.0008 | 0.0005 | 0.2500 | 0.1400 | 0.0095 | 671 |
| 80 | 16 | NUC | 176 | TVVRRRGR | V | R | 8 | 0.0003 | 0.0001 | | | | 672 |
| 80 | 16 | NUC | 176 | TVVRRRGRSPR | V | R | 11 | | | | | | 673 |
| 90 | 18 | X | 133 | VLGGCRHK | L | K | 8 | 0.0150 | 0.0002 | −0.0005 | −0.0009 | 0.0001 | 674 |
| 80 | 16 | ENV | 177 | VLQAGFFLLTR | L | R | 11 | | | | | | 675 |
| 90 | 18 | NUC | 120 | VSFGVWIR | S | R | 8 | 0.0040 | 0.0290 | 0.0750 | 0.0270 | 0.0360 | 676 |
| 100 | 20 | POL | 48 | VSIPWTHK | S | K | 8 | 0.0130 | 0.0170 | 0.0031 | 0.0013 | 0.0004 | 677 |
| 100 | 20 | POL | 358 | VTGGVFLVDK | T | K | 10 | 0.0390 | 0.0920 | 0.0002 | 0.0006 | 0.0022 | 678 |
| 100 | 20 | POL | 378 | VVDFSQFSR | V | R | 9 | 0.0015 | 0.0750 | 0.0013 | 0.0170 | 0.0330 | 679 |
| 80 | 16 | NUC | 177 | VVRRRGRSPR | V | R | 10 | 0.0027 | 0.0001 | | | | 680 |
| 80 | 16 | NUC | 177 | VVRRRGRSPRR | V | R | 11 | | | | | | 681 |
| 95 | 19 | NUC | 125 | VVIRTPPAYR | I | R | 9 | 0.0008 | 0.0005 | | | | 682 |
| 90 | 18 | POL | 314 | WLQFRNSK | L | K | 8 | −0.0002 | 0.0005 | 0.0020 | 0.0052 | 0.0001 | 683 |
| 85 | 17 | NUC | 26 | WLWGMDIDPYK | L | K | 11 | 0.0030 | 0.0013 | −0.0003 | 0.0039 | 0.0490 | 684 |
| 100 | 20 | POL | 122 | YLPLDKGIK | L | K | 9 | 0.0001 | 0.0001 | 0.0006 | 0.0006 | 0.0002 | 685 |
| 90 | 18 | NUC | 118 | YLVSFGVWIR | L | R | 10 | 0.0005 | 0.0002 | | | | 686 |
| 90 | 18 | POL | 538 | YMDDVVLGAK | M | K | 10 | 0.0330 | 0.0043 | 0.0002 | 0.0006 | 0.0001 | 687 |
| 80 | 16 | POL | 493 | YSHPIILGFR | S | R | 10 | | | | | | 688 |
| 80 | 16 | POL | 493 | YSHPIILGFRK | S | K | 11 | | | | | | 689 |

TABLE X
HBV A24 SUPER MOTIF (With binding information)

| | | | | | | | |
|---|---|---|---|---|---|---|---|
| Conservancy | Freq | Protein | Position | Sequence | String | A*2401 | SEQ ID NO: |

| | | | | | | | |
|---|---|---|---|---|---|---|---|
| 95 | 19 | POL | 529 | AFPHCLAF | XFXXXXXF | | 690 |
| 95 | 19 | POL | 529 | AFPHCLAFSY | XFXXXXXXXY | | 691 |
| 95 | 19 | POL | 529 | AFPHCLAFSYM | XFXXXXXXXXM | | 692 |
| 95 | 19 | X | 62 | AFSSAGPCAL | XFXXXXXXXL | 0.0012 | 693 |
| 90 | 18 | POL | 535 | AFSYMDDVVL | XFXXXXXXXL | 0.0009 | 694 |
| 95 | 19 | POL | 655 | AFTFSPTY | XFXXXXXY | | 695 |
| 95 | 19 | POL | 655 | AFTFSPTYKAF | XFXXXXXXXXF | | 696 |
| 95 | 19 | POL | 521 | AICSVVRRAF | XIXXXXXXXF | | 697 |
| 90 | 18 | NUC | 58 | AILCWGEL | XIXXXXXL | | 698 |
| 90 | 18 | NUC | 58 | AILCWGELM | XIXXXXXXM | | 699 |
| 95 | 19 | POL | 642 | ALMPLYACI | XLXXXXXXI | | 700 |
| 95 | 19 | NUC | 54 | ALRQAILCW | XLXXXXXXW | | 701 |
| 80 | 16 | ENV | 108 | AMQWNSTTF | XMXXXXXXF | | 702 |
| 95 | 19 | POL | 690 | ATPTGWGL | XTXXXXXL | | 703 |
| 75 | 15 | POL | 690 | ATPTGWGLAI | XTXXXXXXXI | | 704 |
| 95 | 19 | POL | 397 | AVPNLQSL | XVXXXXXL | | 705 |
| 95 | 19 | POL | 397 | AVPNLQSLTNL | XVXXXXXXXXL | | 706 |
| 100 | 20 | NUC | 131 | AYRPPNAPI | XYXXXXXXI | 0.0260 | 707 |
| 100 | 20 | NUC | 131 | AYRPPNAPIL | XYXXXXXXXL | 0.0220 | 708 |
| 75 | 15 | POL | 607 | CFRKLPVNRPI | XFXXXXXXXX1 | | 709 |
| 100 | 20 | ENV | 312 | CIPIPSSW | XIXXXXXW | | 710 |
| 100 | 20 | ENV | 312 | CIPIPSSWAF | XIXXXXXXXF | | 711 |
| 85 | 17 | NUC | 23 | CLGWLWGM | XLXXXXXM | | 712 |
| 85 | 17 | NUC | 23 | CLGWLWGMDI | XLXXXXXXXI | | 713 |
| 100 | 20 | ENV | 253 | CLIFLLVL | XLXXXXXL | | 714 |
| 100 | 20 | ENV | 253 | CLIFLLVLL | XLXXXXXXL | | 715 |
| 95 | 19 | ENV | 253 | CLIFLLVLLDY | XLXXXXXXXXY | | 716 |
| 95 | 19 | ENV | 239 | CLRRFIIF | XLXXXXXF | | 717 |
| 95 | 19 | ENV | 239 | CLRRFIIFL | XLXXXXXXL | | 718 |
| 75 | 15 | ENV | 239 | CLRRFIIFLF | XLXXXXXXXF | | 719 |
| 75 | 15 | ENV | 239 | CLRRFIIFLFI | XLXXXXXXXXI | | 720 |
| 100 | 20 | ENV | 310 | CTCIPIPSSW | XTXXXXXXXW | | 721 |
| 90 | 18 | NUC | 31 | DIDPYKEF | XIXXXXXF | | 722 |
| 85 | 17 | NUC | 29 | DLLDTASAL | XLXXXXXXL | | 723 |
| 85 | 17 | NUC | 29 | DLLDTASALY | XLXXXXXXXY | | 724 |
| 95 | 19 | POL | 40 | DLNLGNLNVSI | XLXXXXXXXXI | | 725 |
| 80 | 16 | NUC | 32 | DTASALYREAL | XTXXXXXXXXL | | 726 |
| 85 | 17 | POL | 618 | DWKVCQRI | XWXXXXXI | | 727 |
| 85 | 17 | POL | 618 | DWKVCQRIVGL | XWXXXXXXXXL | | 728 |
| 90 | 18 | ENV | 262 | DYQGMLPVCPL | XYXXXXXXXXL | 0.0002 | 729 |
| 80 | 16 | X | 122 | ELGEEIRL | XLXXXXXL | | 730 |
| 95 | 19 | NUC | 43 | ELLSFLPSDF | XLXXXXXXXF | | 731 |
| 95 | 19 | NUC | 43 | ELLSFLPSDFF | XLXXXXXXXXF | | 732 |
| 90 | 18 | NUC | 117 | EYLVSFGVW | XYXXXXXXW | | 733 |
| 90 | 18 | NUC | 117 | EYLVSFGVWI | XYXXXXXXXI | 0.0340 | 734 |
| 100 | 20 | ENV | 382 | FFCLWVYI | XFXXXXXI | | 735 |
| 80 | 16 | ENV | 182 | FFLLTRIL | XFXXXXXL | | 736 |
| 80 | 16 | ENV | 182 | FFLLTRILTI | XFXXXXXXXI | | 737 |
| 85 | 17 | ENV | 13 | FFPDHQLDPAF | XFXXXXXXXXF | | 738 |
| 80 | 16 | ENV | 243 | FIIFLFIL | XIXXXXXL | | 739 |
| 80 | 16 | ENV | 243 | FIIFLFILL | XIXXXXXXL | | 740 |
| 80 | 16 | ENV | 243 | FIIFLFILLL | XIXXXXXXXL | | 741 |
| 80 | 16 | ENV | 248 | FILLLCLI | XIXXXXXI | | 742 |
| 80 | 16 | ENV | 248 | FILLLCLIF | XIXXXXXXF | | 743 |
| 80 | 16 | ENV | 248 | FILLLCLIFL | XIXXXXXXXL | | 744 |
| 80 | 16 | ENV | 248 | FILLLCLIFLL | XIXXXXXXXXL | | 745 |
| 80 | 16 | ENV | 246 | FLFILLLCL | XLXXXXXXL | | 746 |
| 80 | 16 | ENV | 246 | FLFILLLCLI | XLXXXXXXXI | | 747 |
| 80 | 16 | ENV | 246 | FLFILLLCLIF | XLXXXXXXXXF | | 748 |
| 75 | 15 | ENV | 171 | FLGPLLVL | XLXXXXXL | | 749 |
| 95 | 19 | POL | 513 | FLLAQFTSAI | XLXXXXXXXI | | 750 |
| 95 | 19 | POL | 562 | FLLSLGIHL | XLXXXXXXL | | 751 |
| 80 | 16 | ENV | 183 | FLLTRILTI | XLXXXXXXI | | 752 |
| 95 | 19 | ENV | 256 | FLLVLLDY | XLXXXXXY | | 753 |
| 95 | 19 | ENV | 256 | FLLVLLDYQGM | XLXXXXXXXXM | | 754 |
| 95 | 19 | POL | 656 | FTFSPTYKAF | XTXXXXXXXF | | 755 |
| 95 | 19 | POL | 656 | FTFSPTYKAFL | XTXXXXXXXXL | | 756 |
| 95 | 19 | POL | 635 | FTQCGYPAL | XTXXXXXXL | | 757 |
| 95 | 19 | POL | 635 | FTQCGYPALM | XTXXXXXXXM | | 758 |
| 95 | 19 | ENV | 346 | FVGLSPTVW | XVXXXXXXW | | 759 |
| 95 | 19 | ENV | 346 | FVGLSPTVWL | XVXXXXXXXL | | 760 |
| 90 | 18 | X | 132 | FVLGGCRHKL | XVXXXXXXXL | | 761 |
| 95 | 19 | ENV | 342 | FVQWFVGL | XVXXXXXL | | 762 |
| 90 | 18 | POL | 766 | FVYVPSAL | XVXXXXXL | | 763 |
| 95 | 19 | POL | 630 | GFAAPFTQCGY | XFXXXXXXXXY | | 764 |
| 80 | 16 | ENV | 181 | GFFLLTRI | XFXXXXXI | | 765 |
| 80 | 16 | ENV | 181 | GFFLLTRIL | XFXXXXXXL | | 766 |
| 80 | 16 | ENV | 181 | GFFLLTRILTI | XFXXXXXXXXI | | 767 |
| 95 | 19 | ENV | 12 | GFFPDHQL | XFXXXXXL | | 768 |
| 75 | 15 | ENV | 170 | GFLGPLLVL | XFXXXXXXL | | 769 |
| 80 | 16 | POL | 500 | GFRKIPMGVGL | XFXXXXXXXXL | | 770 |
| 95 | 19 | POL | 627 | GLLGFAAPF | XLXXXXXXF | | 771 |
| 95 | 19 | POL | 509 | GLSPFLLLAQF | XLXXXXXXXF | | 772 |
| 100 | 20 | ENV | 348 | GLSPTVWL | XLXXXXXL | | 773 |
| 75 | 15 | ENV | 348 | GLSPTVWLSVI | XLXXXXXXXXI | | 774 |
| 85 | 17 | NUC | 29 | GMDIDPYKEF | XMXXXXXXXF | | 775 |
| 90 | 18 | ENV | 265 | GMLPVCPL | XMXXXXXL | | 776 |
| 90 | 18 | POL | 735 | GTDNSVVL | XTXXXXXL | | 777 |
| 75 | 15 | ENV | 13 | GTNLSVPNPL | XTXXXXXXXL | | 778 |
| 80 | 16 | POL | 763 | GTSFVYVPSAL | XTXXXXXXXXL | | 779 |
| 80 | 16 | POL | 507 | GVGLSPFL | XVXXXXXL | | 780 |
| 80 | 16 | POL | 507 | GVGLSPFLL | XVXXXXXXL | | 781 |
| 95 | 19 | NUC | 123 | GVWIRTPPAY | XVXXXXXXXY | | 782 |
| 85 | 17 | NUC | 25 | GWLWGMDI | XWXXXXXI | | 783 |
| 85 | 17 | NUC | 25 | GWLWGMDIDPY | XWXXXXXXXXY | | 784 |
| 85 | 17 | ENV | 65 | GWSPQAQGI | XWXXXXXXI | 0.0024 | 785 |
| 85 | 17 | ENV | 65 | GWSPQAQGIL | XWXXXXXXXL | 0.0003 | 786 |
| 95 | 19 | POL | 639 | GYPALMPL | XYXXXXXL | | 787 |
| 95 | 19 | POL | 639 | GYPALMPLY | XYXXXXXXY | 0.0490 | 788 |
| 95 | 19 | ENV | 234 | GYRWMCLRRF | XYXXXXXXXF | 0.0110 | 789 |
| 95 | 19 | ENV | 234 | GYRWMCLRRFI | XYXXXXXXXXI | | 790 |
| 85 | 17 | POL | 579 | GYSLNFMGY | XYXXXXXXY | 0.0002 | 791 |
| 75 | 15 | POL | 579 | GYSLNFMGYVI | XYXXXXXXXXI | | 792 |
| 80 | 16 | POL | 820 | HFASPLHVAW | XFXXXXXXXW | | 793 |
| 75 | 15 | POL | 7 | HFRKLLLL | XFXXXXXL | | 794 |
| 80 | 16 | POL | 435 | HLLVGSSGL | XLXXXXXXL | | 795 |
| 75 | 15 | POL | 569 | HLNPNKTKRW | XLXXXXXXXW | | 796 |
| 80 | 16 | POL | 491 | HLYSHPII | XLXXXXXI | | 797 |
| 80 | 16 | POL | 491 | HLYSHPIIL | XLXXXXXXL | | 798 |
| 80 | 16 | POL | 491 | HLYSHPIILGF | XLXXXXXXXXF | | 799 |
| 85 | 17 | POL | 715 | HTAELLAACF | XTXXXXXXXF | | 800 |
| 100 | 20 | NUC | 52 | HTALRQAI | XTXXXXXI | | 801 |
| 95 | 19 | NUC | 52 | HTALRQAIL | XTXXXXXXL | | 802 |
| 95 | 19 | NUC | 52 | HTALRQAILCW | XTXXXXXXXXW | | 803 |
| 100 | 20 | POL | 149 | HTLWKAGI | XTXXXXXI | | 804 |
| 100 | 20 | POL | 149 | HTLWKAGIL | XTXXXXXXL | | 805 |
| 100 | 20 | POL | 149 | HTLWKAGILY | XTXXXXXXXY | | 806 |
| 100 | 20 | POL | 146 | HYLHTLWKAGI | XYXXXXXXXXI | | 807 |
| 100 | 20 | ENV | 381 | IFFCLWVY | XFXXXXXY | | 808 |
| 100 | 20 | ENV | 381 | IFFCLWVYI | XFXXXXXXI | 0.0087 | 809 |
| 80 | 16 | ENV | 245 | IFLFILLL | XFXXXXXL | | 810 |
| 80 | 16 | ENV | 245 | IFLFILLLCL | XFXXXXXXXL | | 811 |
| 80 | 16 | ENV | 245 | IFLFILLLCLI | XFXXXXXXXXI | | 812 |
| 95 | 19 | ENV | 255 | IFLLVLLDY | XFXXXXXXY | | 813 |
| 80 | 16 | ENV | 244 | IIFLFILL | XIXXXXXL | | 814 |
| 80 | 16 | ENV | 244 | IIFLFILLL | XIXXXXXXL | | 815 |
| 80 | 16 | ENV | 244 | IIFLFILLLCL | XIXXXXXXXXL | | 816 |
| 80 | 16 | POL | 497 | IILGFRKI | XIXXXXXI | | 817 |
| 80 | 16 | POL | 497 | IILGFRKIPM | XIXXXXXXXM | | 818 |
| 90 | 18 | NUC | 59 | ILCWGELM | XLXXXXXM | | 819 |
| 80 | 16 | POL | 498 | ILGFRKIPM | XLXXXXXXM | | 820 |
| 100 | 20 | ENV | 249 | ILLLCLIF | XLXXXXXF | | 821 |
| 100 | 20 | ENV | 249 | ILLLCLIFL | XLXXXXXXL | | 822 |
| 100 | 20 | ENV | 249 | ILLLCLIFLL | XLXXXXXXXL | | 823 |
| 80 | 16 | POL | 760 | ILRGTSFVY | XLXXXXXXY | | 824 |
| 95 | 19 | ENV | 188 | ILTIPQSL | XLXXXXXL | | 825 |
| 90 | 18 | ENV | 188 | ILTIPQSLDSW | XLXXXXXXXXW | | 826 |
| 90 | 18 | POL | 625 | IVGLLGFAAPF | XVXXXXXXXXF | | 827 |
| 85 | 17 | ENV | 358 | IWMMWYWGPS | XWXXXXXXXXL | 0.0004 | 828 |
| 95 | 19 | POL | 395 | KFAVPNLQSL | XFXXXXXXXL | 0.0020 | 829 |
| 80 | 16 | POL | 503 | KIPMGVGL | XIXXXXXL | | 830 |
| 80 | 16 | POL | 503 | KIPMGVGLSPF | XIXXXXXXXXF | | 831 |
| 85 | 17 | NUC | 21 | KLCLGWLW | XLXXXXXW | | 832 |
| 85 | 17 | NUC | 21 | KLCLGWLWGM | XLXXXXXXXM | | 833 |
| 95 | 19 | POL | 489 | KLHLYSHPI | XLXXXXXXI | | 834 |
| 80 | 16 | POL | 489 | KLHLYSHPII | XLXXXXXXXI | | 835 |
| 80 | 16 | POL | 489 | KLHLYSHPIIL | XLXXXXXXXXL | | 836 |
| 75 | 15 | POL | 108 | KLIMPARF | XLXXXXXF | | 837 |
| 75 | 15 | POL | 108 | KLIMPARFY | XLXXXXXXY | | 838 |
| 80 | 16 | POL | 610 | KLPVNRPI | XLXXXXXI | | 839 |
| 80 | 16 | POL | 610 | KLPVNRPIDW | XLXXXXXXXW | | 840 |
| 95 | 19 | POL | 574 | KTKRWGYSL | XTXXXXXXL | | 841 |
| 85 | 17 | POL | 574 | KTKRWGYSLNF | XTXXXXXXXXF | | 842 |
| 85 | 17 | POL | 620 | KVCQRIVGL | XVXXXXXXL | | 843 |
| 85 | 17 | POL | 620 | KVCQRIVGLL | XVXXXXXXXL | | 844 |
| 95 | 19 | POL | 55 | KVGNFTGL | XVXXXXXL | | 845 |
| 95 | 19 | POL | 55 | KVGNFTGLY | XVXXXXXXY | | 846 |
| 85 | 17 | X | 91 | KVLHKRTL | XVXXXXXL | | 847 |
| 85 | 17 | X | 91 | KVLHKRTLGL | XVXXXXXXXL | | 848 |
| 100 | 20 | POL | 121 | KYLPLDKGI | XYXXXXXXI | 0.0028 | 849 |
| 85 | 17 | POL | 745 | KYTSFPWL | XYXXXXXL | | 850 |
| 85 | 17 | POL | 745 | KYTSFPWLL | XYXXXXXXL | 3.6000 | 851 |
| 80 | 16 | ENV | 247 | LFILLLCL | XFXXXXXL | | 852 |
| 80 | 16 | ENV | 247 | LFILLLCLI | XFXXXXXXI | | 853 |
| 80 | 16 | ENV | 247 | LFILLLCLIF | XFXXXXXXXF | | 854 |
| 80 | 16 | ENV | 247 | LFILLLCLIFL | XFXXXXXXXXL | | 855 |
| 100 | 20 | ENV | 254 | LIFLLVLL | XIXXXXXL | | 856 |
| 95 | 19 | ENV | 254 | LIFLLVLLDY | XIXXXXXXXY | | 857 |
| 100 | 20 | POL | 109 | LIMPARFY | XIXXXXXY | | 858 |
| 95 | 19 | POL | 514 | LLAQFTSAI | XLXXXXXXI | | 859 |
| 100 | 20 | ENV | 251 | LLCLIFLL | XLXXXXXL | | 860 |
| 100 | 20 | ENV | 251 | LLCLIFLLVL | XLXXXXXXXL | | 861 |
| 100 | 20 | ENV | 251 | LLCLIFLLVLL | XLXXXXXXXXL | | 862 |
| 85 | 17 | NUC | 30 | LLDTASAL | XLXXXXXL | | 863 |
| 85 | 17 | NUC | 30 | LLDTASALY | XLXXXXXXY | | 864 |
| 95 | 19 | ENV | 260 | LLDYQGML | XLXXXXXL | | 865 |
| 80 | 16 | POL | 752 | LLGCAANW | XLXXXXXW | | 866 |
| 80 | 16 | POL | 752 | LLGCAANWI | XLXXXXXXI | | 867 |
| 80 | 16 | POL | 752 | LLGCAANWIL | XLXXXXXXXL | | 868 |
| 95 | 19 | POL | 628 | LLGFAAPF | XLXXXXXF | | 869 |
| 75 | 15 | ENV | 63 | LLGWSPQAQGI | XLXXXXXXXXI | | 870 |
| 100 | 20 | ENV | 250 | LLLCLIFL | XLXXXXXL | | 871 |
| 100 | 20 | ENV | 250 | LLLCLIFLL | XLXXXXXXL | | 872 |
| 100 | 20 | ENV | 250 | LLLCLIFLLVL | XLXXXXXXXXL | | 873 |
| 100 | 20 | ENV | 378 | LLPIFFCL | XLXXXXXL | | 874 |
| 100 | 20 | ENV | 378 | LLPIFFCLW | XLXXXXXXW | | 875 |
| 100 | 20 | ENV | 378 | LLPIFFCLWVY | XLXXXXXXXXY | | 876 |
| 95 | 19 | NUC | 44 | LLSFLPSDF | XLXXXXXXF | | 877 |
| 95 | 19 | NUC | 44 | LLSFLPSDFF | XLXXXXXXXF | | 878 |
| 95 | 19 | POL | 563 | LLSLGIHL | XLXXXXXL | | 879 |
| 90 | 18 | POL | 407 | LLSSNLSW | XLXXXXXW | | 880 |
| 90 | 18 | POL | 407 | LLSSNLSWL | XLXXXXXXL | | 881 |
| 90 | 18 | POL | 407 | LLSSNLSWLSL | XLXXXXXXXXL | | 882 |
| 80 | 16 | ENV | 184 | LLTRILTI | XLXXXXXI | | 883 |
| 80 | 16 | POL | 436 | LLVGSSGL | XLXXXXXL | | 884 |
| 95 | 19 | ENV | 257 | LLVLLDYQGM | XLXXXXXXXM | | 885 |
| 95 | 19 | ENV | 257 | LLVLLDYQGML | XLXXXXXXXXL | | 886 |
| 95 | 19 | ENV | 175 | LLVLQAGF | XLXXXXXF | | 887 |
| 95 | 19 | ENV | 175 | LLVLQAGFF | XLXXXXXXF | | 888 |
| 90 | 18 | ENV | 175 | LLVLQAGFFL | XLXXXXXXXL | | 889 |
| 90 | 18 | ENV | 175 | LLVLQAGFFLL | XLXXXXXXXXL | | 890 |
| 100 | 20 | ENV | 338 | LLVPFVQW | XLXXXXXW | | 891 |
| 100 | 20 | ENV | 338 | LLVPFVQWF | XLXXXXXXF | | 892 |
| 90 | 18 | NUC | 100 | LLWFHISCL | XLXXXXXXL | | 893 |
| 85 | 17 | NUC | 100 | LLWFHISCLTF | XLXXXXXXXXF | | 894 |
| 95 | 19 | POL | 643 | LMPLYACI | XMXXXXXI | | 895 |
| 75 | 15 | NUC | 137 | LTFGRETVL | XTXXXXXXL | | 896 |
| 75 | 15 | NUC | 137 | LTFGRETVLEY | XTXXXXXXXXY | | 897 |
| 90 | 18 | ENV | 189 | LTIPQSLDSW | XTXXXXXXXW | | 898 |
| 90 | 18 | ENV | 189 | LTIPQSLDSWW | XTXXXXXXXXW | | 899 |
| 90 | 18 | POL | 404 | LTNLLSSNL | XTXXXXXXL | | 900 |
| 90 | 18 | POL | 404 | LTNLLSSNLSW | XTXXXXXXXXW | | 901 |
| 80 | 16 | ENV | 185 | LTRILTIPQSL | XTXXXXXXXXL | | 902 |
| 85 | 17 | POL | 99 | LTVNEKRRL | XTXXXXXXL | | 903 |
| 95 | 19 | ENV | 258 | LVLLDYQGM | XVXXXXXXM | | 904 |
| 95 | 19 | ENV | 258 | LVLLDYQGML | XVXXXXXXXL | | 905 |
| 95 | 19 | ENV | 176 | LVLQAGFF | XVXXXXXF | | 906 |
| 90 | 18 | ENV | 176 | LVLQAGFFL | XVXXXXXXL | | 907 |
| 90 | 18 | ENV | 176 | LVLQAGFFLL | XVXXXXXXXL | | 908 |
| 100 | 20 | ENV | 339 | LVPFVQWF | XVXXXXXF | | 909 |
| 95 | 19 | ENV | 339 | LVPFVQWFVGL | XVXXXXXXXXL | | 910 |
| 90 | 18 | NUC | 119 | LVSFGVWI | XVXXXXXI | | 911 |
| 100 | 20 | POL | 377 | LVVDFSQF | XVXXXXXF | | 912 |
| 90 | 18 | NUC | 101 | LWFHISCL | XWXXXXXL | | 913 |
| 85 | 17 | NUC | 101 | LWFHISCLTF | XWXXXXXXXF | | 914 |
| 85 | 17 | NUC | 27 | LWGMDIDPY | XWXXXXXXY | | 915 |
| 100 | 20 | POL | 151 | LWKAGILY | XWXXXXXY | | 916 |
| 80 | 16 | POL | 492 | LYSHPIIL | XYXXXXXL | | 917 |
| 80 | 16 | POL | 492 | LYSHPIILGF | XYXXXXXXXF | 1.1000 | 918 |
| 85 | 17 | ENV | 360 | MMWYWGPSL | XMXXXXXXL | 0.0012 | 919 |
| 85 | 17 | ENV | 360 | MMWYWGPSLY | XMXXXXXXXY | 0.0001 | 920 |
| 85 | 17 | ENV | 361 | MWYWGPSL | XWXXXXXL | | 921 |
| 85 | 17 | ENV | 361 | MWYWGPSLY | XWXXXXXXY | 0.0027 | 922 |
| 95 | 19 | POL | 561 | NFLLSLGI | XFXXXXXI | | 923 |
| 95 | 19 | POL | 561 | NFLLSLGIHL | XFXXXXXXXL | 0.0099 | 924 |
| 95 | 19 | POL | 42 | NLGNLNVSI | XLXXXXXXI | | 925 |
| 95 | 19 | POL | 42 | NLGNLNVSIPW | XLXXXXXXXXW | | 926 |
| 90 | 18 | POL | 406 | NLLSSNLSW | XLXXXXXXW | | 927 |
| 90 | 18 | POL | 406 | NLLSSNLSWL | XLXXXXXXXL | | 928 |
| 95 | 19 | POL | 45 | NLNVSIPW | XLXXXXXW | | 929 |
| 100 | 20 | POL | 400 | NLQSLTNL | XLXXXXXL | | 930 |
| 100 | 20 | POL | 400 | NLQSLTNLL | XLXXXXXXL | | 931 |
| 75 | 15 | ENV | 15 | NLSVPNPL | XLXXXXXL | | 932 |
| 75 | 15 | ENV | 15 | NLSVPNPLGF | XLXXXXXXXF | | 933 |
| 80 | 16 | POL | 758 | NWILRGTSF | XWXXXXXXF | | 934 |
| 80 | 16 | POL | 758 | NWILRGTSFVY | XWXXXXXXXXY | | 935 |
| 95 | 19 | POL | 512 | PFLLAQFTSAI | XFXXXXXXXXI | | 936 |
| 95 | 19 | POL | 634 | PFTQCGYPAL | XFXXXXXXXL | 0.0002 | 937 |
| 95 | 19 | POL | 634 | PFTQCGYPALM | XFXXXXXXXXM | | 938 |
| 95 | 19 | ENV | 341 | PFVQWFVGL | XFXXXXXXL | 0.0003 | 939 |
| 85 | 17 | POL | 616 | PIDWKVCQRI | XIXXXXXXXI | | 940 |
| 100 | 20 | ENV | 380 | PIFFCLWVY | XIXXXXXXY | | 941 |
| 100 | 20 | ENV | 380 | PIFFFCLWVYI | XIXXXXXXXI | | 942 |
| 85 | 17 | POL | 713 | PIHTAELL | XIXXXXXL | | 943 |
| 80 | 16 | POL | 496 | PIILGFRKI | XIXXXXXXI | | 944 |
| 80 | 16 | POL | 496 | PIILGFRKIPM | XIXXXXXXXXM | | 945 |
| 100 | 20 | ENV | 314 | PIPSSWAF | XIXXXXXF | | 946 |
| 100 | 20 | POL | 124 | PLDKGIKPY | XLXXXXXXY | | 947 |
| 100 | 20 | POL | 124 | PLDKGIKPYY | XLXXXXXXXY | | 948 |
| 95 | 19 | POL | 20 | PLEEELPRL | XLXXXXXXL | | 949 |
| 95 | 19 | ENV | 10 | PLGFFPDHQL | XLXXXXXXXL | | 950 |
| 100 | 20 | POL | 427 | PLHPAAMPHL | XLXXXXXXXL | | 951 |
| 100 | 20 | POL | 427 | PLHPAAMPHLL | XLXXXXXXXXL | | 952 |
| 100 | 20 | ENV | 377 | PLLPIFFCL | XLXXXXXXL | | 953 |
| 100 | 20 | ENV | 377 | PLLPIFFCLW | XLXXXXXXXW | | 954 |
| 95 | 19 | ENV | 174 | PLLVLQAGF | XLXXXXXXF | | 955 |
| 95 | 19 | ENV | 174 | PLLVLQAGFF | XLXXXXXXXF | | 956 |
| 90 | 18 | ENV | 174 | PLLVLQAGFFL | XLXXXXXXXXL | | 957 |
| 80 | 16 | POL | 711 | PLPIHTAEL | XLXXXXXXL | | 958 |
| 80 | 16 | POL | 711 | PLPIHTAELL | XLXXXXXXXL | | 959 |
| 75 | 15 | POL | 2 | PLSYQHFRKL | XLXXXXXXXL | | 960 |
| 75 | 15 | POL | 2 | PLSYQHFRKLL | XLXXXXXXXXL | | 961 |
| 85 | 17 | POL | 98 | PLTVNEKRRL | XLXXXXXXXL | | 962 |
| 80 | 16 | POL | 505 | PMGVGLSPF | XMXXXXXXF | | 963 |
| 80 | 16 | POL | 505 | PMGVGLSPFL | XMXXXXXXXL | | 964 |
| 80 | 16 | POL | 505 | PMGVGLSPFLL | XMXXXXXXXXL | | 965 |
| 75 | 15 | POL | 692 | PTGWGLAI | XTXXXXXI | | 966 |
| 85 | 17 | POL | 797 | PTTGRTSL | XTXXXXXL | | 967 |
| 85 | 17 | POL | 797 | PTTGRTSLY | XTXXXXXXY | | 968 |
| 80 | 16 | NUC | 15 | PTVQASKL | XTXXXXXL | | 969 |
| 80 | 16 | NUC | 15 | PTVQASKLCL | XTXXXXXXXL | | 970 |
| 75 | 15 | ENV | 351 | PTVWLSVI | XTXXXXXI | | 971 |
| 75 | 15 | ENV | 351 | PTVWLSVIW | XTXXXXXXW | | 972 |
| 75 | 15 | ENV | 351 | PTVWLSVIWM | XTXXXXXXXM | | 973 |
| 85 | 17 | POL | 612 | PVNRPIDW | XVXXXXXW | | 974 |
| 80 | 16 | POL | 750 | PWLLGCAANW | XWXXXXXXXW | | 975 |
| 80 | 16 | POL | 750 | PWLLGCAANWI | XWXXXXXXXXI | | 976 |
| 100 | 20 | POL | 51 | PWTHKVGNF | XWXXXXXXF | 0.0290 | 977 |
| 80 | 16 | X | 8 | QLDPARDVL | XLXXXXXXL | | 978 |
| 80 | 16 | X | 8 | QLDPARDVLCL | XLXXXXXXXXL | | 979 |
| 90 | 18 | NUC | 99 | QLLWFHISCL | XLXXXXXXXL | | 980 |
| 95 | 19 | POL | 685 | QVFADATPTGW | XVXXXXXXXXW | | 981 |
| 95 | 19 | ENV | 344 | QWFVGLSPTVW | XWXXXXXXXX | | 982 |
| 75 | 15 | ENV | 242 | RFIIFLFI | XFXXXXXI | | 983 |
| 75 | 15 | ENV | 242 | RFIIFLFIL | XFXXXXXXL | | 984 |
| 75 | 15 | ENV | 242 | RFIIFLFILL | XFXXXXXXXL | | 985 |
| 75 | 15 | ENV | 242 | RFIIFLFILLL | XFXXXXXXXXL | | 986 |
| 100 | 20 | ENV | 332 | RFSWLSLL | XFXXXXXL | | 987 |
| 100 | 20 | ENV | 332 | RFSWLSLLVPF | XFXXXXXXXXF | | 988 |
| 80 | 16 | ENV | 187 | RILTIPQSL | XIXXXXXXL | | 989 |
| 90 | 18 | POL | 624 | RIVGLLGF | XIXXXXXF | | 990 |
| 75 | 15 | POL | 106 | RLKLIMPARF | XLXXXXXXXF | | 991 |
| 75 | 15 | POL | 106 | RLKLIMPARFY | XLXXXXXXXXY | | 992 |
| 95 | 19 | POL | 376 | RLVVDFSQF | XLXXXXXXF | | 993 |
| 90 | 18 | POL | 353 | RTPARVTGGVF | XTXXXXXXXXF | | 994 |
| 95 | 19 | POL | 36 | RVAEDLNL | XVXXXXXL | | 995 |
| 90 | 18 | POL | 36 | RVAEDLNLGNL | XVXXXXXXXXL | | 996 |
| 80 | 16 | POL | 818 | RVHFASPL | XVXXXXXL | | 997 |
| 100 | 20 | POL | 357 | RVTGGVFL | XVXXXXXL | | 998 |
| 85 | 17 | POL | 577 | RWGYSLNF | XWXXXXXF | | 999 |
| 85 | 17 | POL | 577 | RWGYSLNFM | XWXXXXXXM | | 1000 |
| 85 | 17 | POL | 577 | RWGYSLNFMGY | XWXXXXXXXXY | | 1001 |
| 95 | 19 | ENV | 236 | RWMCLRRF | XWXXXXXF | | 1002 |
| 95 | 19 | ENV | 236 | RWMCLRRFI | XWXXXXXXI | 0.0710 | 1003 |
| 95 | 19 | ENV | 236 | RWMCLRRFII | XWXXXXXXXI | 1.1000 | 1004 |
| 95 | 19 | ENV | 236 | RWMCLRRFIIF | XWXXXXXXXXF | | 1005 |
| 100 | 20 | POL | 167 | SFCGSPYSW | XFXXXXXXW | 0.0710 | 1006 |
| 95 | 19 | NUC | 46 | SFLPSDFF | XFXXXXXF | | 1007 |
| 80 | 16 | POL | 765 | SFVYVPSAL | XFXXXXXXL | | 1008 |
| 100 | 20 | POL | 49 | SIPWTHKVGNF | XIXXXXXXXXF | | 1009 |
| 95 | 19 | ENV | 194 | SLDSWWTSL | XLXXXXXXL | | 1010 |
| 95 | 19 | ENV | 194 | SLDSWWTSLNF | XLXXXXXXXXF | | 1011 |
| 95 | 19 | POL | 416 | SLDVSAAF | XLXXXXXF | | 1012 |
| 95 | 19 | POL | 416 | SLDVSAAFY | XLXXXXXXY | | 1013 |
| 100 | 20 | ENV | 337 | SLLVPFVQW | XLXXXXXXW | | 1014 |
| 100 | 20 | ENV | 337 | SLLVPFVQWF | XLXXXXXXXF | | 1015 |
| 75 | 15 | POL | 581 | SLNFMGYVI | XLXXXXXXI | | 1016 |
| 95 | 19 | X | 54 | SLRGLPVCAF | XLXXXXXXXF | | 1017 |
| 90 | 18 | POL | 403 | SLTNLLSSNL | XLXXXXXXXL | | 1018 |
| 75 | 15 | X | 104 | STTDLEAY | XTXXXXXY | | 1019 |
| 75 | 15 | X | 104 | STTDLEAYF | XTXXXXXXF | | 1020 |
| 75 | 15 | ENV | 17 | SVPNPLGF | XVXXXXXF | | 1021 |
| 85 | 17 | POL | 548 | SVQHLESL | XVXXXXXL | | 1022 |
| 80 | 16 | ENV | 330 | SVRFSWLSL | XVXXXXXXL | | 1023 |
| 80 | 16 | ENV | 330 | SVPFSWLSLL | XVXXXXXXXL | | 1024 |
| 90 | 18 | POL | 739 | SVVLSRKY | XVXXXXXY | | 1025 |
| 85 | 17 | POL | 739 | SVVLSRKYTSF | XVXXXXXXXXF | | 1026 |
| 95 | 19 | POL | 524 | SVVRRAFPHCL | XVXXXXXXXXL | | 1027 |
| 95 | 19 | POL | 413 | SWLSLDVSAAF | XWXXXXXXXXF | | 1028 |
| 100 | 20 | ENV | 334 | SWLSLLVPF | XWXXXXXXF | 0.3900 | 1029 |
| 95 | 19 | POL | 392 | SWPKFAVPNL | XWXXXXXXXL | 5.6000 | 1030 |
| 100 | 20 | ENV | 197 | SWWTSLNF | XWXXXXXF | | 1031 |
| 95 | 19 | ENV | 197 | SWWTSLNFL | XWXXXXXXL | 0.3800 | 1032 |
| 90 | 18 | POL | 537 | SYMDDVVL | XYXXXXXL | | 1033 |
| 75 | 15 | POL | 4 | SYQHFRKL | XYXXXXXL | | 1034 |
| 75 | 15 | POL | 4 | SYQHFRKLL | XYXXXXXXL | 0.0051 | 1035 |
| 75 | 15 | POL | 4 | SYQHFRKLLL | XYXXXXXXXL | 0.0660 | 1036 |
| 75 | 15 | POL | 4 | SYQHFRKLLLL | XYXXXXXXXXL | | 1037 |
| 75 | 15 | NUC | 138 | TFGRETVL | XFXXXXXL | | 1038 |
| 75 | 15 | NUC | 138 | TFGRETVLEY | XFXXXXXXY | | 1039 |
| 75 | 15 | NUC | 138 | TFGRETVLEYL | XFXXXXXXXXL | | 1040 |
| 95 | 19 | POL | 657 | TFSPTYKAF | XFXXXXXXF | 0.0060 | 1041 |
| 95 | 19 | POL | 657 | TFSPTYKAF | XFXXXXXXL | 0.0043 | 1042 |
| 90 | 18 | ENV | 190 | TIPQSLDSW | XIXXXXXXW | | 1043 |
| 90 | 18 | ENV | 190 | TIPQSLDSWW | XIXXXXXXXW | | 1044 |
| 100 | 20 | POL | 150 | TLWKAGIL | XLXXXXXL | | 1045 |
| 100 | 20 | POL | 150 | TLWKAGILY | XLXXXXXXY | | 1046 |
| 75 | 15 | X | 105 | TTDLEAYF | XTXXXXXF | | 1047 |
| 85 | 17 | POL | 798 | TTGRTSLY | XTXXXXXY | | 1048 |
| 85 | 17 | POL | 100 | TVNEKRRL | XVXXXXXL | | 1049 |
| 80 | 16 | NUC | 16 | TVQASKLCL | XVXXXXXXL | | 1050 |
| 80 | 16 | NUC | 16 | TVQASKLCLGW | XVXXXXXXXXW | | 1051 |
| 75 | 15 | ENV | 352 | TVWLSVIW | XVXXXXXW | | 1052 |
| 75 | 15 | ENV | 352 | TVWLSVIWM | XVXXXXXXM | | 1053 |
| 95 | 19 | POL | 686 | VFADATPTGW | XFXXXXXXXW | 0.0180 | 1054 |
| 75 | 15 | X | 131 | VFVLGGCRHKL | XFXXXXXXXXL | | 1055 |
| 85 | 17 | POL | 543 | VLGAKSVQHL | XLXXXXXXXL | | 1056 |
| 90 | 18 | X | 133 | VLGGCRHKL | XLXXXXXXL | | 1057 |
| 85 | 17 | X | 92 | VLHKRTLGL | XLXXXXXXL | | 1058 |
| 95 | 19 | ENV | 259 | VLLDYQGM | XLXXXXXM | | 1059 |
| 95 | 19 | ENV | 259 | VLLDYQGML | XLXXXXXXL | | 1060 |
| 95 | 19 | ENV | 177 | VLQAGFFL | XLXXXXXL | | 1061 |
| 95 | 19 | ENV | 177 | VLQAGFFLL | XLXXXXXXL | | 1062 |
| 85 | 17 | POL | 741 | VLSRKYTSF | XLXXXXXXF | | 1063 |
| 85 | 17 | POL | 741 | VLSRKYTSFPW | XLXXXXXXXXW | | 1064 |
| 80 | 16 | POL | 542 | VVLGAKSVQHL | XVXXXXXXXXL | | 1065 |
| 85 | 17 | POL | 740 | VVLSRKYTSF | XVXXXXXXXF | | 1066 |
| 95 | 19 | POL | 525 | VVRRAFPHCL | XVXXXXXXXL | | 1067 |
| 95 | 19 | NUC | 124 | VWIRTPPAY | XWXXXXXXY | | 1068 |
| 75 | 15 | ENV | 353 | VWLSVIWM | XWXXXXXM | | 1069 |
| 90 | 18 | NUC | 102 | WFHISCLTF | XFXXXXXXF | 0.0300 | 1070 |
| 95 | 19 | ENV | 345 | WFVGLSPTVW | XFXXXXXXXW | 0.0120 | 1071 |
| 95 | 19 | ENV | 345 | WFVGLSPTVWL | XFXXXXXXXXL | | 1072 |
| 80 | 16 | POL | 759 | WILRGTSF | XIXXXXXF | | 1073 |
| 80 | 16 | POL | 759 | WILRGTSFVY | XIXXXXXXXY | | 1074 |
| 95 | 19 | NUC | 125 | WIRTPPAY | XIXXXXXY | | 1075 |
| 80 | 16 | POL | 751 | WLLGCAANW | XLXXXXXXW | | 1076 |
| 80 | 16 | POL | 751 | WLLGCAANWI | XLXXXXXXXI | | 1077 |
| 80 | 16 | POL | 751 | WLLGCAANWIL | XLXXXXXXXXL | | 1078 |
| 95 | 19 | POL | 414 | WLSLDVSAAF | XLXXXXXXXF | | 1079 |
| 95 | 19 | POL | 414 | WLSLDVSAAFY | XLXXXXXXXXY | | 1080 |
| 100 | 20 | ENV | 335 | WLSLLVPF | XLXXXXXF | | 1081 |
| 100 | 20 | ENV | 335 | WLSLLVPFVQW | XLXXXXXXXXW | | 1082 |
| 85 | 17 | NUC | 26 | WLWGMDIDPY | XLXXXXXXXY | | 1083 |
| 95 | 19 | ENV | 237 | WMCLRRFI | XMXXXXXI | | 1084 |
| 95 | 19 | ENV | 237 | WMCLRRFII | XMXXXXXXI | 0.0230 | 1085 |
| 95 | 19 | ENV | 237 | WMCLRRFIIF | XMXXXXXXXF | 0.0013 | 1086 |
| 95 | 19 | ENV | 237 | WMCLRRFIIFL | XMXXXXXXXXL | | 1087 |
| 85 | 17 | ENV | 359 | WMMWYWGPSL | XMXXXXXXXL | 0.0005 | 1088 |
| 85 | 17 | ENV | 359 | WMMWYWGPSL | XMXXXXXXXXY | | 1089 |
| 100 | 20 | POL | 52 | WTHKVGNF | XTXXXXXF | | 1090 |
| 95 | 19 | POL | 52 | WTHKVGNFTGL | XTXXXXXXXXL | | 1091 |
| 95 | 19 | ENV | 198 | WWTSLNFL | XWXXXXXL | | 1092 |
| 85 | 17 | ENV | 362 | WYWGPSLY | XYXXXXXY | 0.0001 | 1093 |
| 100 | 20 | POL | 147 | YLHTLWKAGI | XLXXXXXXXI | | 1094 |
| 100 | 20 | POL | 147 | YLHTLWKAGIL | XLXXXXXXXXL | | 1095 |
| 100 | 20 | POL | 122 | YLPLDKGI | XLXXXXXI | | 1096 |
| 100 | 20 | POL | 122 | YLPLDKGIKPY | XLXXXXXXXXY | | 1097 |
| 90 | 18 | NUC | 118 | YLVSFGVW | XLXXXXXW | | 1098 |
| 90 | 18 | NUC | 118 | YLVSFGVWI | XLXXXXXXI | | 1099 |
| 85 | 17 | POL | 746 | YTSFPWLL | XTXXXXXL | | 1100 |

TABLE XI
HBV B07 SUPER MOTIF (With binding information)

| | | | | | | | | | | | | | |
|---|---|---|---|---|---|---|---|---|---|---|---|---|---|
| Conserv- | Fre- | | | | | C- | | | | | | | |
| ancy | quency | Protein | Position | Sequence | P2 | term | AA | B*0702 | B*3501 | B*5101 | B*5301 | B*5401 | SEQ ID NO |

| | | | | | | | | | | | | | |
|---|---|---|---|---|---|---|---|---|---|---|---|---|---|
| 75 | 15 | X | 146 | APCNFFTSA | P | A | 9 | | | | | | 1101 |
| 95 | 19 | POL | 633 | APFTQCGY | P | Y | 8 | 0.0001 | 0.0012 | 0.0019 | 0.0002 | 0.0002 | 1102 |
| 95 | 19 | POL | 633 | APFTQCGYPA | P | A | 10 | 0.0029 | 0.0001 | | 0.0002 | 1.4000 | 1103 |
| 95 | 19 | POL | 633 | APFTQCGYPAL | P | L | 11 | 0.2300 | 0.0010 | 0.0004 | −0.0003 | 0.0093 | 1104 |
| 100 | 20 | ENV | 232 | CPGYRWMCL | P | L | 9 | | | | | | 1105 |
| 80 | 16 | NUC | 14 | CPTVQASKL | P | L | 9 | | | | | | 1106 |
| 80 | 16 | NUC | 14 | CPTVQASKLCL | P | L | 11 | | | | | | 1107 |
| 80 | 16 | X | 10 | DPARDVLCL | P | L | 9 | | | | | | 1108 |
| 80 | 16 | ENV | 122 | DPRVRGLY | P | Y | 8 | | | | | | 1109 |
| 90 | 18 | POL | 778 | DPSRGRLGL | P | L | 9 | 0.0120 | 0.0001 | 0.0001 | 0.0001 | 0.0001 | 1110 |
| 90 | 18 | NUC | 33 | DPYKEFGA | P | A | 8 | 0.0001 | 0.0001 | 0.0019 | 0.0002 | 0.0019 | 1111 |
| 75 | 15 | ENV | 130 | FPAGGSSSGTV | P | V | 11 | | | | | | 1112 |
| 90 | 18 | ENV | 14 | FPDHQLDPA | P | A | 9 | | | | | | 1113 |
| 85 | 17 | ENV | 14 | FPDHQLDPAF | P | F | 10 | 0.0002 | 0.0016 | 0.0003 | 0.0011 | 0.0021 | 1114 |
| 95 | 19 | POL | 530 | FPHCLAFSY | P | Y | 9 | 0.0001 | 0.5250 | 0.0665 | 0.5400 | 0.0199 | 1115 |
| 95 | 19 | POL | 530 | FPHCLAFSYM | P | M | 10 | 0.0990 | 0.2200 | 0.0900 | 0.0790 | 0.0480 | 1116 |
| 75 | 15 | POL | 749 | FPWLLGCA | P | A | 8 | | | | | | 1117 |
| 75 | 15 | POL | 749 | FPWLLGCAA | P | A | 9 | | | | | | 1118 |
| 75 | 15 | POL | 749 | FPWLLGCAANW | P | W | 11 | | | | | | 1119 |
| 90 | 18 | X | 67 | GPCALRFTSA | P | A | 10 | 0.0900 | 0.0001 | 0.0001 | 0.0002 | 0.0035 | 1120 |
| 95 | 19 | POL | 19 | GPLEEELPRL | P | L | 10 | 0.0001 | 0.0001 | 0.0002 | 0.0001 | 0.0002 | 1121 |
| 90 | 18 | POL | 19 | GPLEEELPRLA | P | A | 11 | −0.0002 | 0.0001 | 0.0001 | −0.0003 | 0.0001 | 1122 |
| 95 | 19 | ENV | 173 | GPLLVLQA | P | A | 8 | 0.0003 | 0.0001 | 0.0110 | 0.0002 | 0.0065 | 1123 |
| 95 | 19 | ENV | 173 | GPLLVLQAGF | P | F | 10 | 0.0001 | 0.0001 | 0.0002 | 0.0001 | 0.0002 | 1124 |
| 95 | 19 | ENV | 173 | GPLLVLQAGFF | P | F | 11 | 0.0011 | 0.0001 | 0.0001 | 0.0008 | 0.0009 | 1125 |
| 95 | 17 | POL | 97 | GPLTVNEKRRL | P | L | 11 | 0.0031 | 0.0001 | 0.0001 | −0.0003 | 0.0001 | 1126 |
| 100 | 20 | POL | 429 | HPAAMPHL | P | L | 8 | 0.0650 | 0.0004 | 0.3100 | 0.0037 | 0.0160 | 1127 |
| 100 | 20 | POL | 429 | HPAAMPHLL | P | L | 9 | 0.0980 | 0.0270 | 0.0110 | 0.0500 | 0.0120 | 1128 |
| 85 | 17 | POL | 429 | HPAAMPHLLV | P | V | 10 | 0.0160 | 0.0020 | 0.0078 | 0.0140 | 0.0170 | 1129 |
| 80 | 16 | POL | 495 | HPIILGFRKI | P | I | 10 | | | | | | 1130 |
| 100 | 20 | ENV | 313 | IPIPSSWA | P | A | 8 | 0.0004 | 0.0004 | 0.0019 | 0.0002 | 0.0600 | 1131 |
| 100 | 20 | ENV | 313 | IPIPSSWAF | P | F | 9 | 0.1300 | 2.7679 | 2.3500 | 0.7450 | 0.0034 | 1132 |
| 80 | 16 | ENV | 313 | IPIPSSWAFA | P | A | 10 | 0.0013 | 0.0024 | | 0.0014 | 0.4500 | 1133 |
| 80 | 16 | POL | 504 | IPMGVGLSPF | P | F | 10 | | | | | | 1134 |
| 80 | 16 | POL | 504 | IPMGVGLSPFL | P | L | 11 | | | | | | 1135 |
| 90 | 18 | ENV | 191 | IPQSLDSW | P | W | 8 | | | | | | 1136 |
| 90 | 18 | ENV | 191 | IPQSLDSWW | P | W | 9 | | | | | | 1137 |
| 80 | 16 | ENV | 315 | IPSSWAFA | P | A | 8 | | | | | | 1138 |
| 100 | 20 | POL | 50 | IPWTHKVGNF | P | F | 10 | 0.0013 | 0.0001 | 0.0007 | 0.0001 | 0.0002 | 1139 |
| 100 | 20 | ENV | 379 | LPIFFCLW | P | W | 8 | 0.0001 | 0.0001 | 0.0360 | 0.1400 | 0.0035 | 1140 |
| 100 | 20 | ENV | 379 | LPIFFCLWV | P | V | 9 | | | | | | 1141 |
| 100 | 20 | ENV | 379 | LPIFFCLWVY | P | Y | 10 | 0.0002 | 0.0079 | 0.0002 | 0.0006 | 0.0002 | 1142 |
| 100 | 20 | ENV | 379 | LPIFFCLWVYI | P | I | 11 | 0.0002 | 0.0001 | 0.0043 | 0.0139 | 0.0021 | 1143 |
| 85 | 17 | POL | 712 | LPIHTAEL | P | L | 8 | | | | | | 1144 |
| 85 | 17 | POL | 712 | LPIHTAELL | P | L | 9 | 0.0040 | 0.0630 | 0.0052 | 0.3100 | 0.0005 | 1145 |
| 85 | 17 | POL | 712 | LPIHTAELLA | P | A | 10 | 0.0018 | 0.0011 | | 0.0016 | 0.3300 | 1146 |
| 85 | 17 | POL | 712 | LPIHTAELLAA | P | A | 11 | 0.0090 | 0.0027 | −0.0003 | 0.0120 | 2.7500 | 1147 |
| 80 | 16 | X | 89 | LPKVLHKRTL | P | L | 10 | | | | | | 1148 |
| 100 | 20 | POL | 123 | LPLDKGIKPY | P | Y | 10 | 0.0001 | 0.0290 | 0.0002 | 0.0003 | 0.0002 | 1149 |
| 100 | 20 | POL | 123 | LPLDKGIKPYY | P | Y | 11 | −0.0002 | 0.0009 | 0.0001 | 0.0007 | 0.0001 | 1150 |
| 95 | 19 | X | 58 | LPVCAFSSA | P | A | 9 | 0.0480 | 0.0710 | 0.0110 | 0.0009 | 19.0000 | 1151 |
| 80 | 16 | POL | 611 | LPVNRPIDW | P | W | 9 | | | | | | 1152 |
| 80 | 16 | POL | 611 | LPVNRPIDWKV | P | V | 11 | | | | | | 1153 |
| 80 | 16 | POL | 433 | MPHLLVGSSGL | P | L | 11 | | | | | | 1154 |
| 100 | 20 | POL | 1 | MPLSYQHF | P | F | 8 | 0.0001 | 0.0097 | 0.0120 | 0.0370 | 0.0190 | 1155 |
| 75 | 15 | POL | 1 | MPLSYQHFRKL | P | L | 11 | | | | | | 1156 |
| 90 | 18 | POL | 774 | NPADDPSRGRL | P | L | 11 | 0.0120 | 0.0001 | 0.0001 | −0.0003 | 0.0001 | 1157 |
| 95 | 19 | ENV | 9 | NPLGFFPDHQL | P | L | 11 | 0.0012 | 0.0021 | 0.0001 | 0.0028 | 0.0001 | 1158 |
| 75 | 15 | POL | 571 | NPNKTKRW | P | W | 8 | | | | | | 1159 |
| 75 | 15 | POL | 571 | NPNKTKRWGY | P | Y | 10 | | | | | | 1160 |
| 95 | 19 | NUC | 129 | PPAYRPPNA | P | A | 9 | 0.0001 | 0.0001 | 0.0001 | 0.0002 | 0.0003 | 1161 |
| 95 | 19 | NUC | 129 | PPAYRPPNAPI | P | I | 11 | 0.0003 | 0.0001 | 0.0001 | −0.0003 | 0.0001 | 1162 |
| 85 | 17 | ENV | 58 | PPHGGLLGW | P | W | 9 | 0.0001 | 0.0002 | 0.0001 | 0.0003 | 0.0002 | 1163 |
| 100 | 20 | NUC | 134 | PPNAPILSTL | P | L | 10 | 0.0001 | 0.0001 | 0.0035 | 0.0001 | 0.0002 | 1164 |
| 80 | 16 | POL | 615 | RPIDWKVCQRI | P | I | 11 | | | | | | 1165 |
| 100 | 20 | NUC | 133 | RPPNAPIL | P | L | 8 | 0.0076 | 0.0001 | 0.0280 | 0.0002 | 0.0002 | 1166 |
| 100 | 20 | NUC | 133 | RPPNAPILSTL | P | L | 11 | 0.1300 | 0.0001 | 0.0018 | −0.0003 | 0.0001 | 1167 |
| 100 | 20 | NUC | 44 | SPEHCSPHHTA | P | A | 11 | −0.0002 | 0.0001 | 0.0001 | −0.0003 | 0.0011 | 1168 |
| 95 | 19 | POL | 511 | SPFLLAQF | P | F | 8 | 0.5500 | 0.0009 | 0.0180 | 0.0009 | 0.0093 | 1169 |
| 95 | 19 | POL | 511 | SPFLLAQFTSA | P | A | 11 | 0.0820 | 0.0001 | 0.0001 | −0.0003 | 12.0500 | 1170 |
| 100 | 20 | NUC | 49 | SPHHTALRQA | P | A | 10 | 0.0012 | 0.0001 | | 0.0002 | 0.0035 | 1171 |
| 100 | 20 | NUC | 49 | SPHHTALRQAI | P | I | 11 | 0.5800 | 0.0001 | 0.0004 | 0.0005 | 0.0002 | 1172 |
| 85 | 17 | ENV | 67 | SPQAQGIL | P | L | 8 | | | | | | 1173 |
| 85 | 17 | POL | 808 | SPSVPSHL | P | L | 8 | | | | | | 1174 |
| 75 | 15 | ENV | 350 | SPTVWLSV | P | V | 8 | | | | | | 1175 |
| 75 | 15 | ENV | 350 | SPTVWLSVI | P | I | 9 | | | | | | 1176 |
| 75 | 15 | ENV | 350 | SPTVWLSVIW | P | W | 10 | | | | | | 1177 |
| 75 | 15 | ENV | 350 | SPTVWLSVIWM | P | M | 11 | | | | | | 1178 |
| 95 | 19 | POL | 659 | SPTYKAFL | P | L | 8 | 0.3900 | 0.0001 | 0.0019 | 0.0002 | 0.0002 | 1179 |
| 90 | 18 | POL | 354 | TPARVTGGV | P | V | 9 | 0.0078 | 0.0001 | 0.0013 | 0.0001 | 0.0015 | 1180 |
| 90 | 18 | POL | 354 | TPARVTGGVF | P | F | 10 | 0.3200 | 0.1000 | 0.0001 | 0.0099 | 0.0006 | 1181 |
| 90 | 18 | POL | 354 | TPARVTGGVFL | P | L | 11 | 0.0950 | 0.0001 | 0.0001 | 0.0005 | 0.0005 | 1182 |
| 95 | 19 | NUC | 128 | TPPAYRPPNA | P | A | 10 | 0.0001 | 0.0001 | | 0.0002 | 0.0100 | 1183 |
| 75 | 15 | ENV | 57 | TPPHGGLL | P | L | 8 | | | | | | 1184 |
| 75 | 15 | ENV | 57 | TPPHGGLLGW | P | W | 10 | | | | | | 1185 |
| 80 | 16 | POL | 691 | TPTGWGLA | P | A | 8 | | | | | | 1186 |
| 75 | 15 | POL | 691 | TPTGWGLAI | P | I | 9 | | | | | | 1187 |
| 95 | 19 | ENV | 340 | VPFVQWFV | P | V | 8 | 0.0010 | 0.0001 | 19.0000 | 0.0002 | 0.1100 | 1188 |
| 95 | 19 | ENV | 340 | VPFVQWFVGL | P | L | 10 | 0.0011 | 0.0001 | 0.0100 | 0.0001 | 0.0025 | 1189 |
| 95 | 19 | POL | 398 | VPNLQSLTNL | P | L | 10 | 0.0006 | 0.0001 | 0.0004 | 0.0001 | 0.0002 | 1190 |
| 95 | 19 | POL | 398 | VPNLQSLTNLL | P | L | 11 | 0.0004 | 0.0001 | 0.0001 | −0.0003 | 0.0002 | 1191 |
| 90 | 18 | POL | 769 | VPSALNPA | P | A | 8 | 0.0011 | 0.0001 | 0.0070 | 0.0002 | 1.0000 | 1192 |
| 95 | 19 | POL | 393 | WPKFAVPNL | P | L | 9 | 0.0054 | 0.0002 | 0.0015 | 0.0001 | 0.0015 | 1193 |
| 95 | 19 | POL | 640 | YPALMPLY | P | Y | 8 | 0.0004 | 0.2600 | 0.4100 | 0.0450 | 0.0056 | 1194 |
| 95 | 19 | POL | 640 | YPALMPLYA | P | A | 9 | 0.0180 | 0.0480 | 0.0340 | 0.0140 | 16.0000 | 1195 |
| 95 | 19 | POL | 640 | YPALMPLYACI | P | I | 11 | 0.0040 | 0.0001 | 0.0470 | 0.0320 | 0.0700 | 1196 |

TABLE XII
HBV B27 Super Motif (No binding data available)

| | | | | | | |
|---|---|---|---|---|---|---|
| | | Position in | No. of | Sequence | Conservancy | |
| Protein | Sequence | HBV | Amino Acids | Frequency | (%) | SeqID Num |

| | | | | | | |
|---|---|---|---|---|---|---|
| | | | | | | 1197 |
| AYW | AHLSLRGL | 51 | 8 | 19 | 95 | 1198 |
| AYW | ARVTGGVF | 356 | 8 | 18 | 90 | 1199 |
| AYW | DHGAHLSL | 48 | 8 | 19 | 95 | 1200 |
| AYW | DHQLDPAF | 16 | 8 | 18 | 90 | 1201 |
| AYW | DKGIKPYY | 126 | 8 | 20 | 100 | 1202 |
| AYW | FHISCLTF | 103 | 8 | 18 | 90 | 1203 |
| AYW | FRKIPMGV | 501 | 8 | 16 | 80 | 1204 |
| AYR | GRETVLEY | 140 | 8 | 15 | 75 | 1205 |
| AYW | HHTALRQA | 51 | 8 | 20 | 100 | 1206 |
| AYW | IHTAELLA | 714 | 8 | 17 | 85 | 1207 |
| AYW | LHKRTLGL | 93 | 8 | 18 | 90 | 1208 |
| AYW | LHLYSHPI | 490 | 8 | 19 | 95 | 1209 |
| AYW | LRGLPVCA | 55 | 8 | 19 | 95 | 1210 |
| AYW | LRGTSFVY | 761 | 8 | 16 | 80 | 1211 |
| AYW | LRQAILCW | 55 | 8 | 19 | 95 | 1212 |
| AYW | LRRFIIFL | 240 | 8 | 19 | 95 | 1213 |
| AYW | NKTKRWGY | 573 | 8 | 15 | 75 | 1214 |
| AYW | NRPIDWKV | 614 | 8 | 18 | 90 | 1215 |
| AYW | NRRVAEDL | 34 | 8 | 17 | 85 | 1216 |
| AYW | PHCLAFSY | 531 | 8 | 19 | 95 | 1217 |
| AYW | PHGGLLGW | 59 | 8 | 17 | 85 | 1218 |
| AYW | PKFAVPNL | 394 | 8 | 19 | 95 | 1219 |
| AYR | QHFRKLLL | 6 | 8 | 15 | 75 | 1220 |
| AYW | RHYLHTLW | 145 | 8 | 20 | 100 | 1221 |
| AYW | RKYTSFPW | 744 | 8 | 17 | 85 | 1222 |
| AYW | RRAFPHCL | 527 | 8 | 19 | 95 | 1223 |
| AYW | RRFIIFLF | 241 | 8 | 15 | 75 | 1224 |
| AYW | SHPIILGF | 494 | 8 | 16 | 80 | 1225 |
| AYW | SKLCLGWL | 20 | 8 | 18 | 90 | 1226 |
| AYW | SRNLYVSL | 472 | 8 | 16 | 80 | 1227 |
| AYW | TKRWGYSL | 575 | 8 | 19 | 95 | 1228 |
| AYW | TRHYLHTL | 144 | 8 | 20 | 100 | 1229 |
| AYW | VRFSWLSL | 331 | 8 | 16 | 80 | 1230 |
| AYW | WKVCQRIV | 619 | 8 | 17 | 85 | 1231 |
| AYW | YRPPNAPI | 132 | 8 | 20 | 100 | 1232 |
| AYW | ARVTGGVFL | 356 | 9 | 18 | 90 | 1233 |
| AYW | EHCSPHHTA | 46 | 9 | 20 | 100 | 1234 |
| AYR | GRETVLEYL | 140 | 9 | 15 | 75 | 1235 |
| AYW | HHTALRQAI | 51 | 9 | 20 | 100 | 1236 |
| AYW | HKVGNFTGL | 54 | 9 | 19 | 95 | 1237 |
| AYW | IHTAELLAA | 714 | 9 | 17 | 85 | 1238 |
| AYW | KRWGYSLNF | 576 | 9 | 17 | 85 | 1239 |
| AYW | LHLYSHPII | 490 | 9 | 16 | 80 | 1240 |
| AYW | LHPAAMPHL | 428 | 9 | 20 | 100 | 1241 |
| AYW | LHTLWKAGI | 148 | 9 | 20 | 100 | 1242 |
| AYR | LKLIMPARF | 107 | 9 | 15 | 75 | 1243 |
| AYW | LRGLPVCAF | 55 | 9 | 19 | 95 | 1244 |
| AYW | LRGTSFVYV | 761 | 9 | 16 | 80 | 1245 |
| AYW | LRRFIIFLF | 240 | 9 | 15 | 75 | 1246 |
| AYW | PHCLAFSYM | 531 | 9 | 19 | 95 | 1247 |
| AYW | PHHTALRQA | 50 | 9 | 20 | 100 | 1248 |
| AYW | PKVLHKRTL | 90 | 9 | 17 | 85 | 1249 |
| AYR | QHFRKLLLL | 6 | 9 | 15 | 75 | 1250 |
| AYW | QRIVGLLGF | 623 | 9 | 18 | 90 | 1251 |
| AYW | RKIPMGVGL | 502 | 9 | 16 | 80 | 1252 |
| AYW | RKLPVNRPI | 609 | 9 | 16 | 80 | 1253 |
| AYW | RKYTSFPWL | 744 | 9 | 17 | 85 | 1254 |
| AYW | RRAFPHCLA | 527 | 9 | 19 | 95 | 1255 |
| AYW | RRFIIFLFI | 241 | 9 | 15 | 75 | 1256 |
| AYR | RRLKLIMPA | 105 | 9 | 15 | 75 | 1257 |
| AYW | RRVAEDLNL | 35 | 9 | 18 | 90 | 1258 |
| AYW | SKLCLGWLW | 20 | 9 | 17 | 85 | 1259 |
| AYW | SRKYTSFPW | 743 | 9 | 17 | 85 | 1260 |
| AYW | TRHYLHTLW | 144 | 9 | 20 | 100 | 1261 |
| AYW | VHFASPLHV | 819 | 9 | 16 | 80 | 1262 |
| AYW | VRFSWLSLL | 331 | 9 | 16 | 80 | 1263 |
| AYW | VRRAFPHCL | 526 | 9 | 19 | 95 | 1264 |
| AYW | YRPPNAPIL | 132 | 9 | 20 | 100 | 1265 |
| AYW | YRWMCLRRF | 235 | 9 | 19 | 95 | 1266 |
| AYW | AHLSLRGLPV | 51 | 10 | 18 | 90 | 1267 |
| AYW | AKSVQHLESL | 546 | 10 | 17 | 85 | 1268 |
| AYW | ARDVLCLRPV | 12 | 10 | 15 | 75 | 1269 |
| AYW | ARVTGGVFLV | 356 | 10 | 18 | 90 | 1270 |
| AYW | EHCSPHHTAL | 46 | 10 | 20 | 100 | 1271 |
| AYW | FRKIPMGVGL | 501 | 10 | 16 | 80 | 1272 |
| AYW | FRKLPVNRPI | 608 | 10 | 16 | 80 | 1273 |
| AYR | GRETVLEYLV | 140 | 10 | 15 | 75 | 1274 |
| AYW | HHTALRQAIL | 51 | 10 | 19 | 95 | 1275 |
| AYW | HKVGNFTGLY | 54 | 10 | 19 | 95 | 1276 |
| AYW | KRWGYSLNFM | 576 | 10 | 17 | 85 | 1277 |
| AYW | LHLYSHPIIL | 490 | 10 | 16 | 80 | 1278 |
| AYW | LHPAAMPHLL | 428 | 10 | 20 | 100 | 1279 |
| AYW | LHTLWKAGIL | 148 | 10 | 20 | 100 | 1280 |
| AYR | LKLIMPARFY | 107 | 10 | 15 | 75 | 1281 |
| AYW | LRRFIIFLFI | 240 | 10 | 15 | 75 | 1282 |
| AYW | NKTKRWGYSL | 573 | 10 | 15 | 75 | 1283 |
| AYW | NRRVAEDLNL | 34 | 10 | 17 | 85 | 1284 |
| AYW | PHHTALRQAI | 50 | 10 | 20 | 100 | 1285 |
| AYW | PHLLVGSSGL | 434 | 10 | 16 | 80 | 1286 |
| AYW | QRIVGLLGFA | 623 | 10 | 18 | 90 | 1287 |
| AYW | RHYLHTLWKA | 145 | 10 | 20 | 100 | 1288 |
| AYW | RKYTSFPWLL | 744 | 10 | 17 | 85 | 1289 |
| AYW | RRAFPHCLAF | 527 | 10 | 19 | 95 | 1290 |
| AYW | RRFIIFLFIL | 241 | 10 | 15 | 75 | 1291 |
| AYW | SRKYTSFPWL | 743 | 10 | 17 | 85 | 1292 |
| AYW | SRLVVDFSQF | 375 | 10 | 19 | 95 | 1293 |
| AYW | THKVGNFTGL | 53 | 10 | 19 | 95 | 1294 |
| AYW | TKRWGYSLNF | 575 | 10 | 17 | 85 | 1295 |
| AYW | TKYLPLDKGI | 120 | 10 | 20 | 100 | 1296 |
| AYW | TRILTIPQSL | 186 | 10 | 16 | 80 | 1297 |
| AYW | VHFASPLHVA | 819 | 10 | 16 | 80 | 1298 |
| AYW | VRFSWLSLLV | 331 | 10 | 16 | 80 | 1299 |
| AYW | VRRAFPHCLA | 526 | 10 | 19 | 95 | 1300 |
| AYW | WKVCQRIVGL | 619 | 10 | 17 | 85 | 1301 |
| AYW | YRWMCLRRFI | 235 | 10 | 19 | 95 | 1302 |
| AYW | DHGAHLSLRGL | 48 | 11 | 19 | 95 | 1303 |
| AYW | IHLNPNKTKRW | 568 | 11 | 15 | 75 | 1304 |
| AYW | IHTAELLAACF | 714 | 11 | 17 | 85 | 1305 |
| AYW | LHPAAMPHLLV | 428 | 11 | 17 | 85 | 1306 |
| AYW | LHTLWKAGILY | 148 | 11 | 20 | 100 | 1307 |
| AYW | LRQAILCWGEL | 55 | 11 | 18 | 90 | 1308 |
| AYW | LRRFIIFLFIL | 240 | 11 | 15 | 75 | 1309 |
| AYW | PHHTALRQAIL | 50 | 11 | 19 | 95 | 1310 |
| AYW | PKFAVPNLQSL | 394 | 11 | 19 | 95 | 1311 |
| AYW | PKVLHKRTLGL | 90 | 11 | 17 | 85 | 1312 |
| AYW | PRTPARVTGGV | 352 | 11 | 18 | 90 | 1313 |
| AYW | QRIVGLLGFAA | 623 | 11 | 18 | 90 | 1314 |
| AYW | RKLPVNRPIDW | 609 | 11 | 16 | 80 | 1315 |
| AYW | RRFIIFLFILL | 241 | 11 | 15 | 75 | 1316 |
| AYR | RRLKLIMPARF | 105 | 11 | 15 | 75 | 1317 |
| AYW | SHPIILGFRKI | 494 | 11 | 16 | 80 | 1318 |
| AYW | SKLCLGWLWGM | 20 | 11 | 17 | 85 | 1319 |
| AYW | SRKYTSFPWLL | 743 | 11 | 17 | 85 | 1320 |
| AYW | THKVGNFTGLY | 53 | 11 | 19 | 95 | 1321 |
| AYW | TKRWGYSLNFM | 575 | 11 | 17 | 85 | 1322 |
| AYW | TRHYLHTLWKA | 144 | 11 | 20 | 100 | 1323 |
| AYW | VHFASPLHVAW | 819 | 11 | 16 | 80 | 1324 |
| AYW | VRRAFPHCLAF | 526 | 11 | 19 | 95 | 1325 |
| AYW | WKVCQRIVGLL | 619 | 11 | 17 | 85 | 1326 |
| AYW | YRWMCLRRFII | 235 | 11 | 19 | 95 | 1327 |

TABLE XIII
HBV B58 Super Motif

| | | | | | | |
|---|---|---|---|---|---|---|
| | | | No. of | Sequence | Conservancy | |
| Protein | Sequence | Position | Amino Acids | Frequency | (%) | SEQ ID NO: |

| | | | | | | |
|---|---|---|---|---|---|---|
| POL | AAMPHLLV | 431 | 8 | 17 | 85 | 1328 |
| NUC | ASALYREA | 34 | 8 | 17 | 85 | 1329 |
| POL | ASFCGSPY | 166 | 8 | 20 | 100 | 1330 |
| NUC | ASKLCLGW | 19 | 8 | 18 | 90 | 1331 |
| POL | ASPLHVAW | 822 | 8 | 16 | 80 | 1332 |
| ENV | ASVRFSWL | 329 | 8 | 16 | 80 | 1333 |
| POL | ATPTGWGL | 690 | 8 | 19 | 95 | 1334 |
| X | CALRFTSA | 69 | 8 | 18 | 90 | 1335 |
| NUC | CSPHHTAL | 48 | 8 | 20 | 100 | 1336 |
| POL | CSVVRRAF | 523 | 8 | 19 | 95 | 1337 |
| POL | ESRLVVDF | 374 | 8 | 19 | 95 | 1338 |
| NUC | ETVLEYLV | 142 | 8 | 15 | 75 | 1339 |
| POL | FARSRSGA | 724 | 8 | 17 | 85 | 1340 |
| POL | FASPLHVA | 821 | 8 | 16 | 80 | 1341 |
| POL | FSPTYKAF | 658 | 8 | 19 | 95 | 1342 |
| X | FSSAGPCA | 63 | 8 | 19 | 95 | 1343 |
| ENV | FSWLSLLV | 333 | 8 | 20 | 100 | 1344 |
| POL | FSYMDDVV | 536 | 8 | 18 | 90 | 1345 |
| POL | FTQCGYPA | 635 | 8 | 19 | 95 | 1346 |
| POL | FTSAICSV | 518 | 8 | 19 | 95 | 1347 |
| POL | GAKSVQHL | 545 | 8 | 17 | 85 | 1348 |
| POL | GTDNSVVL | 735 | 8 | 18 | 90 | 1349 |
| POL | HTAELLAA | 715 | 8 | 17 | 85 | 1350 |
| NUC | HTALRQAI | 52 | 8 | 20 | 100 | 1351 |
| POL | HTLWKAGI | 149 | 8 | 20 | 100 | 1352 |
| POL | LAQFTSAI | 515 | 8 | 19 | 95 | 1353 |
| NUC | LSFLPSDF | 45 | 8 | 19 | 95 | 1354 |
| POL | LSLDVSAA | 415 | 8 | 19 | 95 | 1355 |
| ENV | LSLLVPFV | 336 | 8 | 20 | 100 | 1356 |
| X | LSLRGLPV | 53 | 8 | 19 | 95 | 1357 |
| POL | LSRKYTSF | 742 | 8 | 17 | 85 | 1358 |
| POL | LSSNLSWL | 408 | 8 | 18 | 90 | 1359 |
| POL | LSWLSLDV | 412 | 8 | 20 | 100 | 1360 |
| NUC | LTFGRETV | 108 | 8 | 19 | 95 | 1361 |
| X | MSTTDLEA | 103 | 8 | 16 | 80 | 1362 |
| NUC | NAPILSTL | 136 | 8 | 20 | 100 | 1363 |
| POL | PAAMPHLL | 430 | 8 | 20 | 100 | 1364 |
| POL | PALMPLYA | 641 | 8 | 19 | 95 | 1365 |
| X | PARDVLCL | 11 | 8 | 16 | 80 | 1366 |
| POL | PARVTGGV | 355 | 8 | 18 | 90 | 1367 |
| NUC | PAYRPPNA | 130 | 8 | 19 | 95 | 1368 |
| POL | PSRGRLGL | 779 | 8 | 18 | 90 | 1369 |
| POL | PTGWGLAI | 692 | 8 | 15 | 75 | 1370 |
| POL | PTTGRTSL | 797 | 8 | 17 | 85 | 1371 |
| NUC | PTVQASKL | 15 | 8 | 16 | 80 | 1372 |
| ENV | PTVWLSVI | 351 | 8 | 15 | 75 | 1373 |
| POL | RAFPHCLA | 528 | 8 | 19 | 95 | 1374 |
| X | RTLGLSAM | 96 | 8 | 24 | 120 | 1375 |
| NUC | SALYREAL | 35 | 8 | 18 | 90 | 1376 |
| X | SSAGPCAL | 64 | 8 | 19 | 95 | 1377 |
| ENV | SSGTVNPV | 136 | 8 | 15 | 75 | 1378 |
| ENV | SSKPRQGM | 5 | 8 | 18 | 90 | 1379 |
| NUC | STLPETTV | 141 | 8 | 20 | 100 | 1380 |
| X | STTDLEAY | 104 | 8 | 15 | 75 | 1381 |
| NUC | TALRQAIL | 53 | 8 | 19 | 95 | 1382 |
| POL | TSAICSVV | 519 | 8 | 19 | 95 | 1383 |
| ENV | TSGFLGPL | 168 | 8 | 16 | 80 | 1384 |
| X | TTDLEAYF | 105 | 8 | 15 | 75 | 1385 |
| POL | TTGRTSLY | 798 | 8 | 17 | 85 | 1386 |
| POL | VSWPKFAV | 391 | 8 | 19 | 95 | 1387 |
| NUC | VSYVNVNM | 115 | 8 | 20 | 100 | 1388 |
| POL | VTGGVFLV | 358 | 8 | 20 | 100 | 1389 |
| ENV | WSPQAQGI | 66 | 8 | 17 | 85 | 1390 |
| POL | WTHKVGNF | 52 | 8 | 20 | 100 | 1391 |
| POL | YSLNFMGY | 580 | 8 | 17 | 85 | 1392 |
| POL | YTSFPWLL | 746 | 8 | 17 | 85 | 1393 |
| POL | AAPFTQCGY | 632 | 9 | 19 | 95 | 1394 |
| NUC | ASALYREAL | 34 | 9 | 17 | 85 | 1395 |
| NUC | ASKLCLGWL | 19 | 9 | 18 | 90 | 1396 |
| POL | ATPTGWGLA | 690 | 9 | 16 | 80 | 1397 |
| POL | CSRNLYVSL | 471 | 9 | 16 | 80 | 1398 |
| POL | DATPTGWGL | 689 | 9 | 19 | 95 | 1399 |
| ENV | DSWWTSLNF | 196 | 9 | 19 | 95 | 1400 |
| POL | EAGPLEEEL | 17 | 9 | 20 | 100 | 1401 |
| POL | FADATPTGW | 687 | 9 | 19 | 95 | 1402 |
| POL | FASPLHVAW | 821 | 9 | 16 | 80 | 1403 |
| POL | FAVPNLQSL | 396 | 9 | 19 | 95 | 1404 |
| POL | FSPTYKAFL | 658 | 9 | 19 | 95 | 1405 |
| X | FSSAGPCAL | 63 | 9 | 19 | 95 | 1406 |
| POL | FSYMDDVVL | 536 | 9 | 18 | 90 | 1407 |
| POL | FTFSPTYKA | 656 | 9 | 19 | 95 | 1408 |
| POL | FTGLYSSTV | 59 | 9 | 18 | 90 | 1409 |
| POL | FTQCGYPAL | 635 | 9 | 19 | 95 | 1410 |
| POL | FTSAICSVV | 518 | 9 | 19 | 95 | 1411 |
| X | GAHLSLRGL | 50 | 9 | 19 | 95 | 1412 |
| NUC | HTALRQAIL | 52 | 9 | 19 | 95 | 1413 |
| POL | HTLWKAGIL | 149 | 9 | 20 | 100 | 1414 |
| POL | KSVQHLESL | 547 | 9 | 17 | 85 | 1415 |
| POL | KTKRWGYSL | 574 | 9 | 19 | 95 | 1416 |
| POL | LAFSYMDDV | 534 | 9 | 18 | 90 | 1417 |
| NUC | LSFLPSDFF | 45 | 9 | 19 | 95 | 1418 |
| POL | LSLDVSAAF | 415 | 9 | 19 | 95 | 1419 |
| POL | LSPFLLAQF | 510 | 9 | 19 | 95 | 1420 |
| ENV | LSPTVWLSV | 349 | 9 | 15 | 75 | 1421 |
| NUC | LSTLPETTV | 140 | 9 | 20 | 100 | 1422 |
| ENV | LSVPNPLGF | 16 | 9 | 15 | 75 | 1423 |
| POL | LSYQHFRKL | 3 | 9 | 15 | 75 | 1424 |
| NUC | LTFGRETVL | 137 | 9 | 15 | 75 | 1425 |
| POL | LTNLLSSNL | 404 | 9 | 18 | 90 | 1426 |
| POL | LTVNEKRRL | 99 | 9 | 17 | 85 | 1427 |
| X | MSTTDLEAY | 103 | 9 | 15 | 75 | 1428 |
| POL | NSVVLSRKY | 738 | 9 | 18 | 90 | 1429 |
| POL | PAAMPHLLV | 430 | 9 | 17 | 85 | 1430 |
| POL | PARVTGGVF | 355 | 9 | 18 | 90 | 1431 |
| POL | PTTGRTSLY | 797 | 9 | 17 | 85 | 1432 |
| ENV | PTVWLSVIW | 351 | 9 | 15 | 75 | 1433 |
| POL | QAFTFSPTY | 654 | 9 | 19 | 95 | 1434 |
| NUC | QAILCWGEL | 57 | 9 | 18 | 90 | 1435 |
| NUC | QASKLCLGW | 18 | 9 | 16 | 80 | 1436 |
| POL | RAFPHCLAF | 528 | 9 | 19 | 95 | 1437 |
| ENV | RTGDPAPNM | 167 | 9 | 16 | 80 | 1438 |
| X | SAGPCALRF | 65 | 9 | 18 | 90 | 1439 |
| POL | SASFCGSPY | 165 | 9 | 20 | 100 | 1440 |
| POL | SSNLSWLSL | 409 | 9 | 18 | 90 | 1441 |
| ENV | SSSGTVNPV | 135 | 9 | 15 | 75 | 1442 |
| NUC | STLPETTVV | 141 | 9 | 20 | 100 | 1443 |
| X | STTDLEAYF | 104 | 9 | 15 | 75 | 1444 |
| POL | TAELLAACF | 716 | 9 | 17 | 85 | 1445 |
| NUC | TASALYREA | 33 | 9 | 16 | 80 | 1446 |
| POL | TSFVYVPSA | 764 | 9 | 16 | 80 | 1447 |
| ENV | TSGFLGPLL | 168 | 9 | 15 | 75 | 1448 |
| POL | TTGRTSLYA | 798 | 9 | 17 | 85 | 1449 |
| POL | VSIPWTHKV | 48 | 9 | 20 | 100 | 1450 |
| ENV | WSPQAQGIL | 66 | 9 | 17 | 85 | 1451 |
| ENV | WSSKPRQGM | 4 | 9 | 18 | 90 | 1452 |
| POL | YSHPIILGF | 493 | 9 | 16 | 80 | 1453 |
| POL | YSLNFMGYV | 580 | 9 | 15 | 75 | 1454 |
| POL | ASFCGSPYSW | 166 | 10 | 20 | 100 | 1455 |
| NUC | ASKLCLGWLW | 19 | 10 | 17 | 85 | 1456 |
| ENV | ASVRFSWLSL | 329 | 10 | 16 | 80 | 1457 |
| POL | ATPTGWGLAI | 690 | 10 | 15 | 75 | 1458 |
| X | CAFSSAGPCA | 61 | 10 | 19 | 95 | 1459 |
| ENV | CTCIPIPSSW | 310 | 10 | 20 | 100 | 1460 |
| ENV | CTIPAQGTSM | 298 | 10 | 16 | 80 | 1461 |
| POL | DATPTGWGLA | 689 | 10 | 16 | 80 | 1462 |
| ENV | DSWWTSLNFL | 196 | 10 | 18 | 90 | 1463 |
| NUC | DTASALYREA | 32 | 10 | 16 | 80 | 1464 |
| POL | FAAPFTQCGY | 631 | 10 | 19 | 95 | 1465 |
| ENV | FSWLSLLVPF | 333 | 10 | 20 | 100 | 1466 |
| POL | FTFSPTYKAF | 656 | 10 | 19 | 95 | 1467 |
| POL | FTQCGYPALM | 635 | 10 | 38 | 190 | 1468 |
| ENV | GSSSGTVNPV | 134 | 10 | 15 | 75 | 1469 |
| ENV | GTNLSVPNPL | 13 | 10 | 15 | 75 | 1470 |
| POL | GTSFVYVPSA | 763 | 10 | 16 | 80 | 1471 |
| POL | HTAELLAACF | 715 | 10 | 17 | 85 | 1472 |
| POL | HTLWKAGILY | 149 | 10 | 20 | 100 | 1473 |
| POL | LAFSYMDDVV | 534 | 10 | 18 | 90 | 1474 |
| POL | LSLDVSAAFY | 415 | 10 | 19 | 95 | 1475 |
| ENV | LSLLVPFVQW | 336 | 10 | 20 | 100 | 1476 |
| X | LSLRGLPVCA | 53 | 10 | 19 | 95 | 1477 |
| ENV | LSPTVWLSVI | 349 | 10 | 15 | 75 | 1478 |
| POL | LSRKYTSFPW | 742 | 10 | 17 | 85 | 1479 |
| POL | LSSNLSWLSL | 408 | 10 | 18 | 90 | 1480 |
| NUC | LSTLPETTVV | 140 | 10 | 20 | 100 | 1481 |
| POL | LSWLSLDVSA | 412 | 10 | 20 | 100 | 1482 |
| POL | LSYQHFRKLL | 3 | 10 | 15 | 75 | 1483 |
| ENV | LTIPQSLDSW | 189 | 10 | 18 | 90 | 1484 |
| X | MSTTDLEAYF | 103 | 10 | 15 | 75 | 1485 |
| POL | PADDPSRGRL | 775 | 10 | 18 | 90 | 1486 |
| ENV | PAGGSSSGTV | 131 | 10 | 18 | 90 | 1487 |
| POL | PALMPLYACI | 641 | 10 | 19 | 95 | 1488 |
| X | PAPCNFFTSA | 145 | 10 | 15 | 75 | 1489 |
| POL | PARVTGGVFL | 355 | 10 | 18 | 90 | 1490 |
| NUC | PAYRPPNAPI | 130 | 10 | 19 | 95 | 1491 |
| POL | PTTGRTSLYA | 797 | 10 | 17 | 85 | 1492 |
| NUC | PTVQASKLCL | 15 | 10 | 16 | 80 | 1493 |
| ENV | PTVWLSVIWM | 351 | 10 | 30 | 150 | 1494 |
| ENV | QAGFFLLTRI | 179 | 10 | 16 | 80 | 1495 |
| NUC | QAILCWGELM | 57 | 10 | 36 | 180 | 1496 |
| ENV | QAMQWNSTTF | 107 | 10 | 16 | 80 | 1497 |
| NUC | QASKLCLGWL | 18 | 10 | 16 | 80 | 1498 |
| ENV | QSLDSWWTSL | 193 | 10 | 18 | 90 | 1499 |
| POL | RTPARVTGGV | 353 | 10 | 18 | 90 | 1500 |
| POL | SAICSVVRRA | 520 | 10 | 19 | 95 | 1501 |
| X | SSAGPCALRF | 64 | 10 | 18 | 90 | 1502 |
| POL | TAELLAACFA | 716 | 10 | 17 | 85 | 1503 |
| NUC | TALRQAILCW | 53 | 10 | 19 | 95 | 1504 |
| NUC | TASALYREAL | 33 | 10 | 16 | 80 | 1505 |
| POL | TSFPWLLGCA | 747 | 10 | 15 | 75 | 1506 |
| POL | TSFVYVPSAL | 764 | 10 | 16 | 80 | 1507 |
| ENV | TSGFLGPLLV | 168 | 10 | 15 | 75 | 1508 |
| POL | VAEDLNLGNL | 37 | 10 | 19 | 95 | 1509 |
| POL | YSLNFMGYVI | 580 | 10 | 15 | 75 | 1510 |
| POL | AACFARSRSGA | 721 | 11 | 17 | 85 | 1511 |
| POL | AAPFTQCGYPA | 632 | 11 | 19 | 95 | 1512 |
| ENV | ASVRFSWLSLL | 329 | 11 | 16 | 80 | 1513 |
| X | CAFSSAGPCAL | 61 | 11 | 19 | 95 | 1514 |
| X | CALRFTSARRM | 69 | 11 | 26 | 130 | 1515 |
| NUC | CSPHHTALRQA | 48 | 11 | 20 | 100 | 1516 |
| ENV | CTCIPIPSSWA | 310 | 11 | 20 | 100 | 1517 |
| POL | DATPTGWGLAI | 689 | 11 | 15 | 75 | 1518 |
| NUC | DTASALYREAL | 32 | 11 | 16 | 80 | 1519 |
| POL | ESRLVVDFSQF | 374 | 11 | 19 | 95 | 1520 |
| POL | FADATPTGWGL | 687 | 11 | 19 | 95 | 1521 |
| X | FSSAGPCALRF | 63 | 11 | 18 | 90 | 1522 |
| ENV | FSWLSLLVPFV | 333 | 11 | 20 | 100 | 1523 |
| POL | FSYMDDVVLGA | 536 | 11 | 18 | 90 | 1524 |
| POL | FTFSPTYKAFL | 656 | 11 | 19 | 95 | 1525 |
| X | GAHLSLRGLPV | 50 | 11 | 18 | 90 | 1526 |
| POL | GAKSVQHLESL | 545 | 11 | 17 | 85 | 1527 |
| POL | GTSFVYVPSAL | 763 | 11 | 16 | 80 | 1528 |
| POL | HTAELLAACFA | 715 | 11 | 17 | 85 | 1529 |
| NUC | HTALRQAILCW | 52 | 11 | 19 | 95 | 1530 |
| NUC | ISCLTFGRETV | 105 | 11 | 18 | 90 | 1531 |
| POL | KTKRWGYSLNF | 574 | 11 | 17 | 85 | 1532 |
| POL | LAFSYMDDVVL | 534 | 11 | 18 | 90 | 1533 |
| POL | LAQFTSAICSV | 515 | 11 | 19 | 95 | 1534 |
| ENV | LSLLVPFVQWF | 336 | 11 | 20 | 100 | 1535 |
| X | LSLRGLPVCAF | 53 | 11 | 19 | 95 | 1536 |
| ENV | LSPTVWLSVIW | 349 | 11 | 15 | 75 | 1537 |
| POL | LSRKYTSFPWL | 742 | 11 | 17 | 85 | 1538 |
| POL | LSWLSLDVSAA | 412 | 11 | 19 | 95 | 1539 |
| POL | LSYQHFRKLLL | 3 | 11 | 15 | 75 | 1540 |
| NUC | LTFGRETVLEY | 137 | 11 | 15 | 75 | 1541 |
| ENV | LTIPQSLDSWW | 189 | 11 | 18 | 90 | 1542 |
| POL | LTNLLSSNLSW | 404 | 11 | 18 | 90 | 1543 |
| ENV | LTRILTIPQSL | 185 | 11 | 16 | 80 | 1544 |
| X | PARDVLCLRPV | 11 | 11 | 15 | 75 | 1545 |
| POL | PARVTGGVFLV | 355 | 11 | 18 | 90 | 1546 |
| NUC | PAYRPPNAPIL | 130 | 11 | 19 | 95 | 1547 |
| ENV | PTVWLSVIWMM | 351 | 11 | 28 | 140 | 1548 |
| POL | QAFTFSPTYKA | 654 | 11 | 19 | 95 | 1549 |
| ENV | QAGFFLLTRIL | 179 | 11 | 16 | 80 | 1550 |
| NUC | QASKLCLGWLW | 18 | 11 | 15 | 75 | 1551 |
| POL | QSLTNLLSSNL | 402 | 11 | 18 | 90 | 1552 |
| POL | RAFPHCLAFSY | 528 | 11 | 19 | 95 | 1553 |
| POL | RTPARVTGGVF | 353 | 11 | 18 | 90 | 1554 |
| NUC | RTPPAYRPPNA | 127 | 11 | 19 | 95 | 1555 |
| POL | SAICSVVRRAF | 520 | 11 | 19 | 95 | 1556 |
| POL | SASFCGSPYSW | 165 | 11 | 20 | 100 | 1557 |
| POL | SSNLSWLSLDV | 409 | 11 | 18 | 90 | 1558 |
| POL | TSAICSVVRRA | 519 | 11 | 19 | 95 | 1559 |
| POL | TSFPWLLGCAA | 747 | 11 | 15 | 75 | 1560 |
| ENV | TSGFLGPLLVL | 168 | 11 | 15 | 75 | 1561 |
| POL | VSWPKFAVPNL | 391 | 11 | 19 | 95 | 1562 |
| POL | WTHKVGNFTGL | 52 | 11 | 19 | 95 | 1563 |
| POL | YTSFPWLLGCA | 746 | 11 | 15 | 75 | 1564 |

TABLE XIV
HBV B62 Super Motif

| | | | | | | |
|---|---|---|---|---|---|---|
| | | | No. of | Sequence | Conservancy | |
| Protein | Sequence | Position | Amino Acids | Frequency | (%) | SEQ ID NO: |

| | | | | | | |
|---|---|---|---|---|---|---|
| NUC | AILCWGEL | 58 | 8 | 18 | 90 | 1565 |
| POL | APFTQCGY | 633 | 8 | 19 | 95 | 1566 |
| POL | AVPNLQSL | 397 | 8 | 19 | 95 | 1567 |
| ENV | CIPIPSSW | 312 | 8 | 20 | 100 | 1568 |
| NUC | CLGWLWGM | 23 | 8 | 17 | 85 | 1569 |
| ENV | CLIFLLVL | 253 | 8 | 20 | 100 | 1570 |
| ENV | CLRRFIIF | 239 | 8 | 19 | 95 | 1571 |
| POL | CQRIVGLL | 622 | 8 | 17 | 85 | 1572 |
| NUC | DIDPYKEF | 31 | 8 | 18 | 90 | 1573 |
| NUC | DLLDTASA | 29 | 8 | 17 | 85 | 1574 |
| ENV | DPRVRGLY | 122 | 8 | 16 | 80 | 1575 |
| NUC | DPYKEFGA | 33 | 8 | 18 | 90 | 1576 |
| X | DVLCLRPV | 14 | 8 | 19 | 95 | 1577 |
| X | ELGEEIRL | 122 | 8 | 16 | 80 | 1578 |
| POL | ELLAACFA | 718 | 8 | 18 | 90 | 1579 |
| ENV | FIIFLFIL | 243 | 8 | 16 | 80 | 1580 |
| ENV | FILLLCLI | 248 | 8 | 16 | 80 | 1581 |
| ENV | FLGPLLVL | 171 | 8 | 15 | 75 | 1582 |
| ENV | FLLVLLDY | 256 | 8 | 19 | 95 | 1583 |
| POL | FPWLLGCA | 749 | 8 | 15 | 75 | 1584 |
| ENV | FVGLSPTV | 346 | 8 | 19 | 95 | 1585 |
| ENV | FVQWFVGL | 342 | 8 | 19 | 95 | 1586 |
| POL | FVYVPSAL | 766 | 8 | 18 | 90 | 1587 |
| POL | GLSPFLLA | 509 | 8 | 19 | 95 | 1588 |
| ENV | GLSPTVWL | 348 | 8 | 20 | 100 | 1589 |
| ENV | GMLPVCPL | 265 | 8 | 18 | 90 | 1590 |
| ENV | GPLLVLQA | 173 | 8 | 19 | 95 | 1591 |
| POL | GVGLSPFL | 507 | 8 | 16 | 80 | 1592 |
| POL | HLYSHPII | 491 | 8 | 16 | 80 | 1593 |
| POL | HPAAMPHL | 429 | 8 | 20 | 100 | 1594 |
| ENV | IIFLFILL | 244 | 8 | 16 | 80 | 1595 |
| POL | IILGFRKI | 497 | 8 | 16 | 80 | 1596 |
| NUC | ILCWGELM | 59 | 8 | 18 | 90 | 1597 |
| ENV | ILLLCLIF | 249 | 8 | 20 | 100 | 1598 |
| POL | ILRGTSFV | 760 | 8 | 16 | 80 | 1599 |
| ENV | ILTIPQSL | 188 | 8 | 19 | 95 | 1600 |
| ENV | IPIPSSWA | 313 | 8 | 20 | 100 | 1601 |
| ENV | IPQSLDSW | 191 | 8 | 18 | 90 | 1602 |
| ENV | IPSSWAFA | 315 | 8 | 16 | 80 | 1603 |
| POL | IVGLLGFA | 625 | 8 | 18 | 90 | 1604 |
| POL | KIPMGVGL | 503 | 8 | 16 | 80 | 1605 |
| NUC | KLCLGWLW | 21 | 8 | 17 | 85 | 1606 |
| POL | KLIMPARF | 108 | 8 | 15 | 75 | 1607 |
| POL | KLPVNRPI | 610 | 8 | 16 | 80 | 1608 |
| POL | KVGNFTGL | 55 | 8 | 19 | 95 | 1609 |
| X | KVLHKRTL | 91 | 8 | 17 | 85 | 1610 |
| ENV | LIFLLVLL | 254 | 8 | 20 | 100 | 1611 |
| POL | LIMPARFY | 109 | 8 | 20 | 100 | 1612 |
| POL | LLAQFTSA | 514 | 8 | 19 | 95 | 1613 |
| ENV | LLCLIFLL | 251 | 8 | 20 | 100 | 1614 |
| NUC | LLDTASAL | 30 | 8 | 17 | 85 | 1615 |
| ENV | LLDYQGML | 260 | 8 | 19 | 95 | 1616 |
| POL | LLGCAANW | 752 | 8 | 16 | 80 | 1617 |
| POL | LLGFAAPF | 628 | 8 | 19 | 95 | 1618 |
| ENV | LLGWSPQA | 63 | 8 | 17 | 85 | 1619 |
| ENV | LLLCLIFL | 250 | 8 | 20 | 100 | 1620 |
| ENV | LLPIFFCL | 378 | 8 | 20 | 100 | 1621 |
| POL | LLSLGIHL | 563 | 8 | 19 | 95 | 1622 |
| POL | LLSSNLSW | 407 | 8 | 18 | 90 | 1623 |
| ENV | LLTRILTI | 184 | 8 | 16 | 80 | 1624 |
| POL | LLVGSSGL | 436 | 8 | 16 | 80 | 1625 |
| ENV | LLVLQAGF | 175 | 8 | 19 | 95 | 1626 |
| ENV | LLVPFVQW | 338 | 8 | 20 | 100 | 1627 |
| POL | LMPLYACI | 643 | 8 | 19 | 95 | 1628 |
| ENV | LPIFFCLW | 379 | 8 | 20 | 100 | 1629 |
| POL | LPIHTAEL | 712 | 8 | 17 | 85 | 1630 |
| ENV | LQAGFFLL | 178 | 8 | 19 | 95 | 1631 |
| POL | LQSLTNLL | 401 | 8 | 20 | 100 | 1632 |
| ENV | LVLQAGFF | 176 | 8 | 19 | 95 | 1633 |
| ENV | LVPFVQWF | 339 | 8 | 20 | 100 | 1634 |
| NUC | LVSFGVWI | 119 | 8 | 18 | 90 | 1635 |
| POL | LVVDFSQF | 377 | 8 | 20 | 100 | 1636 |
| POL | MPLSYQHF | 1 | 8 | 20 | 100 | 1637 |
| NUC | MQLFHLCL | 1 | 8 | 15 | 75 | 1638 |
| ENV | MQWNSTTF | 109 | 8 | 16 | 80 | 1639 |
| POL | NLNVSIPW | 45 | 8 | 19 | 95 | 1640 |
| POL | NLQSLTNL | 400 | 8 | 20 | 100 | 1641 |
| ENV | NLSVPNPL | 15 | 8 | 15 | 75 | 1642 |
| POL | NPNKTKRW | 571 | 8 | 15 | 75 | 1643 |
| ENV | PIFFCLWV | 380 | 8 | 20 | 100 | 1644 |
| POL | PIHTAELL | 713 | 8 | 17 | 85 | 1645 |
| ENV | PIPSSWAF | 314 | 8 | 20 | 100 | 1646 |
| ENV | PQSLDSWW | 192 | 8 | 18 | 90 | 1647 |
| X | PVCAFSSA | 59 | 8 | 19 | 95 | 1648 |
| POL | PVNRPIDW | 612 | 8 | 17 | 85 | 1649 |
| X | QLDPARDV | 8 | 8 | 16 | 80 | 1650 |
| POL | RIVGLLGF | 624 | 8 | 18 | 90 | 1651 |
| POL | RLKLIMPA | 106 | 8 | 15 | 75 | 1652 |
| NUC | RPPNAPIL | 133 | 8 | 20 | 100 | 1653 |
| NUC | RQLLWFHI | 98 | 8 | 18 | 90 | 1654 |
| POL | RVAEDLNL | 36 | 8 | 19 | 95 | 1655 |
| POL | RVHFASPL | 818 | 8 | 16 | 80 | 1656 |
| POL | RVTGGVFL | 357 | 8 | 20 | 100 | 1657 |
| POL | SIPWTHKV | 49 | 8 | 20 | 100 | 1658 |
| POL | SLDVSAAF | 416 | 8 | 19 | 95 | 1659 |
| POL | SLNFMGYV | 581 | 8 | 15 | 75 | 1660 |
| POL | SPFLLAQF | 511 | 8 | 19 | 95 | 1661 |
| ENV | SPQAQGIL | 67 | 8 | 17 | 85 | 1662 |
| POL | SPSVPSHL | 808 | 8 | 17 | 85 | 1663 |
| ENV | SPTVWLSV | 350 | 8 | 15 | 75 | 1664 |
| POL | SPTYKAFL | 659 | 8 | 19 | 95 | 1665 |
| ENV | SVPNPLGF | 17 | 8 | 15 | 75 | 1666 |
| POL | SVQHLESL | 548 | 8 | 17 | 85 | 1667 |
| POL | SVVLSRKY | 739 | 8 | 18 | 90 | 1668 |
| NUC | TLPETTVV | 142 | 8 | 20 | 100 | 1669 |
| POL | TLWKAGIL | 150 | 8 | 20 | 100 | 1670 |
| ENV | TPPHGGLL | 57 | 8 | 15 | 75 | 1671 |
| POL | TPTGWGLA | 691 | 8 | 16 | 80 | 1672 |
| POL | TQCGYPAL | 636 | 8 | 19 | 95 | 1673 |
| POL | TVNEKRRL | 100 | 8 | 17 | 85 | 1674 |
| ENV | TVWLSVIW | 352 | 8 | 15 | 75 | 1675 |
| ENV | VLLDYQGM | 259 | 8 | 19 | 95 | 1676 |
| ENV | VLQAGFFL | 177 | 8 | 19 | 95 | 1677 |
| ENV | VPFVQWFV | 340 | 8 | 19 | 95 | 1678 |
| POL | VPSALNPA | 769 | 8 | 18 | 90 | 1679 |
| NUC | VQASKLCL | 17 | 8 | 16 | 80 | 1680 |
| POL | VVLGAKSV | 542 | 8 | 18 | 90 | 1681 |
| POL | WILRGTSF | 759 | 8 | 16 | 80 | 1682 |
| NUC | WIRTPPAY | 125 | 8 | 19 | 95 | 1683 |
| POL | WLSLDVSA | 414 | 8 | 20 | 100 | 1684 |
| ENV | WLSLLVPF | 335 | 8 | 20 | 100 | 1685 |
| ENV | WMCLRRFI | 237 | 8 | 19 | 95 | 1686 |
| POL | YLHTLWKA | 147 | 8 | 20 | 100 | 1687 |
| POL | YLPLDKGI | 122 | 8 | 20 | 100 | 1688 |
| NUC | YLVSFGVW | 118 | 8 | 18 | 90 | 1689 |
| POL | YPALMPLY | 640 | 8 | 19 | 95 | 1690 |
| POL | YQHFRKLL | 5 | 8 | 15 | 75 | 1691 |
| POL | AICSVVRRA | 521 | 9 | 19 | 95 | 1692 |
| NUC | AILCWGELM | 58 | 9 | 18 | 90 | 1693 |
| POL | ALMPLYACI | 642 | 9 | 19 | 95 | 1694 |
| NUC | ALRQAILCW | 54 | 9 | 19 | 95 | 1695 |
| ENV | AMQWNSTTF | 108 | 9 | 16 | 80 | 1696 |
| X | AMSTTDLEA | 102 | 9 | 15 | 75 | 1697 |
| X | APCNFFTSA | 146 | 9 | 15 | 75 | 1698 |
| ENV | CIPIPSSWA | 312 | 9 | 20 | 100 | 1699 |
| ENV | CLIFLLVLL | 253 | 9 | 20 | 100 | 1700 |
| ENV | CLRRFIIFL | 239 | 9 | 19 | 95 | 1701 |
| NUC | CLTFGRETV | 107 | 9 | 18 | 90 | 1702 |
| ENV | CPGYRWMCL | 232 | 9 | 20 | 100 | 1703 |
| NUC | CPTVQASKL | 14 | 9 | 16 | 80 | 1704 |
| X | CQLDPARDV | 7 | 9 | 16 | 80 | 1705 |
| NUC | DLLDTASAL | 29 | 9 | 17 | 85 | 1706 |
| POL | DLNLGNLNV | 40 | 9 | 19 | 95 | 1707 |
| X | DPARDVLCL | 10 | 9 | 16 | 80 | 1708 |
| POL | DPSRGRLGL | 778 | 9 | 18 | 90 | 1709 |
| POL | DVVLGAKSV | 541 | 9 | 18 | 90 | 1710 |
| ENV | FIIFLFILL | 243 | 9 | 16 | 80 | 1711 |
| ENV | FILLLCLIF | 248 | 9 | 16 | 80 | 1712 |
| ENV | FLFILLLCL | 246 | 9 | 16 | 80 | 1713 |
| POL | FLLAQFTSA | 513 | 9 | 19 | 95 | 1714 |
| POL | FLLSLGIHL | 562 | 9 | 19 | 95 | 1715 |
| ENV | FLLTRILTI | 183 | 9 | 16 | 80 | 1716 |
| ENV | FPDHQLDPA | 14 | 9 | 18 | 90 | 1717 |
| POL | FPHCLAFSY | 530 | 9 | 19 | 95 | 1718 |
| POL | FPWLLGCAA | 749 | 9 | 15 | 75 | 1719 |
| ENV | FVGLSPTVW | 346 | 9 | 19 | 95 | 1720 |
| POL | GLCQVFADA | 682 | 9 | 17 | 85 | 1721 |
| POL | GLLGFAAPF | 627 | 9 | 19 | 95 | 1722 |
| ENV | GLLGWSPQA | 62 | 9 | 17 | 85 | 1723 |
| POL | GVGLSPFLL | 507 | 9 | 16 | 80 | 1724 |
| NUC | GVWIRTPPA | 123 | 9 | 19 | 95 | 1725 |
| POL | HLLVGSSGL | 435 | 9 | 16 | 80 | 1726 |
| X | HLSLRGLPV | 52 | 9 | 18 | 90 | 1727 |
| POL | HLYSHPIIL | 491 | 9 | 16 | 80 | 1728 |
| POL | HPAAMPHLL | 429 | 9 | 20 | 100 | 1729 |
| ENV | IIFLFILLL | 244 | 9 | 16 | 80 | 1730 |
| POL | ILGFRKIPM | 498 | 9 | 16 | 80 | 1731 |
| ENV | ILLLCLIFL | 249 | 9 | 20 | 100 | 1732 |
| POL | ILRGTSFVY | 760 | 9 | 16 | 80 | 1733 |
| ENV | IPIPSSWAF | 313 | 9 | 20 | 100 | 1734 |
| ENV | IPQSLDSWW | 191 | 9 | 18 | 90 | 1735 |
| POL | IVGLLGFAA | 625 | 9 | 18 | 90 | 1736 |
| POL | KLHLYSHPI | 489 | 9 | 19 | 95 | 1737 |
| POL | KLIMPARFY | 108 | 9 | 15 | 75 | 1738 |
| POL | KVCQRIVGL | 620 | 9 | 17 | 85 | 1739 |
| POL | KVGNFTGLY | 55 | 9 | 19 | 95 | 1740 |
| POL | LLAQFTSAI | 514 | 9 | 19 | 95 | 1741 |
| ENV | LLCLIFLLV | 251 | 9 | 20 | 100 | 1742 |
| NUC | LLDTASALY | 30 | 9 | 17 | 85 | 1743 |
| POL | LLGCAANWI | 752 | 9 | 16 | 80 | 1744 |
| ENV | LLLCLIFLL | 250 | 9 | 20 | 100 | 1745 |
| ENV | LLPIFFCLW | 378 | 9 | 20 | 100 | 1746 |
| NUC | LLSFLPSDF | 44 | 9 | 19 | 95 | 1747 |
| POL | LLSSNLSWL | 407 | 9 | 18 | 90 | 1748 |
| ENV | LLVLQAGFF | 175 | 9 | 19 | 95 | 1749 |
| ENV | LLVPFVQWF | 338 | 9 | 20 | 100 | 1750 |
| NUC | LLWFHISCL | 100 | 9 | 18 | 90 | 1751 |
| ENV | LPIFFCLWV | 379 | 9 | 20 | 100 | 1752 |
| POL | LPIHTAELL | 712 | 9 | 17 | 85 | 1753 |
| X | LPVCAFSSA | 58 | 9 | 19 | 95 | 1754 |
| POL | LPVNRPIDW | 611 | 9 | 16 | 80 | 1755 |
| ENV | LVLLDYQGM | 258 | 9 | 19 | 95 | 1756 |
| ENV | LVLQAGFFL | 176 | 9 | 18 | 90 | 1757 |
| ENV | LVPFVQWFV | 339 | 9 | 19 | 95 | 1758 |
| ENV | MMWYWGPSL | 360 | 9 | 17 | 85 | 1759 |
| POL | NLGNLNVSI | 42 | 9 | 19 | 95 | 1760 |
| POL | NLLSSNLSW | 406 | 9 | 18 | 90 | 1761 |
| POL | NLQSLTNLL | 400 | 9 | 20 | 100 | 1762 |
| POL | NLSWLSLDV | 411 | 9 | 18 | 90 | 1763 |
| ENV | PIFFCLWVY | 380 | 9 | 20 | 100 | 1764 |
| POL | PIHTAELLA | 713 | 9 | 17 | 85 | 1765 |
| POL | PIILGFRKI | 496 | 9 | 16 | 80 | 1766 |
| ENV | PIPSSWAFA | 314 | 9 | 16 | 80 | 1767 |
| POL | PLDKGIKPY | 124 | 9 | 20 | 100 | 1768 |
| POL | PLEEELPRL | 20 | 9 | 19 | 95 | 1769 |
| ENV | PLLPIFFCL | 377 | 9 | 20 | 100 | 1770 |
| ENV | PLLVLQAGF | 174 | 9 | 19 | 95 | 1771 |
| POL | PLPIHTAEL | 711 | 9 | 16 | 80 | 1772 |
| POL | PMGVGLSPF | 505 | 9 | 16 | 80 | 1773 |
| NUC | PPAYRPPNA | 129 | 9 | 19 | 95 | 1774 |
| ENV | PPHGGLLGW | 58 | 9 | 17 | 85 | 1775 |
| X | QLDPARDVL | 8 | 9 | 16 | 80 | 1776 |
| ENV | RILTIPQSL | 187 | 9 | 16 | 80 | 1777 |
| POL | RIVGLLGFA | 624 | 9 | 18 | 90 | 1778 |
| POL | RLVVDFSQF | 376 | 9 | 19 | 95 | 1779 |
| POL | RVTGGVFLV | 357 | 9 | 20 | 100 | 1780 |
| ENV | SLDSWWTSL | 194 | 9 | 19 | 95 | 1781 |
| POL | SLDVSAAFY | 416 | 9 | 19 | 95 | 1782 |
| ENV | SLLVPFVQW | 337 | 9 | 20 | 100 | 1783 |
| POL | SLNFMGYVI | 581 | 9 | 15 | 75 | 1784 |
| X | SLRGLPVCA | 54 | 9 | 19 | 95 | 1785 |
| ENV | SPTVWLSVI | 350 | 9 | 15 | 75 | 1786 |
| ENV | SVRFSWLSL | 330 | 9 | 16 | 80 | 1787 |
| ENV | TIPQSLDSW | 190 | 9 | 18 | 90 | 1788 |
| POL | TLWKAGILY | 150 | 9 | 20 | 100 | 1789 |
| POL | TPARVTGGV | 354 | 9 | 18 | 90 | 1790 |
| POL | TPTGWGLAI | 691 | 9 | 15 | 75 | 1791 |
| POL | TQCGYPALM | 636 | 9 | 19 | 95 | 1792 |
| NUC | TVQASKLCL | 16 | 9 | 16 | 80 | 1793 |
| ENV | TVWLSVIWM | 352 | 9 | 15 | 75 | 1794 |
| X | VLCLRPVGA | 15 | 9 | 19 | 95 | 1795 |
| X | VLGGCRHKL | 133 | 9 | 18 | 90 | 1796 |
| X | VLHKRTLGL | 92 | 9 | 17 | 85 | 1797 |
| ENV | VLLDYQGML | 259 | 9 | 19 | 95 | 1798 |
| ENV | VLQAGFFLL | 177 | 9 | 19 | 95 | 1799 |
| POL | VLSRKYTSF | 741 | 9 | 17 | 85 | 1800 |
| POL | WILRGTSFV | 759 | 9 | 16 | 80 | 1801 |
| POL | WLLGCAANW | 751 | 9 | 16 | 80 | 1802 |
| POL | WLSLDVSAA | 414 | 9 | 19 | 95 | 1803 |
| ENV | WLSLLVPFV | 335 | 9 | 20 | 100 | 1804 |
| ENV | WMCLRRFII | 237 | 9 | 19 | 95 | 1805 |
| POL | WPKFAVPNL | 393 | 9 | 19 | 95 | 1806 |
| NUC | YLVSFGVWI | 118 | 9 | 18 | 90 | 1807 |
| POL | YMDDVVLGA | 538 | 9 | 18 | 90 | 1808 |
| POL | YPALMPLYA | 640 | 9 | 19 | 95 | 1809 |
| POL | YQHFRKLLL | 5 | 9 | 15 | 75 | 1810 |
| POL | YVPSALNPA | 768 | 9 | 18 | 90 | 1811 |
| POL | AICSVVRRAF | 521 | 10 | 19 | 95 | 1812 |
| POL | APFTQCGYPA | 633 | 10 | 19 | 95 | 1813 |
| POL | AQFTSAICSV | 516 | 10 | 19 | 95 | 1814 |
| ENV | CIPIPSSWAF | 312 | 10 | 20 | 100 | 1815 |
| POL | CLAFSYMDDV | 533 | 10 | 18 | 90 | 1816 |
| NUC | CLGWLWGMDI | 23 | 10 | 17 | 85 | 1817 |
| ENV | CLRRFIIFLF | 239 | 10 | 15 | 75 | 1818 |
| X | CQLDPARDVL | 7 | 10 | 16 | 80 | 1819 |
| POL | CQRIVGLLGF | 622 | 10 | 17 | 85 | 1820 |
| NUC | DIDPYKEFGA | 31 | 10 | 18 | 90 | 1821 |
| NUC | DLLDTASALY | 29 | 10 | 17 | 85 | 1822 |
| X | DVLCLRPVGA | 14 | 10 | 19 | 95 | 1823 |
| NUC | ELLSFLPSDF | 43 | 10 | 19 | 95 | 1824 |
| ENV | FIIFLFILLL | 243 | 10 | 16 | 80 | 1825 |
| ENV | FILLLCLIFL | 248 | 10 | 16 | 80 | 1826 |
| ENV | FLFILLLCLI | 246 | 10 | 16 | 80 | 1827 |
| ENV | FLGPLLVLQA | 171 | 10 | 15 | 75 | 1828 |
| POL | FLLAQFTSAI | 513 | 10 | 19 | 95 | 1829 |
| ENV | FPDHQLDPAF | 14 | 10 | 17 | 85 | 1830 |
| POL | FPHCLAFSYM | 530 | 10 | 19 | 95 | 1831 |
| ENV | FVGLSPTVWL | 346 | 10 | 19 | 95 | 1832 |
| X | FVLGGCRHKL | 132 | 10 | 18 | 90 | 1833 |
| X | GLPVCAFSSA | 57 | 10 | 19 | 95 | 1834 |
| POL | GLSPFLLAQF | 509 | 10 | 19 | 95 | 1835 |
| ENV | GLSPTVWLSV | 348 | 10 | 15 | 75 | 1836 |
| NUC | GMDIDPYKEF | 29 | 10 | 17 | 85 | 1837 |
| X | GPCALRFTSA | 67 | 10 | 18 | 90 | 1838 |
| POL | GPLEEELPRL | 19 | 10 | 19 | 95 | 1839 |
| ENV | GPLLVLQAGF | 173 | 10 | 19 | 95 | 1840 |
| POL | GVGLSPFLLA | 507 | 10 | 16 | 80 | 1841 |
| NUC | GVWIRTPPAY | 123 | 10 | 19 | 95 | 1842 |
| POL | HLNPNKTKRW | 569 | 10 | 15 | 75 | 1843 |
| POL | HPAAMPHLLV | 429 | 10 | 17 | 85 | 1844 |
| POL | HPIILGFRKI | 495 | 10 | 16 | 80 | 1845 |
| POL | IILGFRKIPM | 497 | 10 | 16 | 80 | 1846 |
| ENV | ILLLCLIFLL | 249 | 10 | 20 | 100 | 1847 |
| POL | ILRGTSFVYV | 760 | 10 | 16 | 80 | 1848 |
| NUC | ILSTLPETTV | 139 | 10 | 20 | 100 | 1849 |
| ENV | IPIPSSWAFA | 313 | 10 | 16 | 80 | 1850 |
| POL | IPMGVGLSPF | 504 | 10 | 16 | 80 | 1851 |
| POL | IPWTHKVGNF | 50 | 10 | 20 | 100 | 1852 |
| NUC | KLCLGWLWGM | 21 | 10 | 17 | 85 | 1853 |
| POL | KLHLYSHPII | 489 | 10 | 16 | 80 | 1854 |
| POL | KLPVNRPIDW | 610 | 10 | 16 | 80 | 1855 |
| POL | KQAFTFSPTY | 653 | 10 | 19 | 95 | 1856 |
| POL | KVCQRIVGLL | 620 | 10 | 17 | 85 | 1857 |
| X | KVLHKRTLGL | 91 | 10 | 17 | 85 | 1858 |
| ENV | LIFLLVLLDY | 254 | 10 | 19 | 95 | 1859 |
| ENV | LLCLIFLLVL | 251 | 10 | 20 | 100 | 1860 |
| ENV | LLDYQGMLPV | 260 | 10 | 18 | 90 | 1861 |
| POL | LLGCAANWIL | 752 | 10 | 16 | 80 | 1862 |
| ENV | LLCLIFLLV | 250 | 10 | 20 | 100 | 1863 |
| ENV | LLPIFFCLWV | 378 | 10 | 20 | 100 | 1864 |
| NUC | LLSFLPSDFF | 44 | 10 | 19 | 95 | 1865 |
| ENV | LLVLLDYQGM | 257 | 10 | 19 | 95 | 1866 |
| ENV | LLVQAGFFL | 175 | 10 | 18 | 90 | 1867 |
| ENV | LLVPFVQWFV | 338 | 10 | 19 | 95 | 1868 |
| ENV | LPIFFCLWVY | 379 | 10 | 20 | 100 | 1869 |
| POL | LPIHTAELLA | 712 | 10 | 17 | 85 | 1870 |
| X | LPKVLHKRTL | 89 | 10 | 16 | 80 | 1871 |
| POL | LPLDKGIKPY | 123 | 10 | 20 | 100 | 1872 |
| ENV | LVLLDYQGML | 258 | 10 | 19 | 95 | 1873 |
| ENV | LVLQAGFFLL | 176 | 10 | 18 | 90 | 1874 |
| ENV | MMWYWGPSLY | 360 | 10 | 17 | 85 | 1875 |
| POL | NLLSSNLSWL | 406 | 10 | 18 | 90 | 1876 |
| ENV | NLSVPNPLGF | 15 | 10 | 15 | 75 | 1877 |
| POL | NPNKTKRWGY | 571 | 10 | 15 | 75 | 1878 |
| POL | NVSIPWTHKV | 47 | 10 | 20 | 100 | 1879 |
| POL | PIDWKVCQRI | 616 | 10 | 17 | 85 | 1880 |
| ENV | PIFFCLWVYI | 380 | 10 | 20 | 100 | 1881 |
| POL | PIHTAELLAA | 713 | 10 | 17 | 85 | 1882 |
| POL | PLDKGIKPYY | 124 | 10 | 20 | 100 | 1883 |
| POL | PLEEELPRLA | 20 | 10 | 18 | 90 | 1884 |
| ENV | PLGFFDHQL | 10 | 10 | 19 | 95 | 1885 |
| POL | PLHPAAMPHL | 427 | 10 | 20 | 100 | 1886 |
| ENV | PLLPIFFCLW | 377 | 10 | 20 | 100 | 1887 |
| ENV | PLLVLQAGFF | 174 | 10 | 19 | 95 | 1888 |
| POL | PLPIHTAELL | 711 | 10 | 16 | 80 | 1889 |
| POL | PLSYQHFRKL | 2 | 10 | 15 | 75 | 1890 |
| POL | PLTVNEKRRL | 98 | 10 | 17 | 85 | 1891 |
| POL | PMGVGLSPFL | 505 | 10 | 16 | 80 | 1892 |
| NUC | PPNAPILSTL | 134 | 10 | 20 | 100 | 1893 |
| POL | PVNRPIDWKV | 612 | 10 | 17 | 85 | 1894 |
| NUC | QLLWFHISCL | 99 | 10 | 18 | 90 | 1895 |
| POL | RIVGLLGFAA | 624 | 10 | 18 | 90 | 1896 |
| POL | RLKLIMPARF | 106 | 10 | 15 | 75 | 1897 |
| NUC | RQAILCWGEL | 56 | 10 | 18 | 90 | 1898 |
| POL | RVHFASPLHV | 818 | 10 | 15 | 75 | 1899 |
| ENV | SLLVPFVQWF | 337 | 10 | 20 | 100 | 1900 |
| X | SLRGLPVCAF | 54 | 10 | 19 | 95 | 1901 |
| POL | SLTNLLSSNL | 403 | 10 | 18 | 90 | 1902 |
| NUC | SPHHTALRQA | 49 | 10 | 20 | 100 | 1903 |
| ENV | SPTVWLSVIW | 350 | 10 | 15 | 75 | 1904 |
| ENV | SVRFSWLSLL | 330 | 10 | 16 | 80 | 1905 |
| ENV | TIPQSLDSWW | 190 | 10 | 18 | 90 | 1906 |
| POL | TPARVTGGVF | 354 | 10 | 18 | 90 | 1907 |
| NUC | TPPAYRPPNA | 128 | 10 | 19 | 95 | 1908 |
| ENV | TPPHGGLLGW | 57 | 10 | 15 | 75 | 1909 |
| POL | VLGKSVQHL | 543 | 10 | 17 | 85 | 1910 |
| X | VLGGCRHKLV | 133 | 10 | 18 | 90 | 1911 |
| ENV | VPFVQWFVGL | 340 | 10 | 19 | 95 | 1912 |
| POL | VPNLQSLTNL | 398 | 10 | 19 | 95 | 1913 |
| NUC | VQASKLCLGW | 17 | 10 | 16 | 80 | 1914 |
| POL | VVLSRKYTSF | 740 | 10 | 17 | 85 | 1915 |
| POL | VVRRAFPHCL | 525 | 10 | 19 | 95 | 1916 |
| POL | WILRGTSFVY | 759 | 10 | 16 | 80 | 1917 |
| POL | WLLGCAANWI | 751 | 10 | 16 | 80 | 1918 |
| POL | WLSLDVSAAF | 414 | 10 | 19 | 95 | 1919 |
| NUC | WLWGMDIDPY | 26 | 10 | 17 | 85 | 1920 |
| ENV | WMCLRRFIIF | 237 | 10 | 19 | 95 | 1921 |
| ENV | WMMWYWGPSL | 359 | 10 | 17 | 85 | 1922 |
| POL | YLHTLWKAGI | 147 | 10 | 20 | 100 | 1923 |
| ENV | YQGMLPVCPL | 263 | 10 | 18 | 90 | 1924 |
| POL | YQHFRKLLLL | 5 | 10 | 15 | 75 | 1925 |
| POL | APFTQCGYPAL | 633 | 11 | 19 | 95 | 1926 |
| POL | AQFTSAICSVV | 516 | 11 | 19 | 95 | 1927 |
| POL | AVPNLQSLTNL | 397 | 11 | 19 | 95 | 1928 |
| ENV | CIPIPSSWAFA | 312 | 11 | 16 | 80 | 1929 |
| POL | CLAFSYMDDVV | 533 | 11 | 18 | 90 | 1930 |
| ENV | CLIFLLVLLDY | 253 | 11 | 19 | 95 | 1931 |
| ENV | CLRRFIIFLFI | 239 | 11 | 15 | 75 | 1932 |
| NUC | CPTVQASKLCL | 14 | 11 | 16 | 80 | 1933 |
| POL | CQRIVGLLGFA | 622 | 11 | 17 | 85 | 1934 |
| POL | DLNLGNLNVSI | 40 | 11 | 19 | 95 | 1935 |
| NUC | ELLSFLPSDFF | 43 | 11 | 19 | 95 | 1936 |
| ENV | FILLLCLIFLL | 248 | 11 | 16 | 80 | 1937 |
| ENV | FLFILLLCLIF | 246 | 11 | 16 | 80 | 1938 |
| ENV | FLLVLLDYQCM | 256 | 11 | 19 | 95 | 1939 |
| ENV | FPAGGSSSGTV | 130 | 11 | 15 | 75 | 1940 |
| POL | FPWLLGCAANW | 749 | 11 | 15 | 75 | 1941 |
| X | FVLGGCRHKLV | 132 | 11 | 18 | 90 | 1942 |
| POL | FVYVPSALNPA | 766 | 11 | 18 | 90 | 1943 |
| ENV | GLSPTVWLSVI | 348 | 11 | 15 | 75 | 1944 |
| POL | GPLEEELPRLA | 19 | 11 | 18 | 90 | 1945 |
| ENV | GPLLVLQAGFF | 173 | 11 | 19 | 95 | 1946 |
| POL | GPLTVNXEKRRL | 97 | 11 | 17 | 85 | 1947 |
| X | HLSLRGLPVCA | 52 | 11 | 18 | 90 | 1948 |
| POL | HLYSHPIILGF | 491 | 11 | 16 | 80 | 1949 |
| ENV | IIFLFILLLCL | 244 | 11 | 16 | 80 | 1950 |
| ENV | ILLLCLIFLLV | 249 | 11 | 20 | 100 | 1951 |
| NUC | ILSTLPETTVV | 139 | 11 | 20 | 100 | 1952 |
| ENV | ILTIPQSLDSW | 188 | 11 | 18 | 90 | 1953 |
| POL | IPMGVGLSPFL | 504 | 11 | 16 | 80 | 1954 |
| POL | IVGLLGFAAPF | 625 | 11 | 18 | 90 | 1955 |
| POL | KIPMGVGLSPF | 503 | 11 | 16 | 80 | 1956 |
| POL | KLHLYSHPIIL | 489 | 11 | 16 | 80 | 1957 |
| ENV | LLCLIFLLVLL | 251 | 11 | 20 | 100 | 1958 |
| ENV | LLGWSPQAQGI | 63 | 11 | 15 | 75 | 1959 |
| ENV | LLLCLIFLLVL | 250 | 11 | 20 | 100 | 1960 |
| ENV | LLPIFFCLWVY | 378 | 11 | 20 | 100 | 1961 |
| POL | LLSSNLSWLSL | 407 | 11 | 18 | 90 | 1962 |
| ENV | LLVLLDYQGML | 257 | 11 | 19 | 95 | 1963 |
| ENV | LLVLQAGFFLL | 175 | 11 | 18 | 90 | 1964 |
| NUC | LLWFHISCLTF | 100 | 11 | 17 | 85 | 1965 |
| ENV | LPIFFCLWVYI | 379 | 11 | 20 | 100 | 1968 |
| POL | LPIHTAELLAA | 712 | 11 | 17 | 85 | 1967 |
| POL | LPLDKGIKPYY | 123 | 11 | 20 | 100 | 1968 |
| POL | LPVNRPIDWKV | 611 | 11 | 16 | 80 | 1969 |
| ENV | LQAGFFLLTRI | 178 | 11 | 16 | 80 | 1970 |
| ENV | LVPFVQWFVGL | 339 | 11 | 19 | 95 | 1971 |
| POL | MPHLLVGSSGL | 433 | 11 | 16 | 80 | 1972 |
| POL | MPLSYQHFRKL | 1 | 11 | 15 | 75 | 1973 |
| POL | NLGNLNVSIPW | 42 | 11 | 19 | 95 | 1974 |
| POL | NLSWLSLDVSA | 411 | 11 | 18 | 90 | 1975 |
| POL | NPADDPSRGRL | 774 | 11 | 18 | 90 | 1976 |
| ENV | NPLGFFPDHQL | 9 | 11 | 19 | 95 | 1977 |
| POL | PIDWKVCQRIV | 616 | 11 | 17 | 85 | 1978 |
| POL | PIILGFRKIPM | 496 | 11 | 16 | 80 | 1979 |
| NUC | PILSTLPETTV | 138 | 11 | 20 | 100 | 1980 |
| POL | PLHPAAMPHLL | 427 | 11 | 20 | 100 | 1981 |
| ENV | PLLPIFFCLWV | 377 | 11 | 20 | 100 | 1982 |
| ENV | PLLVLQAGFFL | 174 | 11 | 18 | 90 | 1983 |
| POL | PLPIHTAELLA | 711 | 11 | 16 | 80 | 1984 |
| POL | PLSYQHFRKLL | 2 | 11 | 15 | 75 | 1985 |
| POL | PMGVGLSPFLL | 505 | 11 | 16 | 80 | 1986 |
| NUC | PPAYRPPNAPI | 129 | 11 | 19 | 95 | 1987 |
| ENV | PQAMQWNSTTF | 106 | 11 | 16 | 80 | 1988 |
| ENV | PQSLDSWWTSL | 192 | 11 | 18 | 90 | 1989 |
| X | QLDPARDVLCL | 8 | 11 | 16 | 80 | 1990 |
| POL | QVFADATPTGW | 685 | 11 | 19 | 95 | 1991 |
| POL | RLKMPARFY | 106 | 11 | 15 | 75 | 1992 |
| POL | RPIDWKVCQRI | 615 | 11 | 16 | 80 | 1993 |
| NUC | RPPNAPILSTL | 133 | 11 | 20 | 100 | 1994 |
| NUC | RQAILCWGELM | 56 | 11 | 18 | 90 | 1995 |
| NUC | RQLLWFHISCL | 98 | 11 | 18 | 90 | 1996 |
| POL | RVAEDLNLGNL | 36 | 11 | 18 | 90 | 1997 |
| POL | RVHFASPLHVA | 818 | 11 | 15 | 75 | 1998 |
| POL | SIPWTHKVGNF | 49 | 11 | 20 | 100 | 1999 |
| ENV | SLDSWWTSLNF | 194 | 11 | 19 | 95 | 2000 |
| ENV | SLLVPFVQWFV | 337 | 11 | 19 | 95 | 2001 |
| NUC | SPEHCSPHHTA | 44 | 11 | 20 | 100 | 2002 |
| POL | SPFLLAQFTSA | 511 | 11 | 19 | 95 | 2003 |
| NUC | SPHHTALRQAI | 49 | 11 | 20 | 100 | 2004 |
| ENV | SPTVWLSVIWM | 350 | 11 | 15 | 75 | 2005 |
| ENV | SVRFSWLSLLV | 330 | 11 | 16 | 80 | 2006 |
| POL | SVVLSRKYTSF | 739 | 11 | 17 | 85 | 2007 |
| POL | SVVRRAFPHCL | 524 | 11 | 19 | 95 | 2008 |
| POL | TPARVTGGVFL | 354 | 11 | 18 | 90 | 2009 |
| POL | TQCGYPALMPL | 636 | 11 | 19 | 95 | 2010 |
| NUC | TVQASKLCLGW | 16 | 11 | 16 | 80 | 2011 |
| ENV | VLLDYQGMLPV | 259 | 11 | 18 | 90 | 2012 |
| POL | VLSRKYTSFPW | 741 | 11 | 17 | 85 | 2013 |
| POL | VPNLQSLTNLL | 398 | 11 | 19 | 95 | 2014 |
| NUC | VQASKLCLGWL | 17 | 11 | 16 | 80 | 2015 |
| ENV | VQWFVGLSPTV | 343 | 11 | 19 | 95 | 2016 |
| POL | VVLGAKSVQHL | 542 | 11 | 16 | 80 | 2017 |
| POL | VVRRAFPHCLA | 525 | 11 | 19 | 95 | 2018 |
| POL | WILRGTSFVYV | 759 | 11 | 16 | 80 | 2019 |
| POL | WLLGCAANWIL | 751 | 11 | 16 | 80 | 2020 |
| POL | WLSLDVSAAFY | 414 | 11 | 19 | 95 | 2021 |
| ENV | WLSLLVPFVQW | 335 | 11 | 20 | 100 | 2022 |
| ENV | WMCLRRFIIFL | 237 | 11 | 19 | 95 | 2023 |
| ENV | WMMWYWGPSLY | 359 | 11 | 17 | 85 | 2024 |
| POL | YLHTLWKAGIL | 147 | 11 | 20 | 100 | 2025 |
| POL | YLPLDKGIKPY | 122 | 11 | 20 | 100 | 2026 |
| POL | YPALMPLYACI | 640 | 11 | 19 | 95 | 2027 |

TABLE XV
HBV A01 Motif with Binding information

| | | | | | | | |
|---|---|---|---|---|---|---|---|
| Conservancy | Freq. | Protein | Position | Sequence | AA | A*0101 | SEQ ID NO: |

| | | | | | | | |
|---|---|---|---|---|---|---|---|
| 100 | 20 | POL | 166 | ASFCGSPY | 8 | | 2028 |
| 90 | 18 | POL | 737 | DNSVVLSRKY | 10 | 0.0001 | 2029 |
| 95 | 19 | POL | 631 | FAAPFTQCGY | 10 | 0.0680 | 2030 |
| 95 | 19 | POL | 630 | GFAAPFTQCGY | 11 | | 2031 |
| 75 | 15 | NUC | 140 | GRETVLEY | 8 | | 2032 |
| 85 | 17 | POL | 579 | GYSLNFMGY | 9 | | 2033 |
| 100 | 20 | POL | 149 | HTLWKAGILY | 10 | 0.1100 | 2034 |
| 95 | 19 | POL | 653 | KQAFTFSPTY | 10 | 0.0001 | 2035 |
| 85 | 17 | NUC | 30 | LLDTASALY | 9 | 12.0000 | 2036 |
| 95 | 19 | POL | 415 | LSLDVSAAFY | 10 | 0.0150 | 2037 |
| 75 | 15 | NUC | 137 | LTFGRETVLEY | 11 | | 2038 |
| 85 | 17 | ENV | 360 | MMWYWGPSLY | 10 | 0.0810 | 2039 |
| 75 | 15 | X | 103 | MSTTDLEAY | 9 | 0.8500 | 2040 |
| 90 | 18 | POL | 738 | NSVVLSRKY | 9 | 0.0005 | 2041 |
| 100 | 20 | POL | 124 | PLDKGIKPY | 9 | | 2042 |
| 100 | 20 | POL | 124 | PLDKGIKPYY | 10 | 0.1700 | 2043 |
| 85 | 17 | POL | 797 | PTTGRTSLY | 9 | 0.2100 | 2044 |
| 100 | 20 | POL | 165 | SASFCGSPY | 9 | | 2045 |
| 95 | 19 | POL | 416 | SLDVSAAFY | 9 | 5.2000 | 2046 |
| 75 | 15 | X | 104 | STTDLEAY | 8 | | 2047 |
| 85 | 17 | POL | 798 | TTGRTSLY | 8 | | 2048 |
| 95 | 19 | POL | 414 | WLSLDVSAAFY | 11 | | 2049 |
| 85 | 17 | ENV | 359 | WMMWYWGPSLY | 11 | 0.3200 | 2050 |
| 95 | 19 | POL | 640 | YPALMPLY | 8 | | 2051 |
| 85 | 17 | POL | 580 | YSLNFMGY | 8 | | 2052 |

TABLE XVI
HBV A03 Motif With Binding

| | | | | | | | |
|---|---|---|---|---|---|---|---|
| Conservancy | Freq. | Protein | Position | Sequence | AA | A′0301 | SeqID Num |

| | | | | | | | |
|---|---|---|---|---|---|---|---|
| 85 | 17 | POL | 721 | AACFARSR | 8 | 0.0004 | 2053 |
| 85 | 17 | POL | 721 | AACFARSRSGA | 11 | | 2054 |
| 95 | 19 | POL | 632 | AAPFTQCGY | 9 | | 2055 |
| 95 | 19 | POL | 632 | AAPFTQCGYPA | 11 | | 2056 |
| 85 | 17 | POL | 722 | ACFARSRSGA | 10 | | 2057 |
| 80 | 16 | POL | 688 | ADATPTGWGLA | 11 | | 2058 |
| 90 | 18 | POL | 776 | ADDPSRGR | 8 | | 2059 |
| 95 | 19 | POL | 529 | AFPHCLAF | 8 | | 2060 |
| 95 | 19 | POL | 529 | AFPHCLAFSY | 10 | | 2061 |
| 95 | 19 | X | 62 | AFSSAGPCA | 9 | | 2062 |
| 90 | 18 | X | 62 | AFSSAGPCALR | 11 | | 2063 |
| 95 | 19 | POL | 655 | AFTFSPTY | 8 | | 2064 |
| 95 | 19 | POL | 655 | AFTFSPTYK | 9 | 0.2600 | 2065 |
| 95 | 19 | POL | 655 | AFTFSPTYKA | 10 | | 2066 |
| 95 | 19 | POL | 655 | AFTFSPTYKAF | 11 | | 2067 |
| 80 | 16 | ENV | 180 | AGFFLLTR | 8 | | 2068 |
| 90 | 18 | X | 66 | AGPCALRF | 8 | | 2069 |
| 90 | 18 | X | 66 | AGPCALRFTSA | 11 | | 2070 |
| 95 | 19 | POL | 18 | AGPLEEELPR | 10 | 0.0004 | 2071 |
| 95 | 19 | POL | 521 | AICSVVRR | 8 | −0.0002 | 2072 |
| 95 | 19 | POL | 521 | AICSVVRRA | 9 | | 2073 |
| 95 | 19 | POL | 521 | AICSVVRRAF | 10 | | 2074 |
| 95 | 19 | NUC | 41 | ALESPEHCSPH | 11 | | 2075 |
| 90 | 18 | POL | 772 | ALNPADDPSR | 10 | 0.0003 | 2076 |
| 85 | 17 | X | 70 | ALRFTSAR | 8 | 0.0047 | 2077 |
| 80 | 16 | ENV | 108 | AMQWNSTTF | 9 | | 2078 |
| 80 | 16 | ENV | 108 | AMQWNSTTFH | 10 | | 2079 |
| 75 | 15 | X | 102 | AMSITDLEA | 9 | | 2080 |
| 85 | 17 | NUC | 34 | ASALYREA | 8 | | 2081 |
| 100 | 20 | POL | 166 | ASFCGSPY | 8 | 0.0460 | 2082 |
| 80 | 16 | POL | 822 | ASPLHVAWR | 9 | | 2083 |
| 75 | 15 | ENV | 84 | ASTNRQSGR | 9 | 0.0009 | 2084 |
| 80 | 16 | POL | 690 | ATPTGWGLA | 9 | | 2085 |
| 80 | 16 | POL | 755 | CAANWILR | 8 | | 2086 |
| 95 | 19 | X | 61 | CAFSSAGPCA | 10 | | 2087 |
| 90 | 18 | X | 69 | CALRFTSA | 8 | | 2088 |
| 85 | 17 | X | 69 | CALRFTSAR | 9 | 0.0034 | 2089 |
| 80 | 16 | X | 6 | CCQLDPAR | 8 | | 2090 |
| 85 | 17 | POL | 723 | CFARSRSGA | 9 | | 2091 |
| 75 | 15 | POL | 607 | CFRKLPVNR | 9 | | 2092 |
| 95 | 19 | POL | 638 | CGYPALMPLY | 10 | | 2093 |
| 95 | 19 | POL | 638 | CGYPALMPLYA | 11 | | 2094 |
| 100 | 20 | ENV | 312 | CIPIPSSWA | 9 | | 2095 |
| 100 | 20 | ENV | 312 | CIPIPSSWAF | 10 | | 2096 |
| 80 | 16 | EVN | 312 | CIPIPSSWAFA | 11 | | 2097 |
| 95 | 19 | ENV | 253 | CLIFLLVLLDY | 11 | 0.0083 | 2098 |
| 90 | 18 | X | 17 | CLRPVGAESR | 10 | 0.0011 | 2099 |
| 95 | 19 | ENV | 239 | CLRRFIIF | 8 | | 2100 |
| 75 | 15 | ENV | 239 | CLRRFIIFLF | 10 | | 2101 |
| 100 | 20 | NUC | 48 | CSPHHTALR | 9 | 0.0029 | 2102 |
| 100 | 20 | NUC | 48 | CSPHHTALRQA | 11 | | 2103 |
| 95 | 19 | POL | 523 | CSVVRRAF | 8 | | 2104 |
| 95 | 19 | POL | 523 | CSVVRRAFPH | 10 | | 2105 |
| 100 | 20 | ENV | 310 | CTCIPIPSSWA | 11 | | 2106 |
| 80 | 16 | POL | 689 | DATPTGWGLA | 10 | | 2107 |
| 90 | 18 | POL | 540 | DDVVLGAK | 8 | | 2108 |
| 90 | 18 | NUC | 31 | DIDPYKEF | 8 | | 2109 |
| 90 | 18 | NUC | 31 | DIDPYKEFGA | 10 | | 2110 |
| 85 | 17 | NUC | 29 | DLLDTASA | 8 | | 2111 |
| 85 | 17 | NUC | 29 | DLLDTASALY | 10 | 0.0001 | 2112 |
| 85 | 17 | NUC | 29 | DLLDTASALYR | 11 | 0.0042 | 2113 |
| 95 | 19 | ENV | 196 | DSWWTSLNF | 9 | 0.0006 | 2114 |
| 85 | 17 | NUC | 32 | DTASALYR | 8 | 0.0004 | 2115 |
| 80 | 16 | NUC | 32 | DTASALYREA | 10 | | 2116 |
| 95 | 19 | X | 14 | DVLCLRPVGA | 10 | | 2117 |
| 95 | 19 | POL | 418 | DVSAAFYH | 8 | | 2118 |
| 90 | 18 | POL | 541 | DVVLGAKSVQH | 11 | | 2119 |
| 95 | 19 | POL | 17 | EAGPLEEELPR | 11 | −0.0009 | 2120 |
| 90 | 18 | NUC | 40 | EALESPEH | 8 | | 2121 |
| 90 | 18 | POL | 718 | ELLAACFA | 8 | | 2122 |
| 90 | 18 | POL | 718 | ELLAACFAR | 9 | 0.0002 | 2123 |
| 85 | 17 | POL | 718 | ELLAACFARSR | 11 | 0.0082 | 2124 |
| 95 | 19 | NUC | 43 | ELLSFLPSDF | 10 | | 2125 |
| 95 | 19 | NUC | 43 | ELLSFLPSDFF | 11 | | 2126 |
| 95 | 19 | NUC | 43 | ESPEHCSPH | 9 | | 2127 |
| 95 | 19 | NUC | 43 | ESPEHCSPHH | 10 | | 2128 |
| 95 | 19 | POL | 374 | ESRLVVDF | 8 | | 2129 |
| 95 | 19 | POL | 374 | ESRLVVDFSQF | 11 | | 2130 |
| 95 | 19 | NUC | 174 | ETTVVRRR | 8 | 0.0003 | 2131 |
| 80 | 16 | NUC | 174 | ETTVVRRRGR | 10 | 0.0003 | 2132 |
| 95 | 19 | POL | 631 | FAAPFTQCGY | 10 | | 2133 |
| 85 | 17 | POL | 724 | FARSRSGA | 8 | | 2134 |
| 80 | 16 | POL | 821 | FASPLHVA | 8 | | 2135 |
| 80 | 16 | POL | 821 | FASPLHVAWR | 10 | | 2136 |
| 90 | 18 | ENV | 13 | FFPDHQLDPA | 10 | | 2137 |
| 85 | 17 | ENV | 13 | FFPDHQLDPAF | 11 | | 2138 |
| 75 | 15 | NUC | 139 | FGRETVLEY | 9 | | 2139 |
| 75 | 15 | POL | 244 | FGVEPSGSGH | 10 | | 2140 |
| 95 | 19 | NUC | 122 | FGVWIRTPPA | 10 | | 2141 |
| 95 | 19 | NUC | 122 | FGVWIRTPPAY | 11 | | 2142 |
| 80 | 16 | ENV | 248 | FILLLCLIF | 9 | | 2143 |
| 80 | 16 | ENV | 246 | FLFILLLCLIF | 11 | | 2144 |
| 75 | 15 | ENV | 171 | FLGPLLVLQA | 10 | | 2145 |
| 95 | 19 | POL | 513 | FLLAQFTSA | 9 | 0.0006 | 2146 |
| 95 | 19 | POL | 562 | FLLSLGIH | 8 | | 2147 |
| 95 | 19 | ENV | 256 | FLLVLLDY | 8 | 0.0050 | 2148 |
| 100 | 20 | POL | 363 | FLVDKNPH | 8 | | 2149 |
| 95 | 19 | POL | 658 | FSPTYKAF | 8 | | 2150 |
| 95 | 19 | X | 63 | FSSAGPCA | 8 | | 2151 |
| 90 | 18 | X | 63 | FSSAGPCALR | 10 | | 2152 |
| 90 | 18 | X | 63 | FSSAGPCALRF | 11 | | 2153 |
| 100 | 20 | ENV | 333 | FSWLSLLVPF | 10 | 0.0004 | 2154 |
| 90 | 18 | POL | 536 | FSYMDDVVLGA | 11 | | 2155 |
| 95 | 19 | POL | 656 | FTFSPTYK | 8 | 0.0100 | 2156 |
| 95 | 19 | POL | 656 | FTFSPTYKA | 9 | | 2157 |
| 95 | 19 | POL | 656 | FTFSPTYKAF | 10 | 0.0004 | 2158 |
| 95 | 19 | POL | 635 | FTOCGYPA | 8 | | 2159 |
| 95 | 19 | POL | 518 | FTSAICSVVR | 10 | 0.0003 | 2160 |
| 95 | 19 | POL | 518 | FTSAICSVVRR | 11 | 0.0065 | 2161 |
| 95 | 19 | X | 132 | FVLGGCRH | 8 | | 2162 |
| 90 | 18 | X | 132 | FVLGGCRHK | 9 | 0.0430 | 2163 |
| 90 | 18 | POL | 766 | FVYVPSALNPA | 11 | | 2164 |
| 80 | 16 | POL | 754 | GCAANWILR | 9 | | 2165 |
| 95 | 19 | POL | 630 | GFAAPFTOCGY | 11 | | 2166 |
| 90 | 18 | ENV | 12 | GFFPDHOLDPA | 11 | | 2167 |
| 75 | 15 | ENV | 170 | GFLGPLLVLOA | 11 | | 2168 |
| 85 | 17 | ENV | 61 | GGLLGWSPQA | 10 | | 2169 |
| 100 | 20 | POL | 360 | GGVFLVDK | 8 | | 2170 |
| 100 | 20 | POL | 360 | GGVFLVDKNPH | 11 | | 2171 |
| 75 | 15 | POL | 567 | GIHLNPNK | 8 | | 2172 |
| 75 | 15 | POL | 567 | GIHLNPNKTK | 10 | 0.0025 | 2173 |
| 75 | 15 | POL | 567 | GIHLNPNKTKR | 11 | | 2174 |
| 85 | 17 | POL | 682 | GLCQVFADA | 9 | 0.0001 | 2175 |
| 95 | 19 | POL | 627 | GLLGFAAPF | 9 | 0.0006 | 2176 |
| 85 | 17 | ENV | 62 | GLLGWSPOA | 9 | | 2177 |
| 95 | 19 | X | 57 | GLPVCAFSSA | 10 | | 2178 |
| 95 | 19 | POL | 509 | GLSPFLLA | 8 | | 2179 |
| 95 | 19 | POL | 509 | GLSPFLLAQF | 10 | | 2180 |
| 85 | 17 | NUC | 29 | GMDIDPYK | 8 | 0.0006 | 2181 |
| 85 | 17 | NUC | 29 | GMDIDPYKEF | 10 | −0.0003 | 2182 |
| 90 | 18 | POL | 735 | GTDNSVVLSR | 10 | 0.0010 | 2183 |
| 90 | 18 | POL | 735 | GTDNSVVLSRK | 11 | 0.0140 | 2184 |
| 80 | 16 | POL | 763 | GTSFVYVPSA | 10 | | 2185 |
| 80 | 16 | POL | 245 | GVEPSGSGH | 9 | | 2186 |
| 100 | 20 | POL | 361 | GVFLVDKNPH | 10 | | 2187 |
| 80 | 16 | POL | 507 | GVGLSPFLLA | 10 | | 2188 |
| 95 | 19 | NUC | 123 | GVWIRTPPA | 9 | | 2189 |
| 95 | 19 | NUC | 123 | GVWIRTPPAY | 10 | 0.0047 | 2190 |
| 95 | 19 | NUC | 123 | GVWIRTPPAYR | 11 | 0.1900 | 2191 |
| 100 | 20 | NUC | 47 | HCSPHHTA | 8 | | 2192 |
| 100 | 20 | NUC | 47 | HCSPHHTALR | 10 | | 2193 |
| 80 | 16 | POL | 820 | HFASPLHVA | 9 | | 2194 |
| 80 | 16 | POL | 820 | HFASPLHVAWR | 11 | | 2195 |
| 95 | 19 | X | 49 | HGAHLSLR | 8 | | 2196 |
| 85 | 17 | ENV | 60 | HGGLLGWSPQA | 11 | | 2197 |
| 90 | 18 | NUC | 104 | HISCLTFGR | 9 | | 2198 |
| 75 | 15 | POL | 569 | HLNPNKTK | 8 | | 2199 |
| 75 | 15 | POL | 569 | HLNPNKTKR | 9 | | 2200 |
| 90 | 18 | X | 52 | HLSLRGLPVCA | 11 | | 2201 |
| 80 | 16 | POL | 491 | HLYSHPIILGF | 11 | | 2202 |
| 85 | 17 | POL | 715 | HTAELLAA | 8 | | 2203 |
| 85 | 17 | POL | 715 | HTAELLAACF | 10 | | 2204 |
| 85 | 17 | POL | 715 | HTAELLAACFA | 11 | | 2205 |
| 100 | 20 | POL | 149 | HTLWKAGILY | 10 | 0.0440 | 2206 |
| 100 | 20 | POL | 149 | HTLWKAGILYK | 11 | 0.5400 | 2207 |
| 95 | 19 | POL | 522 | ICSVVRRA | 8 | | 2208 |
| 95 | 19 | POL | 522 | ICSVVRRAF | 9 | | 2209 |
| 95 | 19 | POL | 522 | ICSVVRRAFPH | 11 | | 2210 |
| 90 | 18 | NUC | 32 | IDPYKEFGA | 9 | | 2211 |
| 90 | 18 | POL | 617 | IDWKVCQR | 8 | | 2212 |
| 100 | 20 | ENV | 381 | IFFCLWVY | 8 | | 2213 |
| 95 | 19 | ENV | 255 | IFLLVLLDY | 9 | | 2214 |
| 80 | 16 | POL | 734 | IGTDNSVVLSR | 11 | | 2215 |
| 100 | 20 | ENV | 249 | ILLLCLIF | 8 | | 2216 |
| 80 | 16 | POL | 760 | ILRGTSFVY | 9 | 0.0440 | 2217 |
| 90 | 18 | NUC | 105 | ISCLTFGR | 8 | 0.0004 | 2218 |
| 90 | 18 | POL | 625 | IVGLLGFA | 8 | | 2219 |
| 90 | 18 | POL | 625 | IVGLLGFAA | 9 | | 2220 |
| 90 | 18 | POL | 625 | IVGLLGFAAPF | 11 | | 2221 |
| 100 | 20 | POL | 153 | KAGILYKR | 8 | 0.0002 | 2222 |
| 80 | 16 | POL | 503 | KIPMGVGLSPF | 11 | | 2223 |
| 75 | 15 | POL | 108 | KLIMPARF | 8 | | 2224 |
| 75 | 15 | POL | 108 | KLIMPARFY | 9 | | 2225 |
| 80 | 16 | POL | 610 | KLPVNRPIDWK | 11 | | 2226 |
| 85 | 17 | POL | 574 | KTKRWGYSLNF | 11 | | 2227 |
| 75 | 15 | X | 130 | KVFVLGGCR | 9 | 0.0420 | 2228 |
| 75 | 15 | X | 130 | KVFVLGGCRH | 10 | | 2229 |
| 95 | 19 | POL | 55 | KVGNFTGLY | 9 | 0.2100 | 2230 |
| 85 | 17 | POL | 720 | LAACFARSR | 9 | 0.0058 | 2231 |
| 95 | 19 | X | 16 | LCLRPVGA | 8 | | 2232 |
| 90 | 18 | X | 16 | LCLRPVGAESR | 11 | | 2233 |
| 95 | 19 | POL | 683 | LCQVFADA | 8 | | 2234 |
| 100 | 20 | POL | 125 | LDKGIKPY | 8 | | 2235 |
| 100 | 20 | POL | 125 | LDKGIKPYY | 9 | | 2236 |
| 80 | 16 | X | 9 | LDPARDVLCLR | 11 | | 2237 |
| 95 | 19 | ENV | 195 | LDSWWTSLNF | 10 | | 2238 |
| 85 | 17 | NUC | 31 | LDTASALY | 8 | | 2239 |
| 85 | 17 | NUC | 31 | LDTASALYR | 9 | 0.0004 | 2240 |
| 80 | 16 | NUC | 31 | LDTASALYREA | 11 | | 2241 |
| 95 | 19 | POL | 417 | LDVSAAFY | 8 | | 2242 |
| 95 | 19 | POL | 417 | LDVSAAFYH | 9 | | 2243 |
| 80 | 16 | ENV | 247 | LFILLLCLIF | 10 | | 2244 |
| 95 | 19 | POL | 544 | LGAKSVQH | 8 | | 2245 |
| 80 | 16 | POL | 753 | LGCAANWILR | 10 | | 2246 |
| 75 | 15 | POL | 566 | LGIHLNPNK | 9 | | 2247 |
| 75 | 15 | POL | 566 | LGIHLNPNKTK | 11 | | 2248 |
| 95 | 19 | ENV | 172 | LGPLLVLQA | 9 | | 2249 |
| 95 | 19 | ENV | 172 | LGPLLVLQAGF | 11 | | 2250 |
| 95 | 19 | EMI | 254 | LIFLLVLLDY | 10 | 0.0022 | 2251 |
| 100 | 20 | POL | 109 | LIMPARFY | 8 | −0.0002 | 2252 |
| 90 | 18 | POL | 719 | LLAACFAR | 8 | 0.0024 | 2253 |
| 85 | 17 | POL | 719 | LLAACFARSR | 10 | | 2254 |
| 95 | 19 | POL | 514 | LLAQFTSA | 8 | | 2255 |
| 85 | 17 | NUC | 30 | LLDTASALY | 9 | 0.0013 | 2256 |
| 85 | 17 | NUC | 30 | LLDTASALYR | 10 | 0.0050 | 2257 |
| 80 | 16 | POL | 752 | LLGCAANWILR | 11 | | 2258 |
| 95 | 19 | POL | 628 | LLGFAAPF | 8 | | 2259 |
| 85 | 17 | EMI | 63 | LLGWSPQA | 8 | | 2260 |
| 100 | 20 | ETV | 378 | LLPIFFCLWVY | 11 | 0.0230 | 2261 |
| 95 | 19 | NUC | 44 | LLSFLPSDF | 9 | | 2262 |
| 95 | 19 | NUC | 44 | LLSFLPSDFF | 10 | | 2263 |
| 95 | 19 | ENV | 175 | LLVLQAGF | 8 | | 2264 |
| 95 | 19 | ENV | 175 | LLVLQAGFF | 9 | 0.0006 | 2265 |
| 100 | 20 | ENV | 336 | LLVPFVQWF | 9 | | 2266 |
| 85 | 17 | NUC | 100 | LLWFHISCLTF | 11 | | 2267 |
| 95 | 19 | NUC | 45 | LSFLPSDF | 8 | | 2268 |
| 95 | 19 | NUC | 45 | LSFLPSDFF | 9 | 0.0006 | 2269 |
| 95 | 19 | POL | 415 | LSLDVSAA | 8 | | 2270 |
| 95 | 19 | POL | 415 | LSLDVSAAF | 9 | 0.0004 | 2271 |
| 95 | 19 | POL | 415 | LSLDVSAAFY | 10 | | 2272 |
| 95 | 19 | POL | 415 | LSLDVSAAFYII | 11 | | 2273 |
| 75 | 15 | POL | 564 | LSLGIHLNPNK | 11 | | 2274 |
| 100 | 20 | ENV | 336 | LSLLVPFVQWF | 11 | | 2275 |
| 95 | 19 | X | 53 | LSLRGLPVCA | 10 | | 2276 |
| 95 | 19 | X | 53 | LSLRGLPVCAF | 11 | | 2277 |
| 95 | 19 | POL | 510 | LSPFLLAQF | 9 | | 2278 |
| 95 85 | 17 | POL | 742 | LSRKYTSF | 8 | | 2279 |
| 95 | 19 | NUC | 169 | LSTLPETTVVR | 11 | −0.0009 | 2280 |
| 75 | 15 | ENV | 16 | LSVPNPLGF | 9 | | 2281 |
| 100 | 20 | POL | 412 | LSWLSLDVSA | 10 | 0.0048 | 2282 |
| 100 | 19 | POL | 412 | LSWLSLDVSAA | 11 | | 2283 |
| 95 | 15 | POL | 3 | LSYQIIFRK | 8 | | 2284 |
| 75 | 15 | NUC | 137 | LTFGRETVLEY | 11 | | 2285 |
| 85 | 17 | POL | 99 | LTVNEKRR | 8 | −0.0002 | 2286 |
| 95 | 19 | ENV | 176 | LVLQAGFF | 8 | | 2287 |
| 100 | 20 | ENV | 339 | LVPFVQWF | 8 | 0.0028 | 2288 |
| 90 | 18 | NUC | 119 | LVSFGVWIR | 9 | | 2289 |
| 100 | 20 | POL | 377 | LWDFSQF | 8 | 0.0016 | 2290 |
| 100 | 20 | POL | 377 | LWDFSQFSR | 10 | | 2291 |
| 95 | 19 | ENV | 238 | MCLRRFIIF | 9 | | 2292 |
| 75 | 15 | ENV | 238 | MCLRRFIIFLF | 11 | | 2293 |
| 90 | 18 | POL | 539 | MDDWLGA | 8 | | 2294 |
| 90 | 18 | POL | 539 | MDDWLGAK | 9 | | 2295 |
| 90 | 18 | NUC | 30 | MDIDPYKEF | 9 | | 2296 |
| 90 | 18 | NUC | 30 | MDIDPYKEFGA | 11 | | 2297 |
| 80 | 16 | POL | 506 | MGVGLSPF | 8 | | 2298 |
| 80 | 16 | POL | 506 | MGVGLSPFLLA | 11 | | 2299 |
| 85 | 17 | ENV | 360 | MMWYWGPSLY | 10 | 0.0500 | 2300 |
| 80 | 16 | X | 103 | MSTTDLEA | 8 | | 2301 |
| 75 | 15 | X | 103 | MSTTDLEAY | 9 | 0.0008 | 2302 |
| 75 | 15 | X | 103 | MSTTDLEAYF | 10 | | 2303 |
| 75 | 15 | X | 103 | MSTTDLEAYFK | 11 | | 2304 |
| 95 | 19 | POL | 561 | NFLLSLGIH | 9 | | 2305 |
| 90 | 18 | NUC | 75 | NLEDPASR | 8 | −0.0002 | 2306 |
| 95 | 19 | POL | 45 | NLNVSIPWTH | 10 | | 2307 |
| 95 | 19 | POL | 45 | NLNVSIPWTHK | 11 | −0.0009 | 2308 |
| 95 | 15 | ENV | 15 | NLSVPNPLGF | 10 | | 2309 |
| 90 | 18 | POL | 411 | NLSWLSLDVSA | 11 | | 2310 |
| 75 | 15 | ENV | 215 | NSQSPTSNH | 9 | | 2311 |
| 90 | 18 | POL | 738 | NSVVLSRK | 8 | 0.0006 | 2312 |
| 90 | 18 | POL | 738 | NSVVLSRKY | 9 | 0.0020 | 2313 |
| 100 | 20 | POL | 47 | NVSIPWTH | 8 | | 2314 |
| 100 | 20 | POL | 47 | NVSIPWTHK | 9 | 0.0820 | 2315 |
| 90 | 18 | POL | 775 | PADDPSRGR | 9 | 0.0008 | 2316 |
| 95 | 19 | POL | 641 | PALMPLYA | 8 | | 2317 |
| 75 | 15 | X | 145 | PAPCNFFTSA | 10 | | 2318 |
| 80 | 16 | X | 11 | PARDVLCLR | 9 | 0.0002 | 2319 |
| 90 | 18 | POL | 355 | PARVTGGVF | 9 | | 2320 |
| 75 | 15 | ENV | 83 | PASTNRQSGR | 10 | | 2321 |
| 95 | 19 | NUC | 130 | PAYRPPNA | 8 | | 2322 |
| 90 | 18 | X | 68 | PCALRFTSA | 9 | | 2323 |
| 85 | 17 | X | 68 | PCALRFTSAR | 10 | | 2324 |
| 75 | 15 | X | 147 | PCNFFTSA | 8 | | 2325 |
| 95 | 19 | ENV | 15 | PDHQLDPA | 8 | | 2326 |
| 90 | 18 | ENV | 15 | PDHQLDPAF | 9 | | 2327 |
| 95 | 19 | POL | 512 | PFLLAQFTSA | 10 | | 2328 |
| 95 | 19 | POL | 634 | PFTQCGYPA | 9 | | 2329 |
| 100 | 20 | ENV | 233 | PGYRWMCLR | 9 | 0.0008 | 2330 |
| 95 | 19 | ENV | 233 | PGYRWMCLRR | 10 | 0.0048 | 2331 |
| 95 | 19 | ENV | 233 | PGYRWMCLRRF | 11 | | 2332 |
| 90 | 18 | POL | 616 | PIDWKVCQR | 9 | 0.0002 | 2333 |
| 100 | 20 | ENV | 380 | PIFFCLWVY | 9 | 0.0011 | 2334 |
| 85 | 17 | POL | 713 | PIHTAELLA | 9 | | 2335 |
| 85 | 17 | POL | 713 | PIHTAELLAA | 10 | | 2336 |
| 80 | 16 | POL | 496 | PIILGFRK | 8 | | 2337 |
| 100 | 20 | ENV | 314 | PIPSSWAF | 8 | | 2338 |
| 80 | 16 | ENV | 314 | PIPSSWAFA | 9 | | 2339 |
| 100 | 20 | POL | 124 | PLDKGIKPY | 9 | 0.0001 | 2340 |
| 100 | 20 | POL | 124 | PLDKGIKPYY | 10 | 0.0002 | 2341 |
| 95 | 19 | POL | 20 | PLEEELPR | 8 | 0.0002 | 2342 |
| 90 | 16 | POL | 20 | PLEEELPRLA | 10 | | 2343 |
| 90 | 19 | ENV | 10 | PLGFFPDH | 8 | | 2344 |
| 100 | 20 | POL | 427 | PLHPAAMPH | 9 | 0.0012 | 2345 |
| 95 | 19 | ENV | 174 | PLLVLQAGF | 9 | | 2346 |
| 95 | 19 | ENV | 174 | PLLVLQAGFF | 10 | | 2347 |
| 80 | 16 | POL | 711 | PLPIHTAELLA | 11 | | 2348 |
| 100 | 20 | POL | 2 | PLSYQHFR | 8 | −0.0002 | 2349 |
| 75 | 15 | POL | 2 | PLSYQHFRK | 9 | 0.0011 | 2350 |
| 85 | 17 | POL | 98 | PLTVNEKR | 8 | 0.0002 | 2351 |
| 85 | 17 | POL | 98 | PLTVNEKRR | 9 | 0.0008 | 2352 |
| 80 | 16 | POL | 505 | PMGVGLSPF | 9 | | 2353 |
| 85 | 17 | POL | 797 | PTTGRTSLY | 9 | 0.0001 | 2354 |
| 85 | 17 | POL | 797 | PTTGRTSLYA | 10 | | 2355 |
| 95 | 19 | X | 59 | PVCAFSSA | 8 | | 2356 |
| 90 | 18 | X | 20 | PVGAESRGR | 9 | 0.0002 | 2357 |
| 85 | 17 | POL | 612 | PVNRPIDWK | 9 | 0.0310 | 2358 |
| 95 | 19 | POL | 654 | QAFTFSPTY | 9 | 0.0030 | 2359 |
| 95 | 19 | POL | 654 | QAFTFSPTYK | 10 | 0.0450 | 2360 |
| 95 | 19 | POL | 654 | QAFTFSPTYKA | 11 | | 2361 |
| 80 | 16 | ENV | 179 | QAGFFLLTR | 9 | | 2362 |
| 80 | 16 | ENV | 107 | QAMQWNSTTF | 10 | | 2363 |
| 80 | 16 | ENV | 107 | QAMQWNSTTFH | 11 | | 2364 |
| 95 | 19 | POL | 637 | QCGYPALMPLY | 11 | | 2365 |
| 95 | 19 | POL | 517 | QFTSAICSVVR | 11 | | 2366 |
| 75 | 15 | NUC | 169 | QSPRRRRSQSR | 11 | | 2367 |
| 80 | 16 | POL | 189 | QSSGILSR | 8 | | 2368 |
| 95 | 19 | POL | 528 | RAFPHCLA | 8 | | 2369 |
| 95 | 19 | POL | 528 | RAFPHCLAF | 9 | 0.0015 | 2370 |
| 95 | 19 | POL | 528 | RAFPHCIAFSY | 11 | 0.1200 | 2371 |
| 85 | 17 | NUC | 28 | RDLLDTASA | 9 | | 2372 |
| 85 | 17 | NUC | 28 | RDLLDTASALY | 11 | | 2373 |
| 95 | 19 | X | 13 | RDVLCLRPVGA | 11 | | 2374 |
| 100 | 20 | ENV | 332 | RFSWLSLLVPF | 11 | | 2375 |
| 95 | 19 | X | 56 | RGLPVCAF | 8 | | 2376 |
| 95 | 19 | X | 56 | RGLPVCAFSSA | 11 | | 2377 |
| 100 | 20 | NUC | 152 | RGRSPRRR | 8 | | 2378 |
| 80 | 16 | POL | 762 | RGTSFVYVPSA | 11 | | 2379 |
| 90 | 18 | POL | 624 | RNGLLGF | 8 | | 2380 |
| 90 | 18 | POL | 624 | RIVGLLGFA | 9 | | 2381 |
| 90 | 18 | POL | 624 | RIVGLLGFAA | 10 | | 2382 |
| 75 | 15 | POL | 106 | RLKLIMPA | 8 | | 2383 |
| 75 | 15 | POL | 106 | RLKLIMPAR | 9 | 0.0950 | 2384 |
| 75 | 15 | POL | 106 | RLKLIMPARF | 10 | | 2385 |
| 75 | 15 | POL | 106 | RLKLIMPARFY | 11 | | 2386 |
| 75 | 15 | X | 128 | RLKVFVLGGCR | 11 | | 2387 |
| 95 | 19 | POL | 376 | RLVVDFSQF | 9 | 0.0006 | 2388 |
| 95 | 19 | POL | 376 | RLVVDFSQFSR | 11 | 0.2800 | 2389 |
| 95 | 19 | NUC | 163 | RSPRRRTPSPR | 11 | −0.0007 | 2390 |
| 75 | 15 | NUC | 167 | RSQSPRRR | 8 | | 2391 |
| 75 | 15 | NUC | 167 | RSQSPRRRR | 9 | | 2392 |
| 90 | 18 | POL | 353 | RTPARVTGGVF | 11 | | 2393 |
| 95 | 19 | NUC | 127 | RTPPAYRPPNA | 11 | | 2394 |
| 95 | 19 | NUC | 188 | RTPSPRRR | 8 | −0.0002 | 2395 |
| 95 | 19 | NUC | 188 | RTPSPRRRR | 9 | 0.0054 | 2396 |
| 95 | 16 | POL | 818 | RVHFASPLH | 9 | | 2397 |
| 75 | 15 | POL | 818 | RVHFASPLHVA | 11 | | 2398 |
| 100 | 20 | POL | 357 | RVTGGVFIVDK | 11 | 0.0190 | 2399 |
| 90 | 18 | X | 65 | SAGPCALR | 8 | −0.0002 | 2400 |
| 90 | 18 | X | 65 | SAGPCALRF | 9 | −0.0003 | 2401 |
| 95 | 19 | POL | 520 | SAICSVVR | 8 | −0.0002 | 2402 |
| 95 | 19 | POL | 520 | SAICSVVRR | 9 | 0.0058 | 2403 |
| 95 | 19 | POL | 520 | SAICSWRRA | 10 | | 2404 |
| 95 | 19 | POL | 520 | SAICSWRRAF | 11 | | 2405 |
| 95 | 18 | POL | 771 | SALNPADDPSR | 11 | −0.0004 | 2406 |
| 90 | 20 | POI | 165 | SASFCGSPY | 9 | | 2407 |
| 100 | 18 | NUC | 121 | SFGVWIRTPPA | 11 | | 2408 |
| 90 | 19 | NUC | 46 | SFLPSDFF | 8 | | 2409 |
| 95 | 15 | POL | 748 | SFPWLIGCA | 9 | | 2410 |
| 75 | 15 | POL | 740 | SFPWLLGCAA | 10 | | 2411 |
| 75 | 16 | POL | 765 | SFVWPSA | 8 | | 2412 |
| 80 | 20 | POL | 49 | SIPWTHKVGNF | 11 | | 2413 |
| 100 | 19 | ENV | 194 | SLDSWWTSINF | 11 | | 2414 |
| 95 | 19 | POL | 416 | SLDVSAAF | 8 | | 2415 |
| 95 | 19 | POL | 416 | SIQVSAAFY | 9 | 0.0016 | 2416 |
| 95 | 19 | POL | 416 | SLDVSAAFYH | 10 | | 2417 |
| 75 | 15 | POL | 565 | SIGIHLNPNK | 10 | | 2418 |
| 100 | 20 | ENV | 337 | SLLVPFVQWF | 10 | | 2419 |
| 95 | 19 | X | 54 | SLRGLPVCA | 9 | | 2420 |
| 95 | 19 | X | 54 | SLRGLPVCAF | 10 | 0.0004 | 2421 |
| 95 | 18 | X | 64 | SSAGPCALR | 9 | 0.0080 | 2422 |
| 90 | 18 | X | 64 | SSAGPCALRF | 10 | −0.0003 | 2423 |
| 90 | 19 | NUC | 170 | STIPETTVVR | 10 | 0.0007 | 2424 |
| 95 | 19 | NUC | 170 | STLPETTWRR | 11 | 0.0150 | 2425 |
| 95 | 16 | ENV | 85 | STNRQSGR | 8 | | 2426 |
| 80 | 15 | X | 104 | STTDLEAY | 8 | | 2427 |
| 75 | 15 | X | 104 | STTDLEAYF | 9 | | 2428 |
| 75 | 15 | X | 104 | STTDLEAYFK | 10 | 0.0066 | 2429 |
| 75 | 15 | ENV | 17 | SVPNPLGF | 8 | | 2430 |
| 90 | 18 | POL | 739 | SVVLSRKY | 8 | −0.0002 | 2431 |
| 85 | 17 | POL | 739 | SVVLSRKYTSF | 11 | | 2432 |
| 95 | 19 | POL | 524 | SVVRRAFPH | 9 | 0.1100 | 2433 |
| 85 | 17 | POL | 716 | TAELLAACF | 9 | | 2434 |
| 85 | 17 | POL | 716 | TAELLAACFA | 10 | | 2435 |
| 85 | 17 | POL | 716 | TAELLAACFAR | 11 | 0.0006 | 2436 |
| 80 | 16 | NUC | 33 | TASALYREA | 9 | | 2437 |
| 100 | 20 | ENV | 311 | TCIPIPSSWA | 10 | | 2438 |
| 100 | 20 | ENV | 311 | TCIPIPSSWAF | 11 | | 2439 |
| 80 | 16 | X | 106 | TDLEAYFK | 8 | | 2440 |
| 90 | 18 | POL | 736 | TDNSVVLSR | 9 | | 2441 |
| 90 | 18 | POL | 736 | TDNSVVLSRK | 10 | 0.0006 | 2442 |
| 90 | 18 | POL | 736 | TDNSVVLSRKY | 11 | | 2443 |
| 75 | 15 | NUC | 138 | TFGRETVLEY | 10 | | 2444 |
| 95 | 19 | POL | 657 | TFSPTYKA | 8 | | 2445 |
| 95 | 19 | POL | 857 | TFSPTYKAF | 9 | | 2446 |
| 100 | 20 | POL | 359 | TGGVFLVQK | 9 | 0.0007 | 2447 |
| 85 | 17 | POL | 799 | TGRTSLYA | 6 | | 2448 |
| 95 | 19 | NUC | 171 | TLPETTVVR | 9 | 0.0008 | 2449 |
| 95 | 19 | NUC | 171 | TLPETTVVRR | 10 | 0.0007 | 2450 |
| 95 | 19 | NUC | 171 | TLPETTVVRRR | 11 | 0.0005 | 2451 |
| 100 | 20 | POL | 150 | TLWKAGILY | 9 | 0.1300 | 2452 |
| 100 | 20 | POL | 150 | TLWKAGILYK | 10 | 5.3000 | 2453 |
| 100 | 20 | POL | 150 | TLWKAGILYKR | 11 | 0.0082 | 2454 |
| 95 | 19 | POL | 519 | TSAICSVVR | 9 | 0.0005 | 2455 |
| 95 | 19 | POL | 519 | TSAICSVVRR | 10 | 0.0018 | 2456 |
| 95 | 19 | POL | 519 | TSAICSVVRRA | 11 | | 2457 |
| 75 | 15 | POL | 747 | TSFPWLLGCA | 10 | | 2458 |
| 75 | 15 | POL | 747 | TSFPWLLGCAA | 11 | | 2459 |
| 80 | 16 | POL | 764 | TSFVYVPSA | 9 | | 2460 |
| 75 | 15 | X | 105 | TTDLEAYF | 8 | | 2461 |
| 75 | 15 | X | 105 | TTDLEAYFK | 9 | 0.0006 | 2462 |
| 85 | 17 | POL | 798 | TTGRTSLY | 8 | 0.0004 | 2463 |
| 85 | 17 | POL | 798 | TTGRTSLYA | 9 | | 2464 |
| 75 | 15 | ENV | 278 | TTSTGPCK | 8 | | 2465 |
| 80 | 16 | NUC | 175 | TTVVRRRGR | 9 | 0.0008 | 2466 |
| 80 | 16 | NUC | 176 | TVVRRRGR | 8 | 0.0003 | 2467 |
| 80 | 16 | NUC | 176 | TVVRRRGRSPR | 11 | | 2468 |
| 95 | 19 | X | 60 | VCAFSSAGPCA | 11 | | 2469 |
| 85 | 17 | POL | 621 | VCQRNGLLGF | 11 | | 2470 |
| 100 | 20 | POL | 379 | VDFSQFSR | 8 | | 2471 |
| 100 | 20 | POL | 362 | VFLVDKNPH | 9 | | 2472 |
| 80 | 16 | X | 131 | VFVLGGCR | 8 | | 2473 |
| 80 | 16 | X | 131 | VFVLGGCRH | 9 | | 2474 |
| 75 | 15 | X | 131 | VFVLGGCRHK | 10 | | 2475 |
| 95 | 19 | X | 21 | VGAESRGR | 8 | | 2476 |
| 95 | 19 | POL | 626 | VGLLGFAA | 8 | | 2477 |
| 95 | 19 | POL | 626 | VGLLGFAAPF | 10 | | 2478 |
| 80 | 16 | POL | 508 | VGLSPFLLA | 9 | | 2479 |
| 80 | 16 | POL | 508 | VGLSPFLLAQF | 11 | | 2480 |
| 95 | 19 | PO[- | 56 | VGNFTGLY | 8 | | 2481 |
| 85 | 17 | POL | 96 | VGPLTVNEK | 9 | 0.0007 | 2482 |
| 85 | 17 | POL | 96 | VGPLTVNEKR | 10 | | 2483 |
| 85 | 17 | POL | 96 | VGPLTVNEKRR | 11 | | 2484 |
| 95 | 19 | X | 15 | VLCLRPVGA | 9 | | 2485 |
| 95 | 19 | POL | 543 | VLGAKSVQH | 9 | | 2486 |
| 90 | 18 | X | 133 | VLGGCRHK | 8 | 0.0150 | 2487 |
| 80 | 16 | ENV | 177 | VLQAGFFLLTR | 11 | | 2488 |
| 85 | 17 | POL | 741 | VLSRKYTSF | 9 | | 2489 |
| 90 | 18 | NUC | 120 | VSFGVWIR | 8 | 0.0040 | 2490 |
| 100 | 20 | POL | 48 | VSIPWTHK | 8 | 0.0130 | 2491 |
| 100 | 20 | POL | 358 | VTGGVFLVDK | 10 | 0.0390 | 2492 |
| 100 | 20 | POL | 378 | VVDFSQFSR | 9 | 0.0015 | 2493 |
| 90 | 18 | POL | 542 | VVLGAKSVQH | 10 | | 2494 |
| 85 | 17 | POL | 740 | VVLSRKYTSF | 10 | 0.0004 | 2495 |
| 95 | 19 | POL | 525 | VVRRAFPH | 8 | | 2496 |
| 95 | 19 | POL | 525 | VVRRAFPHCLA | 11 | | 2497 |
| 80 | 16 | NUC | 177 | VVRRRGRSPR | 10 | 0.0027 | 2498 |
| 80 | 16 | NUC | 177 | VVRRRGRSPRR | 11 | | 2499 |
| 90 | 18 | NUC | 102 | WFHISCLTF | 9 | | 2500 |
| 90 | 16 | NUC | 102 | WFHISCLTFGR | 11 | | 2501 |
| 85 | 17 | NUC | 28 | WGMDIDPY | 8 | | 2502 |
| 85 | 17 | NUC | 28 | WGMDIDPYK | 9 | −0.0003 | 2503 |
| 85 | 17 | NUC | 28 | WGMDIDPYKEF | 11 | | 2504 |
| 85 | 17 | POL | 578 | WGYSLNFMGY | 10 | | 2505 |
| 80 | 16 | POL | 759 | WILRGTSF | 8 | | 2506 |
| 80 | 16 | POL | 759 | WILRGTSFVY | 10 | 0.0076 | 2507 |
| 95 | 19 | NUC | 125 | WIRTPPAY | 8 | −0.0002 | 2508 |
| 95 | 19 | NX | 125 | WIRTPPAYR | 9 | 0.0008 | 2509 |
| 90 | 18 | POL | 314 | WLQFRNSK | 8 | −0.0002 | 2510 |
| 100 | 20 | POL | 414 | WLSLDVSA | 8 | | 2511 |
| 95 | 19 | POL | 414 | WLSLDVSAA | 9 | | 2512 |
| 95 | 19 | POL | 414 | WLSLDVSAAF | 10 | | 2513 |
| 95 | 19 | POL | 414 | WLSLDVSAAFY | 11 | 0.0034 | 2514 |
| 100 | 20 | ENV | 335 | WLSLLVPF | 8 | | 2515 |
| 85 | 17 | RUC | 26 | WLWGMDIDPY | 10 | 0.0002 | 2516 |
| 85 | 17 | NUC | 26 | WLWGMDIDPYK | 11 | 0.0030 | 2517 |
| 95 | 19 | ENV | 237 | WMCLRRFIIF | 10 | 0.0004 | 2518 |
| 85 | 17 | ENV | 359 | WMMWYWGPSLY | 11 | 0.0009 | 2519 |
| 100 | 20 | POI | 52 | WTHKVGNF | 8 | | 2520 |
| 100 | 20 | POL | 147 | YLHTLWKA | 8 | | 2521 |
| 100 | 20 | POL | 122 | YLPLDKGIK | 9 | 0.0001 | 2522 |
| 100 | 20 | POL | 122 | YLPLDKGIKPY | 11 | −0.0004 | 2523 |
| 90 | 18 | NUC | 118 | YLVSFGVWIR | 10 | 0.0005 | 2524 |
| 90 | 18 | POL | 538 | YMDDVVLGA | 9 | 0.0001 | 2525 |
| 90 | 18 | POL | 538 | YMDDWLGAK | 10 | 0.0330 | 2526 |
| 80 | 16 | POL | 493 | YSHPIILGF | 9 | | 2527 |
| 80 | 16 | FOL | 493 | YSHPIILGFR | 10 | | 2528 |
| 80 | 16 | POL | 493 | YSHIPIILGFRK | 11 | | 2529 |
| 85 | 17 | POL | 580 | YSLNFMGY | 8 | −0.0002 | 2530 |
| 75 | 15 | POL | 746 | YTSFPWLLGCA | 11 | | 2531 |
| 90 | 18 | POL | 768 | YVPSALNPA | 9 | | 2532 |

TABLE XVII
A11 Motif With Binding Information

| | | | | | | | |
|---|---|---|---|---|---|---|---|
| Conservancy | Frequency | Protein | Position | Sequence | AA | A*1101 | SeqID Num |

| | | | | | | |
|---|---|---|---|---|---|---|
| 85 | 17 | POL | 721 | AACFARSR | 8 | 2533 |
| 95 | 19 | POL | 632 | AAPFTQCGY | 9 | 2534 |
| 90 | 18 | | 776 | ADDPSRGR | 8 | 2535 |
| 95 | 19 | POL | 529 | AFPHCLAFSY | 10 | 2536 |
| 90 | 19 | X | 62 | AFSSAGPCALR | 11 | 2537 |
| 95 | 19 | POL | 655 | AFTFSPTY | 8 | 2538 |
| 95 | 19 | POL | 655 | AFTFSPTYK | 9 | 2539 |
| 80 | 16 | ENV | 180 | AGFFLLTR | 8 | 2540 |
| 95 | 19 | POL | 18 | AGPLEEELPR | 10 | 2541 |
| 95 | 19 | POL | 521 | AICSVVRR | 8 | 2542 |
| 95 | 19 | NUC | 41 | ALESPEHCSPH | 11 | 2543 |
| 90 | 18 | POL | 772 | ALNPADDPSR | 10 | 2544 |
| 85 | 17 | X | 70 | ALRFTSAR | 8 | 2545 |
| 80 | 16 | ENV | 108 | AMQWNSTTFH | 10 | 2546 |
| 80 | 8 | POL | 166 | ASFCGSPY | 8 | 2547 |
| 80 | 16 | POL | 822 | ASPLHVAWR | 9 | 2548 |
| 75 | 15 | ENV | 84 | ASTNRQSGR | 9 | 2549 |
| 80 | 16 | POL | 755 | CAANWILR | 8 | 2550 |
| 85 | 17 | X | 69 | CALRFTSAR | 9 | 2551 |
| 80 | 16 | X | 6 | CCQLDPAR | 8 | 2552 |
| 75 | 15 | POL | 607 | CFRKLPVNR | 9 | 2553 |
| 95 | 19 | POL | 638 | CGYPALMPLY | 10 | 2554 |
| 95 | 19 | ENV | 253 | CLIFLLVLLDY | 11 | 2555 |
| 90 | 18 | X | 17 | CLRPVGAESR | 10 | 2556 |
| 100 | 20 | NUC | 48 | CSPHHTALR | 9 | 2557 |
| 95 | 19 | POL | 523 | CSVVRRAFPH | 10 | 2558 |
| 90 | 18 | POL | 540 | DDVVLGAK | 8 | 2559 |
| 85 | 17 | NUC | 29 | DLLDTASALY | 10 | 2560 |
| 85 | 17 | NUC | 29 | DLLDTASALYR | 11 | 2561 |
| 90 | 18 | POL | 737 | DNSVVLSR | 8 | 2562 |
| 90 | 18 | POL | 737 | DNSVVLSRK | 9 | 2562 |
| 90 | 18 | POL | 737 | DNSVVLSRKY | 10 | 2533 |
| 85 | 17 | NUC | 32 | DTASALYR | 8 | 2534 |
| 95 | 19 | POL | 418 | DVSAAFYH | 8 | 2535 |
| 90 | 18 | POL | 541 | DVVLGAKSVQH | 11 | 2536 |
| 95 | 19 | POL | 17 | EAGPLEEELPR | 11 | 2537 |
| 90 | 18 | NUC | 40 | EALESPEH | 8 | 2538 |
| 90 | 18 | POL | 718 | ELLAACFAR | 9 | 2539 |
| 85 | 17 | POL | 718 | ELLAACFARSR | 11 | 2540 |
| 95 | 19 | NUC | 43 | ESPEHCSPH | 9 | 2541 |
| 95 | 19 | NUC | 43 | ESPEHCSPHH | 10 | 2542 |
| 95 | 19 | NUC | 174 | ETTVVRRR | 8 | 2543 |
| 80 | 16 | NUC | 174 | ETTVVRRRGR | 10 | 2544 |
| 95 | 19 | POL | 631 | FAAPFTQCGY | 10 | 2545 |
| 80 | 16 | POL | 821 | FASPLHVAWR | 10 | 2546 |
| 75 | 15 | NUC | 139 | FGRETVLEY | 9 | 2547 |
| 75 | 15 | POL | 244 | FGVEPSGSGH | 10 | 2548 |
| 95 | 19 | NUC | 122 | FGVWIRTPPAY | 11 | 2549 |
| 95 | 19 | POL | 562 | FLLSLGIH | 8 | 2550 |
| 95 | 19 | ENV | 256 | FLLVLLDY | 8 | 2551 |
| 100 | 20 | POL | 363 | FLVDKVPH | 8 | 2552 |
| 90 | 18 | X | 63 | FSSAGPCALR | 10 | 2553 |
| 95 | 19 | POL | 656 | FTFSPTYK | 8 | 2554 |
| 95 | 19 | POL | 518 | FTSAICSVVR | 10 | 2555 |
| 95 | 19 | POL | 518 | FTSAICSVVRR | 11 | 2556 |
| 95 | 19 | X | 132 | FVLGGCRH | 8 | 2557 |
| 90 | 18 | X | 132 | FVLGGCRHK | 9 | 2558 |
| 80 | 16 | POL | 754 | GCAANWILR | 9 | 2559 |
| 95 | 19 | POL | 630 | GFAAPFTQCGY | 11 | 2560 |
| 100 | 20 | POL | 360 | GGVFLVDK | 8 | 2561 |
| 100 | 20 | POL | 360 | GGVFLVDKNPH | 11 | 2562 |
| 75 | 15 | POL | 567 | GIHLNPNK | 8 | 2563 |
| 75 | 15 | POL | 567 | GIHLNPNKTK | 10 | 2564 |
| 75 | 15 | POL | 567 | GIHLNPNKTKR | 11 | 2565 |
| 85 | 17 | NUC | 29 | GMDIDPYK | 8 | 2566 |
| 95 | 19 | POL | 44 | GNLNVSIPWTH | 11 | 2567 |
| 90 | 18 | POL | 735 | GTDNSVVLSR | 10 | 2568 |
| 90 | 18 | POL | 735 | GTDNSVVLSRK | 11 | 2569 |
| 80 | 16 | POL | 245 | GVEPSGSGH | 9 | 2570 |
| 100 | 20 | POL | 361 | GVFLVDKNPH | 10 | 2571 |
| 95 | 19 | NUC | 123 | GVWIRTPPAY | 10 | 2572 |
| 95 | 19 | NUC | 123 | GVWIRTPPAYR | 11 | 2573 |
| 100 | 20 | NUC | 47 | HCSPHHTALR | 10 | 2574 |
| 80 | 16 | POL | 820 | HFASPLHVAWR | 11 | 2575 |
| 95 | 19 | X | 49 | HGAHLSLR | 8 | 2576 |
| 90 | 18 | NUC | 104 | HISCLTFGR | 9 | 2577 |
| 75 | 15 | POL | 569 | HLNPNKTK | 8 | 2578 |
| 75 | 15 | POL | 569 | HLNPNKTKR | 9 | 2579 |
| 100 | 20 | POL | 149 | HTLWKAGILY | 10 | 2580 |
| 100 | 20 | POL | 149 | HTLWKAGILYK | 11 | 2581 |
| 95 | 19 | POL | 522 | ICSVVRRAFPH | 11 | 2582 |
| 90 | 18 | POL | 617 | IDWKVCQR | 8 | 2583 |
| 100 | 20 | ENV | 381 | IFFCLWVY | 8 | 2584 |
| 95 | 19 | ENV | 255 | IFLLVLLDY | 9 | 2585 |
| 80 | 16 | POL | 734 | IGTDNSVVLSR | 11 | 2586 |
| 80 | 16 | POL | 760 | ILRGTSFVY | 9 | 2587 |
| 90 | 18 | NUC | 105 | ISCLTFGR | 8 | 2588 |
| 100 | 20 | POL | 153 | KAGILYKR | 8 | 2589 |
| 75 | 15 | POL | 108 | KLIMPARFY | 9 | 2590 |
| 80 | 16 | POL | 610 | KLPVNRPIDWK | 11 | 2591 |
| 75 | 15 | X | 130 | KVFVLGGCR | 9 | 2592 |
| 75 | 15 | X | 130 | KVFVLGGCRH | 10 | 2593 |
| 95 | 19 | POL | 55 | KVGNFTGLY | 9 | 2594 |
| 85 | 17 | POL | 720 | LAACFARSR | 9 | 2595 |
| 90 | 18 | X | 16 | LCLRPVGAESR | 11 | 2596 |
| 100 | 20 | POL | 125 | LDKGIKPY | 8 | 2597 |
| 100 | 20 | POL | 125 | LDKGIKPYY | 9 | 2598 |
| 80 | 16 | x | 9 | LDPARDVLCLR | 11 | 2599 |
| 85 | 17 | NUC | 31 | LDTASALY | 8 | 2600 |
| 85 | 17 | NUC | 31 | LDTASALYR | 9 | 2601 |
| 95 | 19 | POL | 417 | LDVSAAFY | 8 | 2602 |
| 95 | 19 | POL | 417 | LDVSAAFYH | 9 | 2603 |
| 95 | 19 | POL | 544 | LGAKSVQH | 8 | 2604 |
| 80 | 16 | POL | 753 | LGCAANWILR | 10 | 2605 |
| 75 | 15 | POL | 566 | LGIHLNPNK | 9 | 2606 |
| 75 | 15 | POL | 566 | LGIHLNPNKTK | 11 | 2607 |
| 95 | 19 | ENV | 254 | LIFLLVLLDY | 10 | 2608 |
| 100 | 20 | POL | 109 | LIMPARFY | 8 | 2609 |
| 90 | 18 | POL | 719 | LLAACFAR | 8 | 2610 |
| 85 | 17 | POL | 719 | LLAACFARSR | 10 | 2611 |
| 85 | 17 | NUC | 30 | LLDTASALY | 9 | 2612 |
| 85 | 17 | NUC | 30 | LLDTASALYR | 10 | 2613 |
| 80 | 16 | POL | 752 | LLGCAANWILR | 11 | 2614 |
| 100 | 20 | ENV | 378 | LLPIFFCLWVY | 11 | 2615 |
| 90 | 18 | POL | 773 | LNPADDPSR | 9 | 2616 |
| 90 | 18 | POL | 773 | LNPADDPSRGR | 11 | 2617 |
| 75 | 15 | POL | 570 | LNPNKTKA | 8 | 2618 |
| 75 | 15 | POL | 570 | LNPNKTKRWGY | 11 | 2619 |
| 95 | 19 | POL | 46 | LNVSIPWTH | 11 | 2620 |
| 95 | 19 | POL | 46 | LNVSIPWTHK | 10 | 2621 |
| 95 | 19 | POL | 415 | LSLDVSAAFY | 10 | 2622 |
| 95 | 19 | POL | 415 | LSLDVSAAFYH | 11 | 2623 |
| 75 | 15 | POL | 564 | LSLGIHLNPNK | 11 | 2624 |
| 95 | 19 | NUC | 169 | LSTLPETTVVR | 11 | 2625 |
| 75 | 15 | POL | 3 | LSYQHFRK | 8 | 2626 |
| 75 | 15 | NUC | 137 | LTFGRETVLEY | 11 | 2627 |
| 85 | 17 | POL | 99 | LTVNEKRR | 8 | 2628 |
| 90 | 18 | NUC | 119 | LVSFGVWIR | 9 | 2629 |
| 100 | 20 | POL | 377 | LVVDFSQFSR | 10 | 2630 |
| 90 | 18 | POL | 539 | MDDVVLGAK | 9 | 2631 |
| 85 | 17 | ENV | 360 | MMWYWGPSLY | 10 | 2632 |
| 75 | 15 | X | 103 | MSTTDLEAY | 9 | 2633 |
| 75 | 15 | X | 103 | MSTTDLEAYFK | 11 | 2634 |
| 95 | 19 | POL | 561 | NFLLSLGIH | 9 | 2635 |
| 90 | 18 | NUC | 75 | NLEDPASR | 8 | 2636 |
| 95 | 19 | POL | 45 | NLNVSIPWTH | 10 | 2637 |
| 95 | 19 | POL | 45 | NLNVSIPWTHK | 11 | 2638 |
| 75 | 15 | ENV | 215 | NSQSPTSNH | 9 | 2639 |
| 90 | 18 | POL | 738 | NSVVLSRK | 8 | 2640 |
| 90 | 18 | POL | 738 | NSVVLSRKY | 9 | 2641 |
| 100 | 20 | POL | 47 | NVSIPWTH | 8 | 2642 |
| 100 | 20 | POL | 47 | NVSIPWTHK | 9 | 2643 |
| 90 | 18 | POL | 775 | PADDPSRGR | 9 | 2644 |
| 80 | 16 | X | 11 | PARDVLCLR | 9 | 2645 |
| 75 | 15 | ENV | 83 | PASTNRQSGR | 10 | 2646 |
| 85 | 17 | X | 68 | PCALRFTSAR | 10 | 2647 |
| 100 | 20 | ENV | 233 | PGYRWMCLR | 9 | 2648 |
| 95 | 19 | ENV | 233 | PGYRWMCLRR | 10 | 2649 |
| 90 | 18 | POL | 616 | PIDWKVCQR | 9 | 2650 |
| 100 | 20 | ENV | 380 | PIFFCLWVY | 9 | 2651 |
| 80 | 16 | POL | 496 | PIILGFRK | 8 | 2652 |
| 100 | 20 | POL | 124 | PLDKGIKPY | 9 | 2653 |
| 100 | 20 | POL | 124 | PLDKGIKPYY | 10 | 2654 |
| 95 | 19 | POL | 20 | PLEEELPR | 8 | 2655 |
| 95 | 19 | POL | 10 | PLGFFPDH | 8 | 2656 |
| 100 | 20 | POL | 427 | PLHPAAMPH | 9 | 2657 |
| 100 | 20 | POL | 2 | PLSYQHFR | 8 | 2658 |
| 75 | 15 | POL | 2 | PLSYQHFRK | 9 | 2659 |
| 85 | 17 | POL | 98 | PLTVNEKR | 8 | 2660 |
| 85 | 17 | POL | 98 | PLTVNEKRR | 9 | 2661 |
| 75 | 15 | POL | 572 | PNKTKRWGY | 9 | 2662 |
| 85 | 17 | POL | 797 | PTTGRTSLY | 9 | 2663 |
| 90 | 18 | X | 20 | PVGAESRGR | 9 | 2664 |
| 85 | 17 | POL | 612 | PVNRPIDWK | 9 | 2665 |
| 95 | 19 | POL | 654 | QAFTFSPTY | 9 | 2666 |
| 95 | 19 | POL | 654 | QAFTFSPTYK | 10 | 2667 |
| 80 | 16 | ENV | 179 | QAGFFLLTR | 9 | 2668 |
| 80 | 16 | ENV | 107 | QAMQMNSTTFH | 10 | 2669 |
| 95 | 19 | POL | 637 | QCGYPALMPLY | 11 | 2670 |
| 95 | 19 | POL | 517 | QFTSAICSVVR | 11 | 2671 |
| 75 | 15 | NUC | 169 | QSPRRRRSQSR | 11 | 2672 |
| 80 | 16 | POL | 189 | QSSGILSR | 8 | 2673 |
| 95 | 19 | POL | 528 | RAFPHCLAFSY | 11 | 2674 |
| 85 | 17 | NUC | 28 | RDLLDTASALY | 11 | 2675 |
| 100 | 20 | NUC | 152 | RGRSPRRR | 8 | 2676 |
| 75 | 15 | POL | 108 | RLKLIMPAR | 9 | 2677 |
| 75 | 15 | POL | 106 | RIKLIMPARFY | 11 | 2678 |
| 75 | 15 | X | 128 | RLKVFVLGGCR | 11 | 2679 |
| 95 | 19 | POL | 376 | RLVVDFSQFSR | 11 | 2680 |
| 95 | 19 | NUC | 183 | RSPRRRTPSPR | 11 | 2681 |
| 75 | 15 | NUC | 167 | RSQSPRRR | 8 | 2682 |
| 75 | 15 | NUC | 167 | RSQSPRRRR | 9 | 2683 |
| 95 | 19 | NUC | 188 | RTPSPRRR | 8 | 2684 |
| 95 | 19 | NUC | 188 | RTPSPRRRR | 9 | 2685 |
| 80 | 16 | POL | 818 | RVHFASPLH | 9 | 2686 |
| 100 | 20 | POL | 357 | RVTGGVFLVDK | 11 | 2687 |
| 90 | 18 | X | 65 | SAGPCALR | 8 | 2688 |
| 95 | 19 | POL | 520 | SAICSVVR | 8 | 2689 |
| 95 | 19 | POL | 520 | SAICSVVRR | 9 | 2690 |
| 90 | 18 | POL | 771 | SALNPADDPSR | 11 | 2691 |
| 100 | 20 | POL | 165 | SASFCGSPY | 9 | 2692 |
| 95 | 19 | POL | 416 | SLDVSAAFY | 9 | 2693 |
| 95 | 19 | POL | 416 | SLDVSAAFYH | 10 | 2694 |
| 75 | 15 | POL | 565 | SLGIHLNPNK | 10 | 2695 |
| 90 | 18 | X | 64 | SSAGPCALR | 9 | 2696 |
| 95 | 19 | NUC | 170 | STLPETTVVR | 10 | 2697 |
| 95 | 19 | NUC | 170 | STLPETTVVRR | 11 | 2698 |
| 80 | 16 | ENV | 85 | STNRQSGR | 8 | 2699 |
| 75 | 15 | X | 104 | STTDLEAY | 8 | 2700 |
| 75 | 15 | X | 104 | STTDLEAYFK | 10 | 2701 |
| 90 | 18 | POL | 739 | SVVLSRKY | 8 | 2702 |
| 95 | 19 | POL | 524 | SVVRRAFPH | 9 | 2703 |
| 85 | 17 | POL | 716 | TAELLAACFAR | 11 | 2704 |
| 80 | 16 | X | 106 | TDLEAYFK | 8 | 2705 |
| 90 | 18 | POL | 736 | TDNSVVLSR | 9 | 2706 |
| 90 | 18 | POL | 736 | TDNSVVLSRK | 10 | 2707 |
| 90 | 18 | POL | 736 | TDNSVVLSRKY | 11 | 2708 |
| 75 | 15 | NUC | 138 | TFGRETVLEY | 10 | 2709 |
| 100 | 20 | POL | 359 | TGGVFLVDK | 9 | 2710 |
| 95 | 19 | NUC | 171 | TLPETTVVR | 9 | 2711 |
| 95 | 19 | NUC | 171 | TLPETTVVRR | 10 | 2712 |
| 95 | 19 | NUC | 171 | TLPETTVVRRR | 11 | 2713 |
| 100 | 20 | POL | 150 | TLWKAGILY | 9 | 2714 |
| 100 | 20 | POL | 150 | TLWKAGILYK | 10 | 2715 |
| 100 | 20 | POL | 150 | TLWKAGILYKR | 11 | 2716 |
| 95 | 19 | POL | 560 | TNFLLSLGIH | 10 | 2717 |
| 95 | 19 | POL | 519 | TSAICSVVR | 9 | 2718 |
| 95 | 19 | POL | 519 | TSAICSVVRR | 10 | 2719 |
| 75 | 15 | X | 105 | TTDLEAYFK | 9 | 2720 |
| 85 | 17 | POL | 798 | TTGRTSLY | 8 | 2721 |
| 75 | 15 | ENV | 278 | TTSTGPCK | 8 | 2722 |
| 80 | 16 | NUC | 175 | TTVVRRRGR | 9 | 2723 |
| 80 | 16 | NUC | 176 | TVVRRRGR | 8 | 2724 |
| 80 | 16 | NUC | 176 | TVVRRRGRSPR | 11 | 2725 |
| 100 | 20 | POL | 379 | VDFSQFSR | 8 | 2726 |
| 100 | 20 | POL | 362 | VFLVDKNPH | 9 | 2727 |
| 80 | 16 | X | 131 | VFVLGGCR | 8 | 2728 |
| 80 | 16 | X | 131 | VFVLGGCRH | 9 | 2729 |
| 75 | 15 | X | 131 | VFVLGGCRHK | 10 | 2730 |
| 95 | 19 | X | 21 | VGAESRGR | 8 | 2731 |
| 95 | 19 | POL | 56 | VGNFTGLY | 8 | 2732 |
| 85 | 17 | POL | 96 | VGPLTVNEK | 9 | 2733 |
| 85 | 17 | POL | 96 | VGPLTVNEKR | 10 | 2734 |
| 85 | 17 | POL | 96 | VGPLTVNEKRR | 11 | 2735 |
| 95 | 19 | POL | 543 | VLGAKSVQH | 9 | 2736 |
| 90 | 18 | X | 133 | VLGGCRHK | 8 | 2737 |
| 80 | 16 | ENV | 177 | VLQAGFFLLTR | 11 | 2738 |
| 85 | 17 | POL | 613 | VNRPIDWK | 8 | 2739 |
| 90 | 18 | NUC | 120 | VSFGVWIR | 8 | 2740 |
| 100 | 20 | POL | 48 | VSIPWTHK | 8 | 2741 |
| 100 | 20 | POL | 358 | VTGGVFLVDK | 10 | 2742 |
| 100 | 20 | POL | 378 | VVDFSQFSR | 9 | 2743 |
| 90 | 18 | POL | 542 | VVLGAKSVQH | 10 | 2744 |
| 95 | 19 | POL | 525 | VVRRAFPH | 8 | 2745 |
| 80 | 16 | NUC | 177 | VVRRRGRSPR | 10 | 2746 |
| 80 | 16 | NUC | 177 | VVRRRGRSPRR | 11 | 2747 |
| 90 | 18 | NUC | 102 | WFHISCLTFGR | 11 | 2748 |
| 85 | 17 | NUC | 28 | WGMDIDPY | 8 | 2749 |
| 85 | 17 | NUC | 28 | WGMDIDPYK | 9 | 2750 |
| 85 | 17 | POL | 578 | WGYSLNFMGY | 10 | 2751 |
| 80 | 16 | POL | 759 | WILRGTSFVY | 10 | 2752 |
| 95 | 19 | NUC | 125 | WIRTPPAY | 8 | 2753 |
| 95 | 19 | NUC | 125 | WIRTPPAYR | 9 | 2754 |
| 90 | 18 | POL | 314 | WLQFRNSK | 8 | 2755 |
| 95 | 19 | POL | 414 | WLSLDVSAAFY | 11 | 2756 |
| 85 | 17 | NUC | 26 | WLWGMDIDPY | 10 | 2757 |
| 85 | 17 | NUC | 26 | WLWGMDIDPYK | 11 | 2758 |
| 85 | 17 | ENV | 359 | WMMWYWGPSLY | 11 | 2759 |
| 100 | 20 | POL | 122 | YLPLDKGIK | 9 | 2760 |
| 100 | 20 | POL | 122 | YLPLDKGIKPY | 11 | 2761 |
| 90 | 18 | NUC | 118 | YLVSFGVWIR | 10 | 2762 |
| 90 | 18 | POL | 538 | YMDDVVLGAK | 10 | 2763 |
| 80 | 16 | POL | 493 | YSHPIILGFR | 10 | 2764 |
| 80 | 16 | POL | 493 | YSHPIILGFRK | 11 | 2765 |
| 85 | 17 | POL | 580 | YSLNFMGY | 8 | 2766 |

TABLE XVIII
HBV A24 Motif With Binding Information

| | | | | | | | | |
|---|---|---|---|---|---|---|---|---|
| | | | | | SEQ ID | | | |
| Conservancy | Freq. | Protein | Position | Sequence | NO: | AA | Filed | A*2401 |

| | | | | | | | | |
|---|---|---|---|---|---|---|---|---|
| 95 | 19 | POL | 529 | AFPHCLAF | 2767 | 8 | | |
| 95 | 19 | X | 62 | AFSSAGPCAL | 2768 | 10 | | 0.0012 |
| 90 | 18 | POL | 535 | AFSYMDDVVL | 2769 | 10 | | 0.0009 |
| 95 | 19 | POL | 655 | AFTFSPTYKAF | 2770 | 11 | | |
| 80 | 16 | ENV | 108 | AMQWNSTTF | 2771 | 9 | | |
| 100 | 20 | NUC | 131 | AYRPPNAPI | 2772 | 9 | * | 0.0310 |
| 100 | 20 | NUC | 131 | AYRPPNAPIL | 2773 | 10 | * | 0.0042 |
| 75 | 15 | POL | 607 | CFRKLPVNRPI | 2774 | 11 | | |
| 85 | 17 | POL | 618 | DWKVCQRI | 2775 | 8 | | |
| 85 | 17 | POL | 618 | DWKVCQRIVGL | 2776 | 11 | | |
| 90 | 18 | ENV | 262 | DYQGMLPVCPL | 2777 | 11 | | 0.0002 |
| 90 | 18 | NUC | 117 | EYLVSFGVW | 2778 | 9 | | |
| 90 | 18 | NUC | 117 | EYLVSFGVWI | 2779 | 10 | * | |
| 100 | 20 | ENV | 382 | FFCLWVYI | 2780 | 8 | | |
| 80 | 16 | ENV | 182 | FFLLTRIL | 2781 | 8 | | |
| 80 | 16 | ENV | 182 | FFLLTRILTI | 2782 | 10 | | |
| 85 | 17 | ENV | 13 | FFPDHQLDPAF | 2783 | 11 | | |
| 80 | 16 | ENV | 181 | GFFLLTRI | 2784 | 8 | | |
| 80 | 16 | ENV | 181 | GFFLLTRIL | 2785 | 9 | | |
| 80 | 16 | ENV | 181 | GFFLLTRILTI | 2786 | 11 | | |
| 95 | 19 | ENV | 12 | GFFPDHQL | 2787 | 8 | | |
| 75 | 15 | ENV | 170 | GFLGPLLVL | 2788 | 9 | | |
| 80 | 16 | POL | 500 | GFRKIPMGVGL | 2789 | 11 | | |
| 85 | 17 | NUC | 29 | GMDIDPYKEF | 2790 | 10 | | |
| 90 | 18 | ENV | 265 | GMLPVCPL | 2791 | 8 | | |
| 85 | 17 | NUC | 25 | GWLWGMDI | 2792 | 8 | | |
| 85 | 17 | ENV | 65 | GWSPQAQGI | 2793 | 9 | | 0.0024 |
| 85 | 17 | ENV | 65 | GWSPQAQGIL | 2794 | 10 | | 0.0003 |
| 95 | 19 | POL | 639 | GYPALMPL | 2795 | 8 | | |
| 95 | 19 | ENV | 234 | GYRWMCLRRF | 2796 | 10 | * | 0.0007 |
| 95 | 19 | ENV | 234 | GYRWMCLRRFI | 2797 | 11 | | |
| 75 | 15 | POL | 579 | GYSLNFMGYVI | 2798 | 11 | | |
| 80 | 16 | POL | 820 | HFASPLHVAW | 2799 | 10 | | |
| 75 | 15 | POL | 7 | HFRKLLLL | 2800 | 8 | | |
| 100 | 20 | POL | 146 | HYLHTLWKAGI | 2801 | 11 | | |
| 100 | 20 | ENV | 381 | IFFCLWVYI | 2802 | 9 | | 0.0087 |
| 80 | 16 | ENV | 245 | IFLFILLL | 2803 | 8 | | |
| 80 | 16 | ENV | 245 | IFLFILLLCL | 2804 | 10 | | |
| 80 | 16 | ENV | 245 | IFLFILLLCLI | 2805 | 11 | | |
| 85 | 17 | ENV | 358 | IWMMWYWGPSL | 2806 | 11 | | 0.0004 |
| 95 | 19 | POL | 395 | KFAVPNLQSL | 2807 | 10 | | 0.0020 |
| 100 | 20 | POL | 121 | KYLPLDKGI | 2808 | 9 | | |
| 85 | 17 | POL | 745 | KYTSFPWL | 2809 | 8 | | |
| 85 | 17 | POL | 745 | KYTSFPWLL | 2810 | 9 | * | 5.3000 |
| 80 | 16 | ENV | 247 | LFILLLCL | 2811 | 8 | | |
| 80 | 16 | ENV | 247 | LFILLLCLI | 2812 | 9 | | |
| 80 | 16 | ENV | 247 | LFILLLCLIF | 2813 | 10 | | |
| 80 | 16 | ENV | 247 | LFILLLCLIFL | 2814 | 11 | | |
| 95 | 19 | POL | 643 | LMPLYACI | 2815 | 8 | | |
| 90 | 18 | NUC | 101 | LWFHISCL | 2816 | 8 | | |
| 85 | 17 | NUC | 101 | LWFHISCLTF | 2817 | 10 | | |
| 80 | 16 | POL | 492 | LYSHPIIL | 2818 | 8 | | |
| 80 | 16 | POL | 492 | LYSHPIILGF | 2819 | 10 | * | 1.1000 |
| 85 | 17 | ENV | 360 | MMWYWGPSL | 2820 | 9 | * | 0.0060 |
| 85 | 17 | ENV | 361 | MWYWGPSL | 2821 | 8 | | 0.0005 |
| 95 | 19 | POL | 561 | NFLLSLGI | 2822 | 8 | | |
| 95 | 19 | POL | 561 | NFLLSLGIHL | 2823 | 10 | | 0.0099 |
| 80 | 16 | POL | 758 | NWILRGTSF | 2824 | 9 | | |
| 95 | 19 | POL | 512 | PFLLAQFTSAI | 2825 | 11 | | |
| 95 | 19 | POL | 634 | PFTQCGYPAL | 2826 | 10 | | 0.0002 |
| 95 | 19 | ENV | 341 | PFVQWFVGL | 2827 | 9 | | 0.0003 |
| 80 | 16 | POL | 505 | PMGVGLSPF | 2828 | 9 | | |
| 80 | 16 | POL | 505 | PMGVGLSPFL | 2829 | 10 | | |
| 80 | 16 | POL | 505 | PMGVGLSPFLL | 2830 | 11 | | |
| 80 | 16 | POL | 750 | PWLLGCAANW | 2831 | 10 | | |
| 80 | 16 | POL | 750 | PWLLGCAANWI | 2832 | 11 | | |
| 100 | 20 | POL | 51 | PWTHKVGNF | 2833 | 9 | * | 0.0290 |
| 95 | 19 | ENV | 344 | QWFVGLSPTVW | 2834 | 11 | | |
| 75 | 15 | ENV | 242 | RFIIFLFI | 2835 | 8 | | |
| 75 | 15 | ENV | 242 | RFIIFLFIL | 2836 | 9 | | |
| 75 | 15 | ENV | 242 | RFIIFLFILL | 2837 | 10 | | |
| 75 | 15 | ENV | 242 | RFIIFLFILLL | 2838 | 11 | | |
| 100 | 20 | ENV | 332 | RFSWLSLL | 2839 | 8 | | |
| 100 | 20 | ENV | 332 | RFSWLSLLVPF | 2840 | 11 | | |
| 85 | 17 | POL | 577 | RWGYSLNF | 2841 | 8 | | |
| 95 | 19 | ENV | 236 | RWMCLRRF | 2842 | 8 | | |
| 95 | 19 | ENV | 236 | RWMCLRRFI | 2843 | 9 | * | 0.0710 |
| 95 | 19 | ENV | 236 | RWMCLRRFII | 2844 | 10 | * | 1.1000 |
| 95 | 19 | ENV | 236 | RWMCLRRFIIF | 2845 | 11 | | |
| 100 | 20 | POL | 167 | SFCGSPYSW | 2846 | 9 | * | 0.0710 |
| 95 | 19 | NUC | 46 | SFLPSDFF | 2847 | 8 | | |
| 80 | 16 | POL | 765 | SFVYVPSAL | 2848 | 9 | | |
| 95 | 19 | POL | 413 | SWLSLDVSAAF | 2849 | 11 | | |
| 100 | 20 | ENV | 334 | SWLSLLVPF | 2850 | 9 | * | 0.3900 |
| 95 | 19 | POL | 392 | SWPKFAVPNL | 2851 | 10 | * | 5.6000 |
| 100 | 20 | ENV | 197 | SWWTSLNF | 2852 | 8 | | |
| 95 | 19 | ENV | 197 | SWWTSLNFL | 2853 | 9 | * | 0.3800 |
| 90 | 18 | POL | 537 | SYMDDVVL | 2854 | 8 | | |
| 75 | 15 | POL | 4 | SYQHFRKL | 2855 | 8 | | |
| 75 | 15 | POL | 4 | SYQHFRKLL | 2856 | 9 | | 0.0051 |
| 75 | 15 | POL | 4 | SYQHFRKLLL | 2857 | 10 | * | 0.0660 |
| 75 | 15 | POL | 4 | SYQHFRKLLLL | 2858 | 11 | | |
| 75 | 15 | NUC | 138 | TFGRETVL | 2859 | 8 | | |
| 75 | 15 | NUC | 138 | TFGRETVLEYL | 2860 | 11 | | |
| 95 | 19 | POL | 657 | TFSPTYKAF | 2861 | 9 | | 0.0060 |
| 95 | 19 | POL | 657 | TFSPTYKAFL | 2862 | 10 | | 0.0043 |
| 95 | 19 | POL | 686 | VFADATPTGW | 2863 | 10 | * | 0.0180 |
| 75 | 15 | X | 131 | VFVLGGCRHKL | 2864 | 11 | | |
| 90 | 18 | NUC | 102 | WFHISCLTF | 2865 | 9 | * | 0.0300 |
| 95 | 19 | ENV | 345 | WFVGLSPTVW | 2866 | 10 | * | 0.0120 |
| 95 | 19 | ENV | 345 | WFVGLSPTVWL | 2867 | 11 | | |
| 95 | 19 | ENV | 237 | WMCLRRFI | 2868 | 8 | | |
| 95 | 19 | ENV | 237 | WMCLRRFII | 2869 | 9 | * | |
| 95 | 19 | ENV | 237 | WMCLRRFIIF | 2870 | 10 | | 0.0013 |
| 95 | 19 | ENV | 237 | WMCLRRFIIFL | 2871 | 11 | | |
| 85 | 17 | ENV | 359 | WMMWYWGPSL | 2872 | 10 | * | |
| 95 | 19 | ENV | 198 | WWTSLNFL | 2873 | 8 | | |

TABLE XIXa
HBV DR-SUPER MOTIF

| | | | | | | | | | |
|---|---|---|---|---|---|---|---|---|---|
| | | | | | | | Position | | Exemplary |
| | | | | Core | | | In HBV | Exemplary | Sequence |
| | Core SEQ | Core | Core | Conservancy | Exemplary | Exemplary | Poly- | Sequence | Conservancy |
| Protein | ID NO: | Sequence | Freq. | (%) | SEQ ID NO: | Sequence | Protein | Frequency | (%) |

| | | | | | | | | | |
|---|---|---|---|---|---|---|---|---|---|
| POL | 2874 | FAAPFTQCG | 19 | 95 | 3021 | LLGFAAPFTQCGYPA | 628 | 19 | 95 |
| POL | 2875 | FADATPTGW | 19 | 95 | 3022 | CQVFADATPTGWGLA | 684 | 16 | 80 |
| POL | 2876 | FAVPNLQSL | 19 | 95 | 3023 | WPKFAVPNLQSLTNL | 393 | 19 | 95 |
| NUC | 2877 | FGRETVLEY | 15 | 75 | 3024 | CLTFGRETVLEYLVS | 136 | 14 | 70 |
| POL | 2878 | FGVEPSGSG | 15 | 75 | 3025 | RRSFGVEPSGSGHID | 252 | 6 | 30 |
| NUC | 2879 | FHISCLTFG | 18 | 90 | 3026 | LLWFHISCLTFGRET | 100 | 17 | 85 |
| NUC | 2880 | FHLCLIISC | 16 | 80 | 3027 | MQLFHLCLIISCSCP | 1 | 10 | 50 |
| ENV | 2881 | FILLLCLIF | 16 | 80 | 3028 | IFLFILLLCLIFLLV | 245 | 16 | 80 |
| ENV | 2882 | FLFILLLCL | 16 | 80 | 3029 | FIIFLFILLLCLIFL | 243 | 16 | 80 |
| ENV | 2883 | FLGPLLVLQ | 15 | 75 | 3030 | TSGFLGPLLVLQAGF | 168 | 15 | 75 |
| ENV | 2884 | FLLTRILTI | 16 | 80 | 3031 | AGFFLLTRILTIPQS | 180 | 16 | 80 |
| ENV | 2885 | FLLVLLDYQ | 19 | 95 | 3032 | CLIFLLVLLDYQGML | 253 | 19 | 95 |
| ENV | 2886 | FPAGGSSSG | 15 | 75 | 3033 | GLYFPAGGSSSGTVN | 127 | 11 | 55 |
| ENV | 2887 | FPDHQLDPA | 18 | 90 | 3034 | LGFFPDHQLDPAFGA | 22 | 9 | 45 |
| POL | 2888 | FPHCLAFSY | 19 | 95 | 3035 | RRAFPHCLAFSYMDD | 527 | 19 | 95 |
| POL | 2889 | FRKIPMGVG | 16 | 80 | 3036 | ILGFRKIPMGVGLSP | 498 | 13 | 65 |
| POL | 2890 | FRKLPVNRP | 16 | 80 | 3037 | KQCFRKLPVNRPIDW | 616 | 9 | 45 |
| X | 2891 | FSSAGPCAL | 19 | 95 | 3038 | VCAFSSAGPCALRFT | 60 | 18 | 90 |
| ENV | 2892 | FSWLSLLVP | 20 | 100 | 3039 | SVRFSWLSLLVPFVQ | 330 | 16 | 80 |
| POL | 2893 | FTFSPTYKA | 19 | 95 | 3040 | KQAFTFSPTYKAFLC | 653 | 12 | 60 |
| POL | 2894 | FTGLYSSTV | 18 | 90 | 3041 | VGNFTGLYSSTVPVF | 56 | 11 | 55 |
| POL | 2895 | FTSAICSVV | 19 | 95 | 3042 | LAQFTSAICSVVRRA | 515 | 19 | 95 |
| ENV | 2896 | FVGLSPTVW | 19 | 95 | 3043 | VQWFVGLSPTVWLSV | 343 | 14 | 70 |
| X | 2897 | FVLGGCRHK | 18 | 90 | 3044 | LKVFVLGGCRHKLVC | 129 | 14 | 70 |
| ENV | 2898 | FVQWFVGLS | 19 | 95 | 3045 | LVPFVQWFVGLSPTV | 339 | 19 | 95 |
| POL | 2899 | FVYVPSALN | 18 | 90 | 3046 | GTSFVYVPSALNPAD | 763 | 16 | 80 |
| POL | 2900 | IDWKVCQRI | 17 | 85 | 3047 | NRPIDWKVCQRIVGL | 614 | 16 | 80 |
| ENV | 2901 | IFLFILLLC | 16 | 80 | 3048 | RFIIFLFILLLCLIF | 242 | 15 | 75 |
| ENV | 2902 | IFLLVLLDY | 19 | 95 | 3049 | LCLIFLLVLLDYQGM | 252 | 19 | 95 |
| POL | 2903 | IGTDNSVVL | 16 | 80 | 3050 | AKLIGTDNSVVLSRK | 731 | 13 | 65 |
| POL | 2904 | IHTAELLAA | 17 | 85 | 3051 | PLPIHTAELLAACFA | 711 | 16 | 80 |
| ENV | 2905 | IIFLFILLL | 16 | 80 | 3052 | RRFIIFLFILLLCLI | 241 | 15 | 75 |
| ENV | 2906 | ILLLCLIFL | 20 | 100 | 3053 | FLFILLLCLIFLLVL | 246 | 16 | 80 |
| POL | 2907 | ILRGTSFVY | 16 | 80 | 3054 | ANWILRGTSFVYVPS | 757 | 16 | 80 |
| NUC | 2908 | ILSTLPETT | 20 | 100 | 3055 | NAPILSTLPETTVVR | 165 | 19 | 95 |
| ENV | 2909 | IPIPSSWAF | 20 | 100 | 3056 | CTCIPIPSSWAFARF | 321 | 8 | 40 |
| NUC | 2910 | IRTPPAYRP | 19 | 95 | 3057 | GVWIRTPPAYRPPNA | 123 | 19 | 95 |
| POL | 2911 | LAACFARSR | 17 | 85 | 3058 | AELLAACFARSRSGA | 717 | 16 | 80 |
| POL | 2912 | LAFSYMDDV | 18 | 90 | 3059 | PHCLAFSYMDDVVLG | 531 | 18 | 90 |
| POL | 2913 | LAQFTSAIC | 19 | $$5 | 3060 | PFLLAQFTSAICSVV | 512 | 19 | 95 |
| NUC | 2914 | LCLGWLWGM | 17 | 85 | 3061 | ASKLCLGWLWGMDID | 19 | 17 | 85 |
| ENV | 2915 | LCLIFLLVL | 20 | 100 | 3062 | ILLLCLIFLLVLLDY | 249 | 19 | 95 |
| X | 2916 | LCLRPVGAE | 19 | 95 | 3063 | RDVLCLRPVGAESRG | 13 | 18 | 90 |
| POL | 2917 | LCQVFADAT | 19 | 95 | 3064 | RPGLCQVFADATPTG | 680 | 11 | 55 |
| ENV | 2918 | LDSWWTSLN | 19 | 95 | 3065 | PQSLDSWWTSLNFLG | 192 | 17 | 85 |
| NUC | 2919 | LDTASALYR | 17 | 85 | 3066 | RDLLDTASALYREAL | 28 | 16 | 80 |
| POL | 2920 | LDVSAAFYH | 19 | 95 | 3067 | WLSLDVSAAFYHIPL | 425 | 11 | 55 |
| ENV | 2921 | LDYQGMLPV | 18 | 90 | 3068 | LVLLDYQGMLPVCPL | 258 | 18 | 90 |
| POL | 2922 | LEEELPRLA | 18 | 90 | 3069 | AGPLEEELPRLADEG | 18 | 13 | 65 |
| ENV | 2923 | LFILLLCLI | 16 | 80 | 3070 | IIFLFILLLCLIFLL | 244 | 16 | 80 |
| POL | 2924 | LGAKSVQHL | 17 | 85 | 3071 | DVVLGAKSVQHLESL | 541 | 16 | 80 |
| POL | 2925 | LGFAAPFTQ | 19 | 95 | 3072 | VGLLGFAAPFTQCGY | 626 | 19 | 95 |
| POL | 2926 | LGFRKIPMG | 19 | 95 | 3073 | PIILGFRKIPMGVGL | 496 | 13 | 65 |
| POL | 2927 | LGNLNVSIP | 19 | 95 | 3074 | DUNLGNLNVSIPWTH | 40 | 19 | 95 |
| ENV | 2928 | LGPLLVLQA | 19 | 95 | 3075 | SGFLGPLLVLQAGFF | 169 | 15 | 75 |
| POL | 2929 | LHPAAMPHL | 20 | 100 | 3076 | HLPLHPAAMPHLLVG | 425 | 9 | 45 |
| ENV | 2930 | LIFLLVLLD | 19 | 95 | 3077 | LLCLIFLLVLLDYQG | 251 | 19 | 95 |
| POL | 2931 | LKLIMPARF | 15 | 75 | 3078 | KRRLKLIMPARFYPN | 104 | 7 | 35 |
| X | 2932 | LKVFVLGGC | 15 | 75 | 3079 | EIRLKVFVLGGCRHK | 126 | 13 | 65 |
| POL | 2933 | LLAQFTSAI | 19 | 95 | 3080 | SPFLLAQFTSAICSV | 511 | 19 | 95 |
| NUC | 2934 | LLDTASALY | 17 | 85 | 3081 | IRDLLDTASALYREA | 56 | 9 | 45 |
| POL | 2935 | LLGCAANWI | 16 | 80 | 3082 | FPWLLGCAANWILRG | 749 | 15 | 75 |
| POL | 2936 | LLGFAAPFT | 19 | 95 | 3083 | IVGLLGFAAPFTQCG | 625 | 18 | 90 |
| ENV | 2937 | LLGWSPQAQ | 17 | 85 | 3084 | HGGLLGWSPQAQGIL | 60 | 15 | 75 |
| ENV | 2938 | LLLCLIFLL | 20 | 100 | 3085 | LFILLLCLIFLLVLL | 247 | 16 | 80 |
| NUC | 2939 | LLSFLPSDF | 19 | 95 | 3086 | SVELLSFLPSDFFPS | 41 | 11 | 55 |
| POL | 2940 | LLSLGIHLN | 19 | 95 | 3087 | TNFLLSLGIHLNPNK | 560 | 15 | 75 |
| POL | 2941 | LLSSNLSWL | 18 | 90 | 3088 | LTNLLSSNLSWLSLD | 404 | 18 | 90 |
| ENV | 2942 | LLTRILTIP | 16 | 80 | 3089 | GFFLLTRILTIPQSL | 181 | 16 | 80 |
| ENV | 2943 | LLVLQAGFF | 19 | 95 | 3090 | LGPLLVLQAGFFLLT | 172 | 18 | 90 |
| ENV | 2944 | LLVPFVQWF | 20 | 100 | 3091 | WLSLLVPFVQWFVGL | 335 | 19 | 95 |
| NUC | 2945 | LLWFHISCL | 18 | 90 | 3092 | IRQLLWFHISCLTFG | 126 | 13 | 65 |
| POL | 2946 | LMPLYACIQ | 19 | 95 | 3093 | YPALMPLYACIQSKQ | 640 | 11 | 55 |
| POL | 2947 | LNLGNLNVS | 19 | 95 | 3094 | AEDLNLGNLNVSIPW | 38 | 19 | 95 |
| POL | 2948 | LNPNKTKRW | 15 | 75 | 3095 | GIHLNPNKTKRWGYS | 567 | 15 | 75 |
| POL | 2949 | LNRRVAEDL | 17 | 85 | 3096 | DEGLNRRVAEDLNLG | 30 | 12 | 60 |
| POL | 2950 | LNVSIPWTH | 19 | 95 | 3097 | LGNLNVSIPWTHKVG | 43 | 19 | 95 |
| NUC | 2951 | LPETTVVRR | 19 | 95 | 3098 | LSTLPETTVVRRRGR | 169 | 16 | 80 |
| ENV | 2952 | LPIFFCLWV | 20 | 100 | 3099 | LPLLPIFFCLWVYIZ | 376 | 13 | 65 |
| POL | 2953 | LPIHTAELL | 17 | 85 | 3100 | VAPLPIHTAELLAAC | 709 | 9 | 45 |
| POL | 2954 | LPVNRPIDW | 16 | 80 | 3101 | FRKLPVNRPIDWKVC | 608 | 15 | 75 |
| POL | 2955 | LQFRNSKPC | 18 | 90 | 3102 | CWWLQFRNSKPCSDY | 312 | 10 | 50 |
| X | 2956 | LRGLPVCAF | 19 | 95 | 3103 | HLSLRGLPVCAFSSA | 52 | 18 | 90 |
| X | 2957 | LRPVGAESR | 18 | 90 | 3104 | VLCLRPVGAESRGRP | 15 | 18 | 90 |
| NUC | 2958 | LRQAILCWG | 18 | 90 | 3105 | HTALRQAILCWGELM | 52 | 18 | 90 |
| ENV | 2959 | LRRFIIFLF | 15 | 75 | 3106 | WMCLRRFIIFLFILL | 237 | 15 | 75 |
| NUC | 2960 | LSFLPSDFF | 19 | 95 | 3107 | VELLSFLPSDFFPSI | 42 | 10 | 50 |
| POL | 2961 | LSLDVSAAF | 19 | 95 | 3108 | LSWLSLDVSAAFYHI | 423 | 11 | 55 |
| ENV | 2962 | LSLLVPFVQ | 20 | 100 | 3109 | FSWLSLLVPFVQWFV | 333 | 19 | 95 |
| X | 2963 | LSLRGLPVC | 19 | 95 | 3110 | GAHLSLRGLPVCAFS | 50 | 18 | 90 |
| POL | 2964 | LSPFLLAQF | 19 | 95 | 3111 | GVGLSPFLLAQFTSA | 507 | 16 | 80 |
| POL | 2965 | LSRKYTSFP | 17 | 85 | 3112 | SVVLSRKYTSFPWLL | 739 | 17 | 85 |
| POL | 2966 | LSSNLSWLS | 18 | 90 | 3113 | TNLLSSNLSWLSLDV | 405 | 18 | 90 |
| ENV | 2967 | LSVPNPLGF | 15 | 75 | 3114 | GTNLSVPNPLGFFPD | 13 | 14 | 70 |
| POL | 2968 | LSWLSLDVS | 20 | 100 | 3115 | SSNLSWLSLDVSAAF | 409 | 17 | 85 |
| ENV | 2969 | LTIPQSLDS | 18 | 90 | 3116 | TRILTIPQSLDSWWT | 186 | 15 | 75 |
| POL | 2970 | LTNLLSSNL | 18 | 90 | 3117 | LQSLTNLLSSNLSWL | 401 | 18 | 90 |
| ENV | 2971 | LTRILTIPQ | 16 | 80 | 3118 | FFLLTRILTIPQSLD | 182 | 15 | 75 |
| POL | 2972 | LVDKNPHNT | 20 | 100 | 3119 | GVFLVDKNPHNTTES | 372 | 11 | 55 |
| NUC | 2973 | LVSFGVWIR | 18 | 90 | 3120 | LEYLVSFGVWIRTPP | 145 | 14 | 70 |
| POL | 2974 | LVVDFSQFS | 20 | 100 | 3121 | ESRLVVDFSQFSRGN | 374 | 9 | 45 |
| NUC | 2975 | LWFHISCLT | 17 | 85 | 3122 | RQLLWFHISCLTFGR | 98 | 17 | 85 |
| NUC | 2976 | LWGMDIDPY | 17 | 85 | 3123 | LGWLWGMDIDPYKEF | 24 | 17 | 85 |
| POL | 2977 | LWKAGILYK | 20 | 100 | 3124 | LHTLWKAGILYKRET | 148 | 18 | 90 |
| NUC | 2978 | LYREALESP | 17 | 85 | 3125 | ASALYREALESPEHC | 34 | 17 | 85 |
| POL | 2979 | LYSHPIILG | 16 | 80 | 3126 | KLHLYSHPIILGFRK | 489 | 16 | 80 |
| POL | 2980 | MDDVVLGAK | 18 | 90 | 3127 | FSYMDDVVLGAKSVQ | 536 | 18 | 90 |
| POL | 2981 | MGVGLSPFL | 16 | 80 | 3128 | KIPMGVGLSPFLLAQ | 503 | 16 | 80 |
| POL | 2982 | MPHLLVGSS | 17 | 85 | 3129 | PAAMPHLLVGSSGLS | 430 | 8 | 40 |
| ENV | 2983 | MQWNSTTFH | 16 | 80 | 3130 | PQAMQWNSTTFHQTL | 106 | 8 | 40 |
| X | 2984 | MSTTDLEAY | 15 | 75 | 3131 | LSAMSTTDLEAYFKD | 100 | 9 | 45 |
| ENV | 2985 | MWYWGPSLY | 17 | 85 | 3132 | IWMMWYWGPSLYNIL | 369 | 9 | 45 |
| X | 2986 | VCAFSSAGP | 19 | 95 | 3133 | GLPVCAFSSAGPCAL | 57 | 18 | 90 |
| POL | 2987 | VCQRIVGLL | 17 | 85 | 3134 | DWKVCQRIVGLLGFA | 618 | 17 | 85 |
| POL | 2988 | VFADATPTG | 19 | 95 | 3135 | LCQVFADATPTGWGL | 683 | 19 | 95 |
| ENV | 2989 | VGLSPTVWL | 19 | 95 | 3136 | QWFVGLSPTVWLSVI | 344 | 14 | 70 |
| POL | 2990 | VGPLTVNEK | 17 | 85 | 3137 | QQYVGPLTVNEKRRL | 93 | 8 | 40 |
| POL | 2991 | VHFASPLHV | 16 | 80 | 3138 | PDRVHFASPLHVAWR | 816 | 12 | 60 |
| X | 2992 | VLCLRPVGA | 19 | 95 | 3139 | ARDVLCLRPVGAESR | 12 | 14 | 70 |
| POL | 2993 | VLGAKSVQH | 19 | 95 | 3140 | DDVVLGAKSVQHLES | 540 | 16 | 80 |
| X | 2994 | VLHKRTLGL | 17 | 85 | 3141 | LPKVLHKRTLGLSAM | 89 | 11 | 55 |
| POL | 2995 | VPNLQSLTN | 19 | 95 | 3142 | KFAVPNLQSLTNLLS | 395 | 19 | 95 |
| NUC | 2996 | VQASKLCLG | 16 | 80 | 3143 | CPTVQASKLCLGWLW | 14 | 15 | 75 |
| ENV | 2997 | VRFSWLSLL | 16 | 80 | 3144 | WASVRFSWLSLLVPF | 328 | 13 | 65 |
| POL | 2998 | VRRAFPHCL | 19 | 95 | 3145 | CSVVRRAFPHCLAFS | 523 | 19 | 95 |
| POL | 2999 | VSIPWTHKV | 20 | 100 | 3146 | NLNVSIPWTHKVGNF | 45 | 19 | 95 |
| NUC | 3000 | VWIRTPPAY | 19 | 95 | 3147 | SFGVWIRTPPAYRPP | 121 | 18 | 90 |
| POL | 3001 | VYVPSALNP | 18 | 90 | 3148 | TSFVYVPSALNPADD | 764 | 16 | 80 |
| NUC | 3002 | WFHISCLTF | 18 | 90 | 3149 | QLLWFHISCLTFGRE | 99 | 17 | 85 |
| ENV | 3003 | WFVGLSPTV | 19 | 95 | 3150 | FVQWFVGLSPTVWLS | 342 | 19 | 95 |
| POL | 3004 | WILRGTSFV | 16 | 80 | 3151 | AANWILRGTSFVYVP | 756 | 14 | 70 |
| NUC | 3005 | WIRTPPAYR | 19 | 95 | 3152 | FGVWIRTPPAYRPPN | 122 | 19 | 95 |
| POL | 3006 | WKAGILYKR | 20 | 100 | 3153 | HTLWKAGILYKRETT | 149 | 18 | 90 |
| POL | 3007 | WLLGCAANW | 16 | 80 | 3154 | SFPWLLGCAANWILR | 748 | 15 | 75 |
| POL | 3008 | WLSLDVSAA | 19 | 95 | 3155 | NLSWLSLDVSAAFYH | 411 | 17 | 85 |
| ENV | 3009 | WLSLLVPFV | 20 | 100 | 3156 | RFSWLSLLVPFVQWF | 332 | 20 | 100 |
| POL | 3010 | WPKFAVPNL | 19 | 95 | 3157 | RVSWPKFAVPNLQSL | 390 | 11 | 55 |
| POL | 3011 | YMDDVVLGA | 18 | 90 | 3158 | AFSYMDDVVLGAKSV | 535 | 18 | 90 |
| POL | 3012 | YPALMPLYA | 19 | 95 | 3159 | QCGYPALMPLYACIQ | 637 | 19 | 95 |
| ENV | 3013 | YQGMLPVCP | 18 | 90 | 3160 | LLDYQGMLPVCPLIP | 260 | 10 | 50 |
| NUC | 3014 | YRPPNAPIL | 20 | 100 | 3161 | PPAYRPPNAPILSTL | 129 | 19 | 95 |
| ENV | 3015 | YRWMCLRRF | 19 | 95 | 3162 | CPGYRWMCLRRFIIF | 232 | 19 | 95 |
| POL | 3016 | YSHPIILGF | 16 | 80 | 3163 | LHLYSHPIILGFRKI | 490 | 16 | 80 |
| POL | 3017 | YSLNFMGYV | 15 | 75 | 3164 | RWGYSLNFMGYVIGS | 588 | 11 | 55 |
| POL | 3018 | YVPSALNPA | 18 | 90 | 3165 | SFVYVPSALNPADDP | 765 | 16 | 80 |
| ENV | 3019 | FFCLWVYIZ | 20 | | | | 382 | | |
| ENV | 3020 | MGTNLSVPN | 15 | | | | 12 | | |

TABLE XIXB
HBV DR-SUPER MOTIF With Binding Data

| Core | | | | | | | | | | | | | | | | |
|---|---|---|---|---|---|---|---|---|---|---|---|---|---|---|---|---|
| SEQ ID | | SEQ ID | | | | | | | | | | | | | | |
| NO: | Core Sequence | NO: | Exemplary Sequence | DR1 | DR2w2β1 | DR2w2β2 | DR3 | DR4w4 | DR4w15 | DR5w11 | DR5w12 | DR6w19 | DR7 | DR8W2 | DR9 | Drw53 |
| 2874 | FAAPFTQCG | 3021 | LLGFAAPFTQCGYPA | | | | | | | | | | | | | |
| 2875 | FADATPTGW | 3022 | CQVFADATPTGWGLA | | | | | | 0.2800 | | | | | | | |
| 2876 | FAVPNLQSL | 3023 | WPKFAVPNLQSLTNL | 0.0007 | | 0.0013 | | 0.0023 | | 0.0002 | | | 0.0008 | | | 0.0180 |
| 2877 | FGRETVLEY | 3024 | CLTFGRETVLEYLVS | | | | | | | | | | | | | |
| 2878 | FGVEPSGSG | 3025 | RRSFGVEPSGSGHID | | | | | | | | | | | | | |
| 2879 | FHISCLTFG | 3026 | LLWFHISCLTFGRET | | | | | | | | | | | | | |
| 2880 | FHLCLIISC | 3027 | MQLFHLCLIISCSCP | | | | | | | | | | | | | |
| 2881 | FILLLCLIF | 3028 | IFLFILLLCLIFLLV | 0.0005 | | | | 0.0041 | | | | | 0.0018 | | | |
| 2882 | FLFILLLCL | 3029 | FIIFLFILLLCLIFL | | | | | | | | | | | | | |
| 2883 | FLGPLLVLQ | 3030 | TSGFLGPLLVLQAGF | | | | | | | | | | | | | |
| 2884 | FLLTRILTI | 3031 | AGFFLLTRILTIPQS | 4.6000 | 0.0420 | 0.0190 | 0.0040 | 5.3000 | 0.1500 | 3.6000 | 0.0700 | 0.3700 | 3.1000 | 0.2600 | 1.3000 | |
| 2885 | FLLVLLDYQ | 3032 | CLIFLLVLLDYQGML | | | | | | | | | | | | | |
| 2886 | FPAGGSSSG | 3033 | GLYFPAGGSSSGTVN | | | | | | | | | | | | | |
| 2887 | FPDHQLDPA | 3034 | LGFFPDHQLDPAFGA | | | | | | | | | | | | | |
| 2888 | FPHCLAFSY | 3035 | RRAFPHCLAFSYMDD | 0.0010 | | 0.0010 | | −0.0009 | | 0.0010 | | | 0.0017 | | | |
| 2889 | FRKIPMGVG | 3036 | ILGFRKIPMGVGLSP | | | | | | | | | | | | | |
| 2890 | FRKLPVNRP | 3037 | KQCFRKLPVNRPIDW | 1.5000 | 0.0022 | 0.0210 | −0.0006 | 1.2000 | 0.8500 | 0.0130 | 0.0013 | 0.0043 | 0.4000 | 0.0580 | 0.0250 | |
| 2891 | FSSAGPCAL | 3038 | VCAFSSAGPCALRFT | 0.2100 | | 0.2600 | | 0.0023 | | 0.0003 | | | 0.0200 | | | 0.0150 |
| 2892 | FSWLSLLVP | 3039 | SVRFSWLSLLVPFVQ | 0.9000 | | | | 0.0099 | | | | | 0.0037 | | | |
| 2893 | FTFSPTYKA | 3040 | KQAFTFSPTYKAFLC | 0.5300 | 0.2400 | 0.1400 | 0.0090 | 1.1000 | 0.2200 | 0.2400 | 0.0024 | 0.0200 | 0.3300 | 0.1200 | 0.5400 | |
| 2894 | FTGLYSSTV | 3041 | VGNFTGLYSSTVPVF | 1.7000 | 0.0100 | 0.0016 | | 0.0140 | 0.1700 | 0.0035 | | 0.0580 | 0.5800 | 0.0044 | 0.3100 | |
| 2895 | FTSAICSVV | 3042 | LAQFTSAICSVVRRA | 0.0120 | 0.0065 | 0.1500 | −0.0009 | 0.0150 | 0.0280 | 0.0076 | 0.0091 | 0.0010 | 0.0280 | 0.0150 | 0.0880 | 0.0190 |
| 2896 | FVGLSPTVW | 3043 | VQWFVGLSPTVWLSV | | | | | | | | | | | | | |
| 2897 | FVLGGCRHK | 3044 | LKVFVLGGCRHKLVC | | | | | | | | | | | | | |
| 2898 | FVQWFVGLS | 3045 | LVPFVQWFVGLSPTV | 0.0130 | 0.6900 | 0.0140 | −0.0013 | 0.1500 | 1.4000 | 0.3800 | 0.6600 | 0.0018 | 0.0092 | 0.6600 | 2.5000 | 2.6000 |
| 2899 | FVYVPSALN | 3046 | GTSFVYVPSALNPAD | 0.3500 | 0.0140 | 0.0500 | −0.0006 | 0.3800 | 0.4100 | 0.0470 | −0.0001 | 0.0001 | 0.2700 | 0.0610 | 0.3400 | |
| 2900 | IDWKVCQRI | 3047 | NRPIDWKVCQRIVGL | | | | | | | | | | | | | |
| 2901 | IFLFILLLC | 3048 | RFIIFLFILLLCLIF | | | | | | | | | | | | | |
| 2902 | IFLLVLLDY | 3049 | LCLIFLLVLLDYQGM | 0.0016 | | 0.0060 | | 0.0230 | | 0.0017 | | | 0.0044 | | | |
| 2903 | IGTDNSVVL | 3050 | AKLIGTDNSVVLSRK | | | | | | | | | | | | | |
| 2904 | IHTAELLAA | 3051 | PLPIHTAELLAACFA | 0.0046 | | | | 0.0490 | | | | | −0.0003 | | | |
| 2905 | IIFLFILLL | 3052 | RRFIIFLFILLLCLI | | | | | | | | | | | | | |
| 2906 | ILLLCLIFL | 3053 | FLFILLLCLIFLLVL | | | | | | | | | | | | | |
| 2907 | ILRGTSFVY | 3054 | ANWILRGTSFVYVPS | | | | | | | | | | | | | |
| 2908 | ILSTLPETT | 3055 | NAPILSTLPETTVVR | 0.0009 | | 0.0009 | | −0.0007 | | −0.0002 | | | 0.0005 | | | 0.1600 |
| 2909 | IPIPSSWAF | 3056 | CTCIPIPSSWAFARF | | | | | | | | | | | | | |
| 2910 | IRTPPAYRP | 3057 | GVWIRTPPAYRPPNA | 0.3700 | 0.0420 | 7.2000 | 0.0120 | 3.4000 | 0.5700 | 0.4800 | 0.0140 | −0.0004 | 0.2200 | 0.5300 | 0.0450 | |
| 2911 | LAACFARSR | 3058 | AELLAACFARSRSGA | | | | | | | | | | | | | |
| 2912 | LAFSYMDDV | 3059 | PHCLAFSYMDDVVLG | | | | | | | | | | | | | |
| 2913 | LAQFTSAIC | 3060 | PFLLAQFTSAICSVV | 0.1800 | 0.0270 | 0.0042 | −0.0013 | 0.0800 | 0.1200 | 0.0120 | 0.0016 | 0.0800 | 0.0770 | 0.0580 | 0.0590 | |
| 2914 | LCLGWLWGM | 3061 | ASKLCLGWLWGMDID | 0.0002 | | −0.0005 | | 0.0017 | | −0.0002 | | | 0.0013 | | | 0.0010 |
| 2915 | LCLIFLLVL | 3062 | ILLLCLIFLLVLLDY | 0.0026 | | 0.0069 | | 0.0320 | | 0.0018 | | | 0.0047 | | | |
| 2916 | LCLRPVGAE | 3063 | RDVLCLRPVGAESRG | | | | | | | | | | | | | |
| 2917 | LCQVFADAT | 3064 | RPGLCQVFADATPTG | | | | | | | | | | | | | |
| 2918 | LDSWWTSLN | 3065 | PQSLDSWWTSLNFLG | | | | | | | | | | | | | |
| 2919 | LDTASALYR | 3066 | RDLLDTASALYREAL | 0.0001 | | | | 0.0092 | | | | | 0.0770 | | | |
| 2920 | LDVSAAFYH | 3067 | WLSLDVSAAFYHIPL | | | | | | | | | | | | | |
| 2921 | LDYQGMLPV | 3068 | LVLLDYQGMLPVCPL | 0.0034 | | | | −0.0013 | | | | | 0.0011 | | | |
| 2922 | LEEELPRLA | 3069 | AGPLEEELPRLADEG | | | | 0.0022 | | | | | | | | | |
| 2923 | LFILLLCLI | 3070 | IIFLFILLLCLIFLL | | | | | | | | | | | | | |
| 2924 | LGAKSVQHL | 3071 | DVVLGAKSVQHLESL | | | | | | | | | | | | | |
| 2925 | LGFAAPFTQ | 3072 | VGLLGFAAPFTQCGY | 0.0470 | 0.3100 | 0.0008 | | −0.0014 | | −0.0004 | | −0.0001 | 0.0014 | | 0.5700 | |
| 2926 | LGFRKIPMG | 3073 | PIILGFRKIPMGVGL | | | | | | | | | | | | | |
| 2927 | LGNLNVSIP | 3074 | DLNLGNLNVSIPWTH | 0.0038 | | | | 0.0240 | | | | | 0.0010 | | | |
| 2928 | LGPLLVLQA | 3075 | SGFLGPLLVLQAGFF | | | | | | | | | | | | | |
| 2929 | LHPAAMPHL | 3076 | HLPLHPAAMPHLLVG | | | | | | | | | | | | | |
| 2930 | LIFLLVLLD | 3077 | LLCLIFLLVLLDYQG | | | | | | | | | | | | | |
| 2931 | LKLIMPARF | 3078 | KRRLKLIMPARFYPN | | | | | | | | | | | | | |
| 2932 | LKVFVLGGC | 3079 | EIRLKVFVLGGCRHK | | | | | | | | | | | | | |
| 2933 | LLAQFTSAI | 3080 | SPFLLAQFTSAICSV | 0.1200 | 0.0200 | 0.0085 | −0.0013 | 0.0740 | 0.0190 | −0.0002 | −0.0013 | 0.0540 | 0.0330 | 0.0014 | 0.0380 | 0.2000 |
| 2934 | LLDTASALY | 3081 | IRDLLDTASALYREA | | | | | | | | | | | | | |
| 2935 | LLGCAANWI | 3082 | FPWLLGCAANWILRG | | | | | | | | | | | | | |
| 2936 | LLGFAAPFT | 3083 | IVGLLGFAAPFTQCG | 0.0200 | | −0.0005 | | −0.0007 | | −0.0002 | | | 0.0009 | | | 0.0067 |
| 2937 | LLGWSPQAQ | 3084 | HGGLLGWSPQAQGIL | | | | | | | | | | | | | |
| 2938 | LLLCLIFLL | 3085 | LFILLLCLIFLLVLL | | | | | | | | | | | | | |
| 2939 | LLSFLPSDF | 3086 | SVELLSFLPSDFFPS | | | | | | | | | | | | | |
| 2940 | LLSLGIHLN | 3087 | TNFLLSLGIHLNPNK | 3.5000 | 0.0410 | 0.1200 | | 0.0220 | 0.0360 | 0.0053 | | 0.0160 | 0.2200 | 0.0032 | 0.3800 | |
| 2941 | LLSSNLSWL | 3088 | LTNLLSSNLSWLSLD | 0.0010 | | 0.0083 | | 0.0160 | | 0.0013 | | | 0.0019 | | | 0.0200 |
| 2942 | LLTRILTIP | 3089 | GFFLLTRILTIPQSL | 0.4300 | 0.0150 | 0.0110 | | 3.1000 | 0.4500 | 2.3000 | | 0.0780 | 3.5000 | 1.6000 | 0.5500 | |
| 2943 | LLVLQAGFF | 3090 | LGPLLVLQAGFFLLT | | | | | | | | | | | | | |
| 2944 | LLVPFVQWF | 3091 | WLSLLVPFVQWFVGL | | | | | | | | | | | | | |
| 2945 | LLWFHISCL | 3092 | IRQLLWFHISCLTFG | | | | | | | | | | | | | |
| 2946 | LMPLYACIQ | 3093 | YPALMPLYACIQSKQ | 0.2400 | | | | 0.0014 | | | | | 0.0011 | | | |
| 2947 | LNLGNLNVS | 3094 | AEDLNLGNLNVSIPW | 0.0001 | | −0.0005 | | −0.0007 | | −0.0002 | | | −0.0003 | | | 0.0170 |
| 2948 | LNPNKTKRW | 3095 | GIHLNPNKTKRWGYS | | | | | | | | | | | | | |
| 2949 | LNRRVAEDL | 3096 | DEGLNRRVAEDLNLG | | | | | | | | | | | | | |
| 2950 | LNVSIPWTH | 3097 | LGNLNVSIPWTHKVG | | | | | | | | | | | | | |
| 2951 | LPETTVVRR | 3098 | LSTLPETTVVRRRGR | | | | | | | | | | | | | |
| 2952 | LPIFFCLWV | 3099 | LPLLPIFFCLWVYIZ | | | | | | | | | | | | | |
| 2953 | LPIHTAELL | 3100 | VAPLPIHTAELLAAC | | | | | | | | | | | | | |
| 2954 | LPVNRPIDW | 3101 | FRKLPVNRPIDWKVC | | | | | | | | | | | | | |
| 2955 | LQFRNSKPC | 3102 | CWWLQFRNSKPCSDY | | | | | | | | | | | | | |
| 2956 | LRGLPVCAF | 3103 | HLSLRGLPVCAFSSA | 1.3000 | | | | 0.0028 | | | | | 0.0130 | | | |
| 2957 | LRPVGAESR | 3104 | VLCLRPVGAESRGRP | | | | | | | | | | | | | |
| 2958 | LRQAILCWG | 3105 | HTALRQAILCWGELM | | | | | | | | | | | | | |
| 2959 | LRRFIIFLF | 3106 | WMCLRRFIIFLFILL | | | | | | | | | | | | | |
| 2960 | LSFLPSDFF | 3107 | VELLSFLPSDFFPSI | | | | | | | | | | | | | |
| 2961 | LSLDVSAAF | 3108 | LSWLSLDVSAAFYHI | | | | | | | | | | | | | |
| 2962 | LSLLVPFVQ | 3109 | FSWLSLLVPFVQWFV | | | | | | | | | | | | | |
| 2963 | LSLRGLPVC | 3110 | GAHLSLRGLPVCAFS | 0.7800 | | 0.0042 | −0.0041 | 0.0011 | | 0.0025 | | | 0.0077 | | | 0.0150 |
| 2964 | LSPFLLAQF | 3111 | GVGLSPFLLAQFTSA | | | | | | | | | | | | | |
| 2965 | LSRKYTSFP | 3112 | SVVLSRKYTSFPWLL | 0.0005 | | 0.0057 | 0.2100 | −0.0016 | | 0.5300 | | | 0.0130 | | | |
| 2966 | LSSNLSWLS | 3113 | TNLLSSNLSWLSLDV | 0.0016 | | −0.0005 | | 0.1300 | | 0.0006 | | | 0.0019 | | | 0.0410 |
| 2967 | LSVPNPLGF | 3114 | GTNLSVPNPLGFFPD | | | | | | | | | | | | | |
| 2968 | LSWLSLDVS | 3115 | SSNLSWLSLDVSAAF | 0.1400 | 0.0030 | −0.0005 | 1.5000 | 0.2700 | | 0.0046 | 0.0180 | 0.1000 | 0.0039 | 0.0460 | 0.0110 | 6.2000 |
| 2969 | LTIPQSLDS | 3116 | TRILTIPQSLDSWWT | | | | | | | | | | | | | |
| 2970 | LTNLLSSNL | 3117 | LQSLTNLLSSNLSWL | 2.5000 | 0.4400 | 0.0200 | −0.0013 | 4.8000 | 0.8100 | 0.0680 | 0.7500 | 0.0260 | 0.1500 | 0.0880 | 0.1100 | |
| 2971 | LTRILTIPQ | 3118 | FFLLTRILTIPQSLD | | | | | | | | | | | | | |
| 2972 | LVDKNPHNT | 3119 | GVFLVDKNPHNTTES | | | | | | | | | | | | | |
| 2973 | LVSFGVWIR | 3120 | LEYLVSFGVWIRTPP | | | | | | | | | | | | | |
| 2974 | LVVDFSQFS | 3121 | ESRLVVDFSQFSRGN | 0.0007 | 0.0074 | −0.0010 | 2.6000 | | | −0.0004 | | 0.0040 | −0.0014 | 0.0029 | | |
| 2975 | LWFHISCLT | 3122 | RQLLWFHISCLTFGR | 0.0002 | | 0.0009 | | 0.0140 | | 0.0011 | | | 0.0061 | | | 0.0096 |
| 2976 | LWGMDIDPY | 3123 | LGWLWGMDIDPYKEF | 0.0004 | | 0.0006 | 0.0200 | 0.0280 | | −0.0002 | | | 0.0004 | | | 0.0430 |
| 2977 | LWKAGILYK | 3124 | LHTLWKAGILYKRET | | | | | | | | | | | | | |
| 2978 | LYREALESP | 3125 | ASALYREALESPEHC | | | | | | | | | | | | | |
| 2979 | LYSHPIILG | 3126 | KLHLYSHPIILGFRK | | | | | | | | | | | | | |
| 2980 | MDDVVLGAK | 3127 | FSYMDDVVLGAKSVQ | | | | | | | | | | | | | |
| 2981 | MGVGLSPFL | 3128 | KIPMGVGLSPFLLAQ | | | | | | | | | | | | | |
| 2982 | MPHLLVGSS | 3129 | PAAMPHLLVGSSGLS | | | | | | | | | | | | | |
| 2983 | MQWNSTTFH | 3130 | PQAMQWNSTTFHQTL | 0.0012 | | | | 0.0300 | | | | | 0.1200 | | | |
| 2984 | MSTTDLEAY | 3131 | LSAMSTTDLEAYFKD | | | | | | | | | | | | | |
| 2985 | MWYWGPSLY | 3132 | IWMMWYWGPSLYNIL | | | | | | | | | | | | | |
| 2986 | VCAFSSAGP | 3133 | GLPVCAFSSAGPCAL | | | | | | | | | | | | | |
| 2987 | VCQRIVGLL | 3134 | DWKVCQRIVGLLGFA | 0.0120 | | −0.0026 | | 0.0030 | | 0.2500 | | | 0.0018 | | | 0.0130 |
| 2988 | VFADATPTG | 3135 | LCQVFADATPTGWGL | 0.0020 | | | | 0.9600 | | | | | 0.0013 | | | |
| 2989 | VGLSPTVWL | 3136 | QWFVGLSPTVWLSVI | | | | | | | | | | | | | |
| 2990 | VGPLTVNEK | 3137 | QQYVGPLTVNEKRRL | | | | | | | | | | | | | |
| 2991 | VHFASPLHV | 3138 | PDRVHFASPLHVAWR | 0.0510 | 0.0290 | 0.0008 | | 0.0008 | 0.0054 | 0.0008 | | 0.0190 | 0.0810 | 0.0035 | 0.2400 | |
| 2992 | VLCLRPVGA | 3139 | ARDVLCLRPVGAESR | | | | | | | | | | | | | |
| 2993 | VLGAKSVQH | 3140 | DDVVLGAKSVQHLES | | | | | | | | | | | | | |
| 2994 | VLHKRTLGL | 3141 | LPKVLHKRTLGLSAM | | | | | | | | | | | | | |
| 2995 | VPNLQSLTN | 3142 | KFAVPNLQSLTNLLS | 0.0180 | 0.0005 | −0.0003 | | 0.1300 | | 0.0043 | | 0.0088 | −0.0003 | | 0.0056 | |
| 2996 | VQASKLCLG | 3143 | CPTVQASKLCLGWLW | | | | | | | | | | | | | |
| 2997 | VRFSWLSLL | 3144 | WASVRFSWLSLLVPF | | | | | | | | | | | | | |
| 2998 | VRRAFPHCL | 3145 | CSVVRRAFPHCLAFS | 0.1000 | 0.1024 | 0.0770 | 0.0032 | 0.0016 | −0.0022 | 0.0008 | −0.0013 | 0.0540 | 0.0590 | 0.0250 | 1.2000 | 0.0460 |
| 2999 | VSIPWTHKV | 3146 | NLNVSIPWTHKVGNF | 0.0001 | | −0.0005 | −0.0041 | −0.0007 | | −0.0002 | | | 0.0005 | | | 0.0009 |
| 3000 | VWIRTPPAY | 3147 | SFGVWIRTPPAYRPP | 0.0094 | 0.0110 | 0.4300 | −0.0009 | 0.0780 | 0.0630 | 0.0260 | 0.0071 | 0.0002 | 0.0240 | 0.2500 | 0.0800 | 0.0016 |
| 3001 | VYVPSALNP | 3148 | TSFVYVPSALNPADD | | | | | | | | | | | | | |
| 3002 | WFHISCLTF | 3149 | QLLWFHISCLTFGRE | | | | | | | | | | | | | |
| 3003 | WFVGLSPTV | 3150 | FVQWFVGLSPTVWLS | 0.4700 | 0.0035 | 0.0160 | −0.0013 | 0.0130 | | 0.0072 | 0.0021 | 0.0190 | 0.0690 | 0.0180 | 0.0410 | 0.0044 |
| 3004 | WILRGTSFV | 3151 | AANWILRGTSFVYVP | 0.0920 | 0.0240 | 0.0061 | 0.0023 | 0.0510 | 0.0250 | 0.0140 | 0.3700 | 0.0250 | 0.5800 | 0.2500 | 0.2700 | |
| 3005 | WIRTPPAYR | 3152 | FGVWIRTPPAYRPPN | | | | | | | | | | | | | |
| 3006 | WKAGILYKR | 3153 | HTLWKAGILYKRETT | | | | | | | | | | | | | |
| 3007 | WLLGCAANW | 3154 | SFPWLLGCAANWILR | | | | | | | | | | | | | |
| 3008 | WLSLDVSAA | 3155 | NLSWLSLDVSAAFYH | 0.1400 | 0.0003 | −0.0005 | 1.3000 | 0.2900 | | 0.0033 | 0.0022 | 0.0330 | 0.0041 | 0.0150 | 0.0620 | 2.4000 |
| 3009 | WLSLLVPFV | 3156 | RFSWLSLLVPFVQWF | 0.0430 | | 0.0009 | | −0.0007 | | 0.0002 | | | 0.0005 | | | 0.0031 |
| 3010 | WPKFAVPNL | 3157 | RVSWPKFAVPNLQSL | | | | | | | | | | | | | |
| 3011 | YMDDVVLGA | 3158 | AFSYMDDVVLGAKSV | 0.0027 | | −0.0005 | 0.0130 | 2.9000 | | 0.0006 | | | −0.0003 | | | −0.0005 |
| 3012 | YPALMPLYA | 3159 | QCGYPALMPLYACIQ | 0.0062 | | 0.0018 | | 0.0068 | | 0.0023 | | | 0.0006 | | | |
| 3013 | YQGMLPVCP | 3160 | LLDYQGMLPVCPLIP | | | | | | | | | | | | | |
| 3014 | YRPPNAPIL | 3161 | PPAYRPPNAPILSTL | 0.0056 | | −0.0005 | | 0.0038 | | 0.0022 | | | 0.0024 | | | 0.0015 |
| 3015 | YRWMCLRRF | 3162 | CPGYRWMCLRRFIIF | | | | | | | | | | | | | |
| 3016 | YSHPIILGF | 3163 | LHLYSHPIILGFRKI | 0.0220 | 0.0340 | 0.0400 | 0.0040 | 0.6800 | 0.1600 | 0.0410 | 0.0310 | 0.0002 | 0.0006 | 0.0610 | 0.0490 | |
| 3017 | YSLNFMGYV | 3164 | RWGYSLNFMGYVIGS | | | | | | | | | | | | | |
| 3018 | YVPSALNPA | 3165 | SFVYVPSALNPADDP | | | | | | | | | | | | | |
| 3019 | FFCLWVYIZ | | | | | | | | | | | | | | | |
| 3020 | MGTNLSVPN | | | | | | | | | | | | | | | |

TABLE XXa
HBV DR-3A Motif

| | | | | | | | | | |
|---|---|---|---|---|---|---|---|---|---|
| | Core | | | | | | | | Exemplary |
| | SEQ | | | Core | | | | Exemplary | Sequence |
| | ID | Core | Core | Conservancy | Exemplary | Exemplary | Position in | Sequence | Conservancy |
| Protein | NO: | Sequence | Freq. | (%) | SEQ ID NO: | Sequence | Poly-Protein | Frequency | (%) |

| | | | | | | | | | |
|---|---|---|---|---|---|---|---|---|---|
| ENV | 3166 | FFPDHQLDP | 19 | 95 | 3181 | PLGFFPDHQLDPAFG | 10 | 9 | 95 |
| NUC | 3167 | FGRETVLEY | 15 | 75 | 3182 | CLTFGRETVLEYLVS | 136 | 14 | 75 |
| POL | 3168 | FGVEPSGSG | 15 | 75 | 3183 | RRSFGVEPSGSGHD | 241 | 6 | 75 |
| POL | 3169 | FLVDKNPHN | 20 | 100 | 3184 | GGVFLVDKNPHNTTE | 360 | 11 | 100 |
| POL | 3170 | IGTDNSVVL | 16 | 80 | 3185 | AKLIGTDNSVVLSRK | 731 | 13 | 80 |
| POL | 3171 | LEEELPRLA | 18 | 90 | 3186 | AGPLEEELPRLADEG | 18 | 13 | 90 |
| POL | 3172 | LPLDKGIKP | 20 | 100 | 3187 | TKYLPLDKGIKPYYP | 120 | 20 | 100 |
| POL | 3173 | LSLDVSAAF | 19 | 95 | 3188 | LSWLSLDVSAAFYHI | 412 | 11 | 95 |
| POL | 3174 | LVVDFSQFS | 20 | 100 | 3189 | ESRLVVDFSQFSRGN | 374 | 9 | 100 |
| NUC | 3175 | LYREALESP | 17 | 85 | 3190 | ASALYREALESPEHC | 34 | 17 | 85 |
| NUC | 3176 | MQIDPYKEF | 17 | 85 | 3191 | LWGMDIDPYKEFGAS | 27 | 9 | 85 |
| POL | 3177 | VAEDLNLGN | 20 | 100 | 3192 | NRRVAEDLNLGNLNV | 34 | 17 | 100 |
| POL | 3178 | VFADATPTG | 19 | 95 | 3193 | LCQVFADATPTGWGL | 683 | 19 | 95 |
| ENV | 3179 | VLLDYQGML | 19 | 95 | 3194 | FLLVLLDYQGMLPVC | 256 | 18 | 95 |
| POL | 3180 | YMDDVVLGA | 18 | 90 | 3195 | AFSYMDDVVLGAKSV | 535 | 18 | 90 |

TABLE XXb
HCV DR 3A Motif

| Core SEQ | Core | SEQ ID | Exemplary | | | | | |
|---|---|---|---|---|---|---|---|---|
| ID NO: | Sequence | NO: | Sequence | DR1 | DR2w2β1 | DR2w2β2 | DR3 | DR4w4 |
| 3166 | FFPDHQLDP | 3181 | PLGFFPDHQLDPAFG | | | | | |
| 3167 | FGRETVLEY | 3182 | CLTFGRETVLEYLVS | | | | | |
| 3168 | FGVEPSGSG | 3183 | RRSFGVEPSGSGHID | | | | | |
| 3169 | FLVDKNPHN | 3184 | GGVFLVDKNPHNTTE | | | | 0.0790 | |
| 3170 | IGTDNSVVL | 3185 | AKLIGTDNSVVLSRK | | | | | |
| 3171 | LEEELPRLA | 3186 | AGPLEEELPRLADEG | | | | 0.0022 | |
| 3172 | LPLDKGIKP | 3187 | TKYLPLDKGIKPYYP | | | | −0.0017 | |
| 3173 | LSLDVSAAF | 3188 | LSWLSLDVSAAFYHI | | | | | |
| 3174 | LVVDFSQFS | 3189 | ESRLVVDFSQFSRGN | 0.0007 | 0.0074 | −0.0010 | 2.6000 | |
| 3175 | LYREALESP | 3190 | ASALYREALESPEHC | | | | | |
| 3176 | MDIDPYKEF | 3191 | LWGMDIDPYKEFGAS | | | | | |
| 3177 | VAEDLNLGN | 3192 | NRRVAEDLNLGNLNV | | | | 0.1400 | |
| 3178 | VFADATPTG | 3193 | LCQVFADATPTGWGL | 0.0020 | | | | 0.9600 |
| 3179 | VLLDYQGML | 3194 | FLLVLLDYQGMLPVC | | | | 0.0170 | |
| 3180 | YMDDVVLGA | 3195 | AFSYMDDVVLGAKSV | 0.0027 | | −0.0005 | 0.0130 | 2.9000 |

| Core SEQ | | | | | | | | |
|---|---|---|---|---|---|---|---|---|
| ID NO: | DR4w15 | DR5w11 | DR5w12 | DR6w19 | DR7 | DR8W2 | DR9 | DRW53 |
| 3166 | | | | | | | | |
| 3167 | | | | | | | | |
| 3168 | | | | | | | | |
| 3169 | | | | | | | | |
| 3170 | | | | | | | | |
| 3171 | | | | | | | | |
| 3172 | | | | | | | | |
| 3173 | | | | | | | | |
| 3174 | | −0.0004 | | 0.4000 | −0.0014 | 0.0029 | | |
| 3175 | | | | | | | | |
| 3176 | | | | | | | | |
| 3177 | | | | | | | | |
| 3178 | | | | | 0.0013 | | | |
| 3179 | | | | | | | | |
| 3180 | | 0.0006 | | | −0.0003 | | | −0.0005 |

TABLE XXc
HBV DR-3B Motif

| | | | | | | | | | |
|---|---|---|---|---|---|---|---|---|---|
| | | | | Core | SEQ | | | Exemplary | |
| | Core SEQ | Core | Core | Conservancy | ID | | Position in | Sequence | Exemplary |
| Protein | ID NO: | Sequence | Freq. | (%) | NO: | Exemplary Sequence | HBV Poly-Protein | Frequency | Sequence |

| | | | | | | | | | |
|---|---|---|---|---|---|---|---|---|---|
| X | 3196 | AHLSLRGLP | 18 | 90 | 3202 | DHGAHLSLRGLPVCA | 48 | 18 | 90.00 |
| POL | 3197 | FSPTYKAFL | 19 | 95 | 3203 | AFTFSPTYKAFLCKQ | 655 | 11 | 55.00 |
| POL | 3198 | IPWTHKVGN | 20 | 100 | 3204 | NVSIPWTHKVGNFTG | 47 | 20 | 100.00 |
| POL | 3199 | LTVNEKRRL | 17 | 85 | 3205 | VGPLTVNEKRRLKLI | 96 | 12 | 60.00 |
| X | 3200 | VGAESRGRP | 19 | 95 | 3206 | LRPVGAESRGRPVSG | 18 | 7 | 35.00 |
| POL | 3201 | VVLSRKYTS | 18 | 90 | 3207 | DNSVVLSRKYTSFPW | 737 | 17 | 85.00 |

TABLE XXd
HBV DR-3B Motif With Binding Information

| Core SEQ | Core | SEQ ID | | | | | | |
|---|---|---|---|---|---|---|---|---|
| ID NO: | Sequence | NO: | Exemplary Sequence | DR1 | DR2w2β1 | DR2w2β2 | DR3 | DR4w4 |
| 3196 | AHLSLRGLP | 3202 | DHGAHLSLRGLPVCA | | | | | |
| 3197 | FSPTYKAFL | 3203 | AFTFSPTYKAFLCKQ | | | | 0.0035 | |
| 3198 | IPWTHKVGN | 3204 | NVSIPWTHKVGNFTG | | | | | |
| 3199 | LTVNEKRRL | 3205 | VGPLTVNEKRRLKLI | 0.0006 | 0.0022 | 0.0047 | 2.2000 | |
| 3200 | VGAESRGRP | 3206 | LRPVGAESRGRPVSG | | | | −0.0017 | |
| 3201 | VVLSRKYTS | 3207 | DNSVVLSRKYTSFPW | | | | | |

| Core SEQ | | | | | | | | |
|---|---|---|---|---|---|---|---|---|
| ID NO: | DR4w15 | DR5w11 | DR5w12 | DR6w19 | DR7 | DR8w2 | DR9 | DRw53 |
| 3196 | | | | | | | | |
| 3197 | | | | | | | | |
| 3198 | | | | | | | | |
| 3199 | | 0.0030 | | 0.0009 | −0.0014 | 0.0092 | | |
| 3200 | | | | | | | | |
| 3201 | | | | | | | | |

TABLE XXI
Population coverage with combined HLA Supertypes

| | PHENOTYPIC FREQUENCY | | | | | |
|---|---|---|---|---|---|---|
| | | North | | | | |
| | | American | | | | |
| HLA-SUPERTYPES | Caucasian | Black | Japanese | Chinese | Hispanic | Average |
| a. Individual Supertypes | | | | | | |
| A2 | 45.8 | 39.0 | 42.4 | 45.9 | 43.0 | 43.2 |
| A3 | 37.5 | 42.1 | 45.8 | 52.7 | 43.1 | 44.2 |
| B7 | 38.6 | 52.7 | 48.8 | 35.5 | 47.1 | 44.7 |
| A1 | 47.1 | 16.1 | 21.8 | 14.7 | 26.3 | 25.2 |
| A24 | 23.9 | 38.9 | 58.6 | 40.1 | 38.3 | 40.0 |
| B44 | 43.0 | 21.2 | 42.9 | 39.1 | 39.0 | 37.0 |
| B27 | 28.4 | 26.1 | 13.3 | 13.9 | 35.3 | 23.4 |
| B62 | 12.6 | 4.8 | 36.5 | 25.4 | 11.1 | 18.1 |
| B58 | 10.0 | 25.1 | 1.6 | 9.0 | 5.9 | 10.3 |
| b. Combined Supertypes | | | | | | |
| A2, A3, B7 | 83.0 | 86.1 | 87.5 | 88.4 | 86.3 | 86.2 |
| A2, A3, B7, A24, B44, A1 | 99.5 | 98.1 | 100.0 | 99.5 | 99.4 | 99.3 |
| A2, A3, B7, A24, B44, A1, | 99.9 | 99.6 | 100.0 | 99.8 | 99.9 | 99.8 |
| B27, B62, B58 | | | | | | |

TABLE XXII
HBV ANALOGS

| | | | | | | | | | | |
|---|---|---|---|---|---|---|---|---|---|---|
| | | | | A2 | A3 | | B7 | 1° | | |
| | | Fixed | A1 | Super | Super | A24 | Super | Anchor | | SEQ ID |
| AA | Sequence | Nomen. | Motif | Motif | Motif | Motif | Motif | Fixer | Analog | NO: |

| | | | | | | | | | | |
|---|---|---|---|---|---|---|---|---|---|---|
| 10 | CILLLCLIFL | | N | Y | N | N | N | No | A | 3208 |
| 9 | RMTGGVFLV | VM2.V9 | N | Y | N | N | N | 1 | A | 3209 |
| 9 | LMPFVQWFV | VM2.V9 | N | Y | N | N | N | 1 | A | 3210 |
| 9 | RLTGGVFLV | VL2.V9 | N | Y | N | N | N | 1 | A | 3211 |
| 9 | GLCQVFADV | L2.AV9 | N | Y | N | N | N | 1 | A | 3212 |
| 9 | WLLRGTSFV | IL2.V9 | N | Y | N | N | N | 1 | A | 3213 |
| 9 | NLGNLNVSV | L2.IV9 | N | Y | N | N | N | 1 | A | 3214 |
| 9 | YLPSALNPV | VL2.AV9 | N | Y | N | N | N | 1 | A | 3215 |
| 9 | GLWIRTPPV | VL2.AV9 | N | Y | N | N | N | 1 | A | 3216 |
| 9 | RLSWPKFAV | VL2.V9 | N | Y | N | N | N | 1 | A | 3217 |
| 9 | ILGLLGFAV | VL2.AV9 | N | Y | N | N | N | 1 | A | 3218 |
| 9 | RMLTIPQSV | IM2.LV9 | N | Y | N | N | N | 1 | A | 3219 |
| 9 | SLDSWWTSV | L2.LV9 | N | Y | N | N | N | 1 | A | 3220 |
| 10 | FMLLLCLIFL | IM2.L10 | N | Y | N | Y | N | 1 | A | 3221 |
| 10 | LMLQAGFFLV | VM2.LV | N | Y | N | N | N | 1 | A | 3222 |
| 10 | SMLSPFLPLV | IM2.LV1 | N | Y | N | N | N | 1 | A | 3223 |
| 10 | LMLLDYQGMV | VM2.LV | N | Y | N | N | N | 1 | A | 3224 |
| 10 | FLGLSPTVWV | VL2.LV1 | N | Y | N | N | N | 1 | A | 3225 |
| 8 | FPAAMPHL | | N | N | N | N | Y | | A | 3226 |
| 8 | HPFAMPHL | | N | N | N | N | Y | | A | 3227 |
| 8 | HPAAMPHI | | N | N | N | N | Y | | A | 3228 |
| 8 | FMFSPTYK | | N | N | Y | N | N | | A | 3229 |
| 8 | FVFSPTYK | | N | N | Y | N | N | | A | 3230 |
| 9 | FLLTRILTV | L2.IV9 | N | Y | N | N | N | 1 | A | 3231 |
| 9 | ALMPLYACV | L2.IV9 | N | Y | N | N | N | 1 | A | 3232 |
| 9 | LLAQFTSAV | L2.IV9 | N | Y | N | N | N | 1 | A | 3233 |
| 9 | LLPFVQWFV | VL2.V9 | N | Y | N | N | N | 1 | A | 3234 |
| 9 | FLLAQFTSV | L2.AV9 | N | Y | N | N | N | 1 | A | 3235 |
| 9 | KLHLYSHPV | L2.IV9 | N | Y | N | N | N | 1 | A | 3236 |
| 9 | KLFLYSHPI | | N | Y | N | N | N | No | A | 3237 |
| 9 | LLSSNLSWV | L2.LV9 | N | Y | N | N | N | 1 | A | 3238 |
| 9 | FLLSLGIHV | L2.LV9 | N | Y | N | N | N | 1 | A | 3239 |
| 9 | MMWYWGPSV | M2.LV9 | N | Y | N | N | N | 1 | A | 3240 |
| 9 | VLQAGFFLV | L2.LV9 | N | Y | N | N | N | 1 | A | 3241 |
| 9 | PLLPIFFCV | L2.LV9 | N | Y | N | N | N | 1 | A | 3242 |
| 9 | FLLPIFFCL | | N | Y | N | N | N | No | A | 3243 |
| 9 | VLLDYQGMV | L2.LV9 | N | Y | N | N | N | 1 | A | 3244 |
| 9 | YMFDVVLGA | | N | Y | N | N | N | No | A | 3245 |
| 9 | GLLGWSPQV | L2.AV9 | N | Y | N | N | N | 1 | A | 3246 |
| 9 | FPAAMPHLL | | N | N | N | N | Y | | A | 3247 |
| 9 | HPFAMPHLL | | N | N | N | N | Y | | A | 3248 |
| 9 | HPAAMPHLI | | N | N | N | N | Y | | A | 3249 |
| 9 | FPVCAFSSA | | N | N | N | N | Y | | A | 3250 |
| 9 | LPFCAFSSA | | N | N | N | N | Y | | A | 3251 |
| 9 | LPVCAFSSI | | N | N | N | N | Y | | A | 3252 |
| 9 | FPALMPLYA | | N | N | N | N | Y | | A | 3253 |
| 9 | YPFLMPLYA | | N | N | N | N | Y | | A | 3254 |
| 9 | YPALMPLYI | | N | N | N | N | Y | | A | 3255 |
| 9 | FPSRGRLGL | | N | N | N | N | Y | | A | 3256 |
| 9 | DPFRGRLGL | | N | N | N | N | Y | | A | 3257 |
| 9 | DPSRGRLGI | | N | N | N | N | Y | | A | 3258 |
| 9 | SMICSVVRR | | N | N | Y | N | N | | A | 3259 |
| 9 | SVICSVVRR | | N | N | Y | N | N | | A | 3260 |
| 9 | KVGNFTGLK | | N | N | Y | N | N | | A | 3261 |
| 9 | KVGNFTGLR | | N | N | Y | N | N | | A | 3262 |
| 9 | VVFFSQFSR | | N | N | Y | N | N | | A | 3263 |
| 9 | SVNRPIDWK | | N | N | Y | N | N | | A | 3264 |
| 9 | TLWKAGILK | | N | N | Y | N | N | | A | 3265 |
| 9 | TLWKAGILR | | N | N | Y | N | N | | A | 3266 |
| 9 | TMWKAGILY | | Y | N | Y | N | N | | A | 3267 |
| 9 | TVWKAGILY | | N | N | Y | N | N | | A | 3268 |
| 9 | RMYLHTLWK | | N | N | Y | N | N | | A | 3269 |
| 9 | RVYLHTLWK | | N | N | Y | N | N | | A | 3270 |
| 9 | AMTFSPTYK | | N | N | Y | N | N | | A | 3271 |
| 9 | SVVRRAFPR | | N | N | Y | N | N | | A | 3272 |
| 9 | SVVRRAFPK | | N | N | Y | N | N | | A | 3273 |
| 9 | SAIXSVVRR | | N | N | Y | N | N | | A | 3274 |
| 9 | LPVXAFSSA | | N | N | N | N | Y | | A | 3275 |
| 10 | FLLAQFTSAV | L2.IV10 | N | Y | N | N | N | 1 | A | 3276 |
| 10 | YLFTLWKAGI | | N | Y | N | N | N | No | A | 3277 |
| 10 | YLLTLWKAGI | | N | Y | N | N | N | No | A | 3278 |
| 10 | LLFYQGMLPV | | N | Y | N | N | N | No | A | 3279 |
| 10 | LLLYQGMLPV | | N | Y | N | N | N | No | A | 3280 |
| 10 | LLVLQAGFFV | L2.LV10 | N | Y | N | N | N | 1 | A | 3281 |
| 10 | ILLLCLIFLV | L2.LV10 | N | Y | N | N | N | 1 | A | 3282 |
| 10 | FPFCLAFSYM | | N | N | N | N | Y | | A | 3283 |
| 10 | FPHCLAFSYI | | N | N | N | N | Y | | A | 3284 |
| 10 | FPARVTGGVF | | N | N | N | N | Y | | A | 3285 |
| 10 | TPFRVTGGVF | | N | N | N | N | Y | | A | 3286 |
| 10 | TPARVTGGVI | | N | N | N | N | Y | | A | 3287 |
| 10 | FPCALRFTSA | | N | N | N | N | Y | | A | 3288 |
| 10 | GPFALRFTSA | | N | N | N | N | Y | | A | 3289 |
| 10 | GPCALRFTSI | | N | N | N | N | Y | | A | 3290 |
| 10 | FPAAMPHLLV | | N | N | N | N | Y | | A | 3291 |
| 10 | HPFAMPHLLV | | N | N | N | N | Y | | A | 3292 |
| 10 | HPAAMPHLLI | | N | N | N | N | Y | | A | 3293 |
| 10 | QMFTFSPTYK | | N | N | Y | N | N | | A | 3294 |
| 10 | QVFTFSPTYK | | N | N | Y | N | N | | A | 3295 |
| 10 | TMWKAGILYK | | N | N | Y | N | N | | A | 3296 |
| 10 | TVWKAGILYK | | N | N | Y | N | N | | A | 3297 |
| 10 | VMGGVFLVDK | | N | N | Y | N | N | | A | 3298 |
| 10 | VVGGVFLVDK | | N | N | Y | N | N | | A | 3299 |
| 10 | SMLPETTVVR | | N | N | Y | N | N | | A | 3300 |
| 10 | SVLPETTVVR | | N | N | Y | N | N | | A | 3301 |
| 10 | TMPETTVVRR | | N | N | Y | N | N | | A | 3302 |
| 10 | TVPETTVVRR | | N | N | Y | N | N | | A | 3303 |
| 10 | HTLWKAGILK | | N | N | Y | N | N | | A | 3304 |
| 10 | HTLWKAGILR | | N | N | Y | N | N | | A | 3305 |
| 10 | HMLWKAGILY | | Y | N | Y | N | N | | A | 3306 |
| 10 | HVLWKAGILY | | N | N | Y | N | N | | A | 3307 |
| 10 | GMDNSVVLSR | | N | N | Y | N | N | | A | 3308 |
| 10 | GVDNSVVLSR | | N | N | Y | N | N | | A | 3309 |
| 10 | GTFNSVVLSR | | N | N | Y | N | N | | A | 3310 |
| 10 | YMFDVVLGAK | | N | N | Y | N | N | | A | 3311 |
| 10 | MMWYWGPSLK | | N | N | Y | N | N | | A | 3312 |
| 10 | MMWYWGPSLR | | N | N | Y | N | N | | A | 3313 |
| 9 | ILLLXLIFL | | N | Y | N | N | N | | A | 3314 |
| 9 | LLLXLIFLL | | N | Y | N | N | N | | A | 3315 |
| 9 | LLXLIFLLV | | N | Y | N | N | N | | A | 3316 |
| 9 | PLLPIFFXL | | N | Y | N | N | N | | A | 3317 |
| 9 | ALMPLYAXI | | N | Y | N | N | N | | A | 3318 |
| 9 | GLXQVFADA | | N | Y | N | N | N | | A | 3319 |
| 9 | HISXLTFGR | | N | N | Y | N | N | | A | 3320 |
| 9 | FVLGGXRHK | | N | N | Y | N | N | | A | 3321 |
| 10 | FILLLXLIFL | | N | Y | N | N | N | | A | 3322 |
| 10 | ILLLXLIFLL | | N | Y | N | N | N | | A | 3323 |
| 10 | LLLXLIFLLV | | N | Y | N | N | N | | A | 3324 |
| 10 | LLPIFFXLWV | | N | Y | N | N | N | | A | 3325 |
| 10 | QLLWFHISXL | | N | Y | N | N | N | | A | 3326 |
| 10 | LLGXAANWIL | | N | Y | N | N | N | | A | 3327 |
| 10 | TSAIXSVVRR | | N | N | Y | N | N | | A | 3328 |
| 10 | GYRWMXLRRF | | N | N | N | Y | N | | A | 3329 |
| 10 | GPXALRFTSA | | N | N | N | N | Y | | A | 3330 |
| 10 | FPHXLAFSYM | | N | N | N | N | Y | | A | 3331 |
| 11 | HMLWKAGILYK | | N | N | Y | N | N | | A | 3332 |
| 11 | HVLWKAGILYK | | N | N | Y | N | N | | A | 3333 |
| 11 | SMLPETTVVRR | | N | N | Y | N | N | | A | 3334 |
| 11 | SVLPETTVVRR | | N | N | Y | N | N | | A | 3335 |
| 11 | GMDNSVVLSRK | | N | N | Y | N | N | | A | 3336 |
| 11 | GVDNSVVLSRK | | N | N | Y | N | N | | A | 3337 |
| 11 | GTFNSVVLSRK | | N | N | Y | N | N | | A | 3338 |
| 8 | MPLSYQHI | | N | N | N | N | Y | | A | 3339 |
| 8 | LPIFFCLI | | N | N | N | N | Y | | A | 3340 |
| 8 | SPFLLAQI | | N | N | N | N | Y | | A | 3341 |
| 8 | YPALMPLI | | N | N | N | N | Y | | A | 3342 |
| 8 | VPSALNPI | | N | N | N | N | Y | | A | 3343 |
| 9 | LPIFFCLWI | | N | N | N | N | Y | | A | 3344 |
| 9 | LPIHTAELI | | N | N | N | N | Y | | A | 3345 |
| 10 | VPFVQWFVGI | | N | N | N | N | Y | | A | 3346 |
| 11 | NPLGFFPDHQI | | N | N | N | N | Y | | A | 3347 |
| 11 | LPIHTAELLAI | | N | N | N | N | Y | | A | 3348 |
| 9 | FLPSYFPSA | L2.FV5. | N | Y | N | N | N | Rev3 | A | 3349 |
| 10 | YLHTLWKAGV | L2.IV10 | N | Y | N | N | N | 1 | A | 3350 |
| 11 | STLPETYVVRR | | N | N | Y | N | N | | A | 3351 |
| 9 | YMDDVVLGV | M2.AV9 | N | Y | N | N | N | 1 | A | 3352 |
| 9 | FPIPSSWAF | | N | N | N | N | Y | | A | 3353 |
| 9 | IPITSSWAF | | N | N | N | N | Y | | A | 3354 |
| 9 | IPILSSWAF | | N | N | N | N | Y | | A | 3355 |
| 9 | FPVCLAFSY | | N | N | N | N | Y | | A | 3356 |
| 9 | FPHCLAFAY | | N | N | N | N | Y | | A | 3357 |
| 9 | FPHCLAFSL | | N | N | N | N | Y | | A | 3358 |
| 9 | IPIPMSWAF | | N | N | N | N | Y | | A | 3359 |
| 9 | FPHCLAFAL | | N | N | N | N | Y | | A | 3360 |
| 10 | FLPSZFFPSV | | N | Y | N | N | N | No | A | 3361 |
| 10 | FLPSZFFPSV | | N | Y | N | N | N | No | A | 3362 |
| 9 | IPFPSSWAF | | N | N | N | N | Y | | A | 3363 |
| 9 | IPIPSSWAI | | N | N | N | N | Y | | A | 3364 |
| 9 | FPFCLAFSY | | N | N | N | N | Y | | A | 3365 |
| 9 | FPHCLAFSI | | N | N | N | N | Y | | A | 3366 |
| 9 | FPHCLAFSA | | N | N | N | N | Y | | A | 3367 |
| 10 | FQPSDYFPSV | | N | Y | N | N | N | Rev | A | 3368 |
| 9 | YLLTRILTI | | N | Y | N | N | N | | A | 3369 |
| 9 | FLYTRILTI | | N | Y | N | N | N | | A | 3370 |
| 9 | FLLTYILTI | | N | Y | N | N | N | | A | 3371 |
| 9 | FLLTRILYI | | N | Y | N | N | N | | A | 3372 |
| 11 | FLPSDFFPSVR | | N | N | Y | N | N | | A | 3373 |
| 9 | FLPSDFFPS | | N | N | N | N | N | | A | 3374 |
| 8 | FLPSDFFP | | N | N | N | N | N | | A | 3375 |
| 10 | FLPSDFFPSI | L2.VI10 | N | Y | N | N | N | Rev | A | 3376 |
| 10 | FLPSDYFPSV | | N | Y | N | N | N | No | A | 3377 |
| 12 | YSFLPSDFFPSV | | N | N | N | N | N | | A | 3378 |
| 10 | YNMGLKFRQL | | N | N | N | N | N | | A | 3379 |
| 9 | NMGLKYRQL | | N | Y | N | Y | N | No | A | 3380 |
| 10 | FLPS(X)YFPSV | | N | N | N | N | N | | A | 3381 |
| 10 | FLPSD(X)FPSV | | N | N | N | N | N | | A | 3382 |
| 11 | FLPSDLLPSVR | | N | N | Y | N | N | | A | 3383 |
| 12 | FLPSDFFPSVRD | | N | N | N | N | N | | A | 3384 |
| 12 | LSFLPSDFFPSV | | N | N | N | N | N | | A | 3385 |
| 11 | SFLPSDFFPSV | | N | N | N | N | N | | A | 3386 |
| 8 | PSDFFPSV | | N | N | N | N | N | | A | 3387 |
| 9 | FLMSYFPSV | | N | Y | N | N | N | No | A | 3388 |
| 9 | FLPSYFPSV | L2.FY5. | N | Y | N | N | N | 3 | A | 3389 |
| 10 | FLMSDYFPSV | | N | Y | N | N | N | No | A | 3390 |
| 11 | CILLLCLIFLL | | N | Y | N | N | N | No | A | 3391 |
| 10 | FLPNDFFPSA | L2.SN4. | N | Y | N | N | N | Rev | A | 3392 |
| 10 | FLPDDFFPSA | L2.SD4. | N | Y | N | N | N | Rev | A | 3393 |
| 10 | FLPNDFFPSV | | N | Y | N | N | N | No | A | 3394 |
| 10 | FLPSDFFPSA | L2.VA10 | N | Y | N | N | N | Rev | A | 3395 |
| 10 | FLPDDFFPSV | | N | Y | N | N | N | No | A | 3396 |
| 10 | FLPADFFPSV | | N | Y | N | N | N | No | A | 3397 |
| 10 | FLPVDFFPSV | | N | Y | N | N | N | No | A | 3398 |
| 10 | FLPADFFPSI | L2.SA4. | N | Y | N | N | N | Rev | A | 3399 |
| 10 | FLPVDFFPSI | L2.SV4. | N | Y | N | N | N | Rev | A | 3400 |
| 10 | FLPSDAFPSV | | N | Y | N | N | N | No | A | 3401 |
| 10 | FLPSAFFPSV | | N | Y | N | N | N | No | A | 3402 |
| 10 | FLPSDFAPSV | | N | Y | N | N | N | No | A | 3403 |
| 10 | FLPSDFFASV | | N | Y | N | N | N | No | A | 3404 |
| 10 | FLPSDFFPAV | | N | Y | N | N | N | No | A | 3405 |
| 10 | FLASDFFPSV | | N | Y | N | N | N | No | A | 3406 |
| 10 | FAPSDFFPSV | LA2.V10 | N | Y | N | N | N | Rev | A | 3407 |
| 10 | ALPSDFFPSV | | N | Y | N | N | N | No | A | 3408 |
| 10 | YLPSDFFPSV | | N | Y | N | N | N | No | A | 3409 |
| 10 | FMPSDFFPSV | LM2.V1 | N | Y | N | N | N | 1 | A | 3410 |
| 10 | FLKSDFFPSV | | N | Y | N | N | N | No | A | 3411 |
| 10 | FLPSEFFPSV | | N | Y | N | N | N | No | A | 3412 |
| 10 | FLPSDFYPSV | | N | Y | N | N | N | No | A | 3413 |
| 10 | FLPSDFFKSV | | N | Y | N | N | N | No | A | 3414 |
| 10 | FLPSDFFPKV | | N | Y | N | N | N | No | A | 3415 |
| | FLPSDFFPSV(CONH2) | | | | | | | | | 3416 |
| | VLEYLVSFGV(NH2) | | | | | | | | | 3417 |
| | ATVELLSFLPSDFFPSV-NH2 | | | | | | | | | 3418 |
| | TVELLSFLPSDFFPSV-NH2 | | | | | | | | | 3419 |
| | VELLSFLPSDFFPSV-NH2 | | | | | | | | | 3420 |
| | ELLSFLPSDFFPSV-NH2 | | | | | | | | | 3421 |
| | LLSFLPSDFFPSV-NH2 | | | | | | | | | 3422 |
| | LSFLPSDFFPSV-NH2 | | | | | | | | | 3423 |
| | SFLPSDFFPSV-NH2 | | | | | | | | | 3424 |
| | FLPSDFFPSV-NH2 | | | | | | | | | 3425 |
| | LPSDFFPSV-NH2 | | | | | | | | | 3426 |
| | PSDFFPSV-NH2 | | | | | | | | | 3427 |
| | FLPSDFFPS-NH2 | | | | | | | | | 3428 |
| | FLPSDFFP-NH2 | | | | | | | | | 3429 |
| | FLPSDFF-NH2 | | | | | | | | | 3430 |
| | ALPSDFFPSV-NH2 | | | | | | | | | 3431 |
| | SLNFLGGTTV(NH2) | | | | | | | | | 3432 |
| | FLPSDFFPSVR-NH2 | | | | | | | | | 3433 |
| | ALFKDWEEL | | | | | | | | | 3434 |
| | VLGGSRHKL | | | | | | | | | 3435 |
| | KIKESFRKL | | | | | | | | | 3436 |
| | ALMPLYASI | | | | | | | | | 3437 |
| | FLSKQYLNL | | | | | | | | | 3438 |
| | LLGSAANWI | | | | | | | | | 3439 |
| | NLNNLNVSI | | | | | | | | | 3440 |
| | IIKKSEQFV | | | | | | | | | 3441 |
| | ALSLIVNLL | | | | | | | | | 3442 |
| | RIPRTPRSV | | | | | | | | | 3443 |
| | | | | | | | | | | 3444 |
| | | | | | | | | | | 3445 |

TABLE XXIII
Immunogenicity of HBV-derived peptides

| | | | | | | Immunogenicity | | | |
|---|---|---|---|---|---|---|---|---|---|
| Supermotif | Peptide | Sequence | SEQ ID NO: | Protein | XRN | primary | transgenic | patients | overall¹ |

| | | | | | | | | | |
|---|---|---|---|---|---|---|---|---|---|
| A2 supermotif | 924.07 | FLPSDFFPSV | 3492 | HBV core 18 | 5 | 10/10 | 6/6 | 25/32^{a} | + |
| | 1069.06 | LLVPFVQWFV | 3493 | HBV env 338 | 5 | 3/4 | 6/9 | | + |
| | 1147.13 | FLLAQFTSAI | 3494 | HBV pol 513 | 5 | | 0/3 | | unk |
| | 1090.77 | YMDDVVLGV | 3495 | HBV pol 538 | 5 | | 9/9 | | + |
| | 777.03 | FLLTRILTI | 3496 | HBV env 183 | 4 | | | 14/23^{a} | + |
| | 927.15 | ALMPLYACI | 3497 | HBV pol 642 | 4 | 10/12 | 3/5 | 2/15^{a} | + |
| | 1013.01 | WLSLLVPFV | 3498 | HBV env 335 | 4 | 2/6 | 5/9 | 23/29^{a} | + |
| | 1069.05 | LLAQFTSAI | 3499 | HBV pol 504 | 4 | 0/4 | 0/5 | | unk |
| | 1132.01 | LVPFVQWFV | 3500 | HBV env 339 | 4 | 0/3 | 0/4 | | unk |
| | 1147.14 | VLLDYQGMLPV | 3501 | HBV env 259 | 4 | 4/4 | 6/6 | | + |
| | 927.41 | LLSSNLSWL | 3502 | HBV pol 992 | 3 | 0/4 | 0/3 | | unk |
| | 927.42 | NLSWLSLDV | 3503 | HBV pol 411 | 3 | | 2/8 | | + |
| | 927.46 | KLHLYSHPI | 3504 | HBV pol 489 | 3 | 0/4 | 4/6 | | + |
| | 1069.07 | FLLAQFTSA | 3505 | HBV pol 503 | 3 | 1/2 | 0/3 | | + |
| | 1168.02 | GLSRYVARL | 3506 | HBV pol 455 | 3 | | | 9/13^{a} | + |
| A2 supermotif | 927.11 | FLLSLGIHL | 3507 | HBV pol 562 | 2 | 15/22 | 12/13 | 9/15^{a} | + |
| | 927.47 | HLYSHPIIL | 3508 | HBV pol 1076 | 2 | | 10/14 | | + |
| | 1039.03 | MMWYWGPSL | 3509 | HBV env 360 | 2 | 3/4 | 0/4 | | + |
| | 1069.12 | YLHTLWKAGV | 3510 | HBV pol 147 | 2 | 2/4 | | | + |
| | 1137.02 | LLDYQGMLPV | 3511 | HBV env 260 | 2 | 1/2 | 0/4 | | + |
| | 1142.07 | GLLGWSPQA | 3512 | HBV env 62 | 2 | 3/4 | 5/6 | | + |
| | 1.0573 | ILRGTSFVYV | 3513 | HBV pol 773 | 1 | | | 3/7^{b} | + |
| | 1013.14 | VLQAGFFLL | 3514 | HBV env 177 | 1 | 0/4 | 5/12 | | + |
| | 1069.10 | LLPIFFCLWV | 3515 | HBV env 378 | 1 | 3/3 | 0/4 | 2/5^{c} | + |
| | 1069.13 | PLLPIFFCL | 3516 | HBV env 377 | 1 | 0/4 | 7/12 | | + |
| | 1090.06 | LLVLQAGFFL | 3517 | HBV env 175 | 1 | 1/5 | 0/4 | | + |
| | 1090.12 | YLVSFGVWI | 3518 | HBV nuc 118 | 1 | 9/9 | | | + |
| | 1.0518 | GLSPTVWLSV | 3519 | HBV env 338 | 1 | | | 3/9^{c} | + |
| | 1090.14 | YMDDVVLGA | 3520 | HBV pol 538 | 1 | 2/7 | 2/5 | 2/7^{b} | + |
| A3 supermotif | 1147.16 | HTLWKAGILYK | 3521 | HBV POL 149 | 5 | 0/6 | 3/3 | 1/22 | + |
| | 1083.01 | STLPETTVVRR | 3522 | HBV core 141 | 4 | 3/5 | 6/6 | 8/32 | + |
| | 1150.51 | GSTHVSWPK | 3523 | HBV pol 398 | 4 | | 3/6 | | + |
| | 1.0219 | FVLGGCRHK | 3524 | HBV adr “X” 1550 | 3 | 0/4 | | | unk |
| | 1069.16 | NVSIPWTHK | 3525 | HBV pol 47 | 3 | 0/8 | 0/3 | 1/21 | + |
| | 1069.20 | LVVDFSQFSR | 3526 | HBV pol 388 | 3 | 0/4 | 6/6 | 1/22 | + |
| | 1090.10 | QAFTFSPTYK | 3527 | HBV pol 665 | 3 | 3/6 | 0/3 | 3/21 | + |
| | 1090.11 | SAICSVVRR | 3528 | HBV pol 531 | 3 | 1/4 | | 2/22 | + |
| A3 supermotif | 1069.15 | TLWKAGILYK | 3529 | HBV pol 150 | 2 | 3/8 | 0/3 | 5/28 | + |
| | 1142.05 | KVGNFTGLY | 3530 | HBV adr POL 629 | 2 | | 0/3 | 2/22 | + |
| B7 supermotif | 1147.05 | FPHCLAFSYM | 3531 | HBV POL 530 | 5 | 1/3 | | 0/12 | + |
| | 988.05 | LPSDFFPSV | 3532 | HBV core 19-27 | 4 | | | 2/16 | + |
| | 1145.04 | IPIPSSWAF | 3533 | HBV ENV 313 | 4 | 0/4 | | 1/12 | + |
| | 1147.02 | HPAAMPHLL | 3534 | HBV POL 429 | 4 | 0/5 | | 0/12 | unk |
| | 1147.06 | LPVCAFSSA | 3535 | HBV X 58 | 4 | 1/4 | | | + |
| | 1147.08 | YPALMPLYA | 3536 | HBV POL 640 | 4 | | | 0/12 | unk |
| | 1145.08 | FPHCLAFSYM | 3537 | HBV POL 541 | 3 | 0/4 | | | unk |
| B7 supermotif | 1147.04 | TPARVTGGVF | 3538 | HBV POL 354 | 2 | | | 2/12 | + |

Immunogenicity evaluation derived from primary cultures, acute patients (^{a}Bertoni et al, J Clin Invest 100: 503, ^{b}Rehermann et al., J. Clin. Invest 97: 1655, ^{c}Nayersina et al., J Immunol 150: 4659) or transgenic mice. A positive assessment (+) is assigned when responders have been noted in one of these systems.
Unk = unknown

TABLE XXIV
MHC-peptide binding assays: cell lines and radiolabeled ligands.
A. Class I binding assays

| | | | | Radiolabeled peptide | | |
|---|---|---|---|---|---|---|
| Species | Antigen | Allele | Cell line | Source | Sequence | SEQ ID NO: |
| Human | A1 | A*0101 | Steinlin | Hu. J chain 102-110 | YTAVVPLVY | 3539 |
| | A2 | A*0201 | JY | HBVc 18-27 F6->Y | FLPSDYFPSV | 3540 |
| | A2 | A*0202 | P815 | HBVc 18-27 F6->Y | FLPSDYFPSV | 3540 |
| | | | (transfected) | | | |
| | A2 | A*0203 | FUN | HBVc 18-27 F6->Y | FLPSDYFPSV | 3540 |
| | A2 | A*0206 | CLA | HBVc 18-27 F6->Y | FLPSDYFPSV | 3540 |
| | A2 | A*0207 | 721.221 | HBVc 18-27 F6->Y | FLPSDYFPSV | 3540 |
| | | | (transfected) | | | |
| | A3 | | GM3107 | non-natural (A3CON1) | KVFPYALINK | 3541 |
| | A11 | | BVR | non-natural (A3CON1) | KVFPYALINK | 3541 |
| | A24 | A*2402 | KAS116 | non-natural (A24CON1) | AYIDNYNKF | 3542 |
| | A31 | A*3101 | SPACH | non-natural (A3CON1) | KVFPYALINK | 3541 |
| | A33 | A*3301 | LWAGS | non-natural (A3CON1) | KVFPYALINK | 3541 |
| | A28/68 | A*6801 | C1R | HBVc 141-151 T7->Y | STLPETYVVRR | 3543 |
| | A28/68 | A*6802 | AMAI | HBV pol 646-654 C4->A | FTQAGYPAL | 3544 |
| | B7 | B*0702 | GM3107 | A2 sigal seq. 5-13 (L7->Y) | APRTLVYLL | 3545 |
| | B8 | B*0801 | Steinlin | HIVgp 586-593 Y1->F, Q5->Y | FLKDYQLL | 3546 |
| | B27 | B*2705 | LG2 | R 60s | FRYNGLIHR | 3547 |
| | B35 | B*3501 | C1R, BVR | non-natural (B35CON2) | FPFKYAAAF | 3548 |
| | B35 | B*3502 | TISI | non-natural (B35CON2) | FPFKYAAAF | 3548 |
| | B35 | B*3503 | EHM | non-natural (B35CON2) | FPFKYAAAF | 3548 |
| | B44 | B*4403 | PITOUT | EF-1 G6->Y | AEMGKYSFY | 3549 |
| | B51 | | KAS116 | non-natural (B35CON2) | FPFKYAAAF | 3550 |
| | B53 | B*5301 | AMAI | non-natural (B35CON2) | FPFKYAAAF | 3550 |
| | B54 | B*5401 | KT3 | non-natural (B35CON2) | FPFKYAAAF | 3550 |
| | Cw4 | Cw*0401 | C1R | non-natural (C4CON1) | QYDDAVYKL | 3551 |
| | Cw6 | Cw*0602 | 721.221 | non-natural (C6CON1) | YRHDGGNVL | 3552 |
| | | | transfected | | | |
| | Cw7 | Cw*0702 | 721.221 | non-natural (C6CON1) | YRHDGGNVL | 3552 |
| | | | transfected | | | |
| Mouse | D^{b} | | EL4 | Adenovirus E1A P7->Y | SGPSNTYPEI | 3553 |
| | K^{b} | | EL4 | VSV NP 52-59 | RGYVFQGL | 3554 |
| | D^{d} | | P815 | HIV-IIIB ENV G4->Y | RGPYRAFVTI | 3555 |
| | K^{d} | | P815 | non-natural (KdCON1) | KFNPMKTYI | 3556 |
| | L^{d} | | P815 | HBVs 28-39 | IPQSLDSYWTSL | 3557 |

B. Class II binding assays

| | | | | Radiolabeled peptide | | |
|---|---|---|---|---|---|---|
| Species | Antigen | Allele | Cell line | Source | Sequence | SEQ ID NO: |
| Human | DR1 | DRB1*0101 | LG2 | HA Y307-319 | YPKYVKQNTLKLAT | 3558 |
| | DR2 | DRB1*1501 | L466.1 | MBP 88-102Y | VVHFFKNIVTPRTPPY | 3559 |
| | DR2 | DRB1*1601 | L242.5 | non-natural (760.16) | YAAFAAAKTAAAFA | 3560 |
| | DR3 | DRB1*0301 | MAT | MT 65 kD Y3-13 | YKTIAFDEEARR | 3561 |
| | DR4w4 | DRB1*0401 | Preiss | non-natural (717.01) | YARFQSQTTLKQKT | 3562 |
| | DR4w10 | DRB1*0402 | YAR | non-natural (717.10) | YARFQRQTTLKAAA | 3563 |
| | DR4w14 | DRB1*0404 | BIN 40 | non-natural (717.01) | YARFQSQTTLKQKT | 3562 |
| | DR4w15 | DRB1*0405 | KT3 | non-natural (717.01) | YARFQSQTTLKQKT | 3562 |
| | DR7 | DRB1*0701 | Pitout | Tet. tox. 830-843 | QYIKANSKFIGITE | 3564 |
| | DR8 | DRB1*0802 | OLL | Tet. tox. 830-843 | QYIKANSKFIGITE | 3564 |
| | DR8 | DRB1*0803 | LUY | Tet. tox. 830-843 | QYIKANSKFIGITE | 3564 |
| | DR9 | DRB1*0901 | HID | Tet. tox. 830-843 | QYIKANSKFIGITE | 3564 |
| | DR11 | DRB1*1101 | Sweig | Tet. tox. 830-843 | QYIKANSKFIGITE | 3564 |
| | DR12 | DRB1*1201 | Herluf | unknown eluted peptide | EALIHQLKINPYVLS | 3565 |
| | DR13 | DRB1*1302 | H0301 | Tet. tox. 830-843 S->A | QYIKANAKFIGITE | 3566 |
| | DR51 | DRB5*0101 | GM3107 | Tet. tox. 830-843 | QYIKANAKFIGITE | 3566 |
| | | | or L416.3 | | | |
| | DR51 | DRB5*0201 | L255.1 | HA 307-319 | PKYVKQNTLKLAT | 3567 |
| | DR52 | DRB3*0101 | MAT | Tet. tox. 830-843 | NGQIGNDPNRDIL | 3568 |
| | DR53 | DRB4*0101 | L257.6 | non-natural (717.01) | YARFQSQTTLKQKT | 3569 |
| | DQ3.1 | QA1*0301/DQB1*03 [symbol] | PF | non-natural (ROIV) | AHAAHAAHAAHAAHAA | 3570 |
| Mouse | IA^{b} | | DB27.4 | non-natural (ROIV) | AHAAHAAHAAHAAHAA | 3570 |
| | IA^{d} | | A20 | non-natural (ROIV) | AHAAHAAHAAHAAHAA | 3570 |
| | IA^{k} | | CH-12 | HEL 46-61 | YNTDGSTDYGILQINSR | 3571 |
| | IA^{g} | | LS102.9 | non-natural (ROIV) | AHAAHAAHAAHAAHAA | 3570 |
| | IA^{u} | | 91.7 | non-natural (ROIV) | AHAAHAAHAAHAAHAA | 3570 |
| | IE^{d} | | A20 | Lambda repressor 12-26 | YLEDARRKKAIYEKKK | 3572 |
| | IE^{k} | | CH-12 | Lambda repressor 12-26 | YLEDARRKKAIYEKKK | 3572 |

[symbol]

indicates data missing or illegible when filed

TABLE XXV
Monoclonal antibodies used in MHC purifi

| Monoclonal antibody | Specificity |
|---|---|
| W6/32 | HLA-class I |
| B123.2 | HLA-B and C |
| IVD12 | HLA-DQ |
| LB3.1 | HLA-DR |
| M1/42 | H-2 class I |
| 28-14-8S | H-2 D^{b} and L^{d} |
| 34-5-8S | H-2 D^{d} |
| B8-24-3 | H-2 K^{b} |
| SF1-1.1.1 | H-2 K^{d} |
| Y-3 | H-2 K^{b} |
| 10.3.6 | H-2 IA^{k} |
| 14.4.4 | H-2 IE^{d}, IE^{K} |
| MKD6 | H-2 IA^{d} |
| Y3JP | H-2 IA^{b}, IA^{s}, IA^{u} |

TABLE XXVI
in vitro binding of conserved HBV-derived peptides to
HLA-A2-supertype alleles.

| | |
|---|---|
| A2-supertype binding capacity | |
| (IC50 nM) | Alleles |

| | | | | | | | | | | | | |
|---|---|---|---|---|---|---|---|---|---|---|---|---|
| Peptide | AA | Molecule | 1st Pos | Sequence | SEQ ID NO: | Consv.¹ | A*0201 | A*0202 | A*0203 | A*0206 | A*6802 | bound² |

| | | | | | | | | | | | | |
|---|---|---|---|---|---|---|---|---|---|---|---|---|
| 924.07 | 10 | Core | 18 | FLPSDFFPSV | 3492 | 95 | 2.5 | 2.1 | 6.0 | 3.0 | 36 | 5 |
| 1069.06 | 10 | ENV | 349 | LLVPFVQWFV | 3493 | 95 | 7.5 | 11 | 5.9 | 13 | 286 | 5 |
| 1147.13 | 10 | POL | 524 | FLLAQFTSAI | 3494 | 95 | 24 | 134 | 1.4 | 34 | 455 | 5 |
| 1013.0102 | 9 | ENV | 346 | WLSLLVPFV | 3498 | 100 | 4.6 | 113 | 1.4 | 10 | 1290 | 4 |
| 777.03 | 9 | ENV | 183 | FLLTRILTI | 3496 | 80 | 9.8 | 100 | 1.3 | 19 | —³ | 4 |
| 927.15 | 9 | POL | 653 | ALMPLYACI | 3497 | 95 | 10 | 126 | 3.0 | 160 | 851 | 4 |
| 1069.05 | 9 | POL | 525 | LLAQFTSAI | 3499 | 95 | 50 | 16 | 3.0 | 1538 | 51 | 4 |
| 1132.01 | 9 | ENV | 350 | LVPFVQWFV | 3500 | 95 | 119 | 287 | 2083 | 463 | 14 | 4 |
| 1147.14 | 11 | ENV | 259 | VLLDYQGMLPV | 3501 | 90 | 8.6 | 20 | 2.0 | 13 | 2353 | 4 |
| 1090.77 | 9 | POL | 538 (a) | YMDDVVLGV | 3495 | 90 | 5.1 | 90 | 6.7 | 71 | 1905 | 4 |
| 1069.071 | 9 | POL | 524 | FLLAQFTSA | 3505 | 95 | 6.0 | 1654 | 9.1 | 39 | 870 | 3 |
| 927.46 | 9 | POL | 500 | KLHLYSHPI | 3504 | 95 | 72 | 126 | 3.7 | 627 | 26667 | 3 |
| 927.42 | 9 | POL | 422 | NLSWLSLDV | 3503 | 90 | 77 | 843 | 16 | 2313 | 404 | 3 |
| 1168.02 | 9 | POL | 455 | GLSRYVARL | 3506 | 90 | 79 | 391 | 18 | 12333 | — | 3 |
| 927.41 | 9 | POL | 418 | LLSSNLSWL | 3502 | 90 | 455 | 55 | 2.6 | 1370 | 4000 | 3 |
| 1039.031 | 9 | ENV | 360 | MMWYWGPSL | 3509 | 85 | 5.6 | 5375 | 833 | 112 | 3636 | 2 |
| 927.11 | 9 | POL | 573 | FLLSLGIHL | 3507 | 95 | 7.7 | 4300 | 1000 | 34 | 11429 | 2 |
| 1142.07 | 9 | ENV | 73 | GLLGWSPQA | 3512 | 85 | 13 | 14333 | 286 | 1429 | — | 2 |
| 927.47 | 9 | POL | 502 | HLYSHPIIL | 3508 | 80 | 23 | 14333 | 11 | 2176 | 755 | 2 |
| 1137.02 | 10 | ENV | 271 | LLDYQGMLPV | 3511 | 90 | 51 | — | 500 | 552 | — | 2 |
| 1069.09 | 9 | ENV | 270 | VLLDYQGML | 3573 | 95 | 114 | — | 476 | 4111 | — | 2 |
| 1069.14 | 10 | NUC | 168 | ILSTLPETTV | 3574 | 100 | 238 | 506 | 130 | 1194 | 5970 | 2 |
| 1069.11 | 10 | POL | 147 | YLHTLWKAGI | 3575 | 100 | 313 | 8600 | 18 | 4000 | 1250 | 2 |
| 1142.01 | 9 | NUC | 129 | LLWFHISCL | 3576 | 90 | 385 | 21500 | 238 | 1194 | 4082 | 2 |
| 1090.12 | 9 | NUC | 147 | YLVSFGVWI | 3518 | 90 | 13 | | | | | 1 |
| 1.0518 | 10 | ENV | 359 | GLSPTVWLSV | 3519 | 75 | 18 | | | | | 1 |
| 1013.1402 | 9 | ENV | 177 | VLQAGFFLL | 3514 | 95 | 33 | 2389 | 3704 | 1947 | 6349 | 1 |
| 1069.13 | 9 | ENV | 388 | PLLPIFFCL | 3516 | 100 | 77 | — | 5556 | 3364 | 8511 | 1 |
| 1069.10 | 10 | ENV | 389 | LLPIFFCLWV | 3515 | 100 | 156 | 5375 | 667 | 5000 | — | 1 |
| 1090.06 | 10 | ENV | 175 | LLVLQAGFFL | 3517 | 90 | 161 | 1162 | 2222 | 2467 | 3636 | 1 |
| 1.0895 | 10 | ENV | 248 | FILLLCLIFL | 3577 | 80 | 179 | | | | | 1 |
| 927.24 | 9 | POL | 770 | WILRGTSFV | 3578 | 80 | 185 | | | | | 1 |
| 1090.14 | 9 | POL | 538 | YMDDVVLGA | 3520 | 90 | 200 | — | 4167 | — | — | 1 |
| 3.0205 | 10 | ENV | 171 | FLGPLLVLQA | 3579 | 75 | 263 | | | | | 1 |
| 1069.08 | 10 | ENV | 260 | ILLLCLIFLL | 3580 | 100 | 263 | — | — | 2846 | 26667 | 1 |
| 1.0573 | 10 | POL | 773 | ILRGTSFVYV | 3581 | 80 | 313 | | | | | 1 |

¹Frequency of entire sequence amongst isolates scanned.
²Number of supertpe alleles bound. Peptides binding 3 or more alleles are considered degenerate.
³A dash (—) indicates IC50

TABLE XXVII
in vitro binding of conserved HBV-derived peptides to
HLA-A3-supertype alleles.

| | |
|---|---|
| A3-supertype binding capacity | |
| (IC50 nM) | Alleles |

| | | | | | | | | | | | | |
|---|---|---|---|---|---|---|---|---|---|---|---|---|
| Peptide | AA | Molecule | 1st Pos | Sequence | SEQ ID NO: | Consv.¹ | A*03 | A*11 | A*3101 | A*3301 | A*6801 | bound |

| | | | | | | | | | | | | |
|---|---|---|---|---|---|---|---|---|---|---|---|---|
| 26.0535 | 11 | X NUC FUS | 299 | GVWIRTPPAYR | 3582 | 95 | 58 | 35 | 3.0 | 40 | 12 | 5 |
| 1147.16 | 11 | pol | 149 | HTLWKAGILYK | 3583 | 100 | 20 | 14 | 486 | 403 | 42 | 5 |
| 26.0539 | 11 | POL | 376 | RLVVDFSQFSR | 3584 | 95 | 39 | 2.0 | 7.0 | 24 | 1.0 | 5 |
| 26.0149 | 9 | X | 69 | CALRFTSAR | 3585 | 85 | 3235 | 261 | 12 | 3.6 | 11 | 4 |
| 1.0993 | 9 | X | 130 | KVFVLGGCR | 3586 | 75 | 262 | 73 | 30 | 408 | 2667 | 4 |
| 26.0153 | 9 | X | 64 | SSAGPCALR | 3587 | 90 | 1375 | 43 | 55 | 181 | 11 | 4 |
| 1083.01 | 11 | Core | 141 | STLPETTVVRR | 3588 | 95 | 733 | 4.0 | 180 | 181 | 26 | 4 |
| 20.0130 | 9 | pol | 655 | AFTFSPTYK | 3589 | 95 | 42 | 150 | 3103 | 13182 | 296 | 3 |
| 26.0008 | 8 | POL | 656 | FTFSPTYK | 3590 | 95 | 193 | 136 | 1286 | 1000 | 73 | 3 |
| 1.0219 | 9 | X | 1550 | FVLGGCRHK | 3591 | 80 | 169 | 316 | 1500 | 744 | 103 | 3 |
| 1069.20 | 10 | POL | 388 | LVVDFSQFSR | 3592 | 100 | 6875 | 17 | 692 | 126 | 16 | 3 |
| 1069.16 | 9 | POL | 47 | NVSIPWTHK | 3593 | 100 | 134 | 105 | —³ | 2900 | 250 | 3 |
| 1090.10 | 10 | POL | 665 | QAFTFSPTYK | 3594 | 95 | 244 | 11 | 18000 | 5088 | 6.7 | 3 |
| 1090.11 | 9 | POL | 531 | SAICSVVRR | 3595 | 95 | 1897 | 29 | 1200 | 446 | 21 | 3 |
| 20.0131 | 9 | pol | 524 | SVVRRAFPH | 3596 | 95 | 100 | 10 | 621 | — | 500 | 3 |
| 26.0545 | 11 | X NUC FUS | 318 | TLPETTVVRRR | 3597 | 95 | 22000 | 375 | 2951 | 408 | 13 | 3 |
| 26.0023 | 8 | X NUC FUS | 296 | VSFGVWIR | 3598 | 90 | 2750 | 207 | 240 | 1074 | 222 | 3 |
| 1142.05 | 9 | POL | 55 | KVGNFTGLY | 3599 | 95 | 52 | 353 | — | — | — | 2 |
| 1142.06 | 9 | POL | 623 | PVNRPIDWK | 3600 | 85 | 355 | 43 | — | — | 8889 | 2 |
| 1.0975 | 9 | POL | 106 | RLKLIMPAR | 3601 | 75 | 116 | — | 5.8 | 592 | — | 2 |
| 1.0562 | 10 | POL | 576 | SLGIHLNPNK | 3602 | 75 | 55 | 77 | | | | 2 |
| 1069.21 | 10 | NUC | 170 | STLPETTVVR | 3603 | 95 | 15714 | 100 | 2250 | 1208 | 320 | 2 |
| 1069.22 | 10 | NUC | 171 | TLPETTVVRR | 3604 | 95 | 15714 | 261 | — | 2417 | 182 | 2 |
| 1069.15 | 10 | POL | 150 | TLWKAGILYK | 3605 | 100 | 2.1 | 17 | 3529 | 29000 | 615 | 2 |
| 1.0215 | 9 | X | 105 | TTDLEAYFK | 3606 | 75 | 18333 | 6.5 | — | 24167 | 471 | 2 |
| 1069.17 | 10 | POL | 369 | VTGGVFLVDK | 3607 | 100 | 282 | 65 | — | — | 3636 | 2 |
| 1069.19 | 9 | POL | 389 | VVDFSQFSR | 3608 | 100 | 7333 | 80 | 13846 | 1706 | 242 | 2 |
| 26.0026 | 8 | POL | 168 | ASFCGSPY | 3609 | 100 | 239 | 26 | — | — | 20000 | 2 |
| 26.0549 | 11 | ENV | 389 | LLPIFFCLWVY | 3610 | 100 | 478 | 10000 | 2609 | 644 | 82 | 2 |
| 26.0550 | 11 | POL | 528 | RAFPHCLAFSY | 3611 | 95 | 92 | 15 | 667 | 26364 | 2667 | 2 |
| 1090.04 | 10 | POL | 746 | GTDNSVVLSR | 3612 | 90 | 11000 | 143 | 6000 | 15263 | 10000 | 1 |
| 1069.04 | 10 | POL | 149 | HTLWKAGILY | 3613 | 100 | 250 | 7500 | — | 8529 | 6667 | 1 |
| 1.0205 | 9 | POL | 771 | ILRGTSFVY | 3614 | 80 | 250 | — | — | — | — | 1 |
| 1090.08 | 9 | NUC | 148 | LVSFGVWIR | 3615 | 90 | 3929 | 500 | | | | 1 |
| 1039.01 | 10 | ENV | 360 | MMWYWGPSLY | 3616 | 85 | 220 | 7500 | — | — | 26667 | 1 |
| 1.0584 | 10 | X | 104 | STTDLEAYFK | 3617 | 75 | 1667 | 2.2 | | | | 1 |
| 1147.17 | 11 | pol | 735 | GTDNSVVLSRK | 3618 | 90 | 786 | 11 | — | — | — | 1 |
| 1147.18 | 11 | pol | 357 | RVTGGVFLVDK | 3619 | 100 | 578 | 207 | — | — | — | 1 |
| 1099.03 | 9 | POL | 150 | TLWKAGILY | 3620 | 100 | 85 | 7500 | — | — | — | 1 |
| 1090.15 | 10 | POL | 549 | YMDDVVLGAK | 3621 | 90 | 333 | 1395 | — | — | — | 1 |
| 26.0024 | 8 | POL | 50 | VSIPWTHK | 3622 | 100 | 846 | 353 | 5806 | 22308 | 20000 | 1 |

¹Frequency or entire sequence amongst isolates scanned.
²Number supertpe alleles bound. Peptides binding 3 or more alleles are considered degenerate.
³A dash (—) indicates IC50

TABLE XXVIII
in vitro binding of conserved hbV-derived peptides to
HLA-B7 supertype alleles.

| | | |
|---|---|---|
| | B7-supertype binding capacity | |
| SEQ ID | (IC50 nM) | Alleles |

| | | | | | | | | | | | | |
|---|---|---|---|---|---|---|---|---|---|---|---|---|
| Peptide | AA | Molecule | 1st Pos | Sequence | NO: | Consv.¹ | B*0702 | B*3501 | B*5101 | B*5301 | B*5401 | bound² |

| | | | | | | | | | | | | |
|---|---|---|---|---|---|---|---|---|---|---|---|---|
| 1147.05 | 10 | POL | 541 | FPHCLAFSYM | 3623 | 95 | 56 | 33 | 61 | 118 | 208 | 5 |
| 1145.04 | 9 | ENV | 324 | IPIPSSWAF | 3624 | 100 | 42 | 2.6 | 2.3 | 12 | 2941 | 4 |
| 1147.02 | 9 | POL | 440 | HPAAMPHLL | 3625 | 100 | 56 | 267 | 500 | 186 | 833 | 4 |
| 1147.06 | 9 | X | 58 | LPVCAFSSA | 3626 | 95 | 115 | 101 | 500 | 10333 | 0.53 | 4 |
| 1147.08 | 9 | POL | 651 | YPALMPLYA | 3627 | 95 | 306 | 150 | 162 | 664 | 0.63 | 4 |
| 988.05 | 9 | CORE | 19 | LPSDFFPSV | 3628 | 95 | 1774 | 343 | 9.0 | 120 | 4.8 | 4 |
| 1145.08 | 9 | POL | 541 | FPHCLAFSY | 3629 | 95 | —³ | 14 | 83 | 17 | 503 | 3 |
| 19.0014 | 8 | POL | 640 | YPALMPLY | 3630 | 190 | 13750 | 28 | 13 | 207 | 1786 | 3 |
| 26.0570 | 11 | pol | 640 | YPALMPLYACI | 3631 | 95 | 1375 | — | 117 | 291 | 143 | 3 |
| 1147.04 | 10 | POL | 365 | TPARVTGGVF | 3632 | 90 | 17 | 72 | — | 939 | 16667 | 2 |
| 15.0034 | 9 | ENV | 390 | LPIFFCLWV | 3633 | 100 | — | — | 57 | 2325 | 53 | 2 |
| 20.0140 | 9 | POL | 723 | LPIHTAELL | 3634 | 85 | 1375 | 114 | 1058 | 30 | 20000 | 2 |
| 19.0006 | 8 | ENV | 340 | VPFVQWFV | 3635 | 95 | 5500 | — | 0.29 | — | 91 | 2 |
| 19.0007 | 8 | ENV | 379 | LPIFFCLW | 3636 | 100 | — | — | 153 | 66 | 2857 | 2 |
| 19.0010 | 8 | POL | 1 | MPLSYQHF | 3637 | 100 | — | 742 | 458 | 251 | 526 | 2 |
| 19.0011 | 8 | POL | 429 | HPAAMPHL | 3638 | 100 | 85 | 18000 | 18 | 2514 | 625 | 2 |
| 19.0012 | 8 | POL | 511 | SPFLLAQF | 3639 | 95 | 10 | 8000 | 306 | 10333 | 1075 | 2 |
| 26.0566 | 11 | pol | 511 | SPFLLAQFTSA | 3640 | 95 | 67 | — | — | — | 0.83 | 2 |
| 1147.01 | 9 | POL | 789 | DPSRGRLGL | 3641 | 90 | 458 | — | — | — | — | 1 |
| 16.0182 | 10 | X | 67 | GPCALRFTSA | 3642 | 90 | 61 | — | — | — | 2857 | 1 |
| 20.0273 | 10 | POL | 440 | HPAAMPHLLV | 3643 | 85 | 344 | 3600 | 705 | 664 | 588 | 1 |
| 15.0030 | 9 | ENV | 191 | IPQSLDSWW | 3644 | 90 | — | — | 27500 | 62 | — | 1 |
| 15.0210 | 10 | POL | 123 | LPLDKGIKPY | 3645 | 100 | — | 248 | 27500 | — | — | 1 |
| 16.0006 | 9 | ENV | 25 | FPDHQLDPA | 3646 | 90 | — | 8000 | — | — | 12 | 1 |
| 16.0177 | 10 | ENV | 324 | IPIPSSWAFA | 3647 | 80 | 4231 | 3000 | | 6643 | 22 | 1 |
| 16.0180 | 10 | POL | 644 | APFTQCGYPA | 3648 | 95 | 1897 | — | | — | 7.1 | 1 |
| 16.0181 | 10 | POL | 723 | LPIHTAELLA | 3649 | 85 | 3056 | 6545 | | 5813 | 30 | 1 |
| 19.0003 | 8 | ENV | 173 | GPLLVLQA | 3650 | 95 | 18333 | — | 500 | — | 1538 | 1 |
| 19.0005 | 8 | ENV | 313 | IPIPSSWA | 3651 | 100 | 13750 | 18000 | 2895 | — | 167 | 1 |
| 19.0009 | 8 | NUC | 133 | RPPNAPIL | 3652 | 100 | 724 | — | 196 | — | — | 1 |
| 19.0015 | 8 | POL | 659 | SPTYKAFL | 3653 | 95 | 14 | — | 2895 | — | — | 1 |
| 19.0016 | 8 | POL | 769 | VPSALNPA | 3654 | 90 | 3000 | — | 786 | — | 10 | 1 |
| 26.0554 | 11 | pol | 633 | APFTQCGYPAL | 3655 | 95 | 24 | 7200 | 13750 | — | 1075 | — |
| 26.0559 | 11 | pol | 712 | LPIHTAELLAA | 3656 | 85 | 611 | 2667 | — | 775 | 3.6 | 1 |
| 26.0561 | 11 | pol | 774 | NPADDPSRGRL | 3657 | 90 | 458 | — | — | — | — | 1 |
| 26.0564 | 11 | Core | 133 | RPPNAPILSTL | 3658 | 100 | 42 | — | 3056 | — | — | 1 |
| 26.0567 | 11 | Core | 49 | SPHHTALRQAI | 3659 | 100 | 9.5 | — | 13750 | 18600 | — | 1 |
| 26.0568 | 11 | pol | 354 | TPARVTGGVFL | 3660 | 90 | 58 | — | — | 18600 | 20000 | 1 |

¹Frequency of entire sequence amongst isolates scanned.
²Number of supertpe alleles bound. Peptides binding 3 or more alleles are considered degenerate.
³A dash (—) indicates IC50

TABLE XXIX
HBV derived A1- and A24-motif containing peptides
a. A1-motif peptides

| | | | | | | HLA-A*0101 |
|---|---|---|---|---|---|---|
| Peptide | Molecule | Position | Sequence | SEQ ID NO: | Conserv. | binding (IC50 nM) |
| 1069.01 | Core | 59 | LLDTASALY | 3661 | 75 | 2.1 |
| 1.0519 | Core | 419 | DLLDTASALY | 3662 | 75 | 2.3 |
| 1069.02 | pol | 427 | SLDVSAAFY | 3663 | 95 | 4.8 |
| 2.0239 | | 1000 | LSLDVSAAFY | 3664 | 95 | 6.0 |
| 2.0126 | | 1521 | MSTTDLEAY | 3665 | 75 | 29 |
| 1039.06 | ENV | 359 | WMMWYWGPSLY | 3666 | 85 | 78 |
| 1090.14 | pol | 538 | YMDDVVLGA | 3667 | 90 | 96 |
| 1090.09 | pol | 808 | PTTGRTSLY | 3668 | 85 | 119 |
| 1069.03 | pol | 124 | PLDKGIKPYY | 3669 | 100 | 147 |
| 1069.08 | env | 249 | ILLLCLIFLL | 3670 | 100 | 192 |
| 1069.04 | pol | 149 | HTLWKAGILY | 3671 | 100 | 381 |
| 1039.01 | | 360 | MMWYWGPSLY | 3672 | 85 | 309 |
| 1.0774 | Core | 416 | WLWGMDIDPY | 3673 | 75 | 309 |
| 20.0254 | pol | 631 | FAAPFTQCGY | 3674 | 95 | 368 |
| 1.0166 | pol | 629 | KVGNFTGLY | 3675 | 95 | 368 |

b. A24-motif peptides

| | | | | | | HLA-A*2402 |
|---|---|---|---|---|---|---|
| Peptide | Molecule | Position | Sequence | SEQ ID NO: | Conserv. | binding (IC50 nM) |
| 20.0271 | POL | 392 | SWPKFAVPNL | 3676 | 95 | 2.1 |
| 1069.23 | POL | 745 | KYTSFPWLL | 3677 | 85 | 2.3 |
| 2.0181 | POL | 492 | LYSHPIILGF | 3678 | 80 | 11 |
| 20.0269 | ENV | 236 | RWMCLRRFII | 3679 | 95 | 11 |
| 20.0136 | ENV | 334 | SWLSLLVPF | 3680 | 100 | 31 |
| 20.0137 | ENV | 197 | SWWTSLNFL | 3681 | 95 | 32 |
| 20.0135 | ENV | 236 | RWMCLRRFI | 3682 | 95 | 169 |
| 20.0139 | POL | 167 | SFCGSPYSW | 3683 | 100 | 169 |
| 2.0173 | POL | 4 | SYQHFRKLLL | 3684 | 75 | 182 |
| 2.0060 | | 1224 | GYPALMPLY | 3685 | 95 | 245 |
| 13.0129 | NUC | 117 | EYLVSFGVWI | 3686 | 90 | 353 |
| 1090.02 | core | 131 | AYRPPNAPI | 3687 | 90 | 387 |
| 13.0073 | NUC | 102 | WFHISCLTF | 3688 | 80 | 400 |
| 20.0138 | POL | 51 | PWTHKVGNF | 3689 | 100 | 414 |

A dash indicates IC50 nM.

TABLE XXXa
Immunogenicity of HBV-derived A2-supermotif cross-reactive peptides

| | | | | | Immunogenicity | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 924.07 | FLPSDFFPSV | 3690 | HBV core 18 | 5 | 10/10 | 6/6 | 25/32^{a} | + |
| 1069.06 | LLVPFVQWFV | 3691 | HBV env 338 | 5 | 3/4 | 6/9 | | + |
| 1147.13 | FLLAQFTSAI | 3692 | HBV pol 513 | 5 | | 0/3 | | − |
| 1090.77 | YMDDVVLGV | 3693 | HBV pol 538 | 5 | | 9/9 | | + |
| 777.03 | FLLTRILTI | 3694 | HBV env 183 | 4 | | | 14/23^{a} | + |
| 927.15 | ALMPLYACI | 3695 | HBV pol 642 | 4 | 10/12 | 3/5 | 2/15^{a} | + |
| 1013.01 | WLSLLVPFV | 3696 | HBV env 335 | 4 | 2/6 | 5/9 | 23/29^{a} | + |
| 1069.05 | LLAQFTSAI | 3697 | HBV pol 504 | 4 | 0/4 | 0/5 | | − |
| 1132.01 | LVPFVQWFV | 3698 | HBV env 339 | 4 | 0/3 | 0/4 | | − |
| 1147.14 | VLLDYQGMLPV | 3699 | HBV env 259 | 4 | 4/4 | 6/6 | | + |
| 927.41 | LLSSNLSWL | 3700 | HBV pol 992 | 3 | 0/4 | 0/3 | | − |
| 927.42 | NLSWLSLDV | 3701 | HBV pol 411 | 3 | | 2/8 | | + |
| 927.46 | KLHLYSHPI | 3702 | HBV pol 489 | 3 | 0/4 | 4/6 | | + |
| 1069.07 | FLLAQFTSA | 3703 | HBV pol 503 | 3 | 1/2 | 0/3 | | + |
| 1168.02 | GLSRYVARL | 3704 | HBV pol 455 | 3 | | | 9/13^{a} | + |

Immunogenicity evaluation derived from primary cultures, acute patients (^{a}Bertoni et al, J Clin Invest 100: 503, ^{b}Rehermann et al., J. Clin. Invest 97: 1655, c- Nayersina et al., J Immunol 150: 4659) or transgenic mice. A positive assessment (+) is assigned when responders have been noted in one of these systems.

TABLE XXXb
Immunogenicity of non-crossreactive HBV A2-supermotif peptides

| | | | | | Immunogenicity | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 927.11 | FLLSLGIHL | 3705 | HBV pol 562 | 2 | 15/22 | 12/13 | 9/15^{a} | + |
| 927.47 | HLYSHPIIL | 3706 | HBV pol 1076 | 2 | | 10/14 | | + |
| 1039.03 | MMWYWGPSL | 3707 | HBV env 360 | 2 | 3/4 | 0/4 | | + |
| 1069.12 | YLHTLWKAGV | 3708 | HBV pol 147 | 2 | 2/4 | | | + |
| 1137.02 | LLDYQGMLPV | 3709 | HBV env 260 | 2 | 1/2 | 0/4 | | + |
| 1142.07 | GLLGWSPQA | 3710 | HBV env 62 | 2 | 3/4 | 5/6 | | + |
| 1.0573 | ILRGTSFVYV | 3711 | HBV pol 773 | 1 | | | 3/7^{b} | + |
| 1013.14 | VLQAGFFLL | 3712 | HBV env 177 | 1 | 0/4 | 5/12 | | + |
| 1069.10 | LLPIFFCLWV | 3713 | HBV env 378 | 1 | 3/3 | 0/4 | 2/5^{c} | + |
| 1069.13 | PLLPIFFCL | 3714 | HBV env 377 | 1 | 0/4 | 7/12 | | + |
| 1090.06 | LLVLQAGFFL | 3715 | HBV env 175 | 1 | 1/5 | 0/4 | | + |
| 1090.12 | YLVSFGVWI | 3716 | HBV nuc 118 | 1 | 9/9 | | | + |
| 1.0518 | GLSPTVWLSV | 3717 | HBV env 338 | 1 | | | 3/9^{c} | + |
| 1090.14 | YMDDVVLGA | 3718 | HBV pol 538 | 1 | 2/7 | 2/5 | 2/7^{b} | + |

Immunogenicity evaluation derived from primary cultures, acute patients (^{a}Bertoni et al, J Clin Invest 100: 503, ^{b}Rehermann et al., J. Clin. Invest 97: 1655, ^{c}Nayersina et al., J Immunol 150: 4659) or transgenic mice. A positive assessment (+) is assigned when responders have been noted in one of these systems.

TABLE XXXc
Cross-recognition of HBV pol 538 and a Lamivudine
induced pol 538 variant by CTL induced with a
pol 538 analog^{a}.

| | Day 6 CTL response (ΔLU) | |
|---|---|---|
| | HBV pol 538 | HBV pol 538 mutant |
| Stimulating peptide | (YMDDVVLGA)^{b} | (YVDDVVLGA) |
| HBV pol 538 | 27.8 | 54.2 |
| HBV pol 538 mutant | 35.3 | 27.9 |

^{a}CTLs were induced using the 1090.77 analog of HBV pol 538 (peptide 1090.14). 1090.77 was encoded in the DNA minigene pEP2.AOS.
^{b}Values shown represent the geometric mean of ΔLU from 2 independent cultures. Peptides loaded onto target cells were 1090.14 (HBV pol 538) or 1353.02 (a Lamivudine induced mutant of pol 538).

TABLE XXXIa
Immunogenicity of HBV-derived A3-supermotif cross-reactive peptides

| | | | | | Immunogenicity | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 1147.16 | HTLWKAGILYK | 3719 | HBV POL 149 | 5 | 0/6 | 3/3 | 1/22 | + |
| 1083.01 | STLPETTVVRR | 3720 | HBV core 141 | 4 | 3/5 | 6/6 | 8/32 | + |
| 1150.51 | GSTHVSWPK | 3721 | HBV pol 398 | 4 | | 3/6 | | + |
| 1.0219 | FVLGGCRHK | 3722 | HBV adr “X” 1550 | 3 | 0/4 | | | − |
| 1069.16 | NVSIPWTHK | 3723 | HBV pol 47 | 3 | 0/8 | 0/3 | 1/21 | + |
| 1069.20 | LVVDFSQFSR | 3724 | HBV pol 388 | 3 | 0/4 | 6/6 | 1/22 | + |
| 1090.10 | QAFTFSPTYK | 3725 | HBV pol 665 | 3 | 3/6 | 0/3 | 3/21 | + |
| 1090.11 | SAICSVVRR | 3726 | HBV pol 531 | 3 | 1/4 | | 2/22 | + |

¹Immunogenicity evaluation derived from primary cultures, Bertoni et al, J Clin Invest 100: 503 or transgenic mice. A positive assessment (+) is assigned when responders have been noted in one of these systems. A negative assessment (−) indicates that no responders when examined.

TABLE XXXIb
Immunogenicity of non-crossreactive HBV A3-supermotif peptides

| | | | | | Immunogenicity | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 1069.15 | TLWKAGILYK | 3727 | HBV pol 150 | 2 | 3/8 | 0/3 | 5/28 | + |
| 1142.05 | KVGNFTGLY | 3728 | HBV adr POL 629 | 2 | | 0/3 | 2/22 | + |

¹Immunogenicity evaluation derived from primary cultures, Bertoni et al, J Clin Invest 100: 503 or transgenic mice. A positive assessment (+) is assigned when responders have been noted in one of these systems. A negative assessment (−) indicates that no responders when examined.

TABLE XXXIIa
Immunogenicity of HBV B7-supermotif cross-reactive peptides

| | | | | | Immunogenicity | | | |
|---|---|---|---|---|---|---|---|---|
| Peptide | Sequence | SEQ ID NO: | Protein | XRN | primary | transgenic | patients | overall¹ |

| | | | | | | | |
|---|---|---|---|---|---|---|---|
| 1147.05 | FPHCLAFSYM | 3729 | HBV POL 530 | 5 | 1/3 | 0/12 | + |
| 988.05 | LPSDFFPSV | 3730 | HBV core 19-27 | 4 | | 2/16 | + |
| 1145.04 | IPIPSSWAF | 3731 | HBV ENV 313 | 4 | 0/4 | 1/12 | + |
| 1147.02 | HPAAMPHLL | 3732 | HBV POL 429 | 4 | 0/5 | 0/12 | − |
| 1147.06 | LPVCAFSSA | 3733 | HBV X 58 | 4 | 1/4 | | + |
| 1147.08 | YPALMPLYA | 3734 | HBV POL 640 | 4 | | 0/12 | − |
| 1145.08 | FPHCLAFSY | 3735 | HBV POL 541 | 3 | 0/4 | | − |

¹Immunogenicity evaluation derived from primary cultures, Bertoni et at, J Clin Invest 100: 503 or transgenic mice. A positive assessment (+) is assigned when responders have been noted in one of these systems. A negative assessment (−) indicates that no responders when examined.

TABLE XXXIIb
Immunogenicity of non-crossreactive HBV B7-supermotif peptides

| | | | | | Immunogenicity | | | |
|---|---|---|---|---|---|---|---|---|
| Peptide | Sequence | SEQ ID NO: | Protein | XRN | primary | transgenic | patients | overall¹ |
| 1147.04 | TPARVTGGVF | 3736 | HBV POL 354 | 2 | | | 2/12 | + |

¹Immunogenicity evaluation derived from primary cultures, Bertoni et al, J Clin Invest 100: 503 or transgenic mice. A positive assessment (+) is assigned when responders have been noted in one of these systems. A negative assessment (−) indicates that no responders when examined.

TABLE XXXIII
Candidate HBV-derived HTL epitopes

| Selection | | | | Conservancy | | | SEQ |
|---|---|---|---|---|---|---|---|
| criteria | Peptide | Mol | 1st Pos | Core | Total | Sequence | ID NO: |

| | | | | | | | |
|---|---|---|---|---|---|---|---|
| DR-supermotif | F107.01 | ENV | 249 | 100 | 95 | ILLLCLIFLLVLLDY | 3737 |
| | F107.02 | ENV | 252 | 95 | 95 | LCLIFLLVLLDYQGM | 3738 |
| | 1280.17 | ENV | 258 | 90 | 90 | LVLLDYQGMLPVCPL | 3739 |
| | 1186.22 | ENV | 332 | 100 | 100 | RFSWLSLLVPFVQWF | 3740 |
| | 1186.15 | ENV | 339 | 95 | 95 | LVPFVQWFVGLSPTV | 3741 |
| | 1186.06 | ENV | 342 | 95 | 95 | FVQWFVGLSPTVWLS | 3742 |
| | 1186.03 | NUC | 19 | 85 | 85 | ASKLCLGWLWGMDID | 3743 |
| | 1186.12 | NUC | 24 | 85 | 85 | LGWLWGMDIDPYKEF | 3744 |
| | 857.02 | NUC | 50 | | 90 | PHHTALRQAILCWGELMTLA | 3745 |
| | 1186.23 | NUC | 98 | 85 | 85 | RQLLWFHISCLTFGR | 3746 |
| | 27.0279 | NUC | 117 | | 90 | EYLVSFGVWIRTPPA | 3747 |
| | 27.0280 | NUC | 123 | 95 | 95 | GVWIRTPPAYRPPNA | 3748 |
| | 1186.20 | NUC | 129 | 100 | 95 | PPAYRPPNAPILSTL | 3749 |
| | 1186.16 | NUC | 136 | 100 | 95 | NAPILSTLPETTVVR | 3750 |
| | 1186.01 | POL | 38 | 95 | 95 | AEDLNLGNLNVSIPW | 3751 |
| | 1186.17 | POL | 45 | 100 | 95 | NLNVSIPWTHKVGNF | 3752 |
| | 27.0281 | POL | 145 | 100 | 100 | RHYLHTLWKAGILYK | 3753 |
| | 1280.13 | POL | 406 | 95 | 95 | KFAVPNLQSLTNLLS | 3754 |
| | 27.0283 | POL | 409 | | 85 | VPNLQSLTNLLSSNL | 3755 |
| | F107.03 | POL | 412 | 90 | 90 | LQSLTNLLSSNLSWL | 3756 |
| | 1186.28 | POL | 416 | 90 | 90 | TNLLSSNLSWLSLDV | 3757 |
| | 1186.27 | POL | 420 | 100 | 85 | SSNLSWLSLDVSAAF | 3758 |
| | F107.04 | POL | 523 | 95 | 95 | PFLLAQFTSAICSVV | 3759 |
| | 1186.10 | POL | 526 | 95 | 95 | LAQFTSAICSVVRRA | 3760 |
| | 1186.04 | POL | 534 | 95 | 95 | CSVVRRAFPHCLAFS | 3761 |
| | F107.05 | POL | 538 | 95 | 95 | RRAFPHCLAFSYMDD | 3762 |
| | 1186.02 | POL | 546 | 90 | 90 | AFSYMDDVVLGAKSV | 3763 |
| | 1186.05 | POL | 629 | 85 | 85 | DWKVCQRIVGLLGFA | 3764 |
| | 1280.21 | POL | 637 | 95 | 95 | VGLLGFAAPFTQCGY | 3765 |
| | 27.0278 | POL | 643 | | 95 | AAPFTQCGYPALMPL | 3766 |
| | 1186.21 | POL | 648 | 95 | 95 | QCGYPALMPLYACIQ | 3767 |
| | 1280.14 | POL | 694 | 95 | 95 | LCQVFADATPTGWGL | 3768 |
| | 27.0282 | POL | 750 | 85 | 85 | SVVLSRKYTSFPWLL | 3769 |
| | | X | 13 | 95 | 90 | RDVLCLRPVGAESRG | 3770 |
| | 1186.07 | X | 50 | 95 | 90 | GAHLSLRGLPVCAFS | 3771 |
| | 1186.29 | X | 60 | 95 | 90 | VCAFSSAGPCALRFT | 3772 |
| Algorithm | 1280.20 | ENV | 330 | 100 | 80 | SVRFSWLSLLVPFVQ | 3773 |
| | 1280.19 | NUC | 28 | 85 | 80 | RDLLDTASALYREAL | 3774 |
| | 1298.02 | POL | 56 | 90 | 55 | VGNFTGLYSSTVPVF | 3775 |
| | 1298.03 | POL | 571 | 95 | 75 | TNFLLSLGIHLNPNK | 3776 |
| | 1298.05 | POL | 651 | 95 | 55 | YPALMPLYACIQSKQ | 3777 |
| | 1298.06 | POL | 664 | 95 | 60 | KQAFTFSPTYKAFLC | 3778 |
| | 1280.181 | POL | 722 | 85 | 80 | PLPIHTAELLAACFA | 3779 |
| | 1280.09 | POL | 774 | 90 | 80 | GTSFVYVPSALNPAD | 3780 |
| DR3-motif | 795.05 | ENV | 10 | | 95 | PLGFFPDHQLDP | 3781 |
| | 35.0090 | ENV | 312 | 95 | 90 | FLLVLLDYQGMLPVC | 3782 |
| | CF-03 | NUC | 28 | 85 | 80 | RDLLDTASALYREALESPEH | 3783 |
| | 35.0091 | POL | 18 | 90 | 65 | AGPLEEELPRLADEG | 3784 |
| | 35.0092 | POL | 34 | 100 | 85 | NRRVAEDLNLGNLNV | 3785 |
| | 35.0093 | POL | 96 | 85 | 60 | VGPLTVNEKRRLKLI | 3786 |
| | 35.0094 | POL | 120 | 100 | 100 | TKYLPLDKGIKPYYP | 3787 |
| | 35.0095 | POL | 371 | 100 | 55 | GGVFLVDKNPHNTTE | 3788 |
| | 35.0096 | POL | 385 | 100 | 45 | ESRLVVDFSQFSRGN | 3789 |
| | 1186.18 | POL | 422 | 95 | 85 | NLSWLSLDVSAAFYH | 3790 |
| | 35.0099 | POL | 666 | 95 | 55 | AFTFSPTYKAFLCKQ | 3791 |
| | 35.0101 | X | 18 | 95 | 35 | LRPVGAESRGRPVSG | 3792 |
| Lower conservancy or miscellaneous | 799.01 | ENV | 11 | 80 | 75 | PLLVLQAGFFLLTRILTIPQ | 3793 |
| | 799.02 | ENV | 31 | 95 | | SLDSWWTSLNFLGGTTVCLG | 3794 |
| | 799.04 | ENV | 71 | 95 | 75 | GYRWMCLRRFIIFLFILLLC | 3795 |
| | 1298.01 | ENV | 117 | 80 | 40 | PQAMQWNSTTFHQTL | 3796 |
| | 1280.06 | ENV | 180 | 80 | 80 | AGFFLLTRILTIPQS | 3797 |
| | 1280.11 | ENV | 245 | 80 | 80 | IFLFILLLCLIFLLV | 3798 |
| | CF-08 | NUC | 120 | | 90 | VSFGVWIRTPPAYRPPNAPI | 3799 |
| | 1186.25 | NUC | 121 | 95 | 90 | SFGVWIRTPPAYRPP | 3800 |
| | 1280.15 | POL | 501 | 80 | 80 | LHLYSHPIILGFRKI | 3801 |
| | 1298.04 | POL | 618 | 80 | 45 | KQCFRKLPVNRPIDW | 3802 |
| | 1298.07 | POL | 767 | 80 | 70 | AANWILRGTSFVYVP | 3803 |
| | 1298.08 | POL | 827 | 80 | 60 | PDRVHFASPLHVAWR | 3804 |

TABLE XXXIV
HLA-DR screening panels

| Screening | | | Representative Assay | | Phenotypic Frequencies | | | | | |
|---|---|---|---|---|---|---|---|---|---|---|
| Panel | Antigen | Alleles | Allele | Alias | Cauc. | Blk. | Jpn. | Chn. | Hisp. | Avg. |
| Primary | DR1 | DRB1*0101-03 | DRB1*0101 | (DR1) | 18.5 | 8.4 | 10.7 | 4.5 | 10.1 | 10.4 |
| | DR4 | DRB1*0401-12 | DRB1*0401 | (DR4w4) | 23.6 | 6.1 | 40.4 | 21.9 | 29.8 | 24.4 |
| | DR7 | DRB1*0701-02 | DRB1*0701 | (DR7) | 26.2 | 11.1 | 1.0 | 15.0 | 16.6 | 14.0 |
| | Panel total | | | | 59.6 | 24.5 | 49.3 | 38.7 | 51.1 | 44.6 |
| Secondary | DR2 | DRB1*1501-03 | DRB1*1501 | (DR2w2 β1) | 19.9 | 14.8 | 30.9 | 22.0 | 15.0 | 20.5 |
| | DR2 | DRB5*0101 | DRB5*0101 | (DR2w2 β2) | — | — | — | — | — | — |
| | DR9 | DRB1*09011, 09012 | DRB1*0901 | (DR9) | 3.6 | 4.7 | 24.5 | 19.9 | 6.7 | 11.9 |
| | DR13 | DRB1*1301-06 | DRB1*1302 | (DR6w19) | 21.7 | 16.5 | 14.6 | 12.2 | 10.5 | 15.1 |
| | Panel total | | | | 42.0 | 33.9 | 61.0 | 48.9 | 30.5 | 43.2 |
| Tertiary | DR4 | DRB1*0405 | DRB1*0405 | (DR4w15) | — | — | — | — | — | — |
| | DR8 | DRB1*0801-5 | DRB1*0802 | (DR8w2) | 5.5 | 10.9 | 25.0 | 10.7 | 23.3 | 15.1 |
| | DR11 | DRB1*1101-05 | DRB1*1101 | (DR5w11) | 17.0 | 18.0 | 4.9 | 19.4 | 18.1 | 15.5 |
| | Panel total | | | | 22.0 | 27.8 | 29.2 | 29.0 | 39.0 | 29.4 |
| Quarternary | DR3 | DRB1*0301-2 | DRB1*0301 | (DR3w17) | 17.7 | 19.5 | 0.4 | 7.3 | 14.4 | 11.9 |
| | DR12 | DRB1*1201-02 | DRB1*1201 | (DR5w12) | 2.8 | 5.5 | 13.1 | 17.6 | 5.7 | 8.9 |
| | Panel total | | | | 20.2 | 24.4 | 13.5 | 24.2 | 19.7 | 20.4 |

TABLE XXXV
HBV-derived cross-reactive HLA-DR binding peptides

| | |
|---|---|
| | HLA-DR binding |
| Conservancy | capacity (IC50 nM) |

| Peptide | Mol | 1st Pos | Core | Total | Sequence | SEQ ID NO: | DR1 | DR2w2 β1 | DR2w2 β2 |
|---|---|---|---|---|---|---|---|---|---|
| F107.03 | POL | 412 | 90 | 90 | LQSLTNLLSSNLSWL | 3805 | 2.0 | 21 | 1000 |
| 1298.06 | POL | 664 | 95 | 60 | KQAFTFSPTYKAFLC | 3806 | 9.4 | 38 | 143 |
| 1280.06 | ENV | 180 | 80 | 80 | AGFFLLTRILTIPQS | 3807 | 1.1 | 217 | 1053 |
| 1280.09 | POL | 774 | 90 | 80 | GTSFVYVPSALNPAD | 3808 | 14 | 650 | 400 |
| 1186.25 | NUC | 121 | 95 | 90 | SFGVWIRTPPAYRPP | 3809 | 532 | 827 | 47 |
| 27.0280 | NUC | 123 | 95 | 95 | GVWIRTPPAYRPPNA | 3810 | 14 | 217 | 2.8 |
| CF-08 | NUC | 120 | | 90 | VSFGVWIRTPPAYRPPNAPI | 3811 | 192 | | 105 |
| 27.0281 | POL | 145 | 100 | 100 | RHYLHTLWKAGILYK | 3812 | 17 | 5.4 | 35 |
| 1186.15 | ENV | 339 | 95 | 95 | LVPFVQWFVGLSPTV | 3813 | 385 | 13 | 1429 |
| 1280.15 | POL | 501 | 80 | 80 | LHLYSHPIILGFRKI | 3814 | 227 | 268 | 500 |
| F107.04 | POL | 523 | 95 | 95 | PFLLAQFTSAICSVV | 3815 | 28 | 337 | 4762 |
| 1298.04 | POL | 618 | 80 | 45 | KQCFRKLPVNRPIDW | 3816 | 3.3 | 4136 | 952 |
| 1298.07 | POL | 767 | 80 | 70 | AANWILRGTSFVYVP | 3817 | 54 | 379 | 3279 |
| 857.02 | NUC | 50 | | 90 | PHHTALRQAILCWGELMTLA | 3818 | 70 | 9.1 | 211 |

| | HLA-DR binding capacity (IC50 nM) | | | | | | | | Total DR |
|---|---|---|---|---|---|---|---|---|---|
| Peptide | DR3 | DR4w4 | DR4w15 | DR5w11 | DR6 | DR7 | DR8 | DR9 | alleles bound |
| F107.03 | —^{a} | 9.4 | 47 | 294 | 135 | 167 | 557 | 682 | 10 |
| 1298.06 | — | 41 | 173 | 83 | 175 | 76 | 408 | 139 | 10 |
| 1280.06 | — | 8.5 | 253 | 5.6 | 9.5 | 8.1 | 188 | 58 | 9 |
| 1280.09 | — | 118 | 93 | 426 | — | 93 | 803 | 221 | 9 |
| 1186.25 | — | 577 | 603 | 769 | 17500 | 1042 | 196 | 938 | 8 |
| 27.0280 | — | 13 | 67 | 42 | — | 114 | 92 | 1667 | 8 |
| CF-08 | | 300 | | 426 | | 124 | | | 5 |
| 27.0281 | — | 2250 | 1462 | 42 | 745 | 61 | 27 | 174 | 8 |
| 1186.15 | — | 300 | 27 | 53 | 1944 | 2717 | 74 | 30 | 7 |
| 1280.15 | — | 66 | 238 | 488 | 17500 | — | 803 | 1531 | 7 |
| F107.04 | — | 563 | 317 | 1667 | 44 | 325 | 845 | 1271 | 7 |
| 1298.04 | — | 38 | 45 | 1538 | 814 | 63 | 845 | 3000 | 7 |
| 1298.07 | — | 882 | 1520 | 1429 | 140 | 43 | 196 | 278 | 7 |
| 857.02 | — | 85 | | 263 | 193000 | 676 | 196 | 2273 | 7 |

^{a}A dash (—) indicates IC50 nM > 20,000.

TABLE XXXVI
HBV-derived DR3-binding peptides

| | | | Conservancy | | | | |
|---|---|---|---|---|---|---|---|
| Peptide | Mol | 1st Pos | Core | Total | Sequence | SEQ ID NO: | DR3 |

| | | | | | | | |
|---|---|---|---|---|---|---|---|
| 1280.14* | POL | 694 | 95 | 95 | LCQVFADATPTGWGL | 3819 | 67 |
| 35.0096 | POL | 385 | 100 | 45 | ESRLVVDFSQFSRGN | 3820 | 115 |
| 35.0093 | POL | 96 | 85 | 60 | VGPLTVNEKRRLKLI | 3821 | 136 |
| 1186.27 | POL | 420 | 100 | 85 | SSNLSWLSLDVSAAF | 3822 | 200 |
| 1186.18 | POL | 422 | 95 | 85 | NLSWLSLDVSAAFYH | 3823 | 231 |

*tested as peptide 35.0100

TABLE XXXVIIa
HBV Preferred CTL Epitopes

| Peptide | Sequence | SEQ ID NO: | Protein | HLA |
|---|---|---|---|---|
| 924.07 | FLPSDFFPSV | 3824 | core 18 | A2 |
| 777.03 | FLLTRILTI | 3825 | env 183 | A2 |
| 927.15 | ALMPLYACI | 3826 | pol 642 | A2 |
| 1013.01 | WLSLLVPFV | 3827 | env 335 | A2 |
| 1090.77 | YMDDVVLGV | 3828 | pol 538 | A2/A1 |
| 1168.02 | GLSRYVARL | 3829 | pol 455 | A2 |
| 927.11 | FLLSLGIHL | 3830 | pol 562 | A2 |
| 1069.10 | LLPIFFCLWV | 3831 | env 378 | A2 |
| 1069.06 | LLVPFVQWFV | 3832 | env 338 | A2 |
| 1147.16 | HTLWKAGILYK | 3833 | pol 149 | A3/A1 |
| 1083.01 | STLPETTVVRR | 3834 | core 141 | A3 |
| 1069.16 | NVSIPWTHK | 3835 | pol 47 | A3 |
| 1069.20 | LVVDFSQFSR | 3836 | pol 388 | A3 |
| 1090.10 | QAFTFSPTYK | 3837 | pol 665 | A3 |
| 1090.11 | SAICSVVRR | 3838 | pol 531 | A3 |
| 1142.05 | KVGNFTGLY | 3839 | pol 629 | A3/A1 |
| 1147.05 | FPHCLAFSYM | 3840 | pol 530 | B7 |
| 988.05 | LPSDFFPSV | 3841 | core 19 | B7 |
| 1145.04 | IPIPSSWAF | 3842 | env 313 | B7 |
| 1147.02 | HPAAMPHLL | 3843 | pol 429 | B7 |
| 26.0570 | YPALMPLYACI | 3844 | pol 640 | B7 |
| 1147.04 | TPARVTGGVF | 3845 | pol 354 | B7 |
| 1.0519 | DLLDTASALY | 3846 | core 419 | A1 |
| 2.0239 | LSLDVSAAFY | 3847 | pol 1000 | A1 |
| 1039.06 | WMMWYWGPSLY | 3848 | env 359 | A1 |
| 20.0269 | RWMCLRRFII | 3849 | env 236 | A24 |
| 20.0136 | SWLSLLVPF | 3850 | env 334 | A24 |
| 20.0137 | SWWTSLNFL | 3851 | env 197 | A24 |
| 13.0129 | EYLVSFGVWI | 3852 | core 117 | A24 |
| 1090.02 | AYRPPNAPI | 3853 | core 131 | A24 |
| 13.0073 | WFHISCLTF | 3854 | core 102 | A24 |
| 20.0271 | SWPKFAVPNL | 3855 | pol 392 | A24 |
| 1069.23 | KYTSFPWLL | 3856 | pol 745 | A24 |
| 2.0181 | LYSHPIILGF | 3857 | pol 492 | A24 |

TABLE XXXVIIb
HBV Preferred HTL epitopes

| Selection | | | | Conservancy | | | |
|---|---|---|---|---|---|---|---|
| Criteria | Peptide | Mol | 1st Pos | Core | Total | SEQ ID NO: | Sequence |

| | | | | | | | |
|---|---|---|---|---|---|---|---|
| DR supermotif | F107.03 | POL | 412 | 90 | 90 | 3838 | LQSLTNLLSSNLSWL |
| | 1298.06 | POL | 664 | 95 | 60 | 3859 | KQAFTFSPTYKAFLC |
| | 1280.06 | ENV | 180 | 80 | 80 | 3860 | AGFFLLTRILTIPQS |
| | 1280.09 | POL | 774 | 90 | 80 | 3861 | GTSFVYVPSALNPAD |
| | CF-08 | CORE | 120 | | 90 | 3862 | VSFGVWIRTPPAYRPPNAPI |
| | 27.0281 | POL | 145 | 100 | 100 | 3863 | RHYLHTLWKAGILYK |
| | 1186.15 | ENV | 339 | 95 | 95 | 3864 | LVPFVQWFVGLSPTV |
| | 1280.15 | POL | 501 | 80 | 80 | 3865 | LHLYSHPIILGFRKI |
| | F107.04 | POL | 523 | 95 | 95 | 3866 | PFLLAQFTSAICSVV |
| | 1298.04 | POL | 618 | 80 | 45 | 3867 | KQCFRKLPVNRPIDW |
| | 1298.07 | POL | 767 | 80 | 70 | 3868 | AANWILRGTSFVYVP |
| | 857.02 | CORE | 50 | | 90 | 3869 | PHHTALRQAILCWGELMTLA |
| DR3 motif | 1280.14 | POL | 694 | 95 | 95 | 3870 | LCQVFADATPTGWGL |
| | 35.0096 | POL | 385 | 100 | 45 | 3871 | ESRLVVDFSQFSRGN |
| | 35.0093 | POL | 96 | 85 | 60 | 3872 | VGPLTVNEKRRLKLI |
| | 1186.27 | POL | 420 | 100 | 85 | 3873 | SSNLSWLSLDVSAAF |

TABLE XXXVIII
Estimated population coverage by a panel of HBV derived HTL epitopes

| | | | | Population coverage | | | | | |
|---|---|---|---|---|---|---|---|---|---|
| | | Representative | No. of | (phenotypic frequency) | | | | | |
| Antigen | Alleles | assay | epitopes² | Cauc. | Blk. | Jpn. | Chn. | Hisp. | Avg. |
| DR1 | DRB1*0101-03 | DR1 | 12 | 18.5 | 8.4 | 10.7 | 4.5 | 10.1 | 10.4 |
| DR2 | DRB1*1501-03 | DR2w2 β1 | 11 | 19.9 | 14.8 | 30.9 | 22.0 | 15.0 | 20.5 |
| DR2 | DRB5*0101 | DR2w2 β2 | 8 | — | — | — | — | — | — |
| DR3 | DRB1*0301-2 | DR3 | 4 | 17.7 | 19.5 | 0.40 | 7.3 | 14.4 | 11.9 |
| DR4 | DRB1*0401-12 | DR4w4 | 11 | 23.6 | 6.1 | 40.4 | 21.9 | 29.8 | 24.4 |
| DR4 | DRB1*0401-12 | DR4w15 | 9 | — | — | — | — | — | — |
| DR7 | DRB1*0701-02 | DR7 | 9 | 26.2 | 11.1 | 1.0 | 15.0 | 16.6 | 14.0 |
| DR8 | DRB1*0801-5 | DR8w2 | 7 | 5.5 | 10.9 | 25.0 | 10.7 | 23.3 | 15.1 |
| DR9 | DRB1*09011, 09012 | DR9 | 10 | 3.6 | 4.7 | 24.5 | 19.9 | 6.7 | 11.9 |
| DR11 | DRB1*1101-05 | DR5w11 | 11 | 17.0 | 18.0 | 4.9 | 19.4 | 18.1 | 15.5 |
| DR13 | DRB1*1301-06 | DR6w19 | 7 | 21.7 | 16.5 | 14.6 | 12.2 | 10.5 | 15.1 |
| Total¹ | | | | 98.5 | 95.1 | 97.1 | 91.3 | 94.3 | 95.1 |

¹Total opulation coverage has been adjusted to acount for the presence of DRX in many ethnic populations. It has been assumed that the range of specificities represented by DRX alleles will mirror those of previously characterized HLA-DR alleles. The proportion of DRX incorporated under each motif is representative of the frequency of the motif in the remainder of the population. Total coverage has not been adjusted to account for unknown gene types.
²Number of epitopes represents a minimal estimate, considering only the epitopes shown in Table 12. Additional alleles possibly bound by nested epitopes have not been accounted.

What is claimed is:

1. A peptide composition of less than 250 amino acid residues comprising a peptide epitope useful for inducing an immune response against hepatitis B virus (HBV) said epitope (a) having an amino acid sequence of about 8 to about 13 amino acid residues that have at least 65% identity with a native amino acid sequence of HBV and, (b) binding to at least one HLA class I HLA allele with an IC₅₀ of less than about 500 nM.

2. The composition of claim 1, further wherein said peptide has at least 77% identity with a native HBV amino acid sequence.

3. The composition of claim 1, further wherein said peptide has 100% identity with a native HBV amino acid sequence.

4. A pharmaceutical composition comprising a peptide and a pharmaceutical carrier, wherein the peptide is a peptide of Table VII (A1 supermotif), Table VIII (A2 supermotif/A2.1 motif), Table IX (A3 supermotif), Table X (A24 supermotif), Table XI (B7 supermotif), Table XII (B27 supermotif), Table XIII (B58 supermotif), Table XIV (B62 supermotif), Table XV (A1 motif), Table XVI (A3 motif), Table XVII (A11 motif), or Table XVIII (A24 motif) comprising an IC₅₀ of less than about 500 nM for at least one HLA class I molecule.

5. The pharmaceutical composition of claim 4 wherein the composition comprises the peptide in a form of nucleic acids that encode the peptide.

6. The pharmaceutical composition of claim 5 wherein the composition comprises the peptide in a form of nucleic acids that encode the epitope and one or more additional peptide(s).

7. The composition of claim 4, wherein the peptide is comprised by a longer peptide, with a proviso that the longer peptide is not an entire native antigen.

8. The pharmaceutical composition of claim 4 wherein the peptide is in a human dose form, and the carrier is in a human unit dose.

9. A peptide composition of claim 1 comprising an analog of a peptide epitope, wherein the peptide epitope is an epitope of Table VII (A1 supermotif), Table VIII (A2 supermotif/A2.1 motif), Table IX (A3 supermotif), Table X (A24 supermotif), Table XI (B7 supermotif), Table XII (B27 supermotif), Table XIII (B58 supermotif), Table XIV (B62 supermotif), Table XV (A1 motif), Table XVI (A3 motif), Table XVII (A11 motif), or Table XVIII (A24 motif), said analog comprising a preferred or less preferred amino acid of Table II substituted in for a starting residue, or having a deleterious residue of Table II substituted out of the starting sequence and replaced by a non-deleterious residue.

10. A peptide composition of claim 1 comprising a peptide of Table XXII.

11. A method for inducing a cytotoxic T lymphocyte response, said method comprising steps of:
providing a peptide that comprises an IC₅₀ of less than about 500 nM for an HLA class I molecule, wherein the peptide is a peptide of Table VII (A1 supermotif), Table VIII (A2 supermotif/A2.1 motif), Table IX (A3 supermotif), Table X (A24 supermotif), Table XI (B7 supermotif), Table XII (B27 supermotif), Table XIII (B58 supermotif), Table XIV (B62 supermotif), Table XV (A1 motif), Table XVI (A3 motif), Table XVII (A11 motif), or Table XVIII (A24 motif); and,
administering said peptide to a human.

12. The method of claim 11, wherein the providing step provides the peptide in a form of nucleic acids that encode the peptide.

13. The method of claim 12, wherein the providing step provides the peptide in a form of nucleic acids that encode the peptide and at least one additional peptide, with a proviso that an additional peptide is not an entire native antigen.

14. The method of claim 11, wherein the providing step provides the peptide comprised by a longer peptide, with a proviso that the longer peptide is not an entire native antigen.

15. A method for inducing a cytotoxic T lymphocyte response, said method comprising steps of:
providing a peptide that induces a cytotoxic T cell response in vitro and/or in vivo, wherein the peptide is a peptide of Table VII (A1 supermotif), Table VIII (A2 supermotif/A2.1 motif), Table IX (A3 supermotif), Table X (A24 supermotif), Table XI (B7 supermotif), Table XII (B27 supermotif, Table XIII (B58 supermotif), Table XIV (B62 supermotif), Table XV (A1 motif), Table XVI (A3 motif), Table XVII (A11 motif), Table XVIII (A24 motif or Table XXIII; and,
administering said pharmaceutical composition to a human.

16. The method of claim 15, wherein the providing step provides the peptide in a form of nucleic acids that encode the peptide.

17. The method of claim 16, wherein the providing step provides the peptide in a form of nucleic acids that encode the peptide and at least one additional peptide, with a proviso that an additional peptide is not an entire native antigen.

18. The method of claim 15, wherein the providing step provides the peptide comprised by a longer peptide, with a proviso that the longer peptide is not an entire native antigen.

19. The method of claim 15, wherein the providing step comprises a peptide that induces a cytotoxic T cell response when complexed with an HLA class I molecule and is presented to an HLA class I-restricted cytotoxic T cell.

20. A peptide composition of less than 250 amino acid residues comprising a peptide epitope useful for inducing an immune response against hepatitis B virus (HBV) said epitope (a) having an amino acid sequence of about 6 to about 25 amino acid residues that have at least 65% identity with a native amino acid sequence of HBV and, (b) binding to at least one HLA class II HLA allele with an IC₅₀ of less than about 1000 mM.

21. The composition of claim 20, further wherein said peptide has at least 77% identity with a native HBV amino acid sequence.

22. The composition of claim 20, further wherein said peptide has 100% identity with a native HBV amino acid sequence.

23. A pharmaceutical composition comprising:
a human dose form of a peptide of Table XIX or Table XX that comprises an IC₅₀ of less than about 1,000 nM for at least one HLA DR molecule of an HLA DR supertype; and,
a human dose of a pharmaceutically acceptable carrier.

24. The pharmaceutical composition of claim 23 wherein the composition comprises the peptide in a form of nucleic acids that encode the peptide.

25. The pharmaceutical composition of claim 24 wherein the composition comprises the peptide in a form of nucleic acids that encode the peptide and at least one additional peptide, with a proviso that an additional peptide is not an entire native antigen.

26. The composition of claim 25, wherein the peptide is comprised by a longer peptide, with a proviso that the longer peptide is not an entire native antigen.

27. A peptide composition of claim 20 comprising an analog of a peptide epitope of Table XIX or Table XX, said analog comprising a preferred or less preferred amino acid of Table III substituted in for a starting residue, and/or having a deleterious residue of Table III substituted out of the starting sequence and replaced by a non-deleterious residue.

28. A method for inducing a helper T lymphocyte response, said method comprising steps of:
providing a peptide that comprises an IC₅₀ of less than about 1,000 nM for an HLA class II molecule, wherein the peptide is a peptide of Table XIX or Table XX; and,
administering said peptide to a human.

29. The method of claim 28, wherein the providing step provides the peptide in a form of nucleic acids that encode the peptide.

30. The method of claim 29, wherein the providing step provides the peptide in a form of nucleic acids that encode the peptide and at least one additional peptide, with a proviso that an additional peptide is not an entire native antigen.

31. The method of claim 28, wherein the providing step provides the peptide comprised by a longer peptide, with a proviso that the longer peptide is not an entire native antigen.

32. A method for inducing a helper T lymphocyte response, said method comprising steps of:
providing a peptide that induces a helper T cell response in vitro and/or in vivo, wherein the peptide is a peptide of Table XIX or Table XX; and,
administering said pharmaceutical composition to a human.

33. The method of claim 32, wherein the providing step provides the peptide in a form of nucleic acids that encode the peptide.

34. The method of claim 33, wherein the providing step provides the peptide in a form of nucleic acids that encode the peptide and at least one additional peptide, with a proviso that an additional peptide is not an entire native antigen.

35. The method of claim 32, wherein the providing step provides the peptide comprised by a longer peptide, with a proviso that the longer peptide is not an entire native antigen.

36. The method of claim 32, wherein the providing step comprises a peptide that induces a helper T cell response when complexed with an HLA class II molecule and is presented to an HLA class I-restricted helper T cell.

37. A vaccine for preventing or treating HBV infection that induces a protective or therapeutic immune response, wherein said vaccine comprises:
at least one peptide selected from Table(s) VII-XX or Table XXII; and,
a pharmaceutically acceptable carrier.

38. A kit for a vaccine that induces a protective or therapeutic immune response to HBV, said vaccine comprising:
at least one peptide selected from Table(s) VII-XX or Table XXII;
a pharmaceutically acceptable carrier; and,
instructions for administration to a patient.

39. A method for monitoring or evaluating an immune response to HBV or an epitope thereof in a patient having a known HLA type, the method comprising:
incubating a T lymphocyte sample from the patient with a peptide selected from Table(s) VII-XX or Table XXII, wherein that peptide bears a motif corresponding to at least one HLA allele present in said patient; and,
detecting the presence of a T lymphocyte that recognizes the peptide.

40. The method of claim 39, wherein the peptide is comprised by a tetrameric complex.
